SEQUENCE LISTING (654 sequences)

SEQ ID NO: 1 - DNA, 9985 bp, Homo sapiens
  misc_feature (1)..(9985): UNC13A reference mRNA NM_001080421.3
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 2 - DNA, 116 bp, Homo sapiens
  misc_feature (1)..(116): Exon 20
acaagcgaactgacaaatctgccgtgtcgggtgccatccggctccacatcagtgtggagatcaaaggcgaggagaaggtggccccgtaccatgtccagtacacctgtctgcatgag

SEQ ID NO: 3 - DNA, 164 bp, Homo sapiens
  misc_feature (1)..(164): Exon 21
aacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgac

SEQ ID NO: 4 - DNA, 1288 bp, Homo sapiens
  misc_feature (1)..(1288): Intron 20-21
gtgagggtcattgctcggcccctcccatgccacttccactcaccattcctgcctgcccagctcttcctctttctggccacaccatccacactctcctggccctctgagactgcccgccatgccattccctttacctggaaaactcctccctatccatcaaagtccagattcagggtcacctcctctgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatgaattaatgaataaacaggcagatggatgatgtaagctgccccagaccctgggacctctgacccccggcgaccccttgcactctccatgacactttctctcccatggtggcag

SEQ ID NO: 5 - DNA, 128 bp, Homo sapiens
  misc_feature (1)..(128): Cryptic Exon 1
ctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 6 - DNA, 179 bp, Homo sapiens
  misc_feature (1)..(179): Cryptic Exon 2
ccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatggg

SEQ ID NO: 7 - DNA, 10113 bp, Homo sapiens
  misc_feature (1)..(10113): Cryptic Exon Splice Variant 1
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagctgcctgggtttcctggaaagaactcttatccccaggaactagtttgttgaataaatgctggtgaatgaatgaatgattgaacagatgaatgagtgatgagtagataaaaggatggatggagagatggaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 8 - protein, 840 aa, Homo sapiens
  MISC_FEATURE (1)..(840): Cryptic Exon Splice Variant 1
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHELPGFPGKNSYPQELVC

SEQ ID NO: 9 - DNA, 10164 bp, Homo sapiens
  misc_feature (1)..(10164): Cryptic Exon Splice Variant 2
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 10 - protein, 825 aa, Homo sapiens
  MISC_FEATURE (1)..(825): Cryptic Exon Splice Variant 2
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHEP

SEQ ID NO: 11 - protein, 1703 aa, Homo sapiens
  MISC_FEATURE (1)..(1703): UNC13A reference protein NP_001073890.2
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHENLFHFVTDVQNNGVVKIPDAKGDDAWKVYYDETAQEIVDEFAMRYGVESIYQAMTHFACLSSKYMCPGVPAVMSTLLANINAYYAHTTASTNVSASDRFAASNFGKERFVKLLDQLHNSLRIDLSMYRNNFPASSPERLQDLKSTVDLLTSITFFRMKVQELQSPPRASQVVKDCVKACLNSTYEYIFNNCHELYSREYQTDPAKKGEVLPEEQGPSIKNLDFWSKLITLIVSIIEEDKNSYTPCLNQFPQELNVGKISAEVMWNLFAQDMKYAMEEHDKHRLCKSADYMNLHFKVKWLYNEYVTELPAFKDRVPEYPAWFEPFVIQWLDENEEVSRDFLHGALERDKKDGFQQTSEHALFSCSVVDVFSQLNQSFEIIKKLECPDPQIVGHYMRRFAKTISNVLLQYADIISKDFASYCSKEKEKVPCILMNNTQQLRVQLEKMFEAMGGKELDAEASDILKELQVKLNNVLDELSRVFATSFQPHIEECVKQMGDILSQVKGTGNVPASACSSVAQDADNVLQPIMDLLDSNLTLFAKICEKTVLKRVLKELWKLVMNTMEKTIVLPPLTDQTMIGNLLRKHGKGLEKGRVKLPSHSDGTQMIFNAAKELGQLSKLKDHMVREEAKSLTPKQCAVVELALDTIKQYFHAGGVGLKKTFLEKSPDLQSLRYALSLYTQATDLLIKTFVQTQSAQGLGVEDPVGEVSVHVELFTHPGTGEHKVTVKVVAANDLKWQTSGIFRPFIEVNIIGPQLSDKKRKFATKSKNNSWAPKYNESFQFTLSADAGPECYELQVCVKDYCFAREDRTVGLAVLQLRELAQRGSAACWLPLGRRIHMDDTGLTVLRILSQRSNDEVAKEFVKLKSDTRSAEEGGAAPAP

SEQ ID NO: 12 - DNA, 101 bp, Homo sapiens
  misc_feature (1)..(101): Exon 20 splice donor, position chr19:17,642,794-17,642,894
gcgaggagaaggtggccccgtaccatgtccagtacacctgtctgcatgaggtgagggtcattgctcggcccctcccatgccacttccactcaccattcctg

SEQ ID NO: 13 - DNA, 101 bp, Homo sapiens
  misc_feature (1)..(101): Exon 20 splice donor, reverse complement
caggaatggtgagtggaagtggcatgggaggggccgagcaatgaccctcacctcatgcagacaggtgtactggacatggtacggggccaccttctcctcgc

SEQ ID NO: 14 - DNA, 25 bp, Artificial Sequence
  Antisense oligonucleotide MTx_ASO_0001
caggaatggtgagtggaagtggcat

SEQ ID NO: 15 - DNA, 25 bp, Artificial Sequence
  Antisense Oligonucleotide MTx_ASO_0002
aggaatggtgagtggaagtggcatg

SEQ ID NO: 16 - DNA, 25 bp, Artificial Sequence
  Antisense Oligonucleotide MTx_ASO_0003
ggaatggtgagtggaagtggcatgg

SEQ ID NO: 17 - DNA, 25 bp, Artificial Sequence
  Antisense Oligonucleotide MTx_ASO_0004
gaatggtgagtggaagtggcatggg

SEQ ID NO: 18 - DNA, 25 bp, Artificial Sequence
  Antisense Oligonucleotide MTx_ASO_0005
aatggtgagtggaagtggcatggga

SEQ ID NO: 19 - DNA, 25 bp, Artificial Sequence
  Antisense Oligonucleotide MTx_ASO_0006
atggtgagtggaagtggcatgggag

SEQ ID NO: 20 - DNA, 25 bp, Artificial Sequence
  Antisense Oligonucleotide MTx_ASO_0007
tggtgagtggaagtggcatgggagg

SEQ ID NO: 21 - DNA, 25 bp, Artificial Sequence
  Antisense Oligonucleotide MTx_ASO_0008
ggtgagtggaagtggcatgggaggg

SEQ ID NO: 22 - DNA, 25 bp, Artificial Sequence
  Antisense Oligonucleotide MTx_ASO_0009
gtgagtggaagtggcatgggagggg

SEQ ID NO: 23 - DNA, 25 bp, Artificial Sequence
  Antisense Oligonucleotide MTx_ASO_0010
tgagtggaagtggcatgggaggggc

SEQ ID NO: 24 - DNA, 25 bp, Artificial Sequence
  Antisense Oligonucleotide MTx_ASO_0011
gagtggaagtggcatgggaggggcc

SEQ ID NO: 25 - DNA, 25 bp, Artificial Sequence
  Antisense Oligonucleotide MTx_ASO_0012
agtggaagtggcatgggaggggccg

SEQ ID NO: 26 - DNA, 25 bp, Artificial Sequence
  Antisense Oligonucleotide MTx_ASO_0013
gtggaagtggcatgggaggggccga

SEQ ID NO: 27 - DNA, 25 bp, Artificial Sequence
  Antisense Oligonucleotide MTx_ASO_0014
tggaagtggcatgggaggggccgag

SEQ ID NO: 28 - DNA, 25 bp, Artificial Sequence
  Antisense Oligonucleotide MTx_ASO_0015
ggaagtggcatgggaggggccgagc

SEQ ID NO: 29 - DNA, 25 bp, Artificial Sequence
  Antisense Oligonucleotide MTx_ASO_0016
gaagtggcatgggaggggccgagca

SEQ ID NO: 30 - DNA, 25 bp, Artificial Sequence
  Antisense Oligonucleotide MTx_ASO_0017
aagtggcatgggaggggccgagcaa

SEQ ID NO: 31 - DNA, 25 bp, Artificial Sequence
  Antisense Oligonucleotide MTx_ASO_0018
agtggcatgggaggggccgagcaat

SEQ ID NO: 32 - DNA, 25 bp, Artificial Sequence
  Antisense Oligonucleotide MTx_ASO_0019
gtggcatgggaggggccgagcaatg

SEQ ID NO: 33 - DNA, 25 bp, Artificial Sequence
  Antisense Oligonucleotide MTx_ASO_0020
tggcatgggaggggccgagcaatga

SEQ ID NO: 34 - DNA, 25 bp, Artificial Sequence
  Antisense Oligonucleotide MTx_ASO_0021
ggcatgggaggggccgagcaatgac

SEQ ID NO: 35 - DNA, 25 bp, Artificial Sequence
  Antisense Oligonucleotide MTx_ASO_0022
gcatgggaggggccgagcaatgacc

SEQ ID NO: 36 - DNA, 25 bp, Artificial Sequence
  Antisense Oligonucleotide MTx_ASO_0023
catgggaggggccgagcaatgaccc

SEQ ID NO: 37 - DNA, 25 bp, Artificial Sequence
  Antisense Oligonucleotide MTx_ASO_0024
atgggaggggccgagcaatgaccct

SEQ ID NO: 38 - DNA, 25 bp, Artificial Sequence
  Antisense Oligonucleotide MTx_ASO_0025
tgggaggggccgagcaatgaccctc

SEQ ID NO: 39 - DNA, 25 bp, Artificial Sequence
  Antisense Oligonucleotide MTx_ASO_0026
gggaggggccgagcaatgaccctca

SEQ ID NO: 40 - DNA, 25 bp, Artificial Sequence
  Antisense Oligonucleotide MTx_ASO_0027
ggaggggccgagcaatgaccctcac

SEQ ID NO: 41 - DNA, 25 bp, Artificial Sequence
  Antisense Oligonucleotide MTx_ASO_0028
gaggggccgagcaatgaccctcacc

SEQ ID NO: 42 - DNA, 25 bp, Artificial Sequence
  Antisense Oligonucleotide MTx_ASO_0029
aggggccgagcaatgaccctcacct

SEQ ID NO: 43 - DNA, 25 bp, Artificial Sequence
  Antisense Oligonucleotide MTx_ASO_0030
ggggccgagcaatgaccctcacctc

SEQ ID NO: 44 - DNA, 25 bp, Artificial Sequence
  Antisense Oligonucleotide MTx_ASO_0031
gggccgagcaatgaccctcacctca

SEQ ID NO: 45 - DNA, 25 bp, Artificial Sequence
  Antisense Oligonucleotide MTx_ASO_0032
ggccgagcaatgaccctcacctcat

SEQ ID NO: 46 - DNA, 25 bp, Artificial Sequence
  Antisense Oligonucleotide MTx_ASO_0033
gccgagcaatgaccctcacctcatg

SEQ ID NO: 47 - DNA, 25 bp, Artificial Sequence
  Antisense Oligonucleotide MTx_ASO_0034
ccgagcaatgaccctcacctcatgc

SEQ ID NO: 48 - DNA, 25 bp, Artificial Sequence
  Antisense Oligonucleotide MTx_ASO_0035
cgagcaatgaccctcacctcatgca

SEQ ID NO: 49 - DNA, 25 bp, Artificial Sequence
  Antisense Oligonucleotide MTx_ASO_0036
gagcaatgaccctcacctcatgcag

SEQ ID NO: 50 - DNA, 25 bp, Artificial Sequence
  Antisense Oligonucleotide MTx_ASO_0037
agcaatgaccctcacctcatgcaga

SEQ ID NO: 51 - DNA, 25 bp, Artificial Sequence
  Antisense Oligonucleotide MTx_ASO_0038
gcaatgaccctcacctcatgcagac

SEQ ID NO: 52 - DNA, 25 bp, Artificial Sequence
  Antisense Oligonucleotide MTx_ASO_0039
caatgaccctcacctcatgcagaca

SEQ ID NO: 53 - DNA, 25 bp, Artificial Sequence
  Antisense Oligonucleotide MTx_ASO_0040
aatgaccctcacctcatgcagacag

SEQ ID NO: 54 - DNA, 25 bp, Artificial Sequence
  Antisense Oligonucleotide MTx_ASO_0041
atgaccctcacctcatgcagacagg

SEQ ID NO: 55 - DNA, 25 bp, Artificial Sequence
  Antisense Oligonucleotide MTx_ASO_0042
tgaccctcacctcatgcagacaggt

SEQ ID NO: 56 - DNA, 25 bp, Artificial Sequence
  Antisense Oligonucleotide MTx_ASO_0043
gaccctcacctcatgcagacaggtg

SEQ ID NO: 57 - DNA, 25 bp, Artificial Sequence
  Antisense Oligonucleotide MTx_ASO_0044
accctcacctcatgcagacaggtgt

SEQ ID NO: 58 - DNA, 25 bp, Artificial Sequence
  Antisense Oligonucleotide MTx_ASO_0045
ccctcacctcatgcagacaggtgta

SEQ ID NO: 59 - DNA, 25 bp, Artificial Sequence
  Antisense Oligonucleotide MTx_ASO_0046
cctcacctcatgcagacaggtgtac

SEQ ID NO: 60 - DNA, 25 bp, Artificial Sequence
  Antisense Oligonucleotide MTx_ASO_0047
ctcacctcatgcagacaggtgtact

SEQ ID NO: 61 - DNA, 25 bp, Artificial Sequence
  Antisense Oligonucleotide MTx_ASO_0048
tcacctcatgcagacaggtgtactg

SEQ ID NO: 62 - DNA, 25 bp, Artificial Sequence
  Antisense Oligonucleotide MTx_ASO_0049
cacctcatgcagacaggtgtactgg

SEQ ID NO: 63 - DNA, 25 bp, Artificial Sequence
  Antisense Oligonucleotide MTx_ASO_0050
acctcatgcagacaggtgtactgga

SEQ ID NO: 64 - DNA, 25 bp, Artificial Sequence
  Antisense Oligonucleotide MTx_ASO_0051
cctcatgcagacaggtgtactggac

SEQ ID NO: 65 - DNA, 25 bp, Artificial Sequence
  Antisense Oligonucleotide MTx_ASO_0052
ctcatgcagacaggtgtactggaca

SEQ ID NO: 66 - DNA, 25 bp, Artificial Sequence
  Antisense Oligonucleotide MTx_ASO_0053
tcatgcagacaggtgtactggacat

SEQ ID NO: 67 - DNA, 25 bp, Artificial Sequence
  Antisense Oligonucleotide MTx_ASO_0054
catgcagacaggtgtactggacatg

SEQ ID NO: 68 - DNA, 25 bp, Artificial Sequence
  Antisense Oligonucleotide MTx_ASO_0055
atgcagacaggtgtactggacatgg

SEQ ID NO: 69 - DNA, 25 bp, Artificial Sequence
  Antisense Oligonucleotide MTx_ASO_0056
tgcagacaggtgtactggacatggt

SEQ ID NO: 70 - DNA, 25 bp, Artificial Sequence
  Antisense Oligonucleotide MTx_ASO_0057
gcagacaggtgtactggacatggta

SEQ ID NO: 71 - DNA, 25 bp, Artificial Sequence
  Antisense Oligonucleotide MTx_ASO_0058
cagacaggtgtactggacatggtac

SEQ ID NO: 72 - DNA, 25 bp, Artificial Sequence
  Antisense Oligonucleotide MTx_ASO_0059
agacaggtgtactggacatggtacg

SEQ ID NO: 73 - DNA, 25 bp, Artificial Sequence
  Antisense Oligonucleotide MTx_ASO_0060
gacaggtgtactggacatggtacgg

SEQ ID NO: 74 - DNA, 25 bp, Artificial Sequence
  Antisense Oligonucleotide MTx_ASO_0061
acaggtgtactggacatggtacggg

SEQ ID NO: 75 - DNA, 25 bp, Artificial Sequence
  Antisense Oligonucleotide MTx_ASO_0062
caggtgtactggacatggtacgggg

SEQ ID NO: 76 - DNA, 25 bp, Artificial Sequence
  Antisense Oligonucleotide MTx_ASO_0063
aggtgtactggacatggtacggggc

SEQ ID NO: 77 - DNA, 25 bp, Artificial Sequence
  Antisense Oligonucleotide MTx_ASO_0064
ggtgtactggacatggtacggggcc

SEQ ID NO: 78 - DNA, 25 bp, Artificial Sequence
  Antisense Oligonucleotide MTx_ASO_0065
gtgtactggacatggtacggggcca

SEQ ID NO: 79 - DNA, 25 bp, Artificial Sequence
  Antisense Oligonucleotide MTx_ASO_0066
tgtactggacatggtacggggccac

SEQ ID NO: 80 - DNA, 25 bp, Artificial Sequence
  Antisense Oligonucleotide MTx_ASO_0067
gtactggacatggtacggggccacc

SEQ ID NO: 81 - DNA, 25 bp, Artificial Sequence
  Antisense Oligonucleotide MTx_ASO_0068
tactggacatggtacggggccacct

SEQ ID NO: 82 - DNA, 25 bp, Artificial Sequence
  Antisense Oligonucleotide MTx_ASO_0069
actggacatggtacggggccacctt

SEQ ID NO: 83 - DNA, 25 bp, Artificial Sequence
  Antisense Oligonucleotide MTx_ASO_0070
ctggacatggtacggggccaccttc

SEQ ID NO: 84 - DNA, 25 bp, Artificial Sequence
  Antisense Oligonucleotide MTx_ASO_0071
tggacatggtacggggccaccttct

SEQ ID NO: 85 - DNA, 25 bp, Artificial Sequence
  Antisense Oligonucleotide MTx_ASO_0072
ggacatggtacggggccaccttctc

SEQ ID NO: 86 - DNA, 25 bp, Artificial Sequence
  Antisense Oligonucleotide MTx_ASO_0073
gacatggtacggggccaccttctcc

SEQ ID NO: 87 - DNA, 25 bp, Artificial Sequence
  Antisense Oligonucleotide MTx_ASO_0074
acatggtacggggccaccttctcct

SEQ ID NO: 88 - DNA, 25 bp, Artificial Sequence
  Antisense Oligonucleotide MTx_ASO_0075
catggtacggggccaccttctcctc

SEQ ID NO: 89 - DNA, 25 bp, Artificial Sequence
  Antisense Oligonucleotide MTx_ASO_0076
atggtacggggccaccttctcctcg

SEQ ID NO: 90 - DNA, 25 bp, Artificial Sequence
  Antisense Oligonucleotide MTx_ASO_0077
tggtacggggccaccttctcctcgc

SEQ ID NO: 91 - DNA, 151 bp, Homo sapiens
  misc_feature (1)..(151): Crytpic exon splice acceptor, position chr19:17,642,491-17,642,641
ctccaggttgactctcactactcatcatcaggttcttccttctattccagccctaaccactcaggattgggccgtttgtgtctgggtatgtctcttccagctgcctgggtttcctggaaagaactcttatccccaggaactagtttgttga

SEQ ID NO: 92 - DNA, 151 bp, Homo sapiens
  misc_feature (1)..(151): Cryptic exon splice acceptor, reverse complement
tcaacaaactagttcctggggataagagttctttccaggaaacccaggcagctggaagagacatacccagacacaaacggcccaatcctgagtggttagggctggaatagaaggaagaacctgatgatgagtagtgagagtcaacctggag

SEQ ID NO: 93 - DNA, 25 bp, Artificial Sequence
  Antisense Oligonucleotide MTx_ASO_0078
tcaacaaactagttcctggggataa

SEQ ID NO: 94 - DNA, 25 bp, Artificial Sequence
  Antisense Oligonucleotide MTx_ASO_0079
caacaaactagttcctggggataag

SEQ ID NO: 95 - DNA, 25 bp, Artificial Sequence
  Antisense Oligonucleotide MTx_ASO_0080
aacaaactagttcctggggataaga

SEQ ID NO: 96 - DNA, 25 bp, Artificial Sequence
  Antisense Oligonucleotide MTx_ASO_0081
acaaactagttcctggggataagag

SEQ ID NO: 97 - DNA, 25 bp, Artificial Sequence
  Antisense Oligonucleotide MTx_ASO_0082
caaactagttcctggggataagagt

SEQ ID NO: 98 - DNA, 25 bp, Artificial Sequence
  Antisense Oligonucleotide MTx_ASO_0083
aaactagttcctggggataagagtt

SEQ ID NO: 99 - DNA, 25 bp, Artificial Sequence
  Antisense Oligonucleotide MTx_ASO_0084
aactagttcctggggataagagttc

SEQ ID NO: 100 - DNA, 25 bp, Artificial Sequence
  Antisense Oligonucleotide MTx_ASO_0085
actagttcctggggataagagttct

SEQ ID NO: 101 - DNA, 25 bp, Artificial Sequence
  Antisense Oligonucleotide MTx_ASO_0086
ctagttcctggggataagagttctt

SEQ ID NO: 102 - DNA, 25 bp, Artificial Sequence
  Antisense Oligonucleotide MTx_ASO_0087
tagttcctggggataagagttcttt

SEQ ID NO: 103 - DNA, 25 bp, Artificial Sequence
  Antisense Oligonucleotide MTx_ASO_0088
agttcctggggataagagttctttc

SEQ ID NO: 104 - DNA, 25 bp, Artificial Sequence
  Antisense Oligonucleotide MTx_ASO_0089
gttcctggggataagagttctttcc

SEQ ID NO: 105 - DNA, 25 bp, Artificial Sequence
  Antisense Oligonucleotide MTx_ASO_0090
ttcctggggataagagttctttcca

SEQ ID NO: 106 - DNA, 25 bp, Artificial Sequence
  Antisense Oligonucleotide MTx_ASO_0091
tcctggggataagagttctttccag

SEQ ID NO: 107 - DNA, 25 bp, Artificial Sequence
  Antisense Oligonucleotide MTx_ASO_0092
cctggggataagagttctttccagg

SEQ ID NO: 108 - DNA, 25 bp, Artificial Sequence
  Antisense Oligonucleotide MTx_ASO_0093
ctggggataagagttctttccagga

SEQ ID NO: 109 - DNA, 25 bp, Artificial Sequence
  Antisense Oligonucleotide MTx_ASO_0094
tggggataagagttctttccaggaa

SEQ ID NO: 110 - DNA, 25 bp, Artificial Sequence
  Antisense Oligonucleotide MTx_ASO_0095
ggggataagagttctttccaggaaa

SEQ ID NO: 111 - DNA, 25 bp, Artificial Sequence
  Antisense Oligonucleotide MTx_ASO_0096
gggataagagttctttccaggaaac

SEQ ID NO: 112 - DNA, 25 bp, Artificial Sequence
  Antisense Oligonucleotide MTx_ASO_0097
ggataagagttctttccaggaaacc

SEQ ID NO: 113 - DNA, 25 bp, Artificial Sequence
  Antisense Oligonucleotide MTx_ASO_0098
gataagagttctttccaggaaaccc

SEQ ID NO: 114 - DNA, 25 bp, Artificial Sequence
  Antisense Oligonucleotide MTx_ASO_0099
ataagagttctttccaggaaaccca

SEQ ID NO: 115 - DNA, 25 bp, Artificial Sequence
  Antisense Oligonucleotide MTx_ASO_0100
taagagttctttccaggaaacccag

SEQ ID NO: 116 - DNA, 25 bp, Artificial Sequence
  Antisense Oligonucleotide MTx_ASO_0101
aagagttctttccaggaaacccagg

SEQ ID NO: 117 - DNA, 25 bp, Artificial Sequence
  Antisense Oligonucleotide MTx_ASO_0102
agagttctttccaggaaacccaggc

SEQ ID NO: 118 - DNA, 25 bp, Artificial Sequence
  Antisense Oligonucleotide MTx_ASO_0103
gagttctttccaggaaacccaggca

SEQ ID NO: 119 - DNA, 25 bp, Artificial Sequence
  Antisense Oligonucleotide MTx_ASO_0104
agttctttccaggaaacccaggcag

SEQ ID NO: 120 - DNA, 25 bp, Artificial Sequence
  Antisense Oligonucleotide MTx_ASO_0105
gttctttccaggaaacccaggcagc

SEQ ID NO: 121 - DNA, 25 bp, Artificial Sequence
  Antisense Oligonucleotide MTx_ASO_0106
ttctttccaggaaacccaggcagct

SEQ ID NO: 122 - DNA, 25 bp, Artificial Sequence
  Antisense Oligonucleotide MTx_ASO_0107
tctttccaggaaacccaggcagctg

SEQ ID NO: 123 - DNA, 25 bp, Artificial Sequence
  Antisense Oligonucleotide MTx_ASO_0108
ctttccaggaaacccaggcagctgg

SEQ ID NO: 124 - DNA, 25 bp, Artificial Sequence
  Antisense Oligonucleotide MTx_ASO_0109
tttccaggaaacccaggcagctgga

SEQ ID NO: 125 - DNA, 25 bp, Artificial Sequence
  Antisense Oligonucleotide MTx_ASO_0110
ttccaggaaacccaggcagctggaa

SEQ ID NO: 126 - DNA, 25 bp, Artificial Sequence
  Antisense Oligonucleotide MTx_ASO_0111
tccaggaaacccaggcagctggaag

SEQ ID NO: 127 - DNA, 25 bp, Artificial Sequence
  Antisense Oligonucleotide MTx_ASO_0112
ccaggaaacccaggcagctggaaga

SEQ ID NO: 128 - DNA, 25 bp, Artificial Sequence
  Antisense Oligonucleotide MTx_ASO_0113
caggaaacccaggcagctggaagag

SEQ ID NO: 129 - DNA, 25 bp, Artificial Sequence
  Antisense Oligonucleotide MTx_ASO_0114
aggaaacccaggcagctggaagaga

SEQ ID NO: 130 - DNA, 25 bp, Artificial Sequence
  Antisense Oligonucleotide MTx_ASO_0115
ggaaacccaggcagctggaagagac

SEQ ID NO: 131 - DNA, 25 bp, Artificial Sequence
  Antisense Oligonucleotide MTx_ASO_0116
gaaacccaggcagctggaagagaca

SEQ ID NO: 132 - DNA, 25 bp, Artificial Sequence
  Antisense Oligonucleotide MTx_ASO_0117
aaacccaggcagctggaagagacat

SEQ ID NO: 133 - DNA, 25 bp, Artificial Sequence
  Antisense Oligonucleotide MTx_ASO_0118
aacccaggcagctggaagagacata

SEQ ID NO: 134 - DNA, 25 bp, Artificial Sequence
  Antisense Oligonucleotide MTx_ASO_0119
acccaggcagctggaagagacatac

SEQ ID NO: 135 - DNA, 25 bp, Artificial Sequence
  Antisense Oligonucleotide MTx_ASO_0120
cccaggcagctggaagagacatacc

SEQ ID NO: 136 - DNA, 25 bp, Artificial Sequence
  Antisense Oligonucleotide MTx_ASO_0121
ccaggcagctggaagagacataccc

SEQ ID NO: 137 - DNA, 25 bp, Artificial Sequence
  Antisense Oligonucleotide MTx_ASO_0122
caggcagctggaagagacataccca

SEQ ID NO: 138 - DNA, 25 bp, Artificial Sequence
  Antisense Oligonucleotide MTx_ASO_0123
aggcagctggaagagacatacccag

SEQ ID NO: 139 - DNA, 25 bp, Artificial Sequence
  Antisense Oligonucleotide MTx_ASO_0124
ggcagctggaagagacatacccaga

SEQ ID NO: 140 - DNA, 25 bp, Artificial Sequence
  Antisense Oligonucleotide MTx_ASO_0125
gcagctggaagagacatacccagac

SEQ ID NO: 141 - DNA, 25 bp, Artificial Sequence
  Antisense Oligonucleotide MTx_ASO_0126
cagctggaagagacatacccagaca

SEQ ID NO: 142 - DNA, 25 bp, Artificial Sequence
  Antisense Oligonucleotide MTx_ASO_0127
agctggaagagacatacccagacac

SEQ ID NO: 143 - DNA, 25 bp, Artificial Sequence
  Antisense Oligonucleotide MTx_ASO_0128
gctggaagagacatacccagacaca

SEQ ID NO: 144 - DNA, 25 bp, Artificial Sequence
  Antisense Oligonucleotide MTx_ASO_0129
ctggaagagacatacccagacacaa

SEQ ID NO: 145 - DNA, 25 bp, Artificial Sequence
  Antisense Oligonucleotide MTx_ASO_0130
tggaagagacatacccagacacaaa

SEQ ID NO: 146 - DNA, 25 bp, Artificial Sequence
  Antisense Oligonucleotide MTx_ASO_0131
ggaagagacatacccagacacaaac

SEQ ID NO: 147 - DNA, 25 bp, Artificial Sequence
  Antisense Oligonucleotide MTx_ASO_0132
gaagagacatacccagacacaaacg

SEQ ID NO: 148 - DNA, 25 bp, Artificial Sequence
  Antisense Oligonucleotide MTx_ASO_0133
aagagacatacccagacacaaacgg

SEQ ID NO: 149 - DNA, 25 bp, Artificial Sequence
  Antisense Oligonucleotide MTx_ASO_0134
agagacatacccagacacaaacggc

SEQ ID NO: 150 - DNA, 25 bp, Artificial Sequence
  Antisense Oligonucleotide MTx_ASO_0135
gagacatacccagacacaaacggcc

SEQ ID NO: 151 - DNA, 25 bp, Artificial Sequence
  Antisense Oligonucleotide MTx_ASO_0136
agacatacccagacacaaacggccc

SEQ ID NO: 152 - DNA, 25 bp, Artificial Sequence
  Antisense Oligonucleotide MTx_ASO_0137
gacatacccagacacaaacggccca

SEQ ID NO: 153 - DNA, 25 bp, Artificial Sequence
  Antisense Oligonucleotide MTx_ASO_0138
acatacccagacacaaacggcccaa

SEQ ID NO: 154 - DNA, 25 bp, Artificial Sequence
  Antisense Oligonucleotide MTx_ASO_0139
catacccagacacaaacggcccaat

SEQ ID NO: 155 - DNA, 25 bp, Artificial Sequence
  Antisense Oligonucleotide MTx_ASO_0140
atacccagacacaaacggcccaatc

SEQ ID NO: 156 - DNA, 25 bp, Artificial Sequence
  Antisense Oligonucleotide MTx_ASO_0141
tacccagacacaaacggcccaatcc

SEQ ID NO: 157 - DNA, 25 bp, Artificial Sequence
  Antisense Oligonucleotide MTx_ASO_0142
acccagacacaaacggcccaatcct

SEQ ID NO: 158 - DNA, 25 bp, Artificial Sequence
  Antisense Oligonucleotide MTx_ASO_0143
cccagacacaaacggcccaatcctg

SEQ ID NO: 159 - DNA, 25 bp, Artificial Sequence
  Antisense Oligonucleotide MTx_ASO_0144
ccagacacaaacggcccaatcctga

SEQ ID NO: 160 - DNA, 25 bp, Artificial Sequence
  Antisense Oligonucleotide MTx_ASO_0145
cagacacaaacggcccaatcctgag

SEQ ID NO: 161 - DNA, 25 bp, Artificial Sequence
  Antisense Oligonucleotide MTx_ASO_0146
agacacaaacggcccaatcctgagt

SEQ ID NO: 162 - DNA, 25 bp, Artificial Sequence
  Antisense Oligonucleotide MTx_ASO_0147
gacacaaacggcccaatcctgagtg

SEQ ID NO: 163 - DNA, 25 bp, Artificial Sequence
  Antisense Oligonucleotide MTx_ASO_0148
acacaaacggcccaatcctgagtgg

SEQ ID NO: 164 - DNA, 25 bp, Artificial Sequence
  Antisense Oligonucleotide MTx_ASO_0149
cacaaacggcccaatcctgagtggt

SEQ ID NO: 165 - DNA, 25 bp, Artificial Sequence
  Antisense Oligonucleotide MTx_ASO_0150
acaaacggcccaatcctgagtggtt

SEQ ID NO: 166 - DNA, 25 bp, Artificial Sequence
  Antisense Oligonucleotide MTx_ASO_0151
caaacggcccaatcctgagtggtta

SEQ ID NO: 167 - DNA, 25 bp, Artificial Sequence
  Antisense Oligonucleotide MTx_ASO_0152
aaacggcccaatcctgagtggttag

SEQ ID NO: 168 - DNA, 25 bp, Artificial Sequence
  Antisense Oligonucleotide MTx_ASO_0153
aacggcccaatcctgagtggttagg

SEQ ID NO: 169 - DNA, 25 bp, Artificial Sequence
  Antisense Oligonucleotide MTx_ASO_0154
acggcccaatcctgagtggttaggg

SEQ ID NO: 170 - DNA, 25 bp, Artificial Sequence
  Antisense Oligonucleotide MTx_ASO_0155
cggcccaatcctgagtggttagggc

SEQ ID NO: 171 - DNA, 25 bp, Artificial Sequence
  Antisense Oligonucleotide MTx_ASO_0156
ggcccaatcctgagtggttagggct

SEQ ID NO: 172 - DNA, 25 bp, Artificial Sequence
  Antisense Oligonucleotide MTx_ASO_0157
gcccaatcctgagtggttagggctg

SEQ ID NO: 173 - DNA, 25 bp, Artificial Sequence
  Antisense Oligonucleotide MTx_ASO_0158
cccaatcctgagtggttagggctgg

SEQ ID NO: 174 - DNA, 25 bp, Artificial Sequence
  Antisense Oligonucleotide MTx_ASO_0159
ccaatcctgagtggttagggctgga

SEQ ID NO: 175 - DNA, 25 bp, Artificial Sequence
  Antisense Oligonucleotide MTx_ASO_0160
caatcctgagtggttagggctggaa

SEQ ID NO: 176 - DNA, 25 bp, Artificial Sequence
  Antisense Oligonucleotide MTx_ASO_0161
aatcctgagtggttagggctggaat

SEQ ID NO: 177 - DNA, 25 bp, Artificial Sequence
  Antisense Oligonucleotide MTx_ASO_0162
atcctgagtggttagggctggaata

SEQ ID NO: 178 - DNA, 25 bp, Artificial Sequence
  Antisense Oligonucleotide MTx_ASO_0163
tcctgagtggttagggctggaatag

SEQ ID NO: 179 - DNA, 25 bp, Artificial Sequence
  Antisense Oligonucleotide MTx_ASO_0164
cctgagtggttagggctggaataga

SEQ ID NO: 180 - DNA, 25 bp, Artificial Sequence
  Antisense Oligonucleotide MTx_ASO_0165
ctgagtggttagggctggaatagaa

SEQ ID NO: 181 - DNA, 25 bp, Artificial Sequence
  Antisense Oligonucleotide MTx_ASO_0166
tgagtggttagggctggaatagaag

SEQ ID NO: 182 - DNA, 25 bp, Artificial Sequence
  Antisense Oligonucleotide MTx_ASO_0167
gagtggttagggctggaatagaagg

SEQ ID NO: 183 - DNA, 25 bp, Artificial Sequence
  Antisense Oligonucleotide MTx_ASO_0168
agtggttagggctggaatagaagga

SEQ ID NO: 184 - DNA, 25 bp, Artificial Sequence
  Antisense Oligonucleotide MTx_ASO_0169
gtggttagggctggaatagaaggaa

SEQ ID NO: 185 - DNA, 25 bp, Artificial Sequence
  Antisense Oligonucleotide MTx_ASO_0170
tggttagggctggaatagaaggaag

SEQ ID NO: 186 - DNA, 25 bp, Artificial Sequence
  Antisense Oligonucleotide MTx_ASO_0171
ggttagggctggaatagaaggaaga

SEQ ID NO: 187 - DNA, 25 bp, Artificial Sequence
  Antisense Oligonucleotide MTx_ASO_0172
gttagggctggaatagaaggaagaa

SEQ ID NO: 188 - DNA, 25 bp, Artificial Sequence
  Antisense Oligonucleotide MTx_ASO_0173
ttagggctggaatagaaggaagaac

SEQ ID NO: 189 - DNA, 25 bp, Artificial Sequence
  Antisense Oligonucleotide MTx_ASO_0174
tagggctggaatagaaggaagaacc

SEQ ID NO: 190 - DNA, 25 bp, Artificial Sequence
  Antisense Oligonucleotide MTx_ASO_0175
agggctggaatagaaggaagaacct

SEQ ID NO: 191 - DNA, 25 bp, Artificial Sequence
  Antisense Oligonucleotide MTx_ASO_0176
gggctggaatagaaggaagaacctg

SEQ ID NO: 192 - DNA, 25 bp, Artificial Sequence
  Antisense Oligonucleotide MTx_ASO_0177
ggctggaatagaaggaagaacctga

SEQ ID NO: 193 - DNA, 25 bp, Artificial Sequence
  Antisense Oligonucleotide MTx_ASO_0178
gctggaatagaaggaagaacctgat

SEQ ID NO: 194 - DNA, 25 bp, Artificial Sequence
  Antisense Oligonucleotide MTx_ASO_0179
ctggaatagaaggaagaacctgatg

SEQ ID NO: 195 - DNA, 25 bp, Artificial Sequence
  Antisense Oligonucleotide MTx_ASO_0180
tggaatagaaggaagaacctgatga

SEQ ID NO: 196 - DNA, 25 bp, Artificial Sequence
  Antisense Oligonucleotide MTx_ASO_0181
ggaatagaaggaagaacctgatgat

SEQ ID NO: 197 - DNA, 25 bp, Artificial Sequence
  Antisense Oligonucleotide MTx_ASO_0182
gaatagaaggaagaacctgatgatg

SEQ ID NO: 198 - DNA, 25 bp, Artificial Sequence
  Antisense Oligonucleotide MTx_ASO_0183
aatagaaggaagaacctgatgatga

SEQ ID NO: 199 - DNA, 25 bp, Artificial Sequence
  Antisense Oligonucleotide MTx_ASO_0184
atagaaggaagaacctgatgatgag

SEQ ID NO: 200 - DNA, 25 bp, Artificial Sequence
  Antisense Oligonucleotide MTx_ASO_0185
tagaaggaagaacctgatgatgagt

SEQ ID NO: 201 - DNA, 25 bp, Artificial Sequence
  Antisense Oligonucleotide MTx_ASO_0186
agaaggaagaacctgatgatgagta

SEQ ID NO: 202 - DNA, 25 bp, Artificial Sequence
  Antisense Oligonucleotide MTx_ASO_0187
gaaggaagaacctgatgatgagtag

SEQ ID NO: 203 - DNA, 25 bp, Artificial Sequence
  Antisense Oligonucleotide MTx_ASO_0188
aaggaagaacctgatgatgagtagt

SEQ ID NO: 204 - DNA, 25 bp, Artificial Sequence
  Antisense Oligonucleotide MTx_ASO_0189
aggaagaacctgatgatgagtagtg

SEQ ID NO: 205 - DNA, 25 bp, Artificial Sequence
  Antisense Oligonucleotide MTx_ASO_0190
ggaagaacctgatgatgagtagtga

SEQ ID NO: 206 - DNA, 25 bp, Artificial Sequence
  Antisense Oligonucleotide MTx_ASO_0191
gaagaacctgatgatgagtagtgag

SEQ ID NO: 207 - DNA, 25 bp, Artificial Sequence
  Antisense Oligonucleotide MTx_ASO_0192
aagaacctgatgatgagtagtgaga

SEQ ID NO: 208 - DNA, 25 bp, Artificial Sequence
  Antisense Oligonucleotide MTx_ASO_0193
agaacctgatgatgagtagtgagag

SEQ ID NO: 209 - DNA, 25 bp, Artificial Sequence
  Antisense Oligonucleotide MTx_ASO_0194
gaacctgatgatgagtagtgagagt

SEQ ID NO: 210 - DNA, 25 bp, Artificial Sequence
  Antisense Oligonucleotide MTx_ASO_0195
aacctgatgatgagtagtgagagtc

SEQ ID NO: 211 - DNA, 25 bp, Artificial Sequence
  Antisense Oligonucleotide MTx_ASO_0196
acctgatgatgagtagtgagagtca

SEQ ID NO: 212 - DNA, 25 bp, Artificial Sequence
  Antisense Oligonucleotide MTx_ASO_0197
cctgatgatgagtagtgagagtcaa

SEQ ID NO: 213 - DNA, 25 bp, Artificial Sequence
  Antisense Oligonucleotide MTx_ASO_0198
ctgatgatgagtagtgagagtcaac

SEQ ID NO: 214 - DNA, 25 bp, Artificial Sequence
  Antisense Oligonucleotide MTx_ASO_0199
tgatgatgagtagtgagagtcaacc

SEQ ID NO: 215 - DNA, 25 bp, Artificial Sequence
  Antisense Oligonucleotide MTx_ASO_0200
gatgatgagtagtgagagtcaacct

SEQ ID NO: 216 - DNA, 25 bp, Artificial Sequence
  Antisense Oligonucleotide MTx_ASO_0201
atgatgagtagtgagagtcaacctg

SEQ ID NO: 217 - DNA, 25 bp, Artificial Sequence
  Antisense Oligonucleotide MTx_ASO_0202
tgatgagtagtgagagtcaacctgg

SEQ ID NO: 218 - DNA, 25 bp, Artificial Sequence
  Antisense Oligonucleotide MTx_ASO_0203
gatgagtagtgagagtcaacctgga

SEQ ID NO: 219 - DNA, 25 bp, Artificial Sequence
  Antisense Oligonucleotide MTx_ASO_0204
atgagtagtgagagtcaacctggag

SEQ ID NO: 220 - DNA, 101 bp, Homo sapiens
  misc_feature (1)..(101): Cryptic exon splice donor, position chr19:17,642,363-17,642,463
tgaacagatgaatgagtgatgagtagataaaaggatggatggagagatgggtgagtacatggatggatagatggatgagttggtgggtagattcgtggcta

SEQ ID NO: 221 - DNA, 101 bp, Homo sapiens
  misc_feature (1)..(101): Cryptic exon splice donor, reverse complement
tagccacgaatctacccaccaactcatccatctatccatccatgtactcacccatctctccatccatccttttatctactcatcactcattcatctgttca

SEQ ID NO: 222 - DNA, 25 bp, Artificial Sequence
  Antisense Oligonucleotide MTx_ASO_0205
tagccacgaatctacccaccaactc

SEQ ID NO: 223 - DNA, 25 bp, Artificial Sequence
  Antisense Oligonucleotide MTx_ASO_0206
agccacgaatctacccaccaactca

SEQ ID NO: 224 - DNA, 25 bp, Artificial Sequence
  Antisense Oligonucleotide MTx_ASO_0207
gccacgaatctacccaccaactcat

SEQ ID NO: 225 - DNA, 25 bp, Artificial Sequence
  Antisense Oligonucleotide MTx_ASO_0208
ccacgaatctacccaccaactcatc

SEQ ID NO: 226 - DNA, 25 bp, Artificial Sequence
  Antisense Oligonucleotide MTx_ASO_0209
cacgaatctacccaccaactcatcc

SEQ ID NO: 227 - DNA, 25 bp, Artificial Sequence
  Antisense Oligonucleotide MTx_ASO_0210
acgaatctacccaccaactcatcca

SEQ ID NO: 228 - DNA, 25 bp, Artificial Sequence
  Antisense Oligonucleotide MTx_ASO_0211
cgaatctacccaccaactcatccat

SEQ ID NO: 229 - DNA, 25 bp, Artificial Sequence
  Antisense Oligonucleotide MTx_ASO_0212
gaatctacccaccaactcatccatc

SEQ ID NO: 230 - DNA, 25 bp, Artificial Sequence
  Antisense Oligonucleotide MTx_ASO_0213
aatctacccaccaactcatccatct

SEQ ID NO: 231 - DNA, 25 bp, Artificial Sequence
  Antisense Oligonucleotide MTx_ASO_0214
atctacccaccaactcatccatcta

SEQ ID NO: 232 - DNA, 25 bp, Artificial Sequence
  Antisense Oligonucleotide MTx_ASO_0215
tctacccaccaactcatccatctat

SEQ ID NO: 233 - DNA, 25 bp, Artificial Sequence
  Antisense Oligonucleotide MTx_ASO_0216
ctacccaccaactcatccatctatc

SEQ ID NO: 234 - DNA, 25 bp, Artificial Sequence
  Antisense Oligonucleotide MTx_ASO_0217
tacccaccaactcatccatctatcc

SEQ ID NO: 235 - DNA, 25 bp, Artificial Sequence
  Antisense Oligonucleotide MTx_ASO_0218
acccaccaactcatccatctatcca

SEQ ID NO: 236 - DNA, 25 bp, Artificial Sequence
  Antisense Oligonucleotide MTx_ASO_0219
cccaccaactcatccatctatccat

SEQ ID NO: 237 - DNA, 25 bp, Artificial Sequence
  Antisense Oligonucleotide MTx_ASO_0220
ccaccaactcatccatctatccatc

SEQ ID NO: 238 - DNA, 25 bp, Artificial Sequence
  Antisense Oligonucleotide MTx_ASO_0221
caccaactcatccatctatccatcc

SEQ ID NO: 239 - DNA, 25 bp, Artificial Sequence
  Antisense Oligonucleotide MTx_ASO_0222
accaactcatccatctatccatcca

SEQ ID NO: 240 - DNA, 25 bp, Artificial Sequence
  Antisense Oligonucleotide MTx_ASO_0223
ccaactcatccatctatccatccat

SEQ ID NO: 241 - DNA, 25 bp, Artificial Sequence
  Antisense Oligonucleotide MTx_ASO_0224
caactcatccatctatccatccatg

SEQ ID NO: 242 - DNA, 25 bp, Artificial Sequence
  Antisense Oligonucleotide MTx_ASO_0225
aactcatccatctatccatccatgt

SEQ ID NO: 243 - DNA, 25 bp, Artificial Sequence
  Antisense Oligonucleotide MTx_ASO_0226
actcatccatctatccatccatgta

SEQ ID NO: 244 - DNA, 25 bp, Artificial Sequence
  Antisense Oligonucleotide MTx_ASO_0227
ctcatccatctatccatccatgtac

SEQ ID NO: 245 - DNA, 25 bp, Artificial Sequence
  Antisense Oligonucleotide MTx_ASO_0228
tcatccatctatccatccatgtact

SEQ ID NO: 246 - DNA, 25 bp, Artificial Sequence
  Antisense Oligonucleotide MTx_ASO_0229
catccatctatccatccatgtactc

SEQ ID NO: 247 - DNA, 25 bp, Artificial Sequence
  Antisense Oligonucleotide MTx_ASO_0230
atccatctatccatccatgtactca

SEQ ID NO: 248 - DNA, 25 bp, Artificial Sequence
  Antisense Oligonucleotide MTx_ASO_0231
tccatctatccatccatgtactcac

SEQ ID NO: 249 - DNA, 25 bp, Artificial Sequence
  Antisense Oligonucleotide MTx_ASO_0232
ccatctatccatccatgtactcacc

SEQ ID NO: 250 - DNA, 25 bp, Artificial Sequence
  Antisense Oligonucleotide MTx_ASO_0233
catctatccatccatgtactcaccc

SEQ ID NO: 251 - DNA, 25 bp, Artificial Sequence
  Antisense Oligonucleotide MTx_ASO_0234
atctatccatccatgtactcaccca

SEQ ID NO: 252 - DNA, 25 bp, Artificial Sequence
  Antisense Oligonucleotide MTx_ASO_0235
tctatccatccatgtactcacccat

SEQ ID NO: 253 - DNA, 25 bp, Artificial Sequence
  Antisense Oligonucleotide MTx_ASO_0236
ctatccatccatgtactcacccatc

SEQ ID NO: 254 - DNA, 25 bp, Artificial Sequence
  Antisense Oligonucleotide MTx_ASO_0237
tatccatccatgtactcacccatct

SEQ ID NO: 255 - DNA, 25 bp, Artificial Sequence
  Antisense Oligonucleotide MTx_ASO_0238
atccatccatgtactcacccatctc

SEQ ID NO: 256 - DNA, 25 bp, Artificial Sequence
  Antisense Oligonucleotide MTx_ASO_0239
tccatccatgtactcacccatctct

SEQ ID NO: 257 - DNA, 25 bp, Artificial Sequence
  Antisense Oligonucleotide MTx_ASO_0240
ccatccatgtactcacccatctctc

SEQ ID NO: 258 - DNA, 25 bp, Artificial Sequence
  Antisense Oligonucleotide MTx_ASO_0241
catccatgtactcacccatctctcc

SEQ ID NO: 259 - DNA, 25 bp, Artificial Sequence
  Antisense Oligonucleotide MTx_ASO_0242
atccatgtactcacccatctctcca

SEQ ID NO: 260 - DNA, 25 bp, Artificial Sequence
  Antisense Oligonucleotide MTx_ASO_0243
tccatgtactcacccatctctccat

SEQ ID NO: 261 - DNA, 25 bp, Artificial Sequence
  Antisense Oligonucleotide MTx_ASO_0244
ccatgtactcacccatctctccatc

SEQ ID NO: 262 - DNA, 25 bp, Artificial Sequence
  Antisense Oligonucleotide MTx_ASO_0245
catgtactcacccatctctccatcc

SEQ ID NO: 263 - DNA, 25 bp, Artificial Sequence
  Antisense Oligonucleotide MTx_ASO_0246
atgtactcacccatctctccatcca

SEQ ID NO: 264 - DNA, 25 bp, Artificial Sequence
  Antisense Oligonucleotide MTx_ASO_0247
tgtactcacccatctctccatccat

SEQ ID NO: 265 - DNA, 25 bp, Artificial Sequence
  Antisense Oligonucleotide MTx_ASO_0248
gtactcacccatctctccatccatc

SEQ ID NO: 266 - DNA, 25 bp, Artificial Sequence
  Antisense Oligonucleotide MTx_ASO_0249
tactcacccatctctccatccatcc

SEQ ID NO: 267 - DNA, 25 bp, Artificial Sequence
  Antisense Oligonucleotide MTx_ASO_0250
actcacccatctctccatccatcct

SEQ ID NO: 268 - DNA, 25 bp, Artificial Sequence
  Antisense Oligonucleotide MTx_ASO_0251
ctcacccatctctccatccatcctt

SEQ ID NO: 269 - DNA, 25 bp, Artificial Sequence
  Antisense Oligonucleotide MTx_ASO_0252
tcacccatctctccatccatccttt

SEQ ID NO: 270 - DNA, 25 bp, Artificial Sequence
  Antisense Oligonucleotide MTx_ASO_0253
cacccatctctccatccatcctttt

SEQ ID NO: 271 - DNA, 25 bp, Artificial Sequence
  Antisense Oligonucleotide MTx_ASO_0254
acccatctctccatccatcctttta

SEQ ID NO: 272 - DNA, 25 bp, Artificial Sequence
  Antisense Oligonucleotide MTx_ASO_0255
cccatctctccatccatccttttat

SEQ ID NO: 273 - DNA, 25 bp, Artificial Sequence
  Antisense Oligonucleotide MTx_ASO_0256
ccatctctccatccatccttttatc

SEQ ID NO: 274 - DNA, 25 bp, Artificial Sequence
  Antisense Oligonucleotide MTx_ASO_0257
catctctccatccatccttttatct

SEQ ID NO: 275 - DNA, 25 bp, Artificial Sequence
  Antisense Oligonucleotide MTx_ASO_0258
atctctccatccatccttttatcta

SEQ ID NO: 276 - DNA, 25 bp, Artificial Sequence
  Antisense Oligonucleotide MTx_ASO_0259
tctctccatccatccttttatctac

SEQ ID NO: 277 - DNA, 25 bp, Artificial Sequence
  Antisense Oligonucleotide MTx_ASO_0260
ctctccatccatccttttatctact

SEQ ID NO: 278 - DNA, 25 bp, Artificial Sequence
  Antisense Oligonucleotide MTx_ASO_0261
tctccatccatccttttatctactc

SEQ ID NO: 279 - DNA, 25 bp, Artificial Sequence
  Antisense Oligonucleotide MTx_ASO_0262
ctccatccatccttttatctactca

SEQ ID NO: 280 - DNA, 25 bp, Artificial Sequence
  Antisense Oligonucleotide MTx_ASO_0263
tccatccatccttttatctactcat

SEQ ID NO: 281 - DNA, 25 bp, Artificial Sequence
  Antisense Oligonucleotide MTx_ASO_0264
ccatccatccttttatctactcatc

SEQ ID NO: 282 - DNA, 25 bp, Artificial Sequence
  Antisense Oligonucleotide MTx_ASO_0265
catccatccttttatctactcatca

SEQ ID NO: 283 - DNA, 25 bp, Artificial Sequence
  Antisense Oligonucleotide MTx_ASO_0266
atccatccttttatctactcatcac

SEQ ID NO: 284 - DNA, 25 bp, Artificial Sequence
  Antisense Oligonucleotide MTx_ASO_0267
tccatccttttatctactcatcact

SEQ ID NO: 285 - DNA, 25 bp, Artificial Sequence
  Antisense Oligonucleotide MTx_ASO_0268
ccatccttttatctactcatcactc

SEQ ID NO: 286 - DNA, 25 bp, Artificial Sequence
  Antisense Oligonucleotide MTx_ASO_0269
catccttttatctactcatcactca

SEQ ID NO: 287 - DNA, 25 bp, Artificial Sequence
  Antisense Oligonucleotide MTx_ASO_0270
atccttttatctactcatcactcat

SEQ ID NO: 288 - DNA, 25 bp, Artificial Sequence
  Antisense Oligonucleotide MTx_ASO_0271
tccttttatctactcatcactcatt

SEQ ID NO: 289 - DNA, 25 bp, Artificial Sequence
  Antisense Oligonucleotide MTx_ASO_0272
ccttttatctactcatcactcattc

SEQ ID NO: 290 - DNA, 25 bp, Artificial Sequence
  Antisense Oligonucleotide MTx_ASO_0273
cttttatctactcatcactcattca

SEQ ID NO: 291 - DNA, 25 bp, Artificial Sequence
  Antisense Oligonucleotide MTx_ASO_0274
ttttatctactcatcactcattcat

SEQ ID NO: 292 - DNA, 25 bp, Artificial Sequence
  Antisense Oligonucleotide MTx_ASO_0275
tttatctactcatcactcattcatc

SEQ ID NO: 293 - DNA, 25 bp, Artificial Sequence
  Antisense Oligonucleotide MTx_ASO_0276
ttatctactcatcactcattcatct

SEQ ID NO: 294 - DNA, 25 bp, Artificial Sequence
  Antisense Oligonucleotide MTx_ASO_0277
tatctactcatcactcattcatctg

SEQ ID NO: 295 - DNA, 25 bp, Artificial Sequence
  Antisense Oligonucleotide MTx_ASO_0278
atctactcatcactcattcatctgt

SEQ ID NO: 296 - DNA, 25 bp, Artificial Sequence
  Antisense Oligonucleotide MTx_ASO_0279
tctactcatcactcattcatctgtt

SEQ ID NO: 297 - DNA, 25 bp, Artificial Sequence
  Antisense Oligonucleotide MTx_ASO_0280
ctactcatcactcattcatctgttc

SEQ ID NO: 298 - DNA, 25 bp, Artificial Sequence
  Antisense Oligonucleotide MTx_ASO_0281
tactcatcactcattcatctgttca

SEQ ID NO: 299 - DNA, 101 bp, Homo sapiens
  misc_feature (1)..(101): Exon 21 splice acceptor, position chr19:17,641,506-17,641,606
cccggcgaccccttgcactctccatgacactttctctcccatggtggcagaacctgttccacttcgtgaccgacgtgcagaacaatggggtcgtgaagatc

SEQ ID NO: 300 - DNA, 101 bp, Homo sapiens
  misc_feature (1)..(101): Exon 21 splice acceptor, reverse complement
gatcttcacgaccccattgttctgcacgtcggtcacgaagtggaacaggttctgccaccatgggagagaaagtgtcatggagagtgcaaggggtcgccggg

SEQ ID NO: 301 - DNA, 25 bp, Artificial Sequence
  Antisense Oligonucleotide MTx_ASO_0282
gatcttcacgaccccattgttctgc

SEQ ID NO: 302 - DNA, 25 bp, Artificial Sequence
  Antisense Oligonucleotide MTx_ASO_0283
atcttcacgaccccattgttctgca

SEQ ID NO: 303 - DNA, 25 bp, Artificial Sequence
  Antisense Oligonucleotide MTx_ASO_0284
tcttcacgaccccattgttctgcac

SEQ ID NO: 304 - DNA, 25 bp, Artificial Sequence
  Antisense Oligonucleotide MTx_ASO_0285
cttcacgaccccattgttctgcacg

SEQ ID NO: 305 - DNA, 25 bp, Artificial Sequence
  Antisense Oligonucleotide MTx_ASO_0286
ttcacgaccccattgttctgcacgt

SEQ ID NO: 306 - DNA, 25 bp, Artificial Sequence
  Antisense Oligonucleotide MTx_ASO_0287
tcacgaccccattgttctgcacgtc

SEQ ID NO: 307 - DNA, 25 bp, Artificial Sequence
  Antisense Oligonucleotide MTx_ASO_0288
cacgaccccattgttctgcacgtcg

SEQ ID NO: 308 - DNA, 25 bp, Artificial Sequence
  Antisense Oligonucleotide MTx_ASO_0289
acgaccccattgttctgcacgtcgg

SEQ ID NO: 309 - DNA, 25 bp, Artificial Sequence
  Antisense Oligonucleotide MTx_ASO_0290
cgaccccattgttctgcacgtcggt

SEQ ID NO: 310 - DNA, 25 bp, Artificial Sequence
  Antisense Oligonucleotide MTx_ASO_0291
gaccccattgttctgcacgtcggtc

SEQ ID NO: 311 - DNA, 25 bp, Artificial Sequence
  Antisense Oligonucleotide MTx_ASO_0292
accccattgttctgcacgtcggtca

SEQ ID NO: 312 - DNA, 25 bp, Artificial Sequence
  Antisense Oligonucleotide MTx_ASO_0293
ccccattgttctgcacgtcggtcac

SEQ ID NO: 313 - DNA, 25 bp, Artificial Sequence
  Antisense Oligonucleotide MTx_ASO_0294
cccattgttctgcacgtcggtcacg

SEQ ID NO: 314 - DNA, 25 bp, Artificial Sequence
  Antisense Oligonucleotide MTx_ASO_0295
ccattgttctgcacgtcggtcacga

SEQ ID NO: 315 - DNA, 25 bp, Artificial Sequence
  Antisense Oligonucleotide MTx_ASO_0296
cattgttctgcacgtcggtcacgaa

SEQ ID NO: 316 - DNA, 25 bp, Artificial Sequence
  Antisense Oligonucleotide MTx_ASO_0297
attgttctgcacgtcggtcacgaag

SEQ ID NO: 317 - DNA, 25 bp, Artificial Sequence
  Antisense Oligonucleotide MTx_ASO_0298
ttgttctgcacgtcggtcacgaagt

SEQ ID NO: 318 - DNA, 25 bp, Artificial Sequence
  Antisense Oligonucleotide MTx_ASO_0299
tgttctgcacgtcggtcacgaagtg

SEQ ID NO: 319 - DNA, 25 bp, Artificial Sequence
  Antisense Oligonucleotide MTx_ASO_0300
gttctgcacgtcggtcacgaagtgg

SEQ ID NO: 320 - DNA, 25 bp, Artificial Sequence
  Antisense Oligonucleotide MTx_ASO_0301
ttctgcacgtcggtcacgaagtgga

SEQ ID NO: 321 - DNA, 25 bp, Artificial Sequence
  Antisense Oligonucleotide MTx_ASO_0302
tctgcacgtcggtcacgaagtggaa

SEQ ID NO: 322 - DNA, 25 bp, Artificial Sequence
  Antisense Oligonucleotide MTx_ASO_0303
ctgcacgtcggtcacgaagtggaac

SEQ ID NO: 323 - DNA, 25 bp, Artificial Sequence
  Antisense Oligonucleotide MTx_ASO_0304
tgcacgtcggtcacgaagtggaaca

SEQ ID NO: 324 - DNA, 25 bp, Artificial Sequence
  Antisense Oligonucleotide MTx_ASO_0305
gcacgtcggtcacgaagtggaacag

SEQ ID NO: 325 - DNA, 25 bp, Artificial Sequence
  Antisense Oligonucleotide MTx_ASO_0306
cacgtcggtcacgaagtggaacagg

SEQ ID NO: 326 - DNA, 25 bp, Artificial Sequence
  Antisense Oligonucleotide MTx_ASO_0307
acgtcggtcacgaagtggaacaggt

SEQ ID NO: 327 - DNA, 25 bp, Artificial Sequence
  Antisense Oligonucleotide MTx_ASO_0308
cgtcggtcacgaagtggaacaggtt

SEQ ID NO: 328 - DNA, 25 bp, Artificial Sequence
  Antisense Oligonucleotide MTx_ASO_0309
gtcggtcacgaagtggaacaggttc

SEQ ID NO: 329 - DNA, 25 bp, Artificial Sequence
  Antisense Oligonucleotide MTx_ASO_0310
tcggtcacgaagtggaacaggttct

SEQ ID NO: 330 - DNA, 25 bp, Artificial Sequence
  Antisense Oligonucleotide MTx_ASO_0311
cggtcacgaagtggaacaggttctg

SEQ ID NO: 331 - DNA, 25 bp, Artificial Sequence
  Antisense Oligonucleotide MTx_ASO_0312
ggtcacgaagtggaacaggttctgc

SEQ ID NO: 332 - DNA, 25 bp, Artificial Sequence
  Antisense Oligonucleotide MTx_ASO_0313
gtcacgaagtggaacaggttctgcc

SEQ ID NO: 333 - DNA, 25 bp, Artificial Sequence
  Antisense Oligonucleotide MTx_ASO_0314
tcacgaagtggaacaggttctgcca

SEQ ID NO: 334 - DNA, 25 bp, Artificial Sequence
  Antisense Oligonucleotide MTx_ASO_0315
cacgaagtggaacaggttctgccac

SEQ ID NO: 335 - DNA, 25 bp, Artificial Sequence
  Antisense Oligonucleotide MTx_ASO_0316
acgaagtggaacaggttctgccacc

SEQ ID NO: 336 - DNA, 25 bp, Artificial Sequence
  Antisense Oligonucleotide MTx_ASO_0317
cgaagtggaacaggttctgccacca

SEQ ID NO: 337 - DNA, 25 bp, Artificial Sequence
  Antisense Oligonucleotide MTx_ASO_0318
gaagtggaacaggttctgccaccat

SEQ ID NO: 338 - DNA, 25 bp, Artificial Sequence
  Antisense Oligonucleotide MTx_ASO_0319
aagtggaacaggttctgccaccatg

SEQ ID NO: 339 - DNA, 25 bp, Artificial Sequence
  Antisense Oligonucleotide MTx_ASO_0320
agtggaacaggttctgccaccatgg

SEQ ID NO: 340 - DNA, 25 bp, Artificial Sequence
  Antisense Oligonucleotide MTx_ASO_0321
gtggaacaggttctgccaccatggg

SEQ ID NO: 341 - DNA, 25 bp, Artificial Sequence
  Antisense Oligonucleotide MTx_ASO_0322
tggaacaggttctgccaccatggga

SEQ ID NO: 342 - DNA, 25 bp, Artificial Sequence
  Antisense Oligonucleotide MTx_ASO_0323
ggaacaggttctgccaccatgggag

SEQ ID NO: 343 - DNA, 25 bp, Artificial Sequence
  Antisense Oligonucleotide MTx_ASO_0324
gaacaggttctgccaccatgggaga

SEQ ID NO: 344 - DNA, 25 bp, Artificial Sequence
  Antisense Oligonucleotide MTx_ASO_0325
aacaggttctgccaccatgggagag

SEQ ID NO: 345 - DNA, 25 bp, Artificial Sequence
  Antisense Oligonucleotide MTx_ASO_0326
acaggttctgccaccatgggagaga

SEQ ID NO: 346 - DNA, 25 bp, Artificial Sequence
  Antisense Oligonucleotide MTx_ASO_0327
caggttctgccaccatgggagagaa

SEQ ID NO: 347 - DNA, 25 bp, Artificial Sequence
  Antisense Oligonucleotide MTx_ASO_0328
aggttctgccaccatgggagagaaa

SEQ ID NO: 348 - DNA, 25 bp, Artificial Sequence
  Antisense Oligonucleotide MTx_ASO_0329
ggttctgccaccatgggagagaaag

SEQ ID NO: 349 - DNA, 25 bp, Artificial Sequence
  Antisense Oligonucleotide MTx_ASO_0330
gttctgccaccatgggagagaaagt

SEQ ID NO: 350 - DNA, 25 bp, Artificial Sequence
  Antisense Oligonucleotide MTx_ASO_0331
ttctgccaccatgggagagaaagtg

SEQ ID NO: 351 - DNA, 25 bp, Artificial Sequence
  Antisense Oligonucleotide MTx_ASO_0332
tctgccaccatgggagagaaagtgt

SEQ ID NO: 352 - DNA, 25 bp, Artificial Sequence
  Antisense Oligonucleotide MTx_ASO_0333
ctgccaccatgggagagaaagtgtc

SEQ ID NO: 353 - DNA, 25 bp, Artificial Sequence
  Antisense Oligonucleotide MTx_ASO_0334
tgccaccatgggagagaaagtgtca

SEQ ID NO: 354 - DNA, 25 bp, Artificial Sequence
  Antisense Oligonucleotide MTx_ASO_0335
gccaccatgggagagaaagtgtcat

SEQ ID NO: 355 - DNA, 25 bp, Artificial Sequence
  Antisense Oligonucleotide MTx_ASO_0336
ccaccatgggagagaaagtgtcatg

SEQ ID NO: 356 - DNA, 25 bp, Artificial Sequence
  Antisense Oligonucleotide MTx_ASO_0337
caccatgggagagaaagtgtcatgg

SEQ ID NO: 357 - DNA, 25 bp, Artificial Sequence
  Antisense Oligonucleotide MTx_ASO_0338
accatgggagagaaagtgtcatgga

SEQ ID NO: 358 - DNA, 25 bp, Artificial Sequence
  Antisense Oligonucleotide MTx_ASO_0339
ccatgggagagaaagtgtcatggag

SEQ ID NO: 359 - DNA, 25 bp, Artificial Sequence
  Antisense Oligonucleotide MTx_ASO_0340
catgggagagaaagtgtcatggaga

SEQ ID NO: 360 - DNA, 25 bp, Artificial Sequence
  Antisense Oligonucleotide MTx_ASO_0341
atgggagagaaagtgtcatggagag

SEQ ID NO: 361 - DNA, 25 bp, Artificial Sequence
  Antisense Oligonucleotide MTx_ASO_0342
tgggagagaaagtgtcatggagagt

SEQ ID NO: 362 - DNA, 25 bp, Artificial Sequence
  Antisense Oligonucleotide MTx_ASO_0343
gggagagaaagtgtcatggagagtg

SEQ ID NO: 363 - DNA, 25 bp, Artificial Sequence
  Antisense Oligonucleotide MTx_ASO_0344
ggagagaaagtgtcatggagagtgc

SEQ ID NO: 364 - DNA, 25 bp, Artificial Sequence
  Antisense Oligonucleotide MTx_ASO_0345
gagagaaagtgtcatggagagtgca

SEQ ID NO: 365 - DNA, 25 bp, Artificial Sequence
  Antisense Oligonucleotide MTx_ASO_0346
agagaaagtgtcatggagagtgcaa

SEQ ID NO: 366 - DNA, 25 bp, Artificial Sequence
  Antisense Oligonucleotide MTx_ASO_0347
gagaaagtgtcatggagagtgcaag

SEQ ID NO: 367 - DNA, 25 bp, Artificial Sequence
  Antisense Oligonucleotide MTx_ASO_0348
agaaagtgtcatggagagtgcaagg

SEQ ID NO: 368 - DNA, 25 bp, Artificial Sequence
  Antisense Oligonucleotide MTx_ASO_0349
gaaagtgtcatggagagtgcaaggg

SEQ ID NO: 369 - DNA, 25 bp, Artificial Sequence
  Antisense Oligonucleotide MTx_ASO_0350
aaagtgtcatggagagtgcaagggg

SEQ ID NO: 370 - DNA, 25 bp, Artificial Sequence
  Antisense Oligonucleotide MTx_ASO_0351
aagtgtcatggagagtgcaaggggt

SEQ ID NO: 371 - DNA, 25 bp, Artificial Sequence
  Antisense Oligonucleotide MTx_ASO_0352
agtgtcatggagagtgcaaggggtc

SEQ ID NO: 372 - DNA, 25 bp, Artificial Sequence
  Antisense Oligonucleotide MTx_ASO_0353
gtgtcatggagagtgcaaggggtcg

SEQ ID NO: 373 - DNA, 25 bp, Artificial Sequence
  Antisense Oligonucleotide MTx_ASO_0354
tgtcatggagagtgcaaggggtcgc

SEQ ID NO: 374 - DNA, 25 bp, Artificial Sequence
  Antisense Oligonucleotide MTx_ASO_0355
gtcatggagagtgcaaggggtcgcc

SEQ ID NO: 375 - DNA, 25 bp, Artificial Sequence
  Antisense Oligonucleotide MTx_ASO_0356
tcatggagagtgcaaggggtcgccg

SEQ ID NO: 376 - DNA, 25 bp, Artificial Sequence
  Antisense Oligonucleotide MTx_ASO_0357
catggagagtgcaaggggtcgccgg

SEQ ID NO: 377 - DNA, 25 bp, Artificial Sequence
  Antisense Oligonucleotide MTx_ASO_0358
atggagagtgcaaggggtcgccggg

SEQ ID NO: 378 - protein, 414 aa, Homo sapiens
  MISC_FEATURE (1)..(414): TDP-43
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 379 - DNA, 20 bp, Artificial Sequence
  Primer UNC13A_CE FWD 5′-3′
tggatggagagatggaacct

SEQ ID NO: 380 - DNA, 22 bp, Artificial Sequence
  Primer UNC13A_CE RVS 5′-3′
gggctgtctcatcgtagtaaac

SEQ ID NO: 381 - DNA, 20 bp, Artificial Sequence
  Primer UNC13A FWD 5′-3′
ggacgtgtggtacaacctgg

SEQ ID NO: 382 - DNA, 21 bp, Artificial Sequence
  Primer UNC13A RVS 5′-3′
gtgtactggacatggtacggg

SEQ ID NO: 383 - DNA, 18 bp, Artificial Sequence
  Primer TARDBP_1 FWD 5′-3′
aattctgcatgccccaga

SEQ ID NO: 384 - DNA, 23 bp, Artificial Sequence
  Primer TARDBP_1 RVS 5′- 3′
gaagcatctgtctcatccatttt

SEQ ID NO: 385 - DNA, 23 bp, Artificial Sequence
  Primer RPLP0_1 FWD 5′-3′
tctacaaccctgaagtgcttgat

SEQ ID NO: 386 - DNA, 21 bp, Artificial Sequence
  Primer RPLP0_1 RVS 5′-3′
caatctgcagacagacactgg

SEQ ID NO: 387 - DNA, 20 bp, Artificial Sequence
  Primer UNC13A_19_21 FWD 5′-3′
caacctggacaagcgaactg

SEQ ID NO: 388 - DNA, 22 bp, Artificial Sequence
  Primer UNC13A_19_21 RVS 5′-3′
gggctgtctcatcgtagtaaac

SEQ ID NO: 389 - DNA, 20 bp, Artificial Sequence
  Primer UNC13A_CE FWD 5′-3′
tggatggagagatggaacct

SEQ ID NO: 390 - DNA, 2314 bp, Homo sapiens
  misc_feature (1)..(2314): STMN2 transcript, NCBI Reference NM_001199214.1 Sequence
agctcctaggaagcttcagggcttaaagctccactctacttggactgtactatcaggcccccaaaatggggggagccgacagggaaggactgatttccatttcaaactgcattctggtactttgtactccagcaccattggccgatcaatatttaatgcttggagattctgactctgcgggagtcatgtcaggggaccttgggagccaatctgcttgagcttctgagtgataattattcatgggctcctgcctcttgctctttctctagcacggtcccactctgcagactcagtgccttattcagtcttctctctcgctctctccgctgctgtagccggaccctttgccttcgccactgctcagcgtctgcacatccctacaatggctaaaacagcaatggcctacaaggaaaaaatgaaggagctgtccatgctgtcactgatctgctcttgcttttacccggaacctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaagctggtcaagtttatttcttctgaactaaaagaatctatagagtctcaatttctggagcttcagagggaaggagagaagcaatgagaggcatgctgcggaggtgcgcaggaacaaggaactccaggttgaactgtctggctgaagcaagggagggtctggcacgccccaccaatagtaaatccccctgcctatattataatggatcatgcgatatcaggatggggaatgtatgacatggtttaaaaagaactcattataaaaaaaaaaaaacaaaaaaaatcaaaaattaaaaaaaatcaatgcggtctctttgcagaatgttttgcttgatgtttaaaaaataccttggatcttattttgtaaatacttacatttttgttaaaaaatacaagtattgcattatgcaagttatttcataatcttacatgtcctgtaacaggcttttgatgttgtgtctttccactcaaatgaatttgctaggtctgttctttttgaagctccccatgtctaactccattccaaaagaaaaatgaggtcagtagacagtctatggtgctagaaacccaccattgcctaatgacctagaaggctttgttgtctctgagcttgactaagaccatacctagatcacaggtattatgactccacatgaaccttcacatttgttcgctcataatctacttactgcctaaaaactacaaaaccaggctaagaaataccaccagtcatagcatttacttctgcttctcctggattatgtgctacaaatgtgctttggctttagaaagggatggatgagaagacagacctgagaccaatctgggtagaagcaaaaagttgaaccttttaaagtgctgaacacaaatccaaattcgaatggttcaagcagccgtgaaatcgctcttcataaagtgggcttaattctctagtttaagttcttttgatggaatgaattaattaatgtgtcaggtggcttatttgtggatgccatgattgatgatgttcattttaagctcttacctatagtacaagtacatgatgctactgaatatttttccacttggaaactgtgagctggttgttgcattaaaacacacatacaaacaaaatcaaaaacactgcggactttcactcaagctggtctttcttccccagtgtaaggcaatcctgcctactaacaacaccaacaacaaaacactccatctgtgaagctgacgcagttaagggggctaggcagggcatttgtgccaactaagaatcaccagatacccaccataagtacctatcgcagttttgaagtcgtttctccccaactcccaactcctgaaggttgctgcctgcatatttactcttcattagtgctattttcctgtatgtcattgtgagcaagctgtgattaataaagaattggagttctgtgaactaataaaggtttggtctgttaaaaaaaaa

SEQ ID NO: 391 - DNA, 21 bp, Artificial Sequence
  shRNA TDP-43
agatcttaagactggtcattc

SEQ ID NO: 392 - DNA, 21 bp, Artificial Sequence
  shRNA scramble
gatatcgcttctactagtaag

SEQ ID NO: 393 - DNA, 25587 bp, Homo sapiens
  misc_feature (1)..(25587): STMN2 Intron 1 Sequence
gtaaggcactgcgcctcgttctccgtcggctctacctggagcccacctctcacctcctctcttgagctctagaagcattcagagatattttataaagaaaaagatgttaatggtaacacaggaccaggaaggacagggcagttctgggggaggtgggagggcagagaagaggtctatggaaatctaaagcgaagaatttcttttaaaaggtagaagcgggtaagttgccctcctatgggtagagaatttattctgtttccatatttaaaattaggactcaatcgtgaggggaggaagctaccttaactgtttgccttaaatgggcttaagggacattttggaaagtgctttataacgaccttttttttttttatttcttctctagtttaagaagaaaataggaaaggggtaaagggaaggtgggagaaaggaaaaagaaaattgcaaagtcaaagcggtcccatcccgctgtttgaaagatgggtggagacggggggaggggatggagagaactgggcacattttacggtattgtctcgtcgaagaaaccgctagtcctggggtgcggtgcagggaggtaagacggcgggggacagggtgggggtaggacctccgctcctttgttttagggcaagggaggggaaggagagaggaagtcgcggagggcgtggagggcgcgggtgggcagctgcaggggcggggaagcgcgcggcagggaggggtggagggacagcggcttcgaaggcgctggggtggggtttctttgtgtgcggaccagcggtcccggggggaggcacctgcagcgctgggcgcacaatgcggacagccccacccagtgcggaaccgcgcagccccgcccccccgcccggtgctgcatcttcattcgaaagggggtcgggtggggagcgcagcgtgacacccaggagcccaaccctgcggggacagcggcgccacgccccgcgctccccgctcccgactccccgccgcggcttccaagagagacctgaccactgaccccgccctccccacgctggcctcattgttctgcttttaagagagatgggaaaagtgggttaacatttttcttttcggaagcaaattacatagagtgtttagacatagacacagataaagggttctttgaagacctttgatcgtttgcgggaaaagcttctagaacctagacatgtgtatgtataataatagagatgacatgaaatcgtatataaagcaaaagaggtcaaagtcttaagttaagccacgcgaaatttccgttttgtgggtcagacagtgccaaatatcggcaatttcataagctcagagagacaagacagtggagacacaggatgaccggaaaagattctggattcagggccttcatccgcaattggtcttgtgccttgagtgcccacggttctggcgctcagtggccccggggtgaaaaggcagggtggggcctggggtcctgtggcagctggaagcacgtgtcccccgggacttggttgcaggatgcggagacagggaaagctgccgaaaggactccatctgcgcggctccgccctgccctaccctccccgcggagccggggagacctcaggctccgagactggcggggaagaggaatatgggaggggcagttgagctgtatgcagtcctggaacctcttttttcagccccgcagtccacaacggcccgagcaccccttgatgtgcgcagacccccggcgtggctctcagccccagcaccgagcccctcccagccaagcgggtggctctgcagaaaagctggctcgagccccgcccggccacacaaaggcgcggccccacccagcccgggcgcgagaccgcagaggtgacccccttcccagggattcagggagggctgtctcttctcgcccacccacggtccgcggagctcggggctttttttcccccagcccaagccccccgcccaccctctgttctctatgattttccagaatggagaccccgcgaggggcttctctaagggagaccctcgctcctccagcggggcgcggctcggccccacccctcccagctgaggcccagagccgcctaccgctggccgggtgggggcgcacgtggcgactgggtgtgtggagcgcagccagccctgcagagccccgcgccgcgccctgcgctcccctccccggagttgggcgctcgcccccgcggtgcagccggggagaccggtttctgcgcagtgtcctgagctacccccgctttccacaattcgcagttcactcgcacgtccagaaaggttctgagaatgggtggtgggggcgatctcgcctcgctttctgcacccctcagaaaggtttccgctgcaggctagtggctgcaaactcatcgtcatcatcagtattattatcatttcaaatcgttgttattatttaatgattcagtagccttgtttgttctcatttgttcaaaagggacgtggattgctcttggttaaggattaacccttgttgcgttcgctttgcttcctcctaattgccctcatccctttcccccacaaaaaggtaaatttgtctccagttgttcattttaagttataaagcaaatatatttttgcttcctgccaggattatgtatgttcatgtggctaagatacatgtgcaagtgcttgctaagagcagggtttgtgtgccaacgattgctggaaaattctctgcaaagaattgtttgtggctgcaatgggtgagaatacacatatataattgagatgatcttcaacataaggttatatctataaatatataaatatagtttatgcacaaaattttaagttttttcccctgaaactgttcttccaactgctgattcttgatacagcctcaatcctacacagatacatggatcgtgaaatggtagccgccatccaaataaaaatcccaccccaaatatgacaaacgcaagcatcctttctggccataatttaactgcatttgcaaatcatgaaaaaaacactacttctgcagtattaaaataatagattttgaaattaattccaatttcaaagataattaattatcagggcgagtgcttttttcctgattcattaaacaattatgtattcagcatgattgtaagaggtgcatataatattccccattatcttttctaatgaagtgggcaccttctgaatggatatataagtaactagaaatgaaaagctgaggatttggtcagaatttcaggataaaactgaaagaaatggcagtagtttatcaattaatctcatgtatttagtttataccaggtgagtaagctgagcctgcaataaacactctctgtcccagtgtaacacgtcgcaggtagctagaatgataggataaattaatagaccttgtggtgtttgtctatgcacgttaaaattctctgagagaaagtatattttaaaatgataattaagattggacatttgtgctattaaaatctacaactttagtcaaaattcacaatggtttttttttacaataatgtgacttacagatttgtagtaaattattctattctaaaagagaaatgagtgtttttattgttacagctattacctcattaatatttttagcaaacttttatttgttgcattgaaagcagttttaattactttgggtttttatttttcaaattactaatggatagatggtggaataagcatttaatcatttggcacaatatgacttccatcaaatagctcattctcagtgattaaaaaatgctacaagaggctacaatttactcagattcaggaaatgtcctttcagagtgccataaggctgattcatataataaaatagttttcttccctataatttaagatcaaatagttacttagttctgtgaatacctagcagtagctatcaaacagaattttaaagttaaatctgtacaactaacaatgaagtggaggatgaatcgatacatattgaatggaagactttgtcattgataaattcaggccatctttaggaaaattccggatttatcaatcaccattattttttacttcaactgagtgtgactgatcacatgctcaggctaccttggtagctcattgctcacaggaggctgaaaaaagctggcctccgagcaggaggaagctcagagcacaaacctaggcctgggcgtggccactgggagctgctgatagcgaaccccagctcacaccagtttcttttttggtcgtgggaagaaaaacacatattatcctgttgtcacaagatctgtgaccttatatgaaaaaatgctagaattttttcattaaaaaagaaaatactgaactagccagtgacccagatgttttcagaacctagactggttctgtccattggaaaacctcggtgtctgcattaacttttcaccacactagagggcaatcatgttctctaaaaaagcagatgattgatgtaaacctagttccaaatattaactgtttaataaaatcttttcttttaccaggaacattcaagtgtttattcaataagctgatgccatgctttaccctagtggatgaacagagcttgtacaattttcaaggagacaggatgaaatgagtggtcataatctgaaagtagatacacgccctggttaattattccctgatggttttacttctcagttttattacattgttattataataccatttatgttacttctgagattttgtagtggataaatagtagaaaaatgtcagtagtaatagcaaagttatttagcagccgaatattttaatgcttaaaaataaaggaataaattaaagaaaatcattgtttacttcttcatcgattgaaatgtgccccctgttcagagcacatctgaatatcagagtctccacctgcagagaacatgcagcttagcgagtaaaacaggcaggtatgtgatactgaggaggtgtaccaaaaactgactgctgttatttttcccatcttctaagtctgtctttcttttccatttaaagatacctttttaaatctaatccaatgtgatttcaatctagttttatcagatttcaacaattattgagcatctccttgtagtggttttctgtttattagaaaatcgatgttaattttaacgaagtaagaagaaatatataagtataaactaattttgggtatcatcaaaagtggattttttaaatatgcattgatagaattattttttgattacattttatgtaattctaatccagctataaaatatttaatagtgtcatattactgtgttcctcaaactttgatgtgcatatgaattacctttgattttcattaaaatgcaaattctgattcaatacatctggcttgaggcagacattctgtcttccgaacaagctcccagatgatgctgattctgaccactaaacacatcagttttagggatattaacttgtaatatacaggtatccctcctggtaagctctggtattatgtcttaacatttttaaatctatggtaatctttacaaaatattttacttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcctgaataccatgtgagaaaattcttaagaatttattgagtatgactgtatatttgaaaagagtgttttcttctgcttatctaagccaataaaggatcttcattattcaattctaactttctaaggaagtcaacctacagatcagaaagaggatcttcaaggaatagcatcaaagacatagtcaggtctcccatgcagtgactggctgaccatgcagccattaccacctttctggaaatattatgctgcaaaaatgatacaatacacgaaatatctcaaattaaaaaatataacatttcccaaatagggcactaaaaacatgatcccaaataaaactagcttcagggtttgcagaatatactgttactcaacacaaagttggactaagtctcaaagttagccattcagttgttgttaacagttcatttcagggtctctcagaagctgggaaactttccatttttgcaatttcttgtacattgaaggaaaggaagacacacttaagacagcattacaaaagtaattcatgttttaaatgtttaattctggcagtcgggcagggctctctgtataacctcatttggagatgacaaaaatctaaacttgagggcctcgagccaataagtcttcctatttctttactcaaacattttcccgcaatggtgctttctttcaactgtttttctggtgtattcataaattccagattctctatgggaagtaacttttattgattgatttaacccttgtatagcacatataacatgcaaggcattgttctaagaactttccacatattaactgtgttaatcacttaataatcctaagtaggttctattacagatatggaaactgaggcacagaaagttgaagtatcttactcaaggtcacacagttagtcagatccagaatttgggcccaggccatctggcttcggaatccatctttcaccgattgctgctagtctcatatctgttccatgttagaggtgagctcccattgcagaggtcacacctgtgatatcaccattttatttaaacagaccagagatggtcttctcctttctgatcacagactcaccttgaagagaaaatacttccaaattgatgcctagttttaatagcttacctggggcttattcaaataattgccatgatttaggctttgggagaaagagagctatgaggccgtgtgggttgtaacgtatgagacacatggcgttctgcaggctcagcacagcatcgatttctggtgggaacacactctgatgaccagttccagaaataacattgacttaatctcctcagtcccatcatggttagcacatttcaaaatgcctccttaactacttccataggccagagatatttagttttaacattttgttgaataaaataaatttacacattcacatttaatataactattagatgttatttcaagattctcttcatattaccatcaaagcaggcaggcaggcaggagagaactgtaggaaggttttgaatcccttgtgaaacatttttaattatcttttaataaaggaatcaggccctgtcatttgtcaaggagacatttgcagtagtaaagcttgtgtttataatatccatttttattagtcatgattaaagataacatttgtgtacatttgttctcacaaaacacttttatatgagtgtaaaggttaattaatgcatttcagccatcattttgctggtcatgtggaaatatagcttctttaggaattgtacttagagtaggagccacatattatactataaaaccataacaaaaatattttaagtttgttctcacttgttgttgacctccagagtaaaatatttaatactctggaaagttatgggtttcaaaatttattttatggcaagaaatagataattacagttctcatagagcacatttaaaataatttatttttatagggcaaaaatattgcctaggactgaatgatttttttttttttacaaagattgtaaagcaacgcctgcaagagtgcccatttagcagttattcttctggaataattgtattttggatgttggagttcgcacattaaccattagtacaagtacccaatataacaatagatcatcaggataataaatctgtccatcttttagttgtatgtctttatatcaggataaagagaattgagtgaaatttatctaaacctagtcccacaaatacttttacaagagagcatgttaaagtgtaaattaaatttttattagcattctactctgtctttggaagttttttttccttatgaaatgcagccataaagtttaacttccattaacaaagctgctcacagtaaacctattataataatagtttcccagtttgggcttcctagtgaggagcaacctaactcacacgaaacaaccccaacttataatatattgactgttacaaaactgagaccagaaaatcccatcaagatggtactgttatcatttccagactctcgggaagaacattaatcatctcaggcacttttaggatagacttattgcagcctccctgggaactctgcttcagaacataattatttttattaatgcagagttactttttatttccaacaaaaatatctattgttattatttaagtcttacagctttatctgagaaattccaattagcacccttctcataataaatattcaaacacatgaaaaattaccaaagttgttctagtcttttaatgacatattacatgatcctgcactcttgtcactttaaaaattatctttttattatatttctgatgatttttttcttatatagttttttaaaaggagcaggcaagcatagaagactaaaaaatgttcaaaagaaaaattaaatcgcatgatctatctatatgggaccttgtcatttttagaaaacattcacctgcttcatccttttgaatcttcatataatccctctgagatgggcatactatacaagttgtcttatttaaagattggtaaatttaagctcaaataatttattcagtggcaagcctcagaggcagactcggaacacaggtctaatatatattatatatatattataacatataatatatatattacatataataaagttgtgtatattatttacctatcaaaatatttatatgtaatatataaatatgttatatatcatgtatgtgcctatttcatacatatatacacattcatgcaaaataaggtttagcactccctccactgtcctgtaataaaacatgcacagtgagaatagtcatacacgaggcatatttgtcttcagtttaaagtcattgatagtcagtgtcactaactaaagtaaaatagattggagcaccaactttgttctgaagcctgtgccaggtattatgagaacaaaaataaaaatgttcctcacccttggtggatttagtcttttgcagaaaaaaagatcctgtacatgtcagaaagttcaatagtaataatggtaatttataactataaatggaagtcaccatctcacaatttcaccatcttaacaattttgttaaactgccctacaatattacaagatagtacataatgatacactagtaacatcaactaggaagtaccaagatccaccaaaaggctgaaaaatttaaatatttaatgagtccatcaaccaatctggccagagaattctttaattaaaatgcttcccaaattttactgagaatcagcagcgtttgaggagctagcctccacccccagaggttctcactctattaggtctgaagcaggtcccatggatttgcatttctaacaagctcccaggtggtgctgatgaggctgattcagaaccacacttggagtagacctaaaacagcagtgacctgtagggtccccaagcagcaggccaggacagcatgtgagttacgtcctctgtggagctctgcaacaaggcgtcaagaggtcagagtctaagtccccatcagctctgcccttctccaccagtgctgctggtgctgcatggaaggaagagcccagaagggattctgagtttcagtctttactcttgctgacgcaccttggtcaggtcaattttcctgtttgttcctctaattcagcatctgtaaaatagccatgtgaactgccttgtccatatcagagggtctttttcagactcaaggaaaaaaacgtgaaagtgattagtgtctgtcaagtagtatataaatgcaagaagttgagtttttaaattgtcattagatataaatacccatgtgcatgcatttagaatgagtaaagagggaacaaggagcgcaatcaaaaactgcgtcatttgctttttgaaaaatactttctatgtaatgaaaagtgaaataaaatgttaattgagtccctctgacaacagcatcagacgttttgcagttcttgtgattagaacccacctggccagcccttcttcctcctaaagaagagccttcttcttcttaaatgaaggttggctcagaagaagcaattaactcattcaacgttttgttacagtcaatccacatccaacttttccccaactcaatctgctttaagggaaggatggtaagtggtggcccaagatggcaaccatcaagcttagagaatctctagaagcaggggtgtccccagcaagtagacactgaaaatatgagagggctgataagccagagataaaactcagtacttactttgcttctagtccatgtctacccctttcttggcaccaccttgacactaccctctgagtccaccttcctgagatggtacaaactctgcttagacaaagcagcccatgtccaaaggtgttagggctcagtttaaagctgccttcaaaagttaaaacagaagtgtaaagttctgtgcaattaaaaataatcagcttgtcttggaactcaaacgaatgtaaaatcctatgaaaattaaaaagcagtaccacaagttaccccaaaagtccttaggtcagtaactgttcctgttacaggtaagagagagcatggattagaggtgggcgtgggtatccagtggacatggttttgaaccatgctccactactactcactatctgagaattcttaaatttattaatcatttctatattataattttctcagttatgaaatgggaaaacaatacctaaatcacatggttgttaagtaagcaattgattgttaagcatttggtcatcaaaaatattaatccccttccctgattccctagataaatgatgaaaatactaaataaaaataataaaaatttaaagtgaacatctcaattcttatactttgttaatttctacatgtattacaaatctactagaaattacttggaattgaggaaatgattactgcttaataattctttgtggtagagggagagttggtatcatatttatgagacagcagccaatatagtatatctcaaaggaaaaaatccattctacataatgccagaatttaatagttaagcattttatctaggtcacagcacaataagcaagatggataattaaaataaaagtatatttctcttgcatatatttctcatttcatgtttccctatcatattttatatcttaccttacttcaaatacatatataccttcaataaaactgagccttcttgcttacccaggaagtttcatcattcagtagaaataaaagatgactttagaaatattaaaatacaaaaatctacactgaggtcttttgaatgcaggaaaaagaattatatcacacacacacgtacacgcacgcatgcatacacacacacagaacctctcgttctttcttaacatcttatcaatccatcagtttcactcccactccgtatcacctgactgtgcacaatatctcattgccacctcccagtcttctccctgcctggcaccctcctgctctcctgcttccactttaaacacccttccttcagctaggtcttttctttcagggatcctcccgttgctttcttatctggatcaatttagccttcctcttctccacccattagtggataagcacgacaaagacactagagtcaaataatacaaacagaatataccttagatgagtatggtgatgaaaaggatatggatacttagagtttagcactattctctcagccactcaggaaagcaacgcctttacaatcaatagtgtttcaggtaccaatcaataatctgttattgctatttttaaaatctataaggtatcagtaaaatgtaattactagagcaacaaagatatcttgtgaaatcaaattagtattcatccagcaactgagtacaaaggtttaagggaggataactaccaataccaaaacattttaagcattttgttttgcctcctaaatatcaaatcatgtaaatgtgtggtacataaattaggaattatatttatgacatagctgcagacatattaagagaaatatgtgcttatatttacaagtatagtacagttctttttcatattagatactgttgatgataatctgcatataaaaatgctcaatattttttcacatttataagccataaaatacagctaataaaatgtgtttctactttctcataaacatggaatagtgacaaacaaggagctttatatgaaagcaccattacaatttaaactctcacaaggtcataatatattgcactaagcaggagagttcagcttatttaaaaaaaaaaataaactctaatgaggttctggaatgcagagccaaagcataaagatggaaataaaagaattgcatgtcttctgaactgacttggttgatgatttttttaaaaaaggttttgtgtcttctgacttggttgatgattttttaaaaaaacgttttgtggtagaacaaataaggtaaatgaaattcagtatttaggatgaaaagtttttctaatttcaggaacaacattgaagaaatattgaactaagcagctttgaaagaatcagattccatttgttgaaatttttctgagaatgaatttttttaagacagtgtacacagttgcagtgtgtattggttatggattgtggcaagctatattacaacttacccaagaaataaggaggctgggcgtggtggctcacacctgtaatcccagcactttgggtggccgaggcgggcggatcacgaggtcaggagatcgagaccatcctggctaacacggtgaaaccccgtctctactaaaagtacaaaaaattagccgggtgtggtggcgggtgcctgtagtcccagctactcgggaggctgaggcaggagaatggcgtgaatccgggagggggagtttgcagtgagccgagattgtaccactgcactccagcctgggcgacagagcgagactccgtctcaaaaaaaaaaaaaaaaaaaaaaaaagaaagaaagaaagaaggaaaaaagtcacttgaaaagaatactggactttgtgtccagcttgcatagctgaaaagaataaaaacctgtccacttaaactcattgcaaaaagaagatgtcactcctacaaatagcaaagagtcatgaaattattctatccagaaaagtatacatttcatccctttggataaattttagaagtgaactatgaatacatacggtgaggatagccagctaagaagtcaagaaggatttctcaaatttgctgctcagaaagatcatactctccacaaaacaaataatagcaggctttccaagtcaaccttgaatccagctttcctttatctttccttcttgtgaactttcactagtttactatctaacaatgaatttgacgatagccacataccatcttatagcaatatttgttatcatatcccttgttatttatcattcacctgctctgcttgagccagctacaagtcacatgtcccacgcactttttcctgtttgattttttacagcactttgagacatgtctcattattcctacttgacaggaaagaagccatggaaagttgagtgacttgctcctgatcacaaatgctggccaaggaagagtcgagtttcaaatctaatgatctttccactgcactctagattcctcattttgaactatttttttattttttgcactatagacttttttccacattttgaactgttttttattttttgcactatagacttttctcttatacccaactatattgatgacttcttttaggctagaaacttgtttcacttactttccctttcttcagattgctgcaatattggccaacatgtattgggtacttactgagtcaagtactgtgattgtgccaagtatcttataggaggattatcatcctcatttttacaggtgagaaaggaaaggaggtaaagtcacacacagccaacaaaaatggtagcaccaggatttgaaacaaatcagtctgacccaagttgactttgttaaccactgtatgcacagtcttcttagacatagtaagagctctaattgtgtttggtgatttgattattatgacaaagtaagtaagggaagcagggagaattataagaaataaggctccacaacacttggctatagcaaagccccttaaaacttcaaaaggtcacccaaagaataaagatcaggctgggagcagtggctcacgcctgtaatcccagcactttgggaggccgaggtgggtggatcacctgagttcaggagttcgagaccagcctggacaacatggtgaaaccctgtctctactaaaaatacaaaaattagctggatgtggtggttgccgcctgtaatcccagctacttgggaggctgaggcagggagaatcgcttgaacccaggaggtggaggttgcagtgagccgagatcatgccactgcactccagcctgggcaacaagagcaaaaaactctgactcaaaaaaataaataaatcaatcaataaaataaagatcaatttggagaaattaatgcttattaataagcaatgtcttgcacagcacttcagtttctcaatacattacctaactcaatccttacaacaacaccctatccccattttgtggataaataaactcatgttcagaaggttgaataaattatctaaggttaatagttcctgacctagagctcaaatcttcagtttctatcatattcttgcccttaccctggggtagctaacattcactcactagtattggagctaaaataagggagagaacatataaatgaatacaaaggagacattcacctgccttctctttctccttacatagagaaggttgattatctgctattgtgaagtttgccttttgaaggatagaaatgagaagactttcttaaattttgcctctacgccaagaaattagagtggtaccaccagtagttccattttcaaactatcactgtagctaaagctatgtggtaagggccaaggaaaagaagtattcttgcacttcaaaatgcactgaaataccagtcagtagcataatataaaggaatttagtggagagaagagttgacctcaatctggctccaacatctcggctcttaacccctaccctacacttgttcttcatggggaagctaattgggccactggaagattcagcagctaccatttgcagctgagggacagcccctccctgcttagcaaccaatggatatgcatttatggaacacctgctaactgcgacacacactcctatgtatgagggaaaatacaaaaaatgttaaaggagatgccttcccttgccctcaggaaacttaagtatagttgcaaagaaatgattagcagcaaacgaaaccatggagaagtaagggctaaggtctgtgaaacaagcctagaaaataaccttgtccttgaaaaacacaaaaagaaagaaagaaagaaaagaaactccaaggcccttgtgaaggaaaccattaagtttgcttcacttctgtgtttaggaagacacaaacccagtcttaatgaacctcaaggccacaactactggagacatttaggaattgtcaccacattctaatgtatatatcctctgtttggcccttcctattaatattttgtaaaatttttgaagatatgagcaatgtttaaaaccatgaatccccctttttttataagtaatatttaggctgaataaacaagagaaaataggacataaaggggagccaacgtgtgccttcatttataatgtattcccaagttgtgagtttggtttatcagcaatttatcatgccaaattccaagtcatatttatctatgcagatcaaacacttgattctatttttgccttaatttttttattgggtatgtttatgaccaagtcatatggtattttctgtgacagataaaatgcacaggttattccaatctggctcagccagtcatagcaacatgtagtccttctcatgtcttaagaatgagtatcaagaattcaaagggagttccagatggcatccaaaaagcttacagtttatgcatcacttattctaacagtagaaaaagaatatttgaagccaaaaatagaccttgcatgtagcatgtggaagagtagaaattgccctgatagttaaacaatttgaaattcaagacattaatttctttatgaagcatttgtcacatcataggtaatattttatgcctatcatatatatacttattatgaaatacaaagaaattattcattctatctaagactttgtatcctttaccaatatctctccattctcccacctccaccctagcccctggaaaccacccttctactctctgcttctatgagttcttttttagtgagatcatgcagtatttgtctttctgttcctgtcttatttcacttgacataatgtccttcaggcttatccatgttgtcacaaatgacagaatttccttcttaaggctgaatagtattccattgtgtgtatgtagcacattttctttattaattcatttgttgatggatactcatattgattccatatcttgggtcttgtgaataatgatgcagtgaacataggagtgcagatatctttttgacatactgattccactttgatgggatatatacccagtagtgggactgctggatcatctagtagttttatttttttttattttttattttttttattttgagacagagccttgctatgtcgcccaggctggagtacagtggtgccatctaggctcactgcaatctctgcctcctgggttcaagcaattttcctgcctcagcctcctgagtagctgggattacaggcacgcaccaccatgcccggctaatttttgtatgtttagtagagacggggtttcaccatgtctcgaactcctgtcttcaagtgatccgtccacctcagactcccaaagtgctgcgattacaggtgtgagccaccacgcctggcctagtagttctgtttttaattttttgaggagcctccatactgctttccataatggctctaggaatttacattccaccagcagtgcacaaggattgcttttctccacattctggctaaccagtctcctgtctttttgagaacagacatttcaacacgtgtgagataatatctcattgtggttttgatttgcatttccctgatgattagtgatcttgtgccttttttcatataactgctggacattaatatgccttcctttgagaactgtgtatacaggagaaaataatcacttctcagaggagctttcatttcaaaatatccgggaaaaaaatagaaaaaatggaaaatttatcctagagtaagttgtcttttatattttgaccctgtttgtgacataaactggatgatacaaaactggaatgcaaaggctttaggaggattacttacttacttgtatattgctttaggttgtttgcagaaaattatactaattgaagttcaggctatgatgtgataaaatctatgtcaggagatgagtctacatgcaaagtttgaggaagtgacatttgagtttcaaaacaaaaaagcaattttcaatgtcatatctaggttaacccaaaagatttctttcaccctatttagctgcctctaagatggatgctgaggataattacactgtagaacaataggacgatgcttcacactcacctcacaggctctgttattcccacatactgccagagatactccaaaataaaatcactgcaacatcaggcagttataaacctcaacggtattattttctatttatatacagtatattttatattttacaagtataaaatagaatatatttattctattctctttgacacaaagtgaccataagacatattacttaagtatgactagcaaagtcatggggcttgtcattcaggaggaaactcttaactaactgttcagtttttgttcactgcaccatttacataagccaaactaatgcttcacactgtgcaaaacaatgcacagtgttgtgaatgaatggctaaaataaaactctaatgagtggggtttgaaaaatgcaactttagaaaactgttgagaaaatgttgcacactgcgcattttacaaaatttcgttgaaggacactggatattctttttaggattatggagggaagcaaaattttggctcctacatgcagtttttgtggcctttgcctgaaatagtcatctcccattaattatttagatatcattcatttcctaagacaacatttagggagactgccttaagtacaatttgtacactacccagataagaattctttttggtgaaacatcgataaatattacttggcagtaacaccaagttaaaatatttgtttcacagtcgacgttaataactattatagataaagtgaattttataagacatactcagatctaaaacagcaatatggagctcttcaaatccattgaaacttcataccagcctacggaagtagaggtttttatgcaaactcttcaagaaatatgctctgaacttttaattccttagattgatagaggaattaaatcatgatataactaataggtttgtggtacaaattgctgctgcttaatctgactctgtgtcttcccagtgttctatatgaattagatattccattatctaaagacaatcaaccccatcccacggtgatagctctaggactccctttgagttcattaaatctgtattctcagtctccaaacttctggttaattcaaacagaaaagtcaactggcccatgaactaaaataaagtcatctgaattttttttttattttgcagtgtgataaaagtctcgcactttttatttctgaaagtttctgctttcactgagagcataataggctatccacccttatgcaatcttacatacaaagtcatagtcaggctaaattcaaaaacacatgtgagatagaagtcaacgtttattttctggagaaaagccacacattacaacaaagtgaacaatgaagctggcatccttatcactggtgaccaaaacatttgtgactctggacattggccccacaaatgcgataaacattctgcataggaagtgagttttgctaattaaaaatggatccaaaatactttctactcttcagccaagaattaaaaagtaatagggaggaattgaaatcacttgggtgctacattgagccattctggagaagcaattcagagaatgtcatggcagcctcaaattgctgctcaggagcatcccagcttagaagattgcaggaaaggaagagcaaagtcattcttacatgagaactgtccttaaccagatgaatagactctccattttttaccctggctttgtctcatttaagtcccaaccaatctagctatcattttaggttttactacctgctagtatttaggagcttagggggataaaaaaatccctcaatactcagaattagacttggtgataaaaatcttgacacataaacagaataaagcgctttcattactcctctaaaccacagtgtcatttggtctctatcaaggactgtaagaatttctttcatcaggggaaagaaaaaaaggacaagagcctgcaagatgtagcggaactctcattaaacacagcaggagctttaactggaatccagagtaaggtgaggtaccaggttacaacaatttactgcttttattacaattttgatcacaaggactgattcatgtcatctagtttcttttccttgtcactatcactggtgctaagaatacatcaaattgaaatttaagagcctcatatgtttctgtataacccagtgatgggttgtactgctttgaccttcttaaatgtccctttatttcatttgatatccattcccatagaaaaactataatgctttggttggtcaaaatattaatctttcaaaacctccctggcttagaaaaccaaatttttgtagagagagatgggtagaatctaattttattctaaagcaattagcattacatcatcacagcagaaatatctagaatattacctcatgtcagtgatcttctgatatgttaaaaagggtattttaaaatctgagttatttctttttctttttaaagttacatcattaattacatactcatcaaccaaaatattttatgctccaaatttgaaccgatatagtatgtaagaagtgttcaaaatgaaattattttggtctattttgtctttgaagaagatcacagggatggacctcccaaaaggatttttaaatgggattacatatctgacttttaaaaaaaattatctgaccttgagttatagtgccccaaagtaagcaaagttccaaacacacagtatcatcagaattgagttaaaattatcaccaggggcttaatttctgaaattaaaaaggaaatgttatttccttatgaaaagaaaaggaaccaaaaatgaacttcaaggtagctgatttctgtctatgttaagacttaggtaatgggagaaagggaaaaggaaggacagaattaggagaggagcagtgtttaacaattgcgggtgcaagactcaagttttttagaatccattagcagagaaccctatttctcccattaactgctgtccttttaaatcctggcaccagctctgaggactgcagggtccatagctagtgccccactctacccagtttaaagacaccactgcctggaaatgacaggggtttttttcttaaggaaagaggtgctttctgccacgtatatataaattggtaagcttcaaataaagtgcttttgtcctttctgtctatcagaaactgtgcaaatcgaattgctgtaaaaccaagggcaagagacatcaatcctgcattctatagcatctgattttatcctttatccccaggcacatttcaaaaggaaaaaaatgaggttgcatttaaattgagtatttgggacttgccaggaaaacctcccgctagactaatatgattgcagggaaaacaagagaaaggaaaagtggagagggagtgtgctaacagatcctgggcctcgtcagcagagccgtcctgagcacaaggccatggtcagacatctggtcccgcgaatgacgttttctttatggtcattaagaacaccagtgtgtcgggacacaaacaagtattcctttcagggattatgacacattttctcccaaagtagtatattaatgacatttccagagcattctttactatcttttatatgtgatcaggaagactaatacatatcactacttcttttacacacagcattagccaaaactaaagtgtcaaatacaattttgcctaggatgaataaacagaagaaatttttatgatactgcactatcaattccaaattaaataacaacaaaatgataagtgttaaaattcatattaatgattgttcccacacaagccggaaaaaatctttctaagaagtctttcatgagttaatcccatctttcaaagtgttcagtggctccgaattcagttactgtttcctatcagttcttctttcattaagtctcttcccttttttttctctttgcactatttcccttagccgggtacataatctgctgtgctttattcatttgtgtcttaagtttgtttcccgatgacatacctttccagcaacgccatctggggagtttgggcaactgtaccacgttaggaggaaacccttcttcacaggagagtgtgcctttgctgcagggaaggaattaggatttgcttggactgtggttgcagctggcttttaaggatctccttagaatgcaagcaactcatcaatgagaatctctgcaatggttgtcactgggtagagtcatgctatgtggggtcatagcctttgaaacaaataacagtaaagataaaaatgctattaaaggaatcaccacccacagaggttaactgggttttgtccccagaccacctcgaacaagaaagaacatttttatcagtcattttcttagttttagctgataaaacaaagtaccatagactaggtggcttataaacaacagaaatttatttttcacagctttggaaactggaagtctgagatcaggccgccagaatgatcagattctagttagggcctactttgcttttgcagactgccaacttctagctgcattttcatgtggcaaaaggagattgagctagctctctggtctcttcttataaggacactaatcccattcatgaaggcttcaccttcatcatctaattactctccaaagaccccacctccaaatactatcacattgggaattagatttcaaatacaaattttgcggggacacaaatattcagtccataatagtaatgattactcattatacatagggctctaaatgtgctagcttctgatagtttttacactcacttctctttattagcttgtcaagcataattagggcagtggccttactgaaaattattgaatttagtttcctaaggacagatattgaggagttttttcttcactaaaaattcacgttccgatacagctttcatctgttactactttgtgagatggaaaatcttttattttatttttatgtttggattgacccttcttaataaagtcggcatgtaatatgcttcatgtgtttctaatatgtgcttaattttgcaaaatgttttgcataccagaatgcatttctcttccaaaaaaggtaccagcctacaaaaccttgctgttactgttttcaattagttcatggaattaaatgtattaaatgttttatgctctggcagaaattatgattctcacttaactccatataaatctggatctgcctgggcctttataagtgacacaatttcattaactgaataaacaaatgatacaaagaaatttggtttagccttctaaaattccaaaggcgttcaacaaaatatctcagaatggatgttccaggacttttatggcacaggacaacatgtattgcttattttaagaaaataagctaaatagtgaggggattcttttagcagatcctcaggatgtgttaggttgaatcataggcaaatgatatttgatcattgcacctgttaacacattgaacctcatcctaaaattgtagagctagaagaaagccttctggcagtttttaaatagattgatttactgcaatttatccagaagcttcaccgttgtcactggctacatgtgactttggcctctgtggggctatatcctcatttgtaaaattggtggtgaggtaggtggacagttgactaaataatctcttagaataattctagtatctgtggatctaaagcatccaggggttgaatatgtttctttctggccaagaaaagatgcacctgtcaataatgcccaaactcatcttctgagaatcctctttcccaagatacccactctcccttgggttatattatagtaatgatcagaagcccctgccaagaagaaactgttaacctgggaggtctatattttatttcacagccatctgtttatactttctcacaagttagtgcacagtatacccatcattttctaccattttccttaatttattaattttactaattgcataattaacaaaagtaagaagattttacctccttatccccatctggtagtttgcagatacttggcctgatgacaactgacagtgatgagatactcaccaagtttaccagggcaggaggcttcctagagaaaaaatgagaaaatgaaatggggaaggggagtgaaggattgaggaggtgacaatctggactcttgcaactgcatggcaaggttggcacacaagctgggttgcaacggagggaaggagatccttatcagatgtaatcagagctcagatcgagggctttggtgtgtgtagaaagagggagagacaaagaacttaaaacagagctgccatttgaccttgcaatcccattacttggtgtatacccaaaggagaataaatcattctattaaaaagacacatgtgcttgtatgttcatggcagcactattcacaatagctaagacatggaatcaaactaggtgtccatctatggcagattggataaagaaaatggggtaaatataaagcatgcaatacaacatggccataagaaaaaatgaaatcatgtcctttgctgcaacatggatgcagttgggacccataatcctaagtgaattaacacaggaacagaaaaccaaatacagcatgttctcacttataagtgggagctaaacactgagcacacatggacataaatatgagaacaataaacactgtggactactagaggggggaaggagagaggtttgtaaaactacctatcaggtgctatgctcaatacctgggtgatgggatttacaccccaaacatcagcatcatttaatattcccatgtaaaaagactgcacatataccccttgtatctaaaataaaacttgaaattaaaaaaaaaagaaagaaagaaagaggctggaaatagaggctcacacctgtaatcccagcactttgggtggccaaggtgggtggattgcttgagcccgggaattcaagaccagcctgagaaacctggtgaaactctgtctgtacaaaaaatacaaaaattatccaggcatggtggagcgcacctgtagtcccagctaatggggaggctgaggggggaacatcacttgagcccaggaggtggaggttgcagtgagctgggatcacaccactgcactacagcctgggtaacagagcaactctgtctcaaagagagagaggaaagaaaaaagaaaagatggacagataagaaaatgcacttggagattaagagaaagcagcaacataggaccctggataatgtgtttgcttaataactatcctgatgagttatctgactattcccaaatgagtacgtggcaattcaggctgaaccatcagagtagccctccggaatcttacttatgtacaatagacctgcatgcacatttactagaatgagcctctctctctggtaatcatgtctgcttccactaattccatctgtttcctctctctccctcctatcctgctagatcttaattccttcgaccttcctttgtttttctaactccctttctttctcttgttatttaacctgctatactatgcaattgatctcctctgcactaaggaacatgcacttcagaattctgttgacatcttgcattcctttatatttagtgaaagaatgcaaaggagtctacctggcaatattcactctgcaggaggcaataattattattcaaattaaaggaagcagtaaagagaaattcagaaaaaatgaaatatactaatcttcagcttttcatttcag

SEQ ID NO: 394-396 - skipped (3 entries)

SEQ ID NO: 397 - DNA, 20 bp, Artificial Sequence
  Primer GAPDH FWD 5′-3′
gttcgacagtcagccgcatc

SEQ ID NO: 398 - DNA, 19 bp, Artificial Sequence
  Primer GAPDH RVS 5′-3′
ggaatttgccatgggtgga

SEQ ID NO: 399 - DNA, 23 bp, Artificial Sequence
  Primer RPLP0_2 FWD 5′-3′
tctacaaccctgaagtgcttgat

SEQ ID NO: 400 - DNA, 21 bp, Artificial Sequence
  RPLP0_2 RVS 5′-3′
caatctgcagacagacactgg

SEQ ID NO: 401-406 - skipped (6 entries)

SEQ ID NO: 407 - DNA, 19 bp, Artificial Sequence
  Primer mCherry FWD 5′-3′
gttcatgcgcttcaaggtg

SEQ ID NO: 408 - DNA, 19 bp, Artificial Sequence
  Primer mCherry RVS 5′-3′
ttggtcaccttcagcttgg

SEQ ID NO: 409 - DNA, 22 bp, Artificial Sequence
  Primer EGFP FWD 5′-3′
acaggtactgtgcctatcaaag

SEQ ID NO: 410 - DNA, 21 bp, Artificial Sequence
  Primer EGFP RVS 5′-3′
tgtggcggatcttgaagttag

SEQ ID NO: 411 - DNA, 31 bp, Artificial Sequence
  UNC13A minigene FWD primer
aggtcatatgcactgctatagtgggaagttc

SEQ ID NO: 412 - DNA, 34 bp, Artificial Sequence
  UNC13A minigene RVS primer
cttacatatgtaataactcaaccacacttccatc

SEQ ID NO: 413 - DNA, 25 bp, Artificial Sequence
  Primer UNC13A_CE_minigene FWD 5′-3′
gattgaacagatgaatgagtgatga

SEQ ID NO: 414 - DNA, 20 bp, Artificial Sequence
  Primer UNC13A_CE_minigene RVS 5′-3′
tgtctggaccaatgttggtg

SEQ ID NO: 415 - DNA, 18 bp, Artificial Sequence
  Primer GFP_OE FWD 5′-3′
gaagcgcgatcacatggt

SEQ ID NO: 416 - DNA, 18 bp, Artificial Sequence
  Primer GFP_OE RVS 5′-3′
ccatgccgagagtgatcc

SEQ ID NO: 417-420 - skipped (4 entries)

SEQ ID NO: 421 - DNA, 21 bp, Artificial Sequence
  Primer TARDBP_2 FWD 5′-3′
tggacgatggtgtgactgcaa

SEQ ID NO: 422 - DNA, 22 bp, Artificial Sequence
  Primer TARDBP_2 RVS 5′-3′
agagaagaactcccgcagctca

SEQ ID NO: 423 - DNA, 18 bp, Artificial Sequence
  Antisense oligonucleotide MTx_ASO_1
tcaacaaactagttcctg

SEQ ID NO: 424 - DNA, 18 bp, Artificial Sequence
  Antisense oligonucleotide MTx_ASO_2
acaaactagttcctgggg

SEQ ID NO: 425 - DNA, 18 bp, Artificial Sequence
  Antisense oligonucleotide MTx_ASO_3
aactagttcctggggata

SEQ ID NO: 426 - DNA, 18 bp, Artificial Sequence
  Antisense oligonucleotide MTx_ASO_4
tagttcctggggataaga

SEQ ID NO: 427 - DNA, 18 bp, Artificial Sequence
  Antisense oligonucleotide MTx_ASO_5
ttcctggggataagagtt

SEQ ID NO: 428 - DNA, 18 bp, Artificial Sequence
  Antisense oligonucleotide MTx_ASO_6
ctggggataagagttctt

SEQ ID NO: 429 - DNA, 18 bp, Artificial Sequence
  Antisense oligonucleotide MTx_ASO_7
gggataagagttctttcc

SEQ ID NO: 430 - DNA, 18 bp, Artificial Sequence
  Antisense oligonucleotide MTx_ASO_8
ataagagttctttccagg

SEQ ID NO: 431 - DNA, 18 bp, Artificial Sequence
  Antisense oligonucleotide MTx_ASO_9
agagttctttccaggaaa

SEQ ID NO: 432 - DNA, 18 bp, Artificial Sequence
  Antisense oligonucleotide MTx_ASO_10
gttctttccaggaaaccc

SEQ ID NO: 433 - DNA, 18 bp, Artificial Sequence
  Antisense oligonucleotide MTx_ASO_11
ctttccaggaaacccagg

SEQ ID NO: 434 - DNA, 18 bp, Artificial Sequence
  Antisense oligonucleotide MTx_ASO_12
tccaggaaacccaggcag

SEQ ID NO: 435 - DNA, 18 bp, Artificial Sequence
  Antisense oligonucleotide MTx_ASO_13
aggaaacccaggcagctg

SEQ ID NO: 436 - DNA, 18 bp, Artificial Sequence
  Antisense oligonucleotide MTx_ASO_14
aaacccaggcagctggaa

SEQ ID NO: 437 - DNA, 18 bp, Artificial Sequence
  Antisense oligonucleotide MTx_ASO_15
cccaggcagctggaagag

SEQ ID NO: 438 - DNA, 18 bp, Artificial Sequence
  Antisense oligonucleotide MTx_ASO_16
aggcagctggaagagaca

SEQ ID NO: 439 - DNA, 18 bp, Artificial Sequence
  Antisense oligonucleotide MTx_ASO_17
cagctggaagagacatac

SEQ ID NO: 440 - DNA, 18 bp, Artificial Sequence
  Antisense oligonucleotide MTx_ASO_18
ctggaagagacataccca

SEQ ID NO: 441 - DNA, 18 bp, Artificial Sequence
  Antisense oligonucleotide MTx_ASO_19
gaagagacatacccagac

SEQ ID NO: 442 - DNA, 18 bp, Artificial Sequence
  Antisense oligonucleotide MTx_ASO_20
gagacatacccagacaca

SEQ ID NO: 443 - DNA, 18 bp, Artificial Sequence
  Antisense oligonucleotide MTx_ASO_21
acatacccagacacaaac

SEQ ID NO: 444 - DNA, 18 bp, Artificial Sequence
  Antisense oligonucleotide MTx_ASO_22
tacccagacacaaacggc

SEQ ID NO: 445 - DNA, 18 bp, Artificial Sequence
  Antisense oligonucleotide MTx_ASO_23
ccagacacaaacggccca

SEQ ID NO: 446 - DNA, 18 bp, Artificial Sequence
  Antisense oligonucleotide MTx_ASO_24
gacacaaacggcccaatc

SEQ ID NO: 447 - DNA, 18 bp, Artificial Sequence
  Antisense oligonucleotide MTx_ASO_25
acaaacggcccaatcctg

SEQ ID NO: 448 - DNA, 18 bp, Artificial Sequence
  Antisense oligonucleotide MTx_ASO_26
aacggcccaatcctgagt

SEQ ID NO: 449 - DNA, 18 bp, Artificial Sequence
  Antisense oligonucleotide MTx_ASO_27
ggcccaatcctgagtggt

SEQ ID NO: 450 - DNA, 18 bp, Artificial Sequence
  Antisense oligonucleotide MTx_ASO_28
ccaatcctgagtggttag

SEQ ID NO: 451 - DNA, 18 bp, Artificial Sequence
  Antisense oligonucleotide MTx_ASO_29
atcctgagtggttagggc

SEQ ID NO: 452 - DNA, 18 bp, Artificial Sequence
  Antisense oligonucleotide MTx_ASO_30
ctgagtggttagggctgg

SEQ ID NO: 453 - DNA, 18 bp, Artificial Sequence
  Antisense oligonucleotide MTx_ASO_31
agtggttagggctggaat

SEQ ID NO: 454 - DNA, 18 bp, Artificial Sequence
  Antisense oligonucleotide MTx_ASO_32
ggttagggctggaataga

SEQ ID NO: 455 - DNA, 18 bp, Artificial Sequence
  Antisense oligonucleotide MTx_ASO_33
tagggctggaatagaagg

SEQ ID NO: 456 - DNA, 18 bp, Artificial Sequence
  Antisense oligonucleotide MTx_ASO_34
ggctggaatagaaggaag

SEQ ID NO: 457 - DNA, 18 bp, Artificial Sequence
  Antisense oligonucleotide MTx_ASO_35
tggaatagaaggaagaac

SEQ ID NO: 458 - DNA, 18 bp, Artificial Sequence
  Antisense oligonucleotide MTx_ASO_36
aatagaaggaagaacctg

SEQ ID NO: 459 - DNA, 18 bp, Artificial Sequence
  Antisense oligonucleotide MTx_ASO_37
agaaggaagaacctgatg

SEQ ID NO: 460 - DNA, 18 bp, Artificial Sequence
  Antisense oligonucleotide MTx_ASO_38
aggaagaacctgatgatg

SEQ ID NO: 461 - DNA, 18 bp, Artificial Sequence
  Antisense oligonucleotide MTx_ASO_39
aagaacctgatgatgagt

SEQ ID NO: 462 - DNA, 18 bp, Artificial Sequence
  Antisense oligonucleotide MTx_ASO_40
aacctgatgatgagtagt

SEQ ID NO: 463 - DNA, 18 bp, Artificial Sequence
  Antisense oligonucleotide MTx_ASO_41
ctgatgatgagtagtgag

SEQ ID NO: 464 - DNA, 18 bp, Artificial Sequence
  Antisense oligonucleotide MTx_ASO_42
atgatgagtagtgagagt

SEQ ID NO: 465 - DNA, 18 bp, Artificial Sequence
  Antisense oligonucleotide MTx_ASO_43
atgagtagtgagagtcaa

SEQ ID NO: 466 - DNA, 18 bp, Artificial Sequence
  Antisense oligonucleotide MTx_ASO_44
agtagtgagagtcaacct

SEQ ID NO: 467 - DNA, 18 bp, Artificial Sequence
  Antisense oligonucleotide MTx_ASO_45
agtgagagtcaacctgga

SEQ ID NO: 468 - DNA, 18 bp, Artificial Sequence
  Antisense oligonucleotide MTx_ASO_121
ggcagctggaagagacat

SEQ ID NO: 469 - DNA, 18 bp, Artificial Sequence
  Antisense oligonucleotide MTx_ASO_122
gcagctggaagagacata

SEQ ID NO: 470 - DNA, 18 bp, Artificial Sequence
  Antisense oligonucleotide MTx_ASO_123
cagctggaagagacatac

SEQ ID NO: 471 - DNA, 18 bp, Artificial Sequence
  Antisense oligonucleotide MTx_ASO_124
agctggaagagacatacc

SEQ ID NO: 472 - DNA, 18 bp, Artificial Sequence
  Antisense oligonucleotide MTx_ASO_125
gctggaagagacataccc

SEQ ID NO: 473 - DNA, 18 bp, Artificial Sequence
  Antisense oligonucleotide MTx_ASO_126
ctggaagagacataccca

SEQ ID NO: 474 - DNA, 18 bp, Artificial Sequence
  Antisense oligonucleotide MTx_ASO_127
tggaagagacatacccag

SEQ ID NO: 475 - DNA, 18 bp, Artificial Sequence
  Antisense oligonucleotide MTx_ASO_128
ggaagagacatacccaga

SEQ ID NO: 476 - DNA, 18 bp, Artificial Sequence
  Antisense oligonucleotide MTx_ASO_129
gaagagacatacccagac

SEQ ID NO: 477 - DNA, 18 bp, Artificial Sequence
  Antisense oligonucleotide MTx_ASO_130
aagagacatacccagaca

SEQ ID NO: 478 - DNA, 18 bp, Artificial Sequence
  Antisense oligonucleotide MTx_ASO_131
agagacatacccagacac

SEQ ID NO: 479 - DNA, 18 bp, Artificial Sequence
  Antisense oligonucleotide MTx_ASO_132
ggcagctggaagagacat

SEQ ID NO: 480 - DNA, 18 bp, Artificial Sequence
  Antisense oligonucleotide MTx_ASO_133
gcagctggaagagacata

SEQ ID NO: 481 - DNA, 18 bp, Artificial Sequence
  Antisense oligonucleotide MTx_ASO_134
cagctggaagagacatac

SEQ ID NO: 482 - DNA, 18 bp, Artificial Sequence
  Antisense oligonucleotide MTx_ASO_135
agctggaagagacatacc

SEQ ID NO: 483 - DNA, 18 bp, Artificial Sequence
  Antisense oligonucleotide MTx_ASO_136
gctggaagagacataccc

SEQ ID NO: 484 - DNA, 18 bp, Artificial Sequence
  Antisense oligonucleotide MTx_ASO_137
ctggaagagacataccca

SEQ ID NO: 485 - DNA, 18 bp, Artificial Sequence
  Antisense oligonucleotide MTx_ASO_138
tggaagagacatacccag

SEQ ID NO: 486 - DNA, 18 bp, Artificial Sequence
  Antisense oligonucleotide MTx_ASO_139
ggaagagacatacccaga

SEQ ID NO: 487 - DNA, 18 bp, Artificial Sequence
  Antisense oligonucleotide MTx_ASO_140
gaagagacatacccagac

SEQ ID NO: 488 - DNA, 18 bp, Artificial Sequence
  Antisense oligonucleotide MTx_ASO_141
aagagacatacccagaca

SEQ ID NO: 489 - DNA, 18 bp, Artificial Sequence
  Antisense oligonucleotide MTx_ASO_142
agagacatacccagacac

SEQ ID NO: 490 - DNA, 18 bp, Artificial Sequence
  Antisense oligonucleotide MTx_ASO_248
catctatccatccatgta

SEQ ID NO: 491 - DNA, 18 bp, Artificial Sequence
  Antisense oligonucleotide MTx_ASO_249
ctatccatccatgtactc

SEQ ID NO: 492 - DNA, 18 bp, Artificial Sequence
  Antisense oligonucleotide MTx_ASO_250
tccatccatgtactcacc

SEQ ID NO: 493 - DNA, 18 bp, Artificial Sequence
  Antisense oligonucleotide MTx_ASO_251
atccatgtactcacccat

SEQ ID NO: 494 - DNA, 18 bp, Artificial Sequence
  Antisense oligonucleotide MTx_ASO_252
catgtactcacccatctc

SEQ ID NO: 495 - DNA, 18 bp, Artificial Sequence
  Antisense oligonucleotide MTx_ASO_253
gtactcacccatctctcc

SEQ ID NO: 496 - DNA, 18 bp, Artificial Sequence
  Antisense oligonucleotide MTx_ASO_254
ctcacccatctctccatc

SEQ ID NO: 497 - DNA, 18 bp, Artificial Sequence
  Antisense oligonucleotide MTx_ASO_255
acccatctctccatccat

SEQ ID NO: 498 - DNA, 18 bp, Artificial Sequence
  Antisense oligonucleotide MTx_ASO_256
catctctccatccatcct

SEQ ID NO: 499 - DNA, 18 bp, Artificial Sequence
  Antisense oligonucleotide MTx_ASO_257
ctctccatccatcctttt

SEQ ID NO: 500 - DNA, 18 bp, Artificial Sequence
  Antisense oligonucleotide MTx_ASO_258
tccatccatccttttatc

SEQ ID NO: 501 - DNA, 18 bp, Artificial Sequence
  Antisense oligonucleotide MTx_ASO_259
atccatccttttatctac

SEQ ID NO: 502 - DNA, 18 bp, Artificial Sequence
  Antisense oligonucleotide MTx_ASO_260
catccttttatctactca

SEQ ID NO: 503 - DNA, 18 bp, Artificial Sequence
  Antisense oligonucleotide MTx_ASO_261
ccttttatctactcatca

SEQ ID NO: 504 - DNA, 18 bp, Artificial Sequence
  Antisense oligonucleotide MTx_ASO_262
tttatctactcatcactc

SEQ ID NO: 505 - DNA, 18 bp, Artificial Sequence
  Antisense oligonucleotide MTx_ASO_263
atctactcatcactcatt

SEQ ID NO: 506 - DNA, 18 bp, Artificial Sequence
  Antisense oligonucleotide MTx_ASO_264
tactcatcactcattcat

SEQ ID NO: 507 - DNA, 18 bp, Artificial Sequence
  Antisense oligonucleotide MTx_ASO_265
tcatcactcattcatctg

SEQ ID NO: 508 - DNA, 18 bp, Artificial Sequence
  Antisense oligonucleotide MTx_ASO_266
tcactcattcatctgttc

SEQ ID NO: 509 - DNA, 18 bp, Artificial Sequence
  Antisense oligonucleotide MTx_ASO_267
ctcattcatctgttcaat

SEQ ID NO: 510 - DNA, 18 bp, Artificial Sequence
  Antisense oligonucleotide MTx_ASO_268
attcatctgttcaatcat

SEQ ID NO: 511 - DNA, 18 bp, Artificial Sequence
  Antisense oligonucleotide MTx_ASO_269
catctgttcaatcattca

SEQ ID NO: 512 - DNA, 18 bp, Artificial Sequence
  Antisense oligonucleotide MTx_ASO_270
ctgttcaatcattcattc

SEQ ID NO: 513 - DNA, 18 bp, Artificial Sequence
  Antisense oligonucleotide MTx_ASO_271
ttcaatcattcattcatt

SEQ ID NO: 514 - DNA, 18 bp, Artificial Sequence
  Antisense oligonucleotide MTx_ASO_272
aatcattcattcattcac

SEQ ID NO: 515 - DNA, 18 bp, Artificial Sequence
  Antisense oligonucleotide MTx_ASO_273
cattcattcattcaccag

SEQ ID NO: 516 - DNA, 18 bp, Artificial Sequence
  Antisense oligonucleotide MTx_ASO_274
tcattcattcaccagcat

SEQ ID NO: 517 - DNA, 18 bp, Artificial Sequence
  Antisense oligonucleotide MTx_ASO_275
ttcattcaccagcattta

SEQ ID NO: 518 - DNA, 18 bp, Artificial Sequence
  Antisense oligonucleotide MTx_ASO_276
attcaccagcatttattc

SEQ ID NO: 519 - DNA, 18 bp, Artificial Sequence
  Antisense oligonucleotide MTx_ASO_277
caccagcatttattcaac

SEQ ID NO: 520 - DNA, 18 bp, Artificial Sequence
  Antisense oligonucleotide MTx_ASO_278
cagcatttattcaacaaa

SEQ ID NO: 521 - DNA, 18 bp, Artificial Sequence
  Antisense oligonucleotide MTx_ASO_279
catttattcaacaaacta

SEQ ID NO: 522 - DNA, 18 bp, Artificial Sequence
  Antisense oligonucleotide MTx_ASO_280
ttattcaacaaactagtt

SEQ ID NO: 523 - DNA, 21 bp, Artificial Sequence
  Antisense oligonucleotide MTx_ASO_306
acaaactagttcctggggata

SEQ ID NO: 524 - DNA, 21 bp, Artificial Sequence
  Antisense oligonucleotide MTx_ASO_307
caaactagttcctggggataa

SEQ ID NO: 525 - DNA, 21 bp, Artificial Sequence
  Antisense oligonucleotide MTx_ASO_308
aaactagttcctggggataag

SEQ ID NO: 526 - DNA, 21 bp, Artificial Sequence
  Antisense oligonucleotide MTx_ASO_309
aactagttcctggggataaga

SEQ ID NO: 527 - DNA, 21 bp, Artificial Sequence
  Antisense oligonucleotide MTx_ASO_310
actagttcctggggataagag

SEQ ID NO: 528 - DNA, 21 bp, Artificial Sequence
  Antisense oligonucleotide MTx_ASO_311
ctagttcctggggataagagt

SEQ ID NO: 529 - DNA, 21 bp, Artificial Sequence
  Antisense oligonucleotide MTx_ASO_312
tagttcctggggataagagtt

SEQ ID NO: 530 - DNA, 21 bp, Artificial Sequence
  Antisense oligonucleotide MTx_ASO_313
agttcctggggataagagttc

SEQ ID NO: 531 - DNA, 21 bp, Artificial Sequence
  Antisense oligonucleotide MTx_ASO_314
gttcctggggataagagttct

SEQ ID NO: 532 - DNA, 21 bp, Artificial Sequence
  Antisense oligonucleotide MTx_ASO_315
ttcctggggataagagttctt

SEQ ID NO: 533 - DNA, 21 bp, Artificial Sequence
  Antisense oligonucleotide MTx_ASO_316
tcctggggataagagttcttt

SEQ ID NO: 534 - DNA, 21 bp, Artificial Sequence
  Antisense oligonucleotide MTx_ASO_317
cctggggataagagttctttc

SEQ ID NO: 535 - DNA, 21 bp, Artificial Sequence
  Antisense oligonucleotide MTx_ASO_318
ctggggataagagttctttcc

SEQ ID NO: 536 - DNA, 21 bp, Artificial Sequence
  Antisense oligonucleotide MTx_ASO_319
tggggataagagttctttcca

SEQ ID NO: 537 - DNA, 21 bp, Artificial Sequence
  Antisense oligonucleotide MTx_ASO_320
ggggataagagttctttccag

SEQ ID NO: 538 - DNA, 21 bp, Artificial Sequence
  Antisense oligonucleotide MTx_ASO_321
gggataagagttctttccagg

SEQ ID NO: 539 - DNA, 21 bp, Artificial Sequence
  Antisense oligonucleotide MTx_ASO_322
ggataagagttctttccagga

SEQ ID NO: 540 - DNA, 21 bp, Artificial Sequence
  Antisense oligonucleotide MTx_ASO_323
gataagagttctttccaggaa

SEQ ID NO: 541 - DNA, 21 bp, Artificial Sequence
  Antisense oligonucleotide MTx_ASO_324
ataagagttctttccaggaaa

SEQ ID NO: 542 - DNA, 21 bp, Artificial Sequence
  Antisense oligonucleotide MTx_ASO_325
taagagttctttccaggaaac

SEQ ID NO: 543 - DNA, 21 bp, Artificial Sequence
  Antisense oligonucleotide MTx_ASO_326
aagagttctttccaggaaacc

SEQ ID NO: 544 - DNA, 21 bp, Artificial Sequence
  Antisense oligonucleotide MTx_ASO_327
agagttctttccaggaaaccc

SEQ ID NO: 545 - DNA, 21 bp, Artificial Sequence
  Antisense oligonucleotide MTx_ASO_328
gagttctttccaggaaaccca

SEQ ID NO: 546 - DNA, 21 bp, Artificial Sequence
  Antisense oligonucleotide MTx_ASO_329
agttctttccaggaaacccag

SEQ ID NO: 547 - DNA, 21 bp, Artificial Sequence
  Antisense oligonucleotide MTx_ASO_330
gttctttccaggaaacccagg

SEQ ID NO: 548 - DNA, 21 bp, Artificial Sequence
  Antisense oligonucleotide MTx_ASO_331
ttctttccaggaaacccaggc

SEQ ID NO: 549 - DNA, 21 bp, Artificial Sequence
  Antisense oligonucleotide MTx_ASO_332
tctttccaggaaacccaggca

SEQ ID NO: 550 - DNA, 21 bp, Artificial Sequence
  Antisense oligonucleotide MTx_ASO_333
ctttccaggaaacccaggcag

SEQ ID NO: 551 - DNA, 21 bp, Artificial Sequence
  Antisense oligonucleotide MTx_ASO_334
tttccaggaaacccaggcagc

SEQ ID NO: 552 - DNA, 21 bp, Artificial Sequence
  Antisense oligonucleotide MTx_ASO_335
ttccaggaaacccaggcagct

SEQ ID NO: 553 - DNA, 21 bp, Artificial Sequence
  Antisense oligonucleotide MTx_ASO_336
tccaggaaacccaggcagctg

SEQ ID NO: 554 - DNA, 21 bp, Artificial Sequence
  Antisense oligonucleotide MTx_ASO_337
ccaggaaacccaggcagctgg

SEQ ID NO: 555 - DNA, 21 bp, Artificial Sequence
  Antisense oligonucleotide MTx_ASO_338
caggaaacccaggcagctgga

SEQ ID NO: 556 - DNA, 21 bp, Artificial Sequence
  Antisense oligonucleotide MTx_ASO_339
aggaaacccaggcagctggaa

SEQ ID NO: 557 - DNA, 21 bp, Artificial Sequence
  Antisense oligonucleotide MTx_ASO_340
ggaaacccaggcagctggaag

SEQ ID NO: 558 - DNA, 21 bp, Artificial Sequence
  Antisense oligonucleotide MTx_ASO_341
gaaacccaggcagctggaaga

SEQ ID NO: 559 - DNA, 21 bp, Artificial Sequence
  Antisense oligonucleotide MTx_ASO_342
aaacccaggcagctggaagag

SEQ ID NO: 560 - DNA, 21 bp, Artificial Sequence
  Antisense oligonucleotide MTx_ASO_343
aacccaggcagctggaagaga

SEQ ID NO: 561 - DNA, 21 bp, Artificial Sequence
  Antisense oligonucleotide MTx_ASO_344
acccaggcagctggaagagac

SEQ ID NO: 562 - DNA, 21 bp, Artificial Sequence
  Antisense oligonucleotide MTx_ASO_345
cccaggcagctggaagagaca

SEQ ID NO: 563 - DNA, 21 bp, Artificial Sequence
  Antisense oligonucleotide MTx_ASO_346
ccaggcagctggaagagacat

SEQ ID NO: 564 - DNA, 21 bp, Artificial Sequence
  Antisense oligonucleotide MTx_ASO_347
caggcagctggaagagacata

SEQ ID NO: 565 - DNA, 21 bp, Artificial Sequence
  Antisense oligonucleotide MTx_ASO_348
aggcagctggaagagacatac

SEQ ID NO: 566 - DNA, 21 bp, Artificial Sequence
  Antisense oligonucleotide MTx_ASO_349
ggcagctggaagagacatacc

SEQ ID NO: 567 - DNA, 21 bp, Artificial Sequence
  Antisense oligonucleotide MTx_ASO_350
gcagctggaagagacataccc

SEQ ID NO: 568 - DNA, 21 bp, Artificial Sequence
  Antisense oligonucleotide MTx_ASO_351
cagctggaagagacataccca

SEQ ID NO: 569 - DNA, 21 bp, Artificial Sequence
  Antisense oligonucleotide MTx_ASO_352
agctggaagagacatacccag

SEQ ID NO: 570 - DNA, 21 bp, Artificial Sequence
  Antisense oligonucleotide MTx_ASO_353
gctggaagagacatacccaga

SEQ ID NO: 571 - DNA, 21 bp, Artificial Sequence
  Antisense oligonucleotide MTx_ASO_354
ctggaagagacatacccagac

SEQ ID NO: 572 - DNA, 21 bp, Artificial Sequence
  Antisense oligonucleotide MTx_ASO_355
catctatccatccatgtactc

SEQ ID NO: 573 - DNA, 21 bp, Artificial Sequence
  Antisense oligonucleotide MTx_ASO_356
atctatccatccatgtactca

SEQ ID NO: 574 - DNA, 21 bp, Artificial Sequence
  Antisense oligonucleotide MTx_ASO_357
tctatccatccatgtactcac

SEQ ID NO: 575 - DNA, 21 bp, Artificial Sequence
  Antisense oligonucleotide MTx_ASO_358
ctatccatccatgtactcacc

SEQ ID NO: 576 - DNA, 21 bp, Artificial Sequence
  Antisense oligonucleotide MTx_ASO_359
tatccatccatgtactcaccc

SEQ ID NO: 577 - DNA, 21 bp, Artificial Sequence
  Antisense oligonucleotide MTx_ASO_360
atccatccatgtactcaccca

SEQ ID NO: 578 - DNA, 21 bp, Artificial Sequence
  Antisense oligonucleotide MTx_ASO_361
tccatccatgtactcacccat

SEQ ID NO: 579 - DNA, 21 bp, Artificial Sequence
  Antisense oligonucleotide MTx_ASO_362
ccatccatgtactcacccatc

SEQ ID NO: 580 - DNA, 21 bp, Artificial Sequence
  Antisense oligonucleotide MTx_ASO_363
catccatgtactcacccatct

SEQ ID NO: 581 - DNA, 21 bp, Artificial Sequence
  Antisense oligonucleotide MTx_ASO_364
atccatgtactcacccatctc

SEQ ID NO: 582 - DNA, 21 bp, Artificial Sequence
  Antisense oligonucleotide MTx_ASO_365
tccatgtactcacccatctct

SEQ ID NO: 583 - DNA, 21 bp, Artificial Sequence
  Antisense oligonucleotide MTx_ASO_366
ccatgtactcacccatctctc

SEQ ID NO: 584 - DNA, 21 bp, Artificial Sequence
  Antisense oligonucleotide MTx_ASO_367
catgtactcacccatctctcc

SEQ ID NO: 585 - DNA, 21 bp, Artificial Sequence
  Antisense oligonucleotide MTx_ASO_368
atgtactcacccatctctcca

SEQ ID NO: 586 - DNA, 21 bp, Artificial Sequence
  Antisense oligonucleotide MTx_ASO_369
tgtactcacccatctctccat

SEQ ID NO: 587 - DNA, 21 bp, Artificial Sequence
  Antisense oligonucleotide MTx_ASO_370
gtactcacccatctctccatc

SEQ ID NO: 588 - DNA, 21 bp, Artificial Sequence
  Antisense oligonucleotide MTx_ASO_371
tactcacccatctctccatcc

SEQ ID NO: 589 - DNA, 21 bp, Artificial Sequence
  Antisense oligonucleotide MTx_ASO_372
actcacccatctctccatcca

SEQ ID NO: 590 - DNA, 21 bp, Artificial Sequence
  Antisense oligonucleotide MTx_ASO_373
ctcacccatctctccatccat

SEQ ID NO: 591 - DNA, 21 bp, Artificial Sequence
  Antisense oligonucleotide MTx_ASO_374
tcacccatctctccatccatc

SEQ ID NO: 592 - DNA, 21 bp, Artificial Sequence
  Antisense oligonucleotide MTx_ASO_375
cacccatctctccatccatcc

SEQ ID NO: 593 - DNA, 21 bp, Artificial Sequence
  Antisense oligonucleotide MTx_ASO_376
acccatctctccatccatcct

SEQ ID NO: 594 - DNA, 21 bp, Artificial Sequence
  Antisense oligonucleotide MTx_ASO_377
cccatctctccatccatcctt

SEQ ID NO: 595 - DNA, 21 bp, Artificial Sequence
  Antisense oligonucleotide MTx_ASO_378
ccatctctccatccatccttt

SEQ ID NO: 596 - DNA, 21 bp, Artificial Sequence
  Antisense oligonucleotide MTx_ASO_379
catctctccatccatcctttt

SEQ ID NO: 597 - DNA, 21 bp, Artificial Sequence
  Antisense oligonucleotide MTx_ASO_380
atctctccatccatcctttta

SEQ ID NO: 598 - DNA, 21 bp, Artificial Sequence
  Antisense oligonucleotide MTx_ASO_381
tctctccatccatccttttat

SEQ ID NO: 599 - DNA, 21 bp, Artificial Sequence
  Antisense oligonucleotide MTx_ASO_382
ctctccatccatccttttatc

SEQ ID NO: 600 - DNA, 21 bp, Artificial Sequence
  Antisense oligonucleotide MTx_ASO_383
tctccatccatccttttatct

SEQ ID NO: 601 - DNA, 21 bp, Artificial Sequence
  Antisense oligonucleotide MTx_ASO_384
ctccatccatccttttatcta

SEQ ID NO: 602 - DNA, 21 bp, Artificial Sequence
  Antisense oligonucleotide MTx_ASO_385
tccatccatccttttatctac

SEQ ID NO: 603 - DNA, 21 bp, Artificial Sequence
  Antisense oligonucleotide MTx_ASO_386
ccatccatccttttatctact

SEQ ID NO: 604 - DNA, 21 bp, Artificial Sequence
  Antisense oligonucleotide MTx_ASO_387
catccatccttttatctactc

SEQ ID NO: 605 - DNA, 21 bp, Artificial Sequence
  Antisense oligonucleotide MTx_ASO_388
atccatccttttatctactca

SEQ ID NO: 606 - DNA, 21 bp, Artificial Sequence
  Antisense oligonucleotide MTx_ASO_389
tccatccttttatctactcat

SEQ ID NO: 607 - DNA, 21 bp, Artificial Sequence
  Antisense oligonucleotide MTx_ASO_390
ccatccttttatctactcatc

SEQ ID NO: 608 - DNA, 21 bp, Artificial Sequence
  Antisense oligonucleotide MTx_ASO_391
catccttttatctactcatca

SEQ ID NO: 609 - DNA, 21 bp, Artificial Sequence
  Antisense oligonucleotide MTx_ASO_392
atccttttatctactcatcac

SEQ ID NO: 610 - DNA, 21 bp, Artificial Sequence
  Antisense oligonucleotide MTx_ASO_393
tccttttatctactcatcact

SEQ ID NO: 611 - DNA, 21 bp, Artificial Sequence
  Antisense oligonucleotide MTx_ASO_394
ccttttatctactcatcactc

SEQ ID NO: 612 - DNA, 21 bp, Artificial Sequence
  Antisense oligonucleotide MTx_ASO_395
cttttatctactcatcactca

SEQ ID NO: 613 - DNA, 21 bp, Artificial Sequence
  Antisense oligonucleotide MTx_ASO_396
ttttatctactcatcactcat

SEQ ID NO: 614 - DNA, 21 bp, Artificial Sequence
  Antisense oligonucleotide MTx_ASO_397
tttatctactcatcactcatt

SEQ ID NO: 615 - DNA, 21 bp, Artificial Sequence
  Antisense oligonucleotide MTx_ASO_398
ttatctactcatcactcattc

SEQ ID NO: 616 - DNA, 21 bp, Artificial Sequence
  Antisense oligonucleotide MTx_ASO_399
tatctactcatcactcattca

SEQ ID NO: 617 - DNA, 21 bp, Artificial Sequence
  Antisense oligonucleotide MTx_ASO_400
atctactcatcactcattcat

SEQ ID NO: 618 - DNA, 21 bp, Artificial Sequence
  Antisense oligonucleotide MTx_ASO_401
tctactcatcactcattcatc

SEQ ID NO: 619 - DNA, 21 bp, Artificial Sequence
  Antisense oligonucleotide MTx_ASO_402
ctactcatcactcattcatct

SEQ ID NO: 620 - DNA, 21 bp, Artificial Sequence
  Antisense oligonucleotide MTx_ASO_403
tactcatcactcattcatctg

SEQ ID NO: 621 - DNA, 21 bp, Artificial Sequence
  Antisense oligonucleotide MTx_ASO_404
actcatcactcattcatctgt

SEQ ID NO: 622 - DNA, 21 bp, Artificial Sequence
  Antisense oligonucleotide MTx_ASO_405
ctcatcactcattcatctgtt

SEQ ID NO: 623 - DNA, 21 bp, Artificial Sequence
  Antisense oligonucleotide MTx_ASO_406
tcatcactcattcatctgttc

SEQ ID NO: 624 - DNA, 21 bp, Artificial Sequence
  Antisense oligonucleotide MTx_ASO_407
catcactcattcatctgttca

SEQ ID NO: 625 - DNA, 21 bp, Artificial Sequence
  Antisense oligonucleotide MTx_ASO_408
atcactcattcatctgttcaa

SEQ ID NO: 626 - DNA, 21 bp, Artificial Sequence
  Antisense oligonucleotide MTx_ASO_409
tcactcattcatctgttcaat

SEQ ID NO: 627 - DNA, 21 bp, Artificial Sequence
  Antisense oligonucleotide MTx_ASO_410
cactcattcatctgttcaatc

SEQ ID NO: 628 - DNA, 21 bp, Artificial Sequence
  Antisense oligonucleotide MTx_ASO_411
actcattcatctgttcaatca

SEQ ID NO: 629 - DNA, 21 bp, Artificial Sequence
  Antisense oligonucleotide MTx_ASO_412
ctcattcatctgttcaatcat

SEQ ID NO: 630 - DNA, 21 bp, Artificial Sequence
  Antisense oligonucleotide MTx_ASO_413
tcattcatctgttcaatcatt

SEQ ID NO: 631 - DNA, 21 bp, Artificial Sequence
  Antisense oligonucleotide MTx_ASO_414
cattcatctgttcaatcattc

SEQ ID NO: 632 - DNA, 21 bp, Artificial Sequence
  Antisense oligonucleotide MTx_ASO_415
attcatctgttcaatcattca

SEQ ID NO: 633 - DNA, 21 bp, Artificial Sequence
  Antisense oligonucleotide MTx_ASO_416
ttcatctgttcaatcattcat

SEQ ID NO: 634 - DNA, 21 bp, Artificial Sequence
  Antisense oligonucleotide MTx_ASO_417
tcatctgttcaatcattcatt

SEQ ID NO: 635 - DNA, 21 bp, Artificial Sequence
  Antisense oligonucleotide MTx_ASO_418
catctgttcaatcattcattc

SEQ ID NO: 636 - DNA, 21 bp, Artificial Sequence
  Antisense oligonucleotide MTx_ASO_419
atctgttcaatcattcattca

SEQ ID NO: 637 - DNA, 21 bp, Artificial Sequence
  Antisense oligonucleotide MTx_ASO_420
tctgttcaatcattcattcat

SEQ ID NO: 638 - DNA, 21 bp, Artificial Sequence
  Antisense oligonucleotide MTx_ASO_421
ctgttcaatcattcattcatt

SEQ ID NO: 639 - DNA, 21 bp, Artificial Sequence
  Antisense oligonucleotide MTx_ASO_422
tgttcaatcattcattcattc

SEQ ID NO: 640 - DNA, 21 bp, Artificial Sequence
  Antisense oligonucleotide MTx_ASO_423
gttcaatcattcattcattca

SEQ ID NO: 641 - DNA, 150 bp, Homo sapiens
  misc_feature (1)..(150): Cryptic Exon Splice Acceptor hg38 chr19:17,642,640-17,642,491
tccaggttgactctcactactcatcatcaggttcttccttctattccagccctaaccactcaggattgggccgtttgtgtctgggtatgtctcttccagctgcctgggtttcctggaaagaactcttatccccaggaactagtttgttga

SEQ ID NO: 642 - DNA, 114 bp, Homo sapiens
  misc_feature (1)..(114): Cryptic Exon Splice Donor hg38 chr19 17,642,504-17,642,391
aactagtttgttgaataaatgctggtgaatgaatgaatgattgaacagatgaatgagtgatgagtagataaaaggatggatggagagatgggtgagtacatggatggatagatg

SEQ ID NO: 643 - DNA, 69 bp, Homo sapiens
  misc_feature (1)..(69): Cryptic Exon Splice Acceptor hg38 chr19 17,642,562-17,642,494
gtctgggtatgtctcttccagctgcctgggtttcctggaaagaactcttatccccaggaactagtttgt

SEQ ID NO: 644 - DNA, 89 bp, Homo sapiens
  misc_feature (1)..(89): Cryptic Exon Splice Donor hg38 chr19 17,642,479-17,642,391
tgaatgaatgaatgattgaacagatgaatgagtgatgagtagataaaaggatggatggagagatgggtgagtacatggatggatagatg

SEQ ID NO: 645 - DNA, 45 bp, Artificial Sequence
  ASOs 1-10 compiled sequence
tcaacaaactagttcctggggataagagttctttccaggaaaccc

SEQ ID NO: 646 - DNA, 30 bp, Artificial Sequence
  ASOs 17-21 compiled sequence
cagctggaagagacatacccagacacaaac

SEQ ID NO: 647 - DNA, 39 bp, Artificial Sequence
  ASOs 249-256 compiled sequence
ctatccatccatgtactcacccatctctccatccatcct

SEQ ID NO: 648 - DNA, 33 bp, Artificial Sequence
  ASOs 260-265 compiled sequence
catccttttatctactcatcactcattcatctg

SEQ ID NO: 649 - DNA, 21 bp, Artificial Sequence
  ASOs 271-272 compiled sequence
ttcaatcattcattcattcac

SEQ ID NO: 650 - DNA, 45 bp, Homo sapiens
  misc_feature (1)..(45): ASOs 1-10 compiled sequence reverse complement
gggtttcctggaaagaactcttatccccaggaactagtttgttga

SEQ ID NO: 651 - DNA, 30 bp, Homo sapiens
  misc_feature (1)..(30): ASOs 17-21 compiled sequence reverse complement
gtttgtgtctgggtatgtctcttccagctg

SEQ ID NO: 652 - DNA, 39 bp, Homo sapiens
  misc_feature (1)..(39): ASOs 249-256 compiled sequence reverse complement
aggatggatggagagatgggtgagtacatggatggatag

SEQ ID NO: 653 - DNA, 33 bp, Homo sapiens
  misc_feature (1)..(33): ASOs 260-265 compiled sequence reverse complement
cagatgaatgagtgatgagtagataaaaggatg

SEQ ID NO: 654 - DNA, 21 bp, Homo sapiens
  misc_feature (1)..(21): ASOs 271-272 compiled sequence reverse complement
gtgaatgaatgaatgattgaa

(19) United States
(12) Patent Application Publication
(10) Pub. No.: US 2025/0011773 A1
(43) Pub. Date: Jan. 9, 2025

(54) COMPOSITIONS AND METHODS FOR TREATING TDP-43 PROTEINOPATHY

(71) Applicant: Maze Therapeutics, Inc., South San Francisco, CA (US)

(72) Inventors: Shila MEKHOUBAD, South San Francisco, CA (US); Georgiana MILLER, South San Francisco, CA (US); Nathan SALLEE, South San Francisco, CA (US); Eric GREEN, South San Francisco, CA (US); David WYATT, San Francisco, CA (US)

(21) Appl. No.: 18/285,802

(22) PCT Filed: Apr. 5, 2022
(86) PCT No.: PCT/US2022/023559

§ 371 Date: Oct. 5, 2023

Related U.S. Application Data
(60) Provisional application No. 63/312,808, filed on Feb. 22, 2022.
(60) Provisional application No. 63/171,522, filed on Apr. 6, 2021.

(51) Int. Cl. C12N 15/113

(52) U.S. Cl. CPC C12N 15/113; C12N 2310/11; C12N 2310/321; C12N 2320/34

(57) ABSTRACT

The present disclosure relates to the use of UNC13A cryptic exon splice variant specific inhibitors for methods of reducing expression of a UNC13A cryptic exon splice variant in a cell, reducing phosphorylated TAR-DNA binding protein-43 (TDP-43) in a cell, treating TAR-DNA binding protein-43 (TDP-43) proteinopathy in a subject, or treating a subject has been identified as having a UNC13A mutation in intron 20-21 of UNC13A. Antisense oligonucleotides directed against UNC13A cryptic splice variant are also contemplated.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 630264_403W0 SEQUENCE_LISTING.txt. The text file is 243 KB, was created on Apr. 5, 2022, and is being submitted electronically via EFS-Web.

### BACKGROUND

The hallmark pathological feature of neurodegenerative diseases amyotrophic lateral sclerosis (ALS) and frontotemporal dementia (FTD) is the depletion of RNA-binding protein TDP-43 from the nucleus of neurons in the brain and spinal cord. TDP-43, encoded by TARDBP, is an abundant, ubiquitously expressed RNA-binding protein that normally localizes to the nucleus. It plays a role in fundamental RNA processing activities including RNA transcription, alternative splicing, and RNA transport (1). TDP-43 can bind to thousands of pre-messenger RNA/mRNA targets (2, 3). Reduction in TDP-43 from an otherwise normal adult nervous system alters the splicing or expression levels of more than 1,500 RNAs, including long intron-containing transcripts (2). A major splicing regulatory function of TDP-43 is to repress the inclusion of cryptic exons during splicing (4-7). Unlike normal conserved exons, these cryptic exons are lurking in introns and normally excluded from mature mRNAs. When TDP-43 is depleted from cells, these cryptic exons get spliced into messenger RNAs, often introducing frame shifts and premature termination or even nonsense-mediated decay of the mRNA. However, cryptic splicing events that are key for disease remains to be identified. Thus, the discovery of cryptic splicing targets that are regulated by TDP-43 and also play a role in the pathogenesis of TDP-43 proteinopathies as therapeutic targets is needed.

### SUMMARY

In one aspect, the present disclosure provides a method of reducing expression of a UNC13A cryptic exon splice variant in a cell comprising administering a UNC13A cryptic exon splice variant specific inhibitor, wherein: (a) the UNC13A cryptic exon splice variant comprises a cryptic exon between exon 20 and exon 21 of the UNC13A cryptic exon splice variant mature mRNA transcript; and (b) the UNC13A cryptic exon splice variant specific inhibitor comprises an antisense oligonucleotide.

In another aspect, the present disclosure provides a method of reducing phosphorylated TAR-DNA binding protein-43 (TDP-43) in a cell comprising administering a UNC13A cryptic exon splice variant specific inhibitor, wherein: (a) the UNC13A cryptic exon splice variant comprises a cryptic exon between exon 20 and exon 21 of the UNC13A cryptic exon splice variant mature mRNA transcript; and (b) the UNC13A cryptic exon splice variant specific inhibitor comprises an antisense oligonucleotide.

In another aspect, the present disclosure provides a method of treating TAR-DNA binding protein-43 (TDP-43) proteinopathy in a subject comprising administering a UNC13A cryptic exon splice variant specific inhibitor to the subject, wherein: (a) the UNC13A cryptic exon splice variant comprises a cryptic exon between exon 20 and exon 21 of the UNC13A cryptic exon splice variant mature mRNA transcript; and (b) the UNC13A cryptic exon splice variant specific inhibitor comprises an antisense oligonucleotide.

In yet another aspect, the present disclosure provides a method of treating a subject that has been identified as having a UNC13A gene mutation in intron 20-21 comprising administering an UNC13A cryptic exon splice variant specific inhibitor to the subject, wherein: (a) the UNC13A cryptic exon splice variant comprises a cryptic exon between exon 20 and exon 21 of the UNC13A cryptic exon splice variant mature mRNA transcript; and (b) the UNC13A cryptic exon splice variant specific inhibitor comprises an antisense oligonucleotide.

In embodiments, the cryptic exon comprises the base sequence of SEQ ID NO:5 or SEQ ID NO:6.

In embodiments, the UNC13A cryptic exon splice variant comprises SEQ ID NO:7 or SEQ ID NO:8.

In embodiments, the UNC13A cryptic exon splice variant specific inhibitor comprises an antisense oligonucleotide that is complementary to: (a) the 5′ end of the cryptic exon having a sequence set forth in SEQ ID NO:641; or (b) the 3′ end of the cryptic exon having a sequence set forth in SEQ ID NO:642.

In embodiments, the UNC13A cryptic exon splice variant specific inhibitor comprises an antisense oligonucleotide that is complementary to: (a) the 5′ end of the cryptic exon having a sequence set forth in SEQ ID NO:643; or (b) the 3′ end of the cryptic exon having a sequence set forth in SEQ ID NO:644.

In embodiments, the UNC13A cryptic exon splice variant specific inhibitor comprises an antisense oligonucleotide that is complementary to: (a) the exon 20 splice donor site region in a preprocessed mRNA encoding UNC13A; (b) the cryptic exon splice acceptor site region in a preprocessed mRNA encoding UNC13A; (c) the cryptic exon splice donor site region in a preprocessed mRNA encoding UNC13A; or (d) the exon 21 splice acceptor site region in a preprocessed mRNA encoding UNC13A.

In embodiments, the exon 20 splice donor site region in the preprocessed mRNA encoding UNC13A comprises or consists of SEQ ID NO:12; the cryptic exon splice acceptor site region in the preprocessed mRNA encoding UNC13A comprises or consists of SEQ ID NO:91; the cryptic exon splice donor site region in the preprocessed mRNA encoding UNC13A comprises or consists of SEQ ID NO:220; or the exon 21 splice acceptor site region in the preprocessed mRNA encoding UNC13A comprises or consists of SEQ ID NO:299.

In embodiments, the antisense oligonucleotide has 15-40 bases. In embodiments, the antisense oligonucleotide has 20-30 bases. In embodiments, the antisense oligonucleotide has 18-25 bases. In embodiments, the antisense oligonucleotide has 18-22 bases.

In embodiments, the antisense oligonucleotide has a base sequence that has at least 80%, 85%, 90%, or 95% identity to any one of SEQ ID NOS:13-90, 92-219, 221-298, 300-377, and 423-640. In embodiments, the antisense oligonucleotide has a base sequence comprising or consisting of any one of SEQ ID NOS: 13-90, 92-219, 221-298, 300-377, and 423-640. In embodiments, the antisense oligonucleotide has a base sequence comprising or consisting of any one of SEQ ID NOS:423-432, 439-443, 491-498, 502-507, and 513-514.

In embodiments, the antisense oligonucleotide: (a) has 18-30 bases, 18-25 bases, or 18-22 bases that are complementary to SEQ ID NO:650; (b) has 18-30 bases, 18-25 bases, or 18-22 bases that are complementary to SEQ ID NO: 651; (c) has 18-30 bases, 18-25 bases, or 18-22 bases that are complementary to SEQ ID NO:652; (d) has 18-30 bases, 18-25 bases, or 18-22 bases that are complementary to SEQ ID NO:653; or (e) has 18-21 bases that are complementary to SEQ ID NO:654.

In embodiments, the antisense oligonucleotide is a modified antisense oligonucleotide. In embodiments, the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

The present disclosure also provides a pharmaceutical composition comprising an antisense oligonucleotide having 15-40 bases and comprising a base sequence that has at least 80% identity to any one of SEQ ID NOS: 13-90, 92-219, 221-298, 300-377, and 423-640, and a pharmaceutically acceptable excipient.

The present disclosure also provides a pharmaceutical composition comprising an antisense oligonucleotide having: (a) 18-30 bases, 18-25 bases, or 18-22 bases that are complementary to SEQ ID NO:650; (b) 18-30 bases, 18-25 bases, or 18-22 bases that are complementary to SEQ ID NO: 651; (c) 18-30 bases, 18-25 bases, or 18-22 bases that are complementary to SEQ ID NO:652; (d) 18-30 bases, 18-25 bases, or 18-22 bases that are complementary to SEQ ID NO:653; or (e) 18-21 bases that are complementary to SEQ ID NO:654; and a pharmaceutically acceptable excipient.

In another aspect, the present disclosure provides a modified antisense oligonucleotide having 15-40 bases and comprising a base sequence that has at least 80% identity to any one of SEQ ID NOS: 13-90, 92-219, 221-298, 300-377, and 423-640.

In yet another aspect, the present disclosure provides a modified antisense oligonucleotide having 15-40 bases, wherein wherein the base sequence is complementary to: (a) the 5′ end of the cryptic exon having a sequence set forth in SEQ ID NO:641; or (b) the 3′ end of the cryptic exon having a sequence set forth in SEQ ID NO:642.

The present disclosure also provides kits comprising the UNC13A cryptic exon splice variant specific antisense oligonucleotide of the present disclosure.

### BRIEF DESCRIPTION OF THE SEVERAL VIEWS OF THE DRAWINGS

FIGS. 1A-1J. Nuclear depletion of TDP-43 causes cryptic exon inclusion in UNC13A RNA and reduced expression of UNC13A protein. FIG. 1A: Splicing analyses were performed on RNA-sequencing results generated from TDP-43-positive and TDP-43-negative neuronal nuclei isolated from frontal cortices of 7 FTD/FTD-ALS patients. FACS, fluorescent-activated cell sorting. FIG. 1B: 65 alternatively spliced genes identified by both MAJIQ (P(ΔΨ>0.1) >0.95)(ΔΨ, changes of local splicing variations between two conditions) and LeafCutter (P<0.05). FIG. 1C: Visualization of RNA-sequencing alignment between exon 20 and exon 21 in UNC13A (hg38). Libraries were generated as described in (FIG. 1A). CE, cryptic exon. FIG. 1D: iCLIP for TDP-43 indicates that TDP-43 binds to intron 20-21. An example of a region in intron 20-21 that is frequently bound by TDP-43. TDP-43 binding motif (UG)n is highlighted in orange. FIG. 1E and FIG. 111: RT-qPCR confirmed the inclusion of cryptic exon in UNC13A mRNA upon TDP-43 depletion in SH-SY5Y cells (5 independent cell culture experiments for each condition) (FIG. 1E) and in 3 independent induced motor neurons (iMNs) (4 independent cell culture experiments for each iMN) (FIG. 111). The locations of the primers spanning the cryptic exon associated region are shown. RPLPO were used to normalize qRT-PCR. (two sided-Welch Two Sample t-test, *P<0.05, **P<0.01, ***P<0.001, ****P<0.0001; mean±s.e.m. ). FIG. 1F and FIG. 1I: Immunoblotting of UNC13A protein and TDP-43 in SH-SY5Y cells (FIG. 1F) and iMNs (FIG. 11) treated with Scramble shRNA or TDP-43 shRNA (n=3). GAPDH served as a loading control. FIG. 1G: Quantification of the blots in (FIG. 1F) (two-sided Welch Two Sample t-test, *P<0.05, **P<0.01). FIG. 1J: RT-qPCR (n=5) analyses confirmed the inclusion of UNC13A cryptic exon upon TDP-43 depletion in neurons derived from human iPS cells (3 independent cell culture experiments). RPLP0 and GAPDH were used to normalize qRT-PCR (two sided-Welch Two Sample t-test;***P<0.001, ****P<0.0001; mean±s.e.m. ).

FIGS. 2A-2D. UNC13A cryptic exon inclusion in human TDP-43 proteinopathies. FIG. 2A: UNC13A cryptic exon expression level is significantly increased in the frontal cortices of FTLD-TDP patients. The qRT-PCR primer pair used for cryptic exon detection is shown on top. GAPDH and RPLPO were used to normalize qRT-PCR (two-tailed Mann-Whitney test, ****P<0.0001; error bars represent 95% confidence intervals). FIG. 2B: UNC13A cryptic exon is detected in nearly 50% of frontal cortical tissues and temporal cortical tissues from neuropathologically confirmed FTLD-TDP patients in NYGC ALS Consortium cohort. The cryptic exon is also notably absent in tissues from healthy controls, FTLD-FUS, FTLD-TAU and ALS-SOD1 patients. FIG. 2C: UNC13A cryptic exon signal is positively correlated with phosphorylated TDP-43 levels in frontal cortices of FTLD-TDP patients in Mayo Clinic brain bank (Spearman's rho=0.572, p-value <0.0001). Data points are colored according to patients' reported genetic mutations. FIG. 2D: Spearman's correlations between UNC13A cryptic exon signal and phosphorylated TDP-43 levels. Rows colored in green indication the correlation within each genetic mutation group. Rows colored in blue shows the correlation within each disease group.

FIGS. 3A-3B. UNC13A cryptic splicing is a pathological feature in human brain associated with loss of nuclear TDP-43. FIG. 3A: BaseScope™ in situ hybridization and immunofluorescence was performed on sections from the medial frontal pole. Representative images illustrate the presence of UNC13A cryptic exons (arrowheads) in neurons showing depletion of nuclear TDP-43. Neurons with normal nuclear TDP-43, in patients and controls, show no cryptic exons (arrows). FIG. 3B: Representative images showing expression of UNC13A mRNA in layer 2-3 neurons from the medial frontal pole. BaseScope™ in situ hybridization was used to visualize UNC13A mRNA, using probes that target the canonical exon20/21 junction, and combined with immunofluorescence for TDP-43 and NeuN. UNC13A mRNA expression is restricted to neurons (arrows). Images are maximum intensity projections of a confocal image Z-stack. Scale bar equals 10 μm.

FIGS. 4A-4J. Risk haplotype associated with ALS/FTD susceptibility potentiates cryptic exon inclusion when TDP-43 is dysfunctional. FIG. 4A: LocusZoom plot showing SNPs associated with ALS/FTD in UNC13A. rs12608932, the most significant GWAS hit is chosen to be the reference. Other SNPs are colored based on their levels of linkage equilibrium with rs12608932 in EUR population. The two SNPs in intron 20-21 (black triangles), rs12608932 and rs12973192 are in strong linkage disequilibrium. FIG. 4B: There is a higher inclusion of the risk allele (G) at rs12973192 in UNC13A splice variant (two-sided paired t-test, **P=0.0094). Both simple linear regression model (FIG. 4C) and multiple regression model (FIG. 4D) show a strong correlation between the abundance of UNC13A cryptic exon and the number of risk alleles. Normality of residuals is tested by Shapiro-Wilk normality test (p-value=0.2604). FIG. 4D: Summary results of the multiple regression analysis using the number of risk alleles at rs12973192, TDP-43 phosphorylation levels, sex, reported genetic mutations as predictor variables. Rows colored in the same color indicate factors within the same variable. Normality of residuals is tested by Shapiro-Wilk normality test (p-value=0.1751). FIG. 4E: Diagram of the location of rs56041637 relative to the two known GWAS hits and UNC13A cryptic exon. FIG. 4F: Design of UNC13A cryptic exon minigene reporter constructs and the location of the primer pair used for RT-PCR. Transcription of GFP and mCherry is controlled by a bidirectional promoter (blue). Black triangles represent the locations of genetic variants as shown in (E). FIG. 4G: Splicing of the minigenes was assessed in WT and TDP-43−/− HEK293T cells. HEK293T cells do not endogenously express UNC13A. The PCR products represented by each band are marked to the left of each gel. In addition to the inclusion of cryptic exon (b), some splice variants have inclusion of the longer version of the cryptic exon (c) (FIG. 5) or the complete intron upstream of the cryptic exon (d). The risk allele-carrying minigene showed an almost complete loss of canonical splicing product (a) and an increase in alternatively spliced products. FIG. 4H: In HeLa cells expressing a different UNC13A minigene reporter, depletion of TDP-43 by siRNA (and cycloheximide (CHX) treatment), resulted in inclusion of the cryptic exon, which can be rescued by over-expressing TDP-43 protein (GFP-TDP-43) but not by the RNA-binding deficient mutant TDP-43 (GFP-TDP-43-5FL). FIG. 4I: Survival curves of FTLD-TDP patients stratified based on the number of the risk haplotypes they carry (0, 1, or 2). Patients who are heterozygous and homozygous for the risk haplotype had shorter survival time after disease onset (n=205, Mayo Clinic brain bank) (Score (logrank) test, p-value=0.01). Dash lines mark the median survival for each genotype. The effect of the risk haplotype is modeled as an additive model using Cox multivariable analysis adjusted for genetic mutations, sex and age at onset. The risk table is shown at the bottom. Summary results of the analysis are in FIG. 15A. FIG. 4J: Model of how UNC13A protein expression level is most significantly decreased in patients who both carry the UNC13A risk haplotype and exhibit TDP-43 pathology.

FIG. 5A-5D. Splicing analysis using MAJIQ demonstrates inclusion of the cryptic exon between exon 20 and exon 21 of UNC13A. FIGS. 5A and 5B: Depletion of TDP-43 introduces two alternative 3′ splicing acceptors in the intron 20-21: one is at chrl9:17642591(ΔΨ=0.05184) and the other one is at chrl9:17642541(ΔΨ=0.48865). FIGS. 5C and 5D: An alternative 5′ splicing donor is also introduced at chrl9:17642414 (ΔΨ=0.772). Since much higher usage of the chrl9:17642541 3′ splicing acceptor was observed (FIG. 5B), the 128 bp cryptic exon defined by this 3′ splicing acceptor and the alternative 5′ splicing donor (FIG. 5C) became the focus. FIGS. 5A and 5C are splice graphs showing the inclusion of the cryptic exon (CE) between exon 20 and exon 21 of UNC13A. FIGS. 5B and 5D: are violin plots corresponding to FIGS. 5A and 5C, respectively. Each violin in (FIGS. 5B and 5D) represents the posterior probability distribution of the expected relative inclusion (PSI or Ψ) for the color matching junction in the splice graph. The tails of each violin represent the 10 th and 90 th percentile. The box represents the interquartile range with the line in the middle indicating the median. The white circles mark the expected PSI (E[Ψ]). The change in the relative inclusion level of each junction between two conditions is referred to as ΔΨ or ΔPSI(12).

FIGS. 6A-6D. Intron 20-21 of UNC13A is conserved among most primates. The Primates Multiz Alignment & Conservation track on UCSC(39) genome browser (http: genome.ucsc.edu ) includes 20 mammals, 17 of which are primates. FIG. 6A: Exon 20 and exon 21 of UNC13A is well conserved among mammals. However, intron 20-21 (FIG. 6B), the cryptic exon (FIG. 6C), and the splicing acceptor site upstream of the cryptic exon (FIG. 6C) and splicing donor site downstream of the cryptic exon (FIG. 6D) are only conserved in primates.

FIGS. 7A-7B. Depletion of TDP-43 from induced motor neurons (iMN) leads to cryptic exon inclusion in UNC13A. FIG. 7A: RT-PCR confirmed the expression of the cryptic exon-containing UNC13A mRNA isoforms upon TDP-43 depletion in three independent iMNs (4 independent cell culture experiments for each iMN and condition). In addition to the splice variant containing the cryptic exon, inclusion of a longer version of the cryptic exon was detected (FIG. 5A) and the complete intron upstream of the cryptic exon (FIG. 4G). The PCR products represented by each band are marked to the left of each gel. The location of the PCR primer pair used is shown on top of each gel image. FIG. 7B: The PCR primer pairs spanning the cryptic exon and exon 21 junction confirms cryptic exon inclusion only occurs upoen TDP-43 knockdown.

FIG. 8. Total UNC13A transcripts do not change significantly in the frontal cortices of most FTLD-TDP patients in Mayo Clinic brain bank. A decrease in total UNC13A transcript was observed in FTD patients with no reported genetic mutations and FTD patients with GRN mutations. This may be due to specific pathologies that are currently unclear. The qRT-PCR primer pair used for the detection is shown on top. GAPDH and RPLPO were used to normalize qRT-PCR (two tailed Mann-Whitney test, ns: P >0.05; **P<0.01; ****P<0.0001; error bars represent 95% confidence intervals).

FIG. 9. UNC13A cryptic exon can also be detected in disease relevant tissues of ALS/FTLD, ALS-TDP and ALS/AD patients. The diagnoses of these patients are not neuropathologically confirmed. Therefore, it is unclear whether TDP-43 mislocalization is present in these patients. ALS patients were categorized based on whether they harbor SOD] mutations (ALS-SOD1 vs. ALS-TDP). ALS-AD refers to ALS patients with suspected Alzheimer's disease. ALS-FTLD refers to patients who have concurrent FTD and ALS.

FIGS. 10A-10H. UNC13A cryptic exon signal and total UNC13A signal is correlated with phosphorylated TDP-43 levels in frontal cortices of FTLD-TDP patients in Mayo Clinic brain bank. FIG. 10A: UNC13A cryptic exon signal is positively correlated with phosphorylated TDP-43 levels in frontal cortices of FTLD-TDP patients in Mayo Clinic Brain bank (Spearman's rho=0.572, p-value <0.0001). Data points are colored according to patients' disease types. FIGS. 10B and 10C: Total UNC13A signal is negatively correlated with phosphorylated TDP-43 levels in the same samples. Data points are colored according to patients' reported genetic mutations (FIG. 10B) and disease types (FIG. 10C) respectively. FIG. 10D: Spearman's correlations between total UNC13A signal and phosphorylated TDP-43 levels. Rows colored in green shows the correlation within each genetic mutation group. Rows colored in blue shows the correlation within each disease group. FIGS. 10E-10H: Scatter plots using untransformed data as input. FIGS. 10E-10F: Cryptic exon signal vs. phosphorylated TDP-43 levels. FIG. 10G-10H: Total UNC13A signal vs. phosphorylated TDP-42 levels. qRT-PCR primer pair is shown on top of each panel.

FIGS. 11A-11E. UNC13A cryptic splicing is associated with loss of nuclear TDP-43 in human brain. FIG. 11A: The design of the UNC13A e20/CE BaseScope™ probe targeting the alternatively spliced UNC13A transcript. FIG. 11B: The design of the UNC13A e20/e21 BaseScope™ probe targeting canonical UNC13A transcript. Each “Z” binds to the transcript independently. Both “Z”s have to be in close proximity for successful signal amplification, ensuring binding specificity. FIG. 11C: BaseScope™ in situ hybridization and immunofluorescence was performed on sections from the medial frontal pole. Representative images illustrate the presence of UNC13A cryptic exons (arrowheads) in neurons showing depletion of nuclear TDP-43 and cytoplasmic aggregation. Neurons with normal nuclear TDP-43, in patients and controls, show no cryptic exons (arrows). FIG. 11D: Representative images showing expression of UNC13A mRNA in layer 2-3 neurons from the medial frontal pole. BaseScope in situ hybridization was used to visualize UNC13A mRNA, using probes that target the exon20-exon 21 junction, and combined with immunofluorescence for TDP-43 and NeuN. UNC13A mRNA expression is restricted to neurons (arrows). Images are maximum intensity projections of a confocal image Z-stack. Scale bar equals 10 μm. FIG. 11E: Six non-overlapping Z-stack images from layer 2-3 of medial frontal pole were captured, per subject, using a 63×oil objective and flattened into a maximum intensity projection image. Puncta counts per image were derived using the “analyze particle” plugin in ImageJ. Each data point represents the number of UNC13A cryptic exon puncta in a single image. The abundance of cryptic exons varies between patients but always exceeds the technical background of the assay, as observed in controls. Data are presented as mean +/−standard deviation.

FIGS. 12A-12C. The levels of cryptic exon inclusion are influenced by the genotype at rs12973192. FIG. 12A: Visualization of RNA-seq alignment between exon 20 and exon 21 of UNC13A. The RNA-seq libraries were generated from TDP-43 negative neuronal nuclei as described in FIG. 1A. FIG. 12B: Samples that are heterozygous (C/G) or homozygous (G/G) at rs12973192 have higher relative inclusion (Ψ) of the cryptic exon with the exception of SRR8571945. FIG. 12C: The percentages of C and G alleles in the UNC13A spliced variants in TDP-43 depleted iMNs and SRR8571950 neuronal nuclei. Exact binomial test was done for each replicate to test whether the observed difference in percentages differ from what was expected if both alleles are equally included in the cryptic exon.

FIG. 13A-13F. The abundance of UNC13A cryptic exon is associated with the number of risk alleles. Simple linear regression model (FIG. 13A) and multiple regression model (FIG. 13B) using untransformed data show a strong correlation between the abundance of UNC13A cryptic exon and the number of risk alleles. FIG. 13B: Summary results of the multiple regression analysis using the number of risk alleles, TDP-43 phosphorylation levels, sex, reported genetic mutations as predictor variables. Rows colored in the same color indicate factors within the same variable. FIGS. 13C and 13E: Simple linear regression models and FIGS. 13D and 13F: multiple regression models using transformed (FIGS. 13A and 13D) and untransformed (E and F) data show the abundance of total UNC13A mRNA transcript is not significantly correlated with the number of risk alleles at rs12971392 in the patient carries. This could be a result of the expression of UNC13A from neurons that are not affected by TDP-43 pathology as shown in FIG. 3B and FIG. 11D. The normality of residuals is tested by Shapiro-Wilk normality test and the results are shown at the bottom of each panel. The qPCR primer pair used for the detection is shown on top of each panel.

FIG. 14. rs56041637 and rs62121687 are in strong linkage disequilibrium with both GWAS hits in intron 20-21 of UNC13A. Using genetic variants identified in whole genome sequencing data from 297 ALS patients of European descent (July 2020, Answer ALS), we looked for other genetic variants in intron 20-21that were not represented in the previous GWASs. Along the axes of the heatplot are all loci that show variation among the 297 patients. Each tile represents the Bonferroni-adjusted p-value from Chi-square test. P-values less than 0.05 are shown in yellow and others are shown in blue or gray. The blue and red blocks highlight the associations of rs 12608932 and rs12973192 with other genetic variants in intron 20-21 respectively. Significant associations that are common to both are circled out in black. Two additional SNPs, rs56041637 (Bonferroni-adjusted p-value <0.0001 with rs12608932, Bonferroni-adjusted p-value <0.0001 with rs12973192), and rs62121687 (Bonferroni-adjusted p-value <0.0001 with rs12608932, Bonferroni-adjusted p<0.0001 with rs12973192) were found that are in LD with both. However, since rs62121687 was included in the GWAS and has a p-value of 0.0186585 (36), it was excluded from further analysis

FIGS. 15A-15E. UNC13A risk haplotype reduces the survival time of FTLD-TDP patients. FIG. 15A: Summary results of Cox multivariable analysis (adjusted for genetic mutations, sex and age at onset) of an additive model. FIGS. 15B and 15D: Survival curves of FTLD-TDP patients (n=205, Mayo Clinic Brain bank), according to a dominant model (FIG. 15B) and a recessive model (FIG. 15C) and their corresponding risk tables. Summary results of Cox multivariable analysis (adjusted for genetic mutations, sex and age at onset) of a dominant model (FIG. 15C) and a recessive model (FIG. 15D). Both the dominant model (FIGS. 15B and 15C) and the recessive model (FIGS. 15D and 15E) show that the presence of a risk haplotype can reduce the survival of FTLD-TDP patients. Dash lines mark the median survival for each genotype. Log rank p-values were calculated using Score test. Rows colored in green indicate factors within one variable.

FIGS. 16A-16F. The effect of UNC13A risk haplotype on survival is more significant in C90RF72 hexanucleotide repeat expansion carriers and GRN mutation carriers. FIGS. 16A, 16C and 16E: Survival curves of FTLD-TDP patients carrying C90RF72 or GRN mutations (n=80, Mayo Clinic Brain bank), according to an additive model (FIG. 16A), a dominant model (FIG. 16C) and a recessive model (FIG. 16E), and their corresponding risk tables. Summary results of Cox multivariable analysis (adjusted for genetic mutations, sex and age at onset) of an additive model (FIG. 16B), a dominant model (FIG. 16D) and a recessive model (FIG. 16F). When we only include FTLD-ALS patients who have mutations that are associated with TDP-43 pathology, both the additive model (FIGS. 16A and 16B) and the dominant model (FIGS. 16C and 16D) indicate that the effect of the risk haplotype on survival time becomes more significant. While the survival distributions of the two groups do not differ significantly (log rank p-value=0.3), the number of risk haplotype is still a strong prognostic factor (p-value=0.03800). Dash lines mark the median survival for each genotype. Log rank p-values were calculated using Score test.

FIG. 17 shows the UNC13A genomic region comprising exon 20, the cryptic exon #1 (128 bp), and exon 21.

FIG. 18 shows the STMN2 exon structure for the reference transcript and a splice variant containing cryptic exon 2a (top) and the exon 2a sequence (bottom).

FIGS. 19A-19D show UNC13A mRNA levels in motor neurons following treatment with UNC13A specific 2′MOE antisense oligonucleotides as measured by qPCR. FIGS. 19A-19B show qPCR results using primers/probes specific for UNC13A cryptic exon inclusion. FIGS. 19C-19D show qPCR results using primer/probes specific for reference UNC13A.

### DETAILED DESCRIPTION

Prior to setting forth this disclosure in more detail, it may be helpful to an understanding thereof to provide definitions of certain terms used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer) or subranges, unless otherwise indicated.

As used herein, the term “about” means+20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

As used herein, “nucleic acid” or “nucleic acid molecule” or “polynucleotide” refers to any of deoxyribonucleic acid (DNA), ribonucleic acid (RNA), oligonucleotide, molecules generated, for example, by the polymerase chain reaction (PCR) or by in vitro translation, and molecules generated by any of ligation, scission, endonuclease action, exonuclease action or mechanical action (e.g., shearing). Nucleic acids may be composed of a plurality of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties (e.g., morpholino nucleotides). Nucleic acid monomers of the polynucleotides can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, or the like. Nucleic acid molecules can be either single stranded or double stranded.

As used herein, “protein” or “polypeptide” as used herein refers to a compound made up of amino acid residues that are covalently linked by peptide bonds. The term “protein” may be synonymous with the term “polypeptide” or may refer, in addition, to a complex of two or more polypeptides. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference 5 polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

The term “isolated” means that a material, complex, compound, or molecule is removed from its original environment (e.g., the natural environment if it is naturally occurring). For example, a naturally occurring polynucleotide or polypeptide present in a living animal is not isolated, but the same polynucleotide or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

The term “gene” means the segment of DNA involved in producing a polypeptide chain; it includes regions preceding and following the coding region “leader and trailer” as well as intervening sequences (introns), if present, between individual coding segments (exons).

As used herein, the term “recombinant” or “genetically engineered” refers to a cell, microorganism, nucleic acid molecule, polypeptide or vector that has been genetically modified by human intervention. For example, a recombinant polynucleotide is modified by human or machine introduction of an exogenous or heterologous nucleic acid molecule, or refers to a cell or microorganism that has been altered by human or machine intervention such that expression of an endogenous nucleic acid molecule or gene is controlled, deregulated or constitutive. Human generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material or encoded products. Exemplary human or machine introduced modifications include those in coding regions or functional fragments thereof of heterologous or homologous polypeptides from a reference or parent molecule.

A “wild-type” gene or gene product is that which is most frequently observed in a population and is thus arbitrarily designed the “normal” or “reference” or “wild-type” form of the gene.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W. H. Freeman and Company.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

“Sequence identity,” as used herein, refers to the percentage of nucleotides (amino acid residues) in one sequence that are identical with the nucleotides (amino acid residues) in another reference polynucleotide (polypeptide) sequence after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values.

As used herein, “UNC13A” refers to a presynaptic protein found in central and neuromuscular synapses that regulates the release of neurotransmitters, peptides, and hormones. UNC13A reference or wildtype mRNA transcript contains 44 exons encoding a 1,703 amino acid protein. In embodiments, NCBI Reference Sequence: NP_001073890.2 (SEQ ID NO:11) is an example of a wildtype or reference UNC13A protein. In embodiments, NCBI Reference Sequence NM_001080421.3 (SEQ ID NO:1) is an example of a wild-type or reference UNC13A mRNA transcript. In embodiments, UNC13A includes all forms of UNC13A including wildtype, splice isoforms, variants, mutants, native conformation, misfolded, and post-translationally modified. In embodiments, UNC13A does not include UNC13A cryptic exon splice variant.

As used herein, the term “pre-processed mRNA” or “pre-mRNA” or “precursor mRNA” refers to a primary transcript synthesized from transcription of a DNA template and that has not undergone processing, e.g., splicing, addition of 5′ cap, and addition of a 3′ polyA tail, in order to become a mature mRNA. The mature mRNA is capable of being translated into protein by the ribosome.

As used herein, the term “cryptic exon” or “pseudoexon” refers to an exon that is absent or not detectably used in wild-type pre-mRNA but are selected in a variant isoform, Cryptic exons may arise as a result of mutations that create new splice sites or remove the existing binding sites for splicing repressors. Cryptic exons can also emerge from transposable elements (e.g., Alu elements).

As used herein, “UNC13A cryptic exon splice variant” refers to a mRNA, or protein encoded by said mRNA, that comprises a cryptic exon between exon 20 and exon 21. The cryptic exon is obtained from intron 20-21 of the UNC13A gene. In embodiments, the cryptic exon has the nucleotide sequence of SEQ ID NO:5 or SEQ ID NO:6. In embodiments, the UNC13A cryptic exon splice variant may have the nucleotide sequence of SEQ ID NO:7, encoding a protein sequence of SEQ ID NO:8, or the nucleotide sequence of SEQ ID NO:9, encoding a protein sequence of SEQ ID NO:10.

As used herein, “transactivation response element DNA-binding protein 43” or “TAR-DNA binding protein-43” or “TDP-43” refers to a protein of typically 414 amino acid residues encoded by TARDBP. In embodiments, wildtype TDP43 amino acid sequence is provided by Uniprot Accession number Q13148 (SEQ ID NO:378). In embodiments, TDP43 includes all forms of TDP-43 including wildtype, splice isoforms, variants, mutants, native conformation, misfolded, and post-translationally modified (e.g., ubiquitinated, phosphorylated, acetylated, sumoylated, or cleaved into C-terminal fragments) proteins.

As used herein, the “TAR-DNA binding protein-43 proteinopathy” or “TDP-43 proteinopathy” refers to a neurodegenerative disease that is characterized by the deposition of TDP-43 positive protein inclusions in the brain and/or spinal cord of subjects. Cytoplasmic inclusions of hyperphosphorylated, ubiquitinated, cleaved form of TDP-43 are a pathological feature of diseases including but not limited to amyotrophic lateral sclerosis (ALS), frontotemporal lobar degeneration (FTLD), primary lateral sclerosis (PLS), progressive muscular atrophy (PMA), facial onset sensory and motor neuronopathy (FOSMN), hippocampal sclerosis (HS), limbic-predominant age-related TDP-43 encephalopathy (LATE), cerebral age-related TDP-43 with sclerosis (CARTS), Guam Parkinson-dementia complex (G-PDC), Guan ALS (G-ALS), Multisystem proteinopathy (MSP), Perry disease, Alzheimer's disease (AD), and chronic traumatic encephalopathy (CTE).

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules, or there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target nucleic acid (e.g., RNA). Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

The terms “antisense oligomer” or “antisense compound” or “antisense oligonucleotide” or “oligonucleotide” are used interchangeably and refer to a short, single-stranded polynucleotide (e.g., 10-50 subunits) made up of DNA, RNA or both, that hybridizes to a target sequence in a nucleic acid (typically an RNA) by Watson-Crick base pairing, to form a nucleic acid:oligomer heteroduplex within the target sequence. An antisense oligonucleotide may comprise unmodified nucleotides or may contain modified nucleotides, non-natural nucleotides, or analog nucleotides, such as morpholino, phosphorothioate, peptide nucleic acid, LNA, 2′-O-Me RNA, 2′F-RNA, 2′-0-MOE-RNA, 2′F-ANA, or any combination thereof.

Such an antisense oligomer can be designed to block or inhibit translation of mRNA or to inhibit natural pre-mRNA splice processing, or induce degradation of targeted mRNAs, and may be said to be “directed to” or “targeted against” a target sequence with which it hybridizes. In embodiments, the target sequence is a region surrounding or including an AUG start codon of an mRNA, a 3′ or 5′ splice site of a pre-processed mRNA, or a branch point. The target sequence may be within an exon or within an intron or a combination thereof. The target sequence for a splice site may include an mRNA sequence having its 5′ end at 1 to about 25 base pairs downstream of a normal splice acceptor junction in a preprocessed mRNA. An exemplary target sequence for a splice site is any region of a preprocessed mRNA that includes a splice site or is contained entirely within an exon coding sequence or spans a splice acceptor or donor site. An oligomer is more generally said to be “targeted against” a biologically relevant target such as, in the present disclosure, a human UNC13A gene pre-mRNA encoding the UNC13A protein, when it is targeted against the nucleic acid of the target in the manner described above. Exemplary targeting sequences include those listed in Tables 2-5.

The term “oligonucleotide analog” refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Exemplary analogs are those having a substantially uncharged, phosphorus containing backbone.

A “subunit” of an oligonucleotide refers to one nucleotide (or nucleotide analog) unit comprising a purine or pyrimidine base pairing moiety. The term may refer to the nucleotide unit with or without the attached intersubunit linkage, although, when referring to a “charged subunit”, the charge typically resides within the intersubunit linkage (e.g., a phosphate or phosphorothioate linkage or a cationic linkage).

The purine or pyrimidine base pairing moiety, also referred to herein simply as a “nucleobases,” “base,” or “bases,” may be adenine, cytosine, guanine, uracil, thymine or inosine. Also included are bases such as pyridin-4-one, pyridin-2-one, phenyl, pseudouracil, 2,4,6-trime115thoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonyhnethyluridine, 5-methyloxyuridine, 5-methyl-2-thiouridine, 2-methylthio-N6-isopentenyladenosine, j-D-mannosylqueosine, uridine-5-oxyacetic acid, 2-thiocytidine, threonine derivatives and others (Burgin et al., 1996, Biochemistry, 35:14090; Uhlman & Peyman, supra). By “modified bases” in this aspect is meant nucleotide bases other than adenine (A), guanine (G), cytosine (C), thymine (T), and uracil (U), as illustrated above; such bases can be used at any position in the antisense molecule. Persons skilled in the art will appreciate that depending on the uses of the oligomers, Ts and Us are interchangeable. For instance, with other antisense chemistries such as 2′-O-methyl antisense oligonucleotides that are more RNA-like, the T bases may be shown as U.

The term “targeting sequence” is the sequence in the oligomer or oligomer analog that is complementary (meaning, in addition, substantially complementary) to the “target sequence” in the RNA genome. The entire sequence, or only a portion, of the antisense oligomer may be complementary to the target sequence. For example, in an oligomer having 20-30 bases, about 6, 7, 8, 9, 10, 11, 12, 13, 14, 15, 16, 17, 18, 19, 20, 21, 22, 23, 24, 25, 26, 27, 28, or 29 may be targeting sequences that are complementary to the target region. Typically, the targeting sequence is formed of contiguous bases in the oligomer, but may alternatively be formed of non-contiguous sequences that when placed together, e.g., from opposite ends of the oligomer, constitute sequence that spans the target sequence.

A “targeting sequence” may have “near” or “substantial” complementarity to the target sequence and still function for the purpose of the present disclosure, that is, still be “complementary.” Preferably, the oligomer analog compounds employed in the present disclosure have at most one mismatch with the target sequence out of 10 nucleotides, and preferably at most one mismatch out of 20. Alternatively, the antisense oligomers employed have at least 90% sequence identity, and preferably at least 95% sequence identity, with the exemplary targeting sequences as designated herein.

An “amino acid subunit” or “amino acid residue” can refer to an α-amino acid residue (—CO—CHR—NH—) or a β- or other amino acid residue (e.g., —CO—(CH₂)ₙCHR—NH—), where R is a side chain (which may include hydrogen) and n is 1 to 7, preferably 1 to 4.

The term “naturally occurring amino acid” refers to an amino acid present in proteins found in nature, such as the 20 (L)-amino acids utilized during protein biosynthesis as well as others such as 4-hydroxyproline, hydroxylysine, desmosine, isodesmosine, homocysteine, citrulline and ornithine. The term “non-natural amino acids” refers to those amino acids not present in proteins found in nature, examples include beta-alanine (β-Ala), 6-aminohexanoic acid (Ahx) and 6-aminopentanoic acid. Additional examples of “non-natural amino acids” include, without limitation, (D)-amino acids, norleucine, norvaline, p-fluorophenylalanine, ethionine and the like, which are known to a person skilled in the art.

The term “target sequence” refers to a portion of the target RNA against which the oligonucleotide or antisense agent is directed, that is, the sequence to which the oligonucleotide will hybridize by Watson-Crick base pairing of a complementary sequence. In embodiments, the target sequence may be a contiguous region of a pre-mRNA that includes both intron and exon target sequence. In embodiments, the target sequence will consist exclusively of either intron or exon sequences.

Target and targeting sequences are described as “complementary” to one another when hybridization occurs in an antiparallel configuration. A targeting sequence may have “near” or “substantial” complementarity to the target sequence and still function for the purpose of the present disclosure, that is, it may still be functionally “complementary.” In certain embodiments, an oligonucleotide may have at most one mismatch with the target sequence out of 10 nucleotides, and preferably at most one mismatch out of 20. Alternatively, an oligonucleotide may have at least 90% sequence identity, and preferably at least 95% sequence identity, with the exemplary antisense targeting sequences described herein.

An oligonucleotide “specifically hybridizes” to a target polynucleotide if the oligomer hybridizes to the target under physiological conditions, with a Tm substantially greater than 45° C., preferably at least 50° C., and typically 60° C.-80° C. or higher. Such hybridization preferably corresponds to stringent hybridization conditions. At a given ionic strength and pH, the Tm is the temperature at which 50% of a target sequence hybridizes to a complementary polynucleotide. Again, such hybridization may occur with “near” or “substantial” complementarity of the antisense oligomer to the target sequence, as well as with exact complementarity.

A “nuclease-resistant” oligomeric molecule (oligomer) refers to one whose backbone is substantially resistant to nuclease cleavage, in non-hybridized or hybridized form; by common extracellular and intracellular nucleases in the body; that is, the oligomer shows little or no nuclease cleavage under normal nuclease conditions in the body to which the oligomer is exposed.

An “effective amount” or “therapeutically effective amount” refers to an amount of therapeutic agent, such as an UNC13A cryptic splice variant inhibitor, administered to a mammalian subject, either as a single dose or as part of a series of doses, which is effective to produce a desired therapeutic effect. For an antisense oligonucleotide, this effect is typically brought about by inhibiting translation or natural splice-processing of a selected target sequence. An “effective amount,” targeted against UNC13A cryptic exon splice variant mRNA, also relates to an amount effective to modulate expression of UNC13A cryptic exon splice variant protein.

The term “inhibit” or “inhibitor” refers to an alteration, interference, reduction, down regulation, blocking, suppression, abrogation or degradation, directly or indirectly, in the expression, amount or activity of a target gene, target protein, or signaling pathway relative to (1) a control, endogenous or reference target or pathway, or (2) the absence of a target or pathway, wherein the alteration, interference, reduction, down regulation, blocking, suppression, abrogation or degradation is statistically, biologically, or clinically significant. The term “inhibit” or “inhibitor” includes gene “knock out” and gene “knock down” methods, such as by chromosomal editing.

For example, a “UNC13A cryptic exon splice variant inhibitor” may block, inactivate, reduce or minimize UNC13A cryptic exon splice variant activity or reduce activity by reducing expression of or promoting degradation of UNC13A cryptic exon splice variant, by about 20%, 25%, 30%, 35%, 40%, 45%, 50%, 55%, 60% , 65%, 70%, 75%, 80%, 85%, 86%, 87%, 88%, 89%, 90%, 91%, 92%, 93%, 94%, 95%, 96%, 97%, 98%, 99%, or more as compared to untreated UNC13A cryptic exon splice variant.

“Treatment” of an individual or a cell is any type of intervention provided as a means to alter the natural course of a disease or pathology in the individual or cell. Treatment includes, but is not limited to, administration of, e.g., a pharmaceutical composition, and may be performed either prophylactically, or subsequent to the initiation of a pathologic event or contact with an etiologic agent. Treatment includes any desirable effect on the symptoms or pathology of a disease or condition associated with inflammation, among others described herein.

Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

Additional definitions are provided in the sections below.

### UNC13A Cryptic Exon Splice Variants

In one aspect, the present disclosure provides novel UNC13A cryptic splice variants that includes a cryptic exon between exons 20 and 21. These cryptic exons are absent from wildtype UNC13A from neuronal nuclei and not present in any of the known isoforms of UNC13A. The cryptic exons are obtained from intron 20-21 of the UNC13A gene (SEQ ID NO:4). Depletion of TDP-43 introduces two alternative 3′ splicing acceptors in intron 20-21, one at chr19 17642591(ΔΨ=0.05184) and the other one is at chr19:17642541(ΔΨ=0.48865). An alternative 5′ splicing donor is also introduced at chr19:17642414 (ΔΨ=0.772). The chrl9:17642541 3′ splicing acceptor, which is more frequently used than the chr19:17642591 3′ splicing acceptor, and alternative 5′ splicing donor results in a 128 bp cryptic exon having a nucleotide sequence as set forth in SEQ ID NO:5 (“cryptic exon #1”) The UNC/3A cryptic exon #1 variant comprises a nucleotide sequence as set forth in SEQ ID NO:7, encoding a protein comprising an amino acid sequence as set forth in SEQ ID NO:8. The chr19:17642591 3′ splicing acceptor and alternative 5′ splicing donor results in a 179 bp cryptic exon having a nucleotide sequence as set forth in SEQ ID O:6 (“cryptic exon #2′”). The UNC13,A cryptic exon #2 variant com rises a nucleotide sequence as set forth in SEQ ID NO:9, encoding a protein comprising an amino acid sequence as set forth in SEQ ID NO: 10.

UNC13A cryptic exon #1 splice variant expression level is significantly increased in frontal cortexes of frontotemporal lobar degeneration with TDP-43 inclusions (FTLD-TDP) patients compared to normal controls. UNC13A cryptic exon #1 splice variant has also been detected in disease relevant tissues of ALS patients. In embodiments, expression of UNC13A cryptic splice variant #1 or UNC13A cryptic splice variant #2 may be used as a biomarker for identifying a subject with a TDP-43 proteinopathy, e.g., FTLD or ALS.

Once TDP-43 becomes depleted from the nucleus and accumulates in the cytoplasm, it becomes phosphorylated. Hyperphosphorylated TDP43 (pTDP-43) is a key feature of pathology of TDP-43 proteinopathies. UNC13A cryptic exon #1 splice variant is strongly associated with phosphorylated TDP-43 levels in FTD/ALS patients. In embodiments, expression of UNC13A cryptic splice variant #1 or UNC13A cryptic splice variant #2 may be used as a biomarker for phosphorylated TDP-43 level in a subject.

Several genetic mutations in intron 20-21 of UNC13A have been identified as promoting UNC13A cryptic exon inclusion upon TDP-43 depletion. Examples of such genetic mutations include rs12608932 (hg38 chrl9:17.641,880 A→C), rs12973192 (hg38 chrl9: 17,642,430 C→G), rs56041637 (hg38 chrl9:17,642,033-17,642,056 CATC 0-2 repeats→3-5 CATC repeats), and rs62121687 (hg38 chrl9:17,642,351 C→A). Moreover, UNC13A genetic mutations that increase cryptic exon inclusion are associated with decreased survival in FTD-ALS patients. In embodiments, identification of a genetic mutation in intron 20-21 of UNC13A in a subject may be used as a biomarker for UNC13A cryptic exon inclusion. In embodiments, identification of a genetic mutation in intron 20-21 of UNC13A in a subject with a TDP-43 proteinopathy (e.g., FTD, ALS) may be used as a biomarker for decreased survival.

TABLE 1
UNC13A Sequences

| | | SEQ |
|---|---|---|
| | | ID |
| Name | Sequence | NO: |
| UNC13A | GCCCCCGGTGCTGAACCAAGATGGCCGGTG | 1 |
| reference | GCGGCCGGGCCCCGGCGTGAGCCAAGCGCG | |
| mRNA | GGCTGCAGCCGGGAGATGCCCCAGCCCAGC | |
| NM_ | GGCCGCTGAGCCCGACCCGACAGAGCCGGC | |
| 001080421.3 | CCGGCCGCCTCCGGCCCACCTGCGAGCTCG | |
| | GAGACATGTCTCTGCTTTGCGTTGGAGTCA | |
| | AAAAAGCCAAGTTTGATGGTGCCCAAGAGA | |
| | AATTCAACACGTACGTGACCCTGAAAGTGC | |
| | AGAATGTCAAGAGCACGACCATCGCGGTGC | |
| | GGGGCAGCCAGCCCAGCTGGGAGCAGGATT | |
| | TCATGTTCGAGATTAACCGTCTGGATTTGG | |
| | GACTGACGGTGGAGGTGTGGAATAAGGGTC | |
| | TCATCTGGGACACAATGGTGGGCACTGTGT | |
| | GGATCCCACTGAGGACCATCCGCCAGTCCA | |
| | ATGAGGAGGGCCCTGGAGAGTGGCTGACGC | |
| | TGGACTCCCAGGTCATCATGGCAGACAGTG | |
| | AGATCTGTGGCACCAAGGACCCCACCTTCC | |
| | ACCGCATCCTCCTGGACACGCGCTTTGAGC | |
| | TACCCTTAGACATTCCTGAAGAGGAGGCTC | |
| | GCTACTGGGCCAAGAAGCTGGAGCAGCTCA | |
| | ATGCTATGCGGGACCAGGATGAATATTCGT | |
| | TCCAAGATGAGCAAGACAAGCCTCTGCCTG | |
| | TCCCCAGCAACCAGTGCTGCAACTGGAATT | |
| | ATTTTGGCTGGGGTGAGCAGCACAACGATG | |
| | ACCCCGACAGTGCAGTGGATGATCGTGACA | |
| | GTGACTACCGCAGTGAAACGAGCAACAGCA | |
| | TCCCGCCGCCCTATTATACTACGTCACAAC | |
| | CCAACGCCTCAGTCCACCAATATTCTGTTC | |
| | GCCCACCACCCCTGGGCTCCCGGGAGTCCT | |
| | ACAGTGACTCCATGCACAGTTACGAGGAGT | |
| | TCTCTGAGCCACAAGCCCTCAGCCCCACGG | |
| | GTAGCAGCCGCTATGCCTCTTCCGGGGAGC | |
| | TGAGCCAGGGAAGCTCTCAGCTGAGCGAGG | |
| | ACTTCGACCCTGACGAGCACAGCCTGCAGG | |
| | GCTCCGACATGGAGGATGAGCGGGACCGGG | |
| | ACTCCTACCACTCCTGCCACAGCTCGGTCA | |
| | GCTACCACAAAGACTCGCCTCGCTGGGACC | |
| | AGGATGAGGAAGAGCTGGAGGAGGACCTGG | |
| | AGGACTTCCTGGAGGAGGAGGAGCTGCCTG | |
| | AAGATGAGGAGGAGCTGGAGGAGGAGGAGG | |
| | AGGAGGTGCCTGACGATTTGGGCAGCTATG | |
| | CCCAGCGTGAAGACGTAGCTGTGGCTGAGC | |
| | CCAAAGACTTCAAACGCATCAGCCTCCCGC | |
| | CAGCTGCCCCAGGGAAGGAGGACAAGGCCC | |
| | CAGTGGCACCCACCGAGGCCCCCGACATGG | |
| | CCAAGGTGGCCCCCAAGCCAGCCACGCCCG | |
| | ACAAGGTGCCTGCAGCTGAGCAGATCCCTG | |
| | AGGCTGAGCCACCCAAGGACGAGGAGAGTT | |
| | TCAGGCCGAGAGAGGATGAGGAAGGCCAGG | |
| | AGGGGCAGGACTCCATGTCCAGGGCCAAGG | |
| | CCAACTGGCTGCGTGCCTTCAACAAGGTGC | |
| | GGATGCAGCTGCAGGAGGCCCGGGGAGAAG | |
| | GAGAGATGTCTAAATCCCTATGGTTCAAAG | |
| | GCGGCCCAGGGGGCGGTCTCATCATCATCG | |
| | ACAGCATGCCAGACATCCGCAAGAGGAAAC | |
| | CTATCCCACTCGTGAGCGACTTGGCCATGT | |
| | CCCTGGTCCAGTCCAGGAAAGCGGGCATCA | |
| | CCTCGGCCTTGGCCTCCAGCACGTTGAACA | |
| | ACGAGGAGCTGAAAAACCACGTTTACAAGA | |
| | AGACCCTGCAAGCCTTAATCTACCCCATCT | |
| | CGTGCACGACGCCACACAACTTCGAAGTGT | |
| | GGACGGCCACCACGCCCACCTACTGCTACG | |
| | AGTGCGAGGGGCTGCTGTGGGGCATCGCGA | |
| | GGCAGGGCATGCGCTGCACCGAGTGCGGTG | |
| | TCAAGTGCCACGAGAAGTGCCAGGACCTGC | |
| | TCAACGCCGACTGCCTGCAGCGGGCTGCGG | |
| | AGAAGAGCTCCAAGCACGGGGCGGAGGACC | |
| | GGACACAGAACATCATCATGGTGCTCAAGG | |
| | ACCGCATGAAGATCCGGGAGCGCAACAAGC | |
| | CCGAGATCTTCGAGCTCATCCAGGAGATCT | |
| | TCGCGGTGACCAAGACGGCGCACACGCAGC | |
| | AGATGAAGGCGGTCAAGCAGAGCGTGCTGG | |
| | ACGGCACGTCCAAGTGGTCCGCCAAGATCA | |
| | GCATCACCGTGGTCTGCGCCCAGGGCTTGC | |
| | AGGCAAAGGACAAGACAGGATCCAGTGACC | |
| | CCTATGTCACCGTCCAGGTCGGGAAGACCA | |
| | AGAAACGGACAAAAACCATCTATGGGAACC | |
| | TCAACCCGGTGTGGGAGGAGAATTTCCACT | |
| | TTGAATGTCACAATTCCTCCGACCGCATCA | |
| | AGGTGCGCGTCTGGGACGAGGATGACGACA | |
| | TCAAATCCCGCGTGAAACAGAGGTTCAAGA | |
| | GGGAATCTGACGATTTCCTGGGGCAGACGA | |
| | TCATTGAGGTGCGGACGCTCAGCGGCGAGA | |
| | TGGACGTGTGGTACAACCTGGACAAGCGAA | |
| | CTGACAAATCTGCCGTGTCGGGTGCCATCC | |
| | GGCTCCACATCAGTGTGGAGATCAAAGGCG | |
| | AGGAGAAGGTGGCCCCGTACCATGTCCAGT | |
| | ACACCTGTCTGCATGAGAACCTGTTCCACT | |
| | TCGTGACCGACGTGCAGAACAATGGGGTCG | |
| | TGAAGATCCCAGATGCCAAGGGTGACGATG | |
| | CCTGGAAGGTTTACTACGATGAGACAGCCC | |
| | AGGAGATTGTGGACGAGTTTGCCATGCGCT | |
| | ACGGCGTCGAGTCCATCTACCAAGCCATGA | |
| | CCCACTTTGCCTGCCTCTCCTCCAAGTATA | |
| | TGTGCCCAGGGGTGCCTGCCGTCATGAGCA | |
| | CCCTGCTCGCCAACATCAATGCCTACTACG | |
| | CACACACCACCGCCTCCACCAACGTGTCTG | |
| | CCTCCGACCGCTTCGCCGCCTCCAACTTTG | |
| | GGAAAGAGCGCTTCGTGAAACTCCTGGACC | |
| | AGCTGCATAACTCCCTGCGGATTGACCTCT | |
| | CCATGTACCGGAATAACTTCCCAGCCAGCA | |
| | GCCCGGAGAGACTCCAGGACCTCAAATCCA | |
| | CTGTGGACCTTCTCACCAGCATCACCTTCT | |
| | TTCGGATGAAGGTACAAGAACTCCAGAGCC | |
| | CGCCCCGAGCCAGCCAGGTGGTAAAGGACT | |
| | GTGTGAAAGCCTGCCTTAATTCTACCTACG | |
| | AGTACATCTTCAATAACTGCCATGAACTGT | |
| | ACAGCCGGGAGTACCAGACAGACCCGGCCA | |
| | AGAAGGGGGAAGTTCTCCCAGAGGAACAGG | |
| | GGCCCAGCATCAAGAACCTCGACTTCTGGT | |
| | CCAAGCTGATTACCCTCATAGTGTCCATCA | |
| | TTGAGGAAGACAAGAATTCCTACACTCCCT | |
| | GCCTCAACCAGTTTCCCCAGGAGCTGAATG | |
| | TGGGTAAAATCAGCGCTGAAGTGATGTGGA | |
| | ATCTGTTTGCCCAAGACATGAAGTACGCCA | |
| | TGGAGGAGCACGACAAGCATCGTCTATGCA | |
| | AGAGTGCCGACTACATGAACCTCCACTTCA | |
| | AGGTGAAATGGCTCTACAATGAGTATGTGA | |
| | CGGAACTTCCCGCCTTCAAGGACCGCGTGC | |
| | CTGAGTACCCTGCATGGTTTGAACCCTTCG | |
| | TCATCCAGTGGCTGGATGAGAATGAGGAGG | |
| | TGTCCCGGGATTTCCTGCACGGTGCCCTGG | |
| | AGCGAGACAAGAAGGATGGGTTCCAGCAGA | |
| | CCTCAGAGCATGCCCTATTCTCCTGCTCCG | |
| | TGGTGGATGTTTTCTCCCAACTCAACCAGA | |
| | GCTTTGAAATCATCAAGAAACTCGAGTGTC | |
| | CCGACCCTCAGATCGTGGGGCACTACATGA | |
| | GGCGCTTTGCCAAGACCATCAGTAATGTGC | |
| | TCCTCCAGTATGCAGACATCATCTCCAAGG | |
| | ACTTTGCCTCCTACTGCTCCAAGGAGAAGG | |
| | AGAAAGTGCCCTGCATTCTCATGAATAACA | |
| | CTCAACAGCTACGAGTTCAGCTGGAGAAGA | |
| | TGTTCGAAGCCATGGGAGGAAAGGAGCTGG | |
| | ATGCTGAAGCCAGTGACATCCTGAAGGAGC | |
| | TTCAGGTGAAACTCAATAACGTCTTGGATG | |
| | AGCTCAGCCGGGTGTTTGCTACCAGCTTCC | |
| | AGCCGCACATTGAAGAGTGTGTCAAACAGA | |
| | TGGGTGACATCCTTAGCCAGGTTAAGGGCA | |
| | CAGGCAATGTGCCAGCCAGTGCCTGCAGCA | |
| | GCGTGGCCCAGGACGCGGACAATGTGTTGC | |
| | AGCCCATCATGGACCTGCTGGACAGCAACC | |
| | TGACCCTCTTTGCCAAAATCTGTGAGAAGA | |
| | CTGTGCTGAAGCGAGTGCTGAAGGAGCTGT | |
| | GGAAGCTGGTTATGAACACCATGGAGAAAA | |
| | CCATCGTCCTGCCGCCCCTCACTGACCAGA | |
| | CGATGATCGGGAACCTCTTGAGAAAACATG | |
| | GCAAGGGATTAGAAAAGGGCAGGGTGAAAT | |
| | TGCCAAGCCACTCAGACGGAACCCAGATGA | |
| | TCTTCAATGCAGCCAAGGAGCTGGGTCAGC | |
| | TGTCCAAACTCAAGGATCACATGGTACGAG | |
| | AAGAAGCCAAGAGCTTGACCCCAAAGCAGT | |
| | GCGCGGTTGTTGAGTTGGCCCTGGACACCA | |
| | TCAAGCAATATTTCCACGCGGGTGGCGTGG | |
| | GCCTCAAGAAGACCTTCCTGGAGAAGAGCC | |
| | CGGACCTGCAATCCTTGCGCTATGCCCTGT | |
| | CGCTCTACACGCAGGCCACCGACCTGCTAA | |
| | TCAAGACCTTTGTACAGACGCAATCGGCCC | |
| | AGGGCTTGGGTGTAGAAGACCCTGTGGGTG | |
| | AAGTCTCTGTCCATGTTGAGCTGTTCACTC | |
| | ATCCAGGAACTGGGGAACACAAGGTCACAG | |
| | TGAAAGTGGTGGCTGCCAATGACCTCAAGT | |
| | GGCAGACTTCTGGCATCTTCCGGCCGTTCA | |
| | TCGAGGTCAACATCATTGGGCCCCAGCTCA | |
| | GCGACAAGAAACGCAAGTTTGCGACCAAAT | |
| | CCAAGAACAATAGCTGGGCTCCCAAGTACA | |
| | ATGAGAGCTTCCAGTTCACGCTGAGCGCCG | |
| | ACGCGGGTCCCGAGTGCTATGAGCTGCAGG | |
| | TGTGCGTCAAGGACTACTGCTTCGCGCGCG | |
| | AGGACCGCACGGTGGGGCTGGCCGTGCTGC | |
| | AGCTGCGTGAGCTGGCCCAGCGCGGGAGCG | |
| | CCGCCTGCTGGCTGCCGCTCGGCCGCCGCA | |
| | TCCACATGGACGACACGGGCCTCACGGTGC | |
| | TGCGAATCCTCTCGCAGCGCAGCAACGACG | |
| | AGGTGGCCAAGGAGTTCGTGAAGCTCAAGT | |
| | CGGACACGCGCTCCGCCGAGGAGGGCGGTG | |
| | CCGCGCCTGCGCCTTAGCGCGGGCGGTCGG | |
| | CCGAGCGGCACTGCGCCTGCGCGGAGGGCG | |
| | CTGGGCGGGGAGGGACGGGGCTTGCGCCTT | |
| | GGTGGGACCTCCCCAGGGGCGGGGCTCGGG | |
| | GGGCTCCACGCCAAGGGTGGGCTGCGCCTA | |
| | CGCCCTTGACTCAGCTTTCCCTTTTGGGGA | |
| | ATTAGGAATGGAGGATGCCCCGCCCTCTCG | |
| | GGAGGCCACGCCCAAGGGCGCGACGAAGGA | |
| | AGGAGCCACATCCCCAACTTGAGGCCACGC | |
| | CCCCAGCACCTAGGGGGCATTTTGAGCTGG | |
| | GATGGGGGAAACCTCGTCCCTATGGAGGAG | |
| | GCCACATCCCGGGGCTCTGGTACCGGGAGG | |
| | CACCACCTCATGTCCCCTGGAAAAGCCATA | |
| | AGATGGGACCCAGACCCCTGGGACCCCAGA | |
| | CCAATTGCCAAGTATGGAAATCTCAGCTCC | |
| | CTCGAGGGGGGGCCCTGGGCAAGGGGTAGG | |
| | GCTCTCTGGAGCGCCCCTCTAGGTGGCCTG | |
| | GGGACTGGAGGGACCAGGATGCTGGTTGGA | |
| | GGGCCCCGGAATACCGGAGTCCCTTTAGAT | |
| | ATTTGTGCAAAAAATAAATGGGGGGAGGGG | |
| | GGAGGATGGGATTTCAAAAGCACATGCGCC | |
| | CTTGGGCGCCCAAACCCTGGGGGCCGAGGG | |
| | GACGGCTCTGGTTCCCCACGCTGCCCCTAC | |
| | TTCCCTTTGGGAGTTTGCCTCTCCCTCTCC | |
| | CCCAACAAACCCAGTCCTCATATCATAGAG | |
| | TTCAACACACCCATTTGACAGATGGCAAAA | |
| | CTGAGGCTTAAAGAGCTGCTTGAGACTTGG | |
| | CCAAGGTTCCAGGTGCCATACCCTCTGTGC | |
| | CCCTCCCTTAGGCCTGTGTGCCCCATGGAA | |
| | GGGTGGGCTGAGATCGGGATGACCTGACAC | |
| | AGCTCCCTATTGCTGCTAATTCCCCCTCGG | |
| | CCTCCTCCAAGGGGTGGGAATTCCAGGCCA | |
| | AGACCCCTACTTCGCCTTTCCTTCTCCGGC | |
| | TGCCAAGCAGGACCTTTGCCCTCAGCCCTT | |
| | TCTCCTGGGATCTCCATGGGGGATGCCATG | |
| | AGGGCCTCCCACCACAAAAGAGAATTTGGG | |
| | ATCCCCTGGTCCCAGGTTTCTCCATCCCTT | |
| | CTTCCTTTTCCAGAATTTTCCAAATAGGAA | |
| | AGAACAGAAGGAGACCAGAAACTCTAGGGG | |
| | GGAGAAAGAGAATGAGAGAAAGAGAATGAG | |
| | AGAGAGAGAAACACAAACACAGTGACACAG | |
| | TGAGAGCTTAGTCTCCAAGAGCCTATTCAT | |
| | TGATTCAAACACCCAAGCCACAGGATACCT | |
| | CAGATGGCCCTCTTGCCAGCTGGAAGCTCT | |
| | TTCTCCAATGAGCAAAGTTACAGTGACCTG | |
| | GCTGGAGTTACCTGGTGCACATAGGACCTT | |
| | AGGGGAAAGTTCAGCGTGGACTACACTTGC | |
| | TCTGGGATCTGCTTTTCCACATGTGTGTAT | |
| | GGCACGCCTTTTTCTGCTGGATTGGGAAGG | |
| | ACAAGATTTTGCTGTGCTAGGGAGAAATGA | |
| | AAACGGGGTGAGCTGAGTAGCTGGGTTTCT | |
| | GGAGGATAGAACATCAGATGGGGAGGCTTT | |
| | CCGAGGTGAAGAATGAGAGGGAACCACTTA | |
| | CTAGAGAGAAAAGAGCTCCAGGCCTGGGGA | |
| | ACAGCACGTGCGAAGGCCAGGAGAGAAGAA | |
| | CTGTTGAAACAACGAGAAGGGTGGCACGGC | |
| | TGGAGCTGAGCCAGCAAGGGGGATCGTGAG | |
| | GAGCCTTGGGGTTGGGGAGATCTGCAGAAG | |
| | CATCAGACCAGGCAGGGCCTCGTACGCAGT | |
| | CCTGAGGAGTTTTACTTTTATTCTAAGACA | |
| | GTTGGGGAGCTCCAGGAGCTGTTTTAAGTT | |
| | GGGGAGAGACTGGATTCCAGCCTGCAAAAG | |
| | CTGTTTTGTGAAGACTAAAACCAGTGAGGA | |
| | GAGGTGGAGGTGCTTTGGGGACACTGAAAT | |
| | GGATTCTTGGAAAGATTCTGAAGGCTGTGT | |
| | TGAAAAGACACCTATAGCTGTGGGGACATG | |
| | ACTATAATCCCAGCATTTGGGGAGACCGAG | |
| | GCTGGCAGATCACTTAAGGTCAGGAGTTTG | |
| | AGACCAGCCTGGCCAACATGGCGAAACCCC | |
| | ATCTCTGCTAAAAATACAAAAATTAGCTGG | |
| | GTGCAGTGGTGCATGCCTGTAGTCCCAGCT | |
| | ACTCAGGAGACTGAGGCGGGAGAATTGTTT | |
| | GAACCCTGGAGGCAGAGGTTGTAGTGAGTC | |
| | GTGATCACACAACTGCACTCCAGCCTGGGC | |
| | AACAGAACAATACTCCATTCCCTCCCCTCT | |
| | ACCCCACCAAAAAAAAAAAAAAATCCTGCC | |
| | CTTAGATGAGCTCTAGGGCTGCTGAGTACA | |
| | GTTGTCCCAGTTGCACAGTGCCCAAGGGTT | |
| | TGGCATTGCTAAGAAGGCCACGTGCAAATC | |
| | CTAGATATTGAGTGTTGTATGTTTGTGACG | |
| | TTGGTTTCCCGACATGTGAATGGCCCAAGT | |
| | GTCTGGAAGAAGTGGCGCCACTTTCTAATT | |
| | TGCTTGGAGATGTTGCATGTCCCTTAAATT | |
| | CAGACAGGTGCAGGTAACTGGAGGTTCTGA | |
| | ACCAAAGGTTAAAATGCAAATTCTCATACA | |
| | GGGTTGGGAAGTTGTAGCCAGGGATAAGCT | |
| | TATGTGACTGTTATATGGACTGAGGAGCAG | |
| | ATGTGAATTTCGAACCATGACATGGCTGAG | |
| | GGTAGGGGTCGGGTGGATGGATGATTCAGG | |
| | GTTGTAACCCATAGAGCCCAAAGGGGAAGT | |
| | GATCTGTGACCTGGGGTGAGGGTGATCTGG | |
| | AAGATTTTTGGATGGCTGGAAAGAAATGGG | |
| | GAAGTCGAGCTGCCTGAGAGAGCCAAGTTA | |
| | TTTCCCAAAAGATTCCTTAGGAGTCTTTCT | |
| | GTTCAAGACCTCCGTGTGTGTGTGTGTGTG | |
| | TTTAGGGTTCCCCAGCAATGGCCCAGGCAT | |
| | GTGAAGGAAACAAGCTTCTTCAGGGAATAT | |
| | TTGTTGAATGAGTTTTCCTGACTCCCAGGC | |
| | TAGAACTGTTTTTGCAATTTCCACCCTCTT | |
| | TTCTTTCCCCCAGAGAACTCCTATTCGTCC | |
| | TTCAAAACCCATCACGGAAACCCCTCTTGG | |
| | AGAAAACCCTCCTTCCTTCCCCTCAGGACT | |
| | TTCCCAGCCACCGTCTCTCCTCCAGTCCAG | |
| | CCTGATGCCATGGGACTGGGGGTTTCTCTG | |
| | TCCAGCTCTGTTTCTCCCAGACTGGGGTCT | |
| | GAGGACTCTCAGGACCCCCAACTTTACCTA | |
| | GCACAGGCTGGGCACAAGTGGGTGACAGGG | |
| | AGTCTACGCCTAGTGGAATTATGTATTGGG | |
| | GCAGGGTCAGTGTGAGAATACACATCCGCA | |
| | TGCATGTCTGTCCATGTCTGTCCGTACCAA | |
| | CCTTCCCCTTCCACACGGACCTGGGCACAT | |
| | AGGAGGTGTCTGAGCCTGACACATGGGACA | |
| | GAGAGTGGACATGGCTGAGACACGGACAGA | |
| | GAAAAGACAAGGAGTCCAGGGGGCTGAAAG | |
| | CCTTTTGAAATCAGGAAGTTCCTGTATTGG | |
| | CAGAACAAAGCCCAGAGAGGAGCAGGGCTT | |
| | TCCTCAACGCCACCCAGCAAGTGGACACAG | |
| | AGCCCGGCCTTGGATGACACCTCCAGGGTT | |
| | CTGAACCCTGGACCTCGCTTTATGCAAGGA | |
| | GCTGGCCCCACATTTCCATGAATCGGGGAA | |
| | ACAGCACAAGAAGGTTGGCCTGTGGCAGGG | |
| | CAAGGGTTAAAGGGGTGACATTGAGGGATG | |
| | CCTCAGAGTCAAAGTCCCCTGACCAAGAGG | |
| | AATAGAGTAGAAAACACAGAGACAGAGGGT | |
| | GAGATCACGCCCCGATGAGGACGGAGAGAG | |
| | ACAGAGATGGAGAGAGACATAGAGGTGGAA | |
| | ATATACAGAGAAAGATAAATGCAGAGACCA | |
| | AGGCAGGGAGTGTCGGGGGAAGTAAAGAGG | |
| | GTGTCCTGAAGAAAGAAGGATCTGTTCACT | |
| | CTTACCAGTCTGTCCTCGAATGATTTGCAT | |
| | AAAATGAGGAGGTGCCTGTCCACACCCCCA | |
| | ATTCCTCTCTCAGGCCCCAGAGCCTGAGAC | |
| | CTCACCATGCCCCCATCAGAGATGCAAAAA | |
| | ACTAAACACCCAACTAGAAATCCTTGGGAC | |
| | CTCTCTCGGCTGGGATCTCAGAGCCTTTCT | |
| | GTCCCCTACCCCTACCCCATGTGCTGTCGA | |
| | TTTTGCAGATGGGGACAACCTGGGGCCTCC | |
| | CGGAACTCTGCCACCCTGGGGAAGTTGGGG | |
| | GAGGGCCTTAGTCCCGGATCACAACCCCGT | |
| | CTGCTCCCCAGAATCCTTTCCTAAGAATCG | |
| | TTGAGGACCAAAGTTGTCTTTGCTGACACG | |
| | TGTTGCTTTTCTCTTTGCCTTTTATTGTTT | |
| | CAGAGAAAAATCAAGTTGACTGTGTCAAGT | |
| | AACACCCCACCCCTTACCCCCGTCCAGCCA | |
| | TAGTGGCTCTCTGGAGACACAGGTCACAGG | |
| | CGGAGGGTCCCCTGATCATCCCCAACCACA | |
| | CAGCCAGGGGGACTTGACCCCTGTCCACCC | |
| | CTGTCTCGTGCTCCCTCAGACCCCCACAAA | |
| | CCGGCCAAGCAGTCCGGGGAGGCTTCCCCT | |
| | CCACACAACTCTTAGCATGTGATTGCAGAT | |
| | GTGAAATCAAAACGTTGTTTGTTTTTTGTT | |
| | TTGTTTTGATTCTACCCCGTCGGTCCAGTG | |
| | TCTGCACAGACGCCTTCATTTCTCTGTAAA | |
| | TATGTGACTTGGAACAAATGTTTAACACAA | |
| | ACGAGAAGTGGTCATGAATGCATGGTGTTG | |
| | AGATGTTTTGCACTATTCTGACTTTTTGGT | |
| | CTCTGTAAAAATATTTTATTAACAGCAGAC | |
| | ATTAAAAAAAGAAAAACCACACACA | |
| UNC13A | MSLLCVGVKKAKFDGAQEKFNTYVTLKVQN | 11 |
| reference | VKSTTIAVRGSQPSWEQDFMFEINRLDLGL | |
| protein | TVEVWNKGLIWDTMVGTVWIPLRTIRQSNE | |
| NP_ | EGPGEWLTLDSQVIMADSEICGTKDPTFHR | |
| 001073890.2 | ILLDTRFELPLDIPEEEARYWAKKLEQLNA | |
| | MRDQDEYSFQDEQDKPLPVPSNQCCNWNYF | |
| | GWGEQHNDDPDSAVDDRDSDYRSETSNSIP | |
| | PPYYTTSQPNASVHQYSVRPPPLGSRESYS | |
| | DSMHSYEEFSEPQALSPTGSSRYASSGELS | |
| | QGSSQLSEDEDPDEHSLQGSDMEDERDRDS | |
| | YHSCHSSVSYHKDSPRWDQDEEELEEDLED | |
| | FLEEEELPEDEEELEEEEEEVPDDLGSYAQ | |
| | REDVAVAEPKDFKRISLPPAAPGKEDKAPV | |
| | APTEAPDMAKVAPKPATPDKVPAAEQIPEA | |
| | EPPKDEESFRPREDEEGQEGQDSMSRAKAN | |
| | WLRAFNKVRMQLQEARGEGEMSKSLWFKGG | |
| | PGGGLIIIDSMPDIRKRKPIPLVSDLAMSL | |
| | VQSRKAGITSALASSTINNEELKNHVYKKT | |
| | LQALIYPISCTTPHNFEVWTATTPTYCYEC | |
| | EGLLWGIARQGMRCTECGVKCHEKCQDLLN | |
| | ADCLQRAAEKSSKHGAEDRTQNIIMVLKDR | |
| | MKIRERNKPEIFELIQEIFAVTKTAHTQQM | |
| | KAVKQSVLDGTSKWSAKISITVVCAQGLQA | |
| | KDKTGSSDPYVTVQVGKTKKRTKTIYGNLN | |
| | PVWEENFHFECHNSSDRIKVRVWDEDDDIK | |
| | SRVKQRFKRESDDFLGQTIIEVRTLSGEMD | |
| | VWYNLDKRTDKSAVSGAIRLHISVEIKGEE | |
| | KVAPYHVQYTCLHENLFHFVTDVQNNGVVK | |
| | IPDAKGDDAWKVYYDETAQEIVDEFAMRYG | |
| | VESIYQAMTHFACLSSKYMCPGVPAVMSTL | |
| | LANINAYYAHTTASTNVSASDRFAASNFGK | |
| | ERFVKLLDQLHNSLRIDLSMYRNNFPASSP | |
| | ERLQDLKSTVDLLTSITFFRMKVQELQSPP | |
| | RASQVVKDCVKACLNSTYEYIENNCHELYS | |
| | REYQTDPAKKGEVLPEEQGPSIKNLDFWSK | |
| | LITLIVSIIEEDKNSYTPCLNQFPQELNVG | |
| | KISAEVMWNLFAQDMKYAMEEHDKHRLCKS | |
| | ADYMNLHFKVKWLYNEYVTELPAFKDRVPE | |
| | YPAWFEPFVIQWLDENEEVSRDELHGALER | |
| | DKKDGFQQTSEHALFSCSVVDVFSQLNQSF | |
| | EIIKKLECPDPQIVGHYMRRFAKTISNVLL | |
| | QYADIISKDFASYCSKEKEKVPCILMNNTQ | |
| | QLRVQLEKMFEAMGGKELDAEASDILKELQ | |
| | VKLNNVLDELSRVFATSFQPHIEECVKQMG | |
| | DILSQVKGTGNVPASACSSVAQDADNVLQP | |
| | IMDLLDSNLTLFAKICEKTVLKRVLKELWK | |
| | LVMNTMEKTIVLPPLTDQTMIGNLLRKHGK | |
| | GLEKGRVKLPSHSDGTQMIFNAAKELGQLS | |
| | KLKDHMVREEAKSLTPKQCAVVELALDTIK | |
| | QYFHAGGVGLKKTFLEKSPDLQSLRYALSL | |
| | YTQATDLLIKTFVQTQSAQGLGVEDPVGEV | |
| | SVHVELFTHPGTGEHKVTVKVVAANDLKWQ | |
| | TSGIFRPFIEVNIIGPQLSDKKRKFATKSK | |
| | NNSWAPKYNESFQFTLSADAGPECYELQVC | |
| | VKDYCFAREDRTVGLAVLQLRELAQRGSAA | |
| | CWLPLGRRIHMDDTGLTVLRILSQRSNDEV | |
| | AKEFVKLKSDTRSAEEGGAAPAP | |
| Exon 20 | ACAAGCGAACTGACAAATCTGCCGTGTCGG | 2 |
| | GTGCCATCCGGCTCCACATCAGTGTGGAGA | |
| | TCAAAGGCGAGGAGAAGGTGGCCCCGTACC | |
| | ATGTCCAGTACACCTGTCTGCATGAG | |
| Exon 21 | AACCTGTTCCACTTCGTGACCGACGTGCAG | 3 |
| | AACAATGGGGTCGTGAAGATCCCAGATGCC | |
| | AAGGGTGACGATGCCTGGAAGGTTTACTAC | |
| | GATGAGACAGCCCAGGAGATTGTGGACGAG | |
| | TTTGCCATGCGCTACGGCGTCGAGTCCATC | |
| | TACCAAGCCATGAC | |
| Intron | GTGAGGGTCATTGCTCGGCCCCTCCCATGC | 4 |
| 20-21 | CACTTCCACTCACCATTCCTGCCTGCCCAG | |
| | CTCTTCCTCTTTCTGGCCACACCATCCACA | |
| | CTCTCCTGGCCCTCTGAGACTGCCCGCCAT | |
| | GCCATTCCCTTTACCTGGAAAACTCCTCCC | |
| | TATCCATCAAAGTCCAGATTCAGGGTCACC | |
| | TCCTCTGGGAAGCCCACCTTGGCCTCCAGG | |
| | TTGACTCTCACTACTCATCATCAGGTTCTT | |
| | CCTTCTATTCCAGCCCTAACCACTCAGGAT | |
| | TGGGCCGTTTGTGTCTGGGTATGTCTCTTC | |
| | CAGCTGCCTGGGTTTCCTGGAAAGAACTCT | |
| | TATCCCCAGGAACTAGTTTGTTGAATAAAT | |
| | GCTGGTGAATGAATGAATGATTGAACAGAT | |
| | GAATGAGTGATGAGTAGATAAAAGGATGGA | |
| | TGGAGAGATGGGTGAGTACATGGATGGATA | |
| | GATGGATGAGTTGGTGGGTAGATTCGTGGC | |
| | TAGATGGATGATGGATGGATGGACAGATGG | |
| | ATGGATATATGATTGAACTATTGAAAGTAT | |
| | AGATGTATGGATGGGTGAATTTGGGGGTAA | |
| | TTGTTAGATGATGGATGAGTATAGATGAAT | |
| | GATGGATGGATAACTTGATGAGTGGATAGA | |
| | TAGATTGCTGGATAGATGATTGACTGGGTG | |
| | GATAGATGAAATGTTGGATGAGCAGATTAA | |
| | GTTGTATTGGATGGGATGGATGGAAGTGTG | |
| | GTTGAGTTATTAGAAGGAAGATTGAGTAGA | |
| | TAGGTGAATTTGTTGATAGTCAGATGGGTA | |
| | GATAGGTAGATGGATGGATGGATGGATGGA | |
| | TGTATAGGCAGATGGACAAATGGATGAATG | |
| | GGTGGGTGGATGAATGGAAGGATGTGTGGT | |
| | TGAACTATTGCAAGTATTGATAATTGGGTT | |
| | CATAATTTCTGAATATTTAGATGGATGGTT | |
| | GTGAGTGGCTGGTGGACAGACGAAAAATGG | |
| | ATGGTTGGATAAATTGATGGGTGGATGGAT | |
| | GGTTGGTTGTATGAAAGAATGAATGATTGG | |
| | GTAGGTGGATTAAGTTGCGGATCAATGTAT | |
| | GGGATGGATGAATGGATGGATGGATGGATG | |
| | TGTGGTTGAATTACTGAAAGGTTGGAAGAG | |
| | TGGATGGGTGAAATTTGGGGTAGTTAGATG | |
| | GGTGGGTGTGTGGATGGATAAAAGAGTAGA | |
| | TGAATGAATTAATGAATAAACAGGCAGATG | |
| | GATGATGTAAGCTGCCCCAGACCCTGGGAC | |
| | CTCTGACCCCCGGCGACCCCTTGCACTCTC | |
| | CATGACACTTTCTCTCCCATGGTGGCAG | |
| Cryptic | CTGCCTGGGTTTCCTGGAAAGAACTCTTAT | 5 |
| Exon 1 | CCCCAGGAACTAGTTTGTTGAATAAATGCT | |
| | GGTGAATGAATGAATGATTGAACAGATGAA | |
| | TGAGTGATGAGTAGATAAAAGGATGGATGG | |
| | AGAGATGG | |
| Cryptic | CCCTAACCACTCAGGATTGGGCCGTTTGTG | 6 |
| Exon 2 | TCTGGGTATGTCTCTTCCAGCTGCCTGGGT | |
| | TTCCTGGAAAGAACTCTTATCCCCAGGAAC | |
| | TAGTTTGTTGAATAAATGCTGGTGAATGAA | |
| | TGAATGATTGAACAGATGAATGAGTGATGA | |
| | GTAGATAAAAGGATGGATGGAGAGATGGG | |
| Cryptic | GCCCCCGGTGCTGAACCAAGATGGCCGGTG | 7 |
| Exon | GCGGCCGGGCCCCGGCGTGAGCCAAGCGCG | |
| Splice | GGCTGCAGCCGGGAGATGCCCCAGCCCAGC | |
| Variant | GGCCGCTGAGCCCGACCCGACAGAGCCGGC | |
| 1 | CCGGCCGCCTCCGGCCCACCTGCGAGCTCG | |
| | GAGACATGTCTCTGCTTTGCGTTGGAGTCA | |
| | AAAAAGCCAAGTTTGATGGTGCCCAAGAGA | |
| | AATTCAACACGTACGTGACCCTGAAAGTGC | |
| | AGAATGTCAAGAGCACGACCATCGCGGTGC | |
| | GGGGCAGCCAGCCCAGCTGGGAGCAGGATT | |
| | TCATGTTCGAGATTAACCGTCTGGATTTGG | |
| | GACTGACGGTGGAGGTGTGGAATAAGGGTC | |
| | TCATCTGGGACACAATGGTGGGCACTGTGT | |
| | GGATCCCACTGAGGACCATCCGCCAGTCCA | |
| | ATGAGGAGGGCCCTGGAGAGTGGCTGACGC | |
| | TGGACTCCCAGGTCATCATGGCAGACAGTG | |
| | AGATCTGTGGCACCAAGGACCCCACCTTCC | |
| | ACCGCATCCTCCTGGACACGCGCTTTGAGC | |
| | TACCCTTAGACATTCCTGAAGAGGAGGCTC | |
| | GCTACTGGGCCAAGAAGCTGGAGCAGCTCA | |
| | ATGCTATGCGGGACCAGGATGAATATTCGT | |
| | TCCAAGATGAGCAAGACAAGCCTCTGCCTG | |
| | TCCCCAGCAACCAGTGCTGCAACTGGAATT | |
| | ATTTTGGCTGGGGTGAGCAGCACAACGATG | |
| | ACCCCGACAGTGCAGTGGATGATCGTGACA | |
| | GTGACTACCGCAGTGAAACGAGCAACAGCA | |
| | TCCCGCCGCCCTATTATACTACGTCACAAC | |
| | CCAACGCCTCAGTCCACCAATATTCTGTTC | |
| | GCCCACCACCCCTGGGCTCCCGGGAGTCCT | |
| | ACAGTGACTCCATGCACAGTTACGAGGAGT | |
| | TCTCTGAGCCACAAGCCCTCAGCCCCACGG | |
| | GTAGCAGCCGCTATGCCTCTTCCGGGGAGC | |
| | TGAGCCAGGGAAGCTCTCAGCTGAGCGAGG | |
| | ACTTCGACCCTGACGAGCACAGCCTGCAGG | |
| | GCTCCGACATGGAGGATGAGCGGGACCGGG | |
| | ACTCCTACCACTCCTGCCACAGCTCGGTCA | |
| | GCTACCACAAAGACTCGCCTCGCTGGGACC | |
| | AGGATGAGGAAGAGCTGGAGGAGGACCTGG | |
| | AGGACTTCCTGGAGGAGGAGGAGCTGCCTG | |
| | AAGATGAGGAGGAGCTGGAGGAGGAGGAGG | |
| | AGGAGGTGCCTGACGATTTGGGCAGCTATG | |
| | CCCAGCGTGAAGACGTAGCTGTGGCTGAGC | |
| | CCAAAGACTTCAAACGCATCAGCCTCCCGC | |
| | CAGCTGCCCCAGGGAAGGAGGACAAGGCCC | |
| | CAGTGGCACCCACCGAGGCCCCCGACATGG | |
| | CCAAGGTGGCCCCCAAGCCAGCCACGCCCG | |
| | ACAAGGTGCCTGCAGCTGAGCAGATCCCTG | |
| | AGGCTGAGCCACCCAAGGACGAGGAGAGTT | |
| | TCAGGCCGAGAGAGGATGAGGAAGGCCAGG | |
| | AGGGGCAGGACTCCATGTCCAGGGCCAAGG | |
| | CCAACTGGCTGCGTGCCTTCAACAAGGTGC | |
| | GGATGCAGCTGCAGGAGGCCCGGGGAGAAG | |
| | GAGAGATGTCTAAATCCCTATGGTTCAAAG | |
| | GCGGCCCAGGGGGCGGTCTCATCATCATCG | |
| | ACAGCATGCCAGACATCCGCAAGAGGAAAC | |
| | CTATCCCACTCGTGAGCGACTTGGCCATGT | |
| | CCCTGGTCCAGTCCAGGAAAGCGGGCATCA | |
| | CCTCGGCCTTGGCCTCCAGCACGTTGAACA | |
| | ACGAGGAGCTGAAAAACCACGTTTACAAGA | |
| | AGACCCTGCAAGCCTTAATCTACCCCATCT | |
| | CGTGCACGACGCCACACAACTTCGAAGTGT | |
| | GGACGGCCACCACGCCCACCTACTGCTACG | |
| | AGTGCGAGGGGCTGCTGTGGGGCATCGCGA | |
| | GGCAGGGCATGCGCTGCACCGAGTGCGGTG | |
| | TCAAGTGCCACGAGAAGTGCCAGGACCTGC | |
| | TCAACGCCGACTGCCTGCAGCGGGCTGCGG | |
| | AGAAGAGCTCCAAGCACGGGGCGGAGGACC | |
| | GGACACAGAACATCATCATGGTGCTCAAGG | |
| | ACCGCATGAAGATCCGGGAGCGCAACAAGC | |
| | CCGAGATCTTCGAGCTCATCCAGGAGATCT | |
| | TCGCGGTGACCAAGACGGCGCACACGCAGC | |
| | AGATGAAGGCGGTCAAGCAGAGCGTGCTGG | |
| | ACGGCACGTCCAAGTGGTCCGCCAAGATCA | |
| | GCATCACCGTGGTCTGCGCCCAGGGCTTGC | |
| | AGGCAAAGGACAAGACAGGATCCAGTGACC | |
| | CCTATGTCACCGTCCAGGTCGGGAAGACCA | |
| | AGAAACGGACAAAAACCATCTATGGGAACC | |
| | TCAACCCGGTGTGGGAGGAGAATTTCCACT | |
| | TTGAATGTCACAATTCCTCCGACCGCATCA | |
| | AGGTGCGCGTCTGGGACGAGGATGACGACA | |
| | TCAAATCCCGCGTGAAACAGAGGTTCAAGA | |
| | GGGAATCTGACGATTTCCTGGGGCAGACGA | |
| | TCATTGAGGTGCGGACGCTCAGCGGCGAGA | |
| | TGGACGTGTGGTACAACCTGGACAAGCGAA | |
| | CTGACAAATCTGCCGTGTCGGGTGCCATCC | |
| | GGCTCCACATCAGTGTGGAGATCAAAGGCG | |
| | AGGAGAAGGTGGCCCCGTACCATGTCCAGT | |
| | ACACCTGTCTGCATGAGCTGCCTGGGTTTC | |
| | CTGGAAAGAACTCTTATCCCCAGGAACTAG | |
| | TTTGTTGAATAAATGCTGGTGAATGAATGA | |
| | ATGATTGAACAGATGAATGAGTGATGAGTA | |
| | GATAAAAGGATGGATGGAGAGATGGAACCT | |
| | GTTCCACTTCGTGACCGACGTGCAGAACAA | |
| | TGGGGTCGTGAAGATCCCAGATGCCAAGGG | |
| | TGACGATGCCTGGAAGGTTTACTACGATGA | |
| | GACAGCCCAGGAGATTGTGGACGAGTTTGC | |
| | CATGCGCTACGGCGTCGAGTCCATCTACCA | |
| | AGCCATGACCCACTTTGCCTGCCTCTCCTC | |
| | CAAGTATATGTGCCCAGGGGTGCCTGCCGT | |
| | CATGAGCACCCTGCTCGCCAACATCAATGC | |
| | CTACTACGCACACACCACCGCCTCCACCAA | |
| | CGTGTCTGCCTCCGACCGCTTCGCCGCCTC | |
| | CAACTTTGGGAAAGAGCGCTTCGTGAAACT | |
| | CCTGGACCAGCTGCATAACTCCCTGCGGAT | |
| | TGACCTCTCCATGTACCGGAATAACTTCCC | |
| | AGCCAGCAGCCCGGAGAGACTCCAGGACCT | |
| | CAAATCCACTGTGGACCTTCTCACCAGCAT | |
| | CACCTTCTTTCGGATGAAGGTACAAGAACT | |
| | CCAGAGCCCGCCCCGAGCCAGCCAGGTGGT | |
| | AAAGGACTGTGTGAAAGCCTGCCTTAATTC | |
| | TACCTACGAGTACATCTTCAATAACTGCCA | |
| | TGAACTGTACAGCCGGGAGTACCAGACAGA | |
| | CCCGGCCAAGAAGGGGGAAGTTCTCCCAGA | |
| | GGAACAGGGGCCCAGCATCAAGAACCTCGA | |
| | CTTCTGGTCCAAGCTGATTACCCTCATAGT | |
| | GTCCATCATTGAGGAAGACAAGAATTCCTA | |
| | CACTCCCTGCCTCAACCAGTTTCCCCAGGA | |
| | GCTGAATGTGGGTAAAATCAGCGCTGAAGT | |
| | GATGTGGAATCTGTTTGCCCAAGACATGAA | |
| | GTACGCCATGGAGGAGCACGACAAGCATCG | |
| | TCTATGCAAGAGTGCCGACTACATGAACCT | |
| | CCACTTCAAGGTGAAATGGCTCTACAATGA | |
| | GTATGTGACGGAACTTCCCGCCTTCAAGGA | |
| | CCGCGTGCCTGAGTACCCTGCATGGTTTGA | |
| | ACCCTTCGTCATCCAGTGGCTGGATGAGAA | |
| | TGAGGAGGTGTCCCGGGATTTCCTGCACGG | |
| | TGCCCTGGAGCGAGACAAGAAGGATGGGTT | |
| | CCAGCAGACCTCAGAGCATGCCCTATTCTC | |
| | CTGCTCCGTGGTGGATGTTTTCTCCCAACT | |
| | CAACCAGAGCTTTGAAATCATCAAGAAACT | |
| | CGAGTGTCCCGACCCTCAGATCGTGGGGCA | |
| | CTACATGAGGCGCTTTGCCAAGACCATCAG | |
| | TAATGTGCTCCTCCAGTATGCAGACATCAT | |
| | CTCCAAGGACTTTGCCTCCTACTGCTCCAA | |
| | GGAGAAGGAGAAAGTGCCCTGCATTCTCAT | |
| | GAATAACACTCAACAGCTACGAGTTCAGCT | |
| | GGAGAAGATGTTCGAAGCCATGGGAGGAAA | |
| | GGAGCTGGATGCTGAAGCCAGTGACATCCT | |
| | GAAGGAGCTTCAGGTGAAACTCAATAACGT | |
| | CTTGGATGAGCTCAGCCGGGTGTTTGCTAC | |
| | CAGCTTCCAGCCGCACATTGAAGAGTGTGT | |
| | CAAACAGATGGGTGACATCCTTAGCCAGGT | |
| | TAAGGGCACAGGCAATGTGCCAGCCAGTGC | |
| | CTGCAGCAGCGTGGCCCAGGACGCGGACAA | |
| | TGTGTTGCAGCCCATCATGGACCTGCTGGA | |
| | CAGCAACCTGACCCTCTTTGCCAAAATCTG | |
| | TGAGAAGACTGTGCTGAAGCGAGTGCTGAA | |
| | GGAGCTGTGGAAGCTGGTTATGAACACCAT | |
| | GGAGAAAACCATCGTCCTGCCGCCCCTCAC | |
| | TGACCAGACGATGATCGGGAACCTCTTGAG | |
| | AAAACATGGCAAGGGATTAGAAAAGGGCAG | |
| | GGTGAAATTGCCAAGCCACTCAGACGGAAC | |
| | CCAGATGATCTTCAATGCAGCCAAGGAGCT | |
| | GGGTCAGCTGTCCAAACTCAAGGATCACAT | |
| | GGTACGAGAAGAAGCCAAGAGCTTGACCCC | |
| | AAAGCAGTGCGCGGTTGTTGAGTTGGCCCT | |
| | GGACACCATCAAGCAATATTTCCACGCGGG | |
| | TGGCGTGGGCCTCAAGAAGACCTTCCTGGA | |
| | GAAGAGCCCGGACCTGCAATCCTTGCGCTA | |
| | TGCCCTGTCGCTCTACACGCAGGCCACCGA | |
| | CCTGCTAATCAAGACCTTTGTACAGACGCA | |
| | ATCGGCCCAGGGCTTGGGTGTAGAAGACCC | |
| | TGTGGGTGAAGTCTCTGTCCATGTTGAGCT | |
| | GTTCACTCATCCAGGAACTGGGGAACACAA | |
| | GGTCACAGTGAAAGTGGTGGCTGCCAATGA | |
| | CCTCAAGTGGCAGACTTCTGGCATCTTCCG | |
| | GCCGTTCATCGAGGTCAACATCATTGGGCC | |
| | CCAGCTCAGCGACAAGAAACGCAAGTTTGC | |
| | GACCAAATCCAAGAACAATAGCTGGGCTCC | |
| | CAAGTACAATGAGAGCTTCCAGTTCACGCT | |
| | GAGCGCCGACGCGGGTCCCGAGTGCTATGA | |
| | GCTGCAGGTGTGCGTCAAGGACTACTGCTT | |
| | CGCGCGCGAGGACCGCACGGTGGGGCTGGC | |
| | CGTGCTGCAGCTGCGTGAGCTGGCCCAGCG | |
| | CGGGAGCGCCGCCTGCTGGCTGCCGCTCGG | |
| | CCGCCGCATCCACATGGACGACACGGGCCT | |
| | CACGGTGCTGCGAATCCTCTCGCAGCGCAG | |
| | CAACGACGAGGTGGCCAAGGAGTTCGTGAA | |
| | GCTCAAGTCGGACACGCGCTCCGCCGAGGA | |
| | GGGCGGTGCCGCGCCTGCGCCTTAGCGCGG | |
| | GCGGTCGGCCGAGCGGCACTGCGCCTGCGC | |
| | GGAGGGCGCTGGGCGGGGAGGGACGGGGCT | |
| | TGCGCCTTGGTGGGACCTCCCCAGGGGCGG | |
| | GGCTCGGGGGGCTCCACGCCAAGGGTGGGC | |
| | TGCGCCTACGCCCTTGACTCAGCTTTCCCT | |
| | TTTGGGGAATTAGGAATGGAGGATGCCCCG | |
| | CCCTCTCGGGAGGCCACGCCCAAGGGCGCG | |
| | ACGAAGGAAGGAGCCACATCCCCAACTTGA | |
| | GGCCACGCCCCCAGCACCTAGGGGGCATTT | |
| | TGAGCTGGGATGGGGGAAACCTCGTCCCTA | |
| | TGGAGGAGGCCACATCCCGGGGCTCTGGTA | |
| | CCGGGAGGCACCACCTCATGTCCCCTGGAA | |
| | AAGCCATAAGATGGGACCCAGACCCCTGGG | |
| | ACCCCAGACCAATTGCCAAGTATGGAAATC | |
| | TCAGCTCCCTCGAGGGGGGGCCCTGGGCAA | |
| | GGGGTAGGGCTCTCTGGAGCGCCCCTCTAG | |
| | GTGGCCTGGGGACTGGAGGGACCAGGATGC | |
| | TGGTTGGAGGGCCCCGGAATACCGGAGTCC | |
| | CTTTAGATATTTGTGCAAAAAATAAATGGG | |
| | GGGAGGGGGGAGGATGGGATTTCAAAAGCA | |
| | CATGCGCCCTTGGGCGCCCAAACCCTGGGG | |
| | GCCGAGGGGACGGCTCTGGTTCCCCACGCT | |
| | GCCCCTACTTCCCTTTGGGAGTTTGCCTCT | |
| | CCCTCTCCCCCAACAAACCCAGTCCTCATA | |
| | TCATAGAGTTCAACACACCCATTTGACAGA | |
| | TGGCAAAACTGAGGCTTAAAGAGCTGCTTG | |
| | AGACTTGGCCAAGGTTCCAGGTGCCATACC | |
| | CTCTGTGCCCCTCCCTTAGGCCTGTGTGCC | |
| | CCATGGAAGGGTGGGCTGAGATCGGGATGA | |
| | CCTGACACAGCTCCCTATTGCTGCTAATTC | |
| | CCCCTCGGCCTCCTCCAAGGGGTGGGAATT | |
| | CCAGGCCAAGACCCCTACTTCGCCTTTCCT | |
| | TCTCCGGCTGCCAAGCAGGACCTTTGCCCT | |
| | CAGCCCTTTCTCCTGGGATCTCCATGGGGG | |
| | ATGCCATGAGGGCCTCCCACCACAAAAGAG | |
| | AATTTGGGATCCCCTGGTCCCAGGTTTCTC | |
| | CATCCCTTCTTCCTTTTCCAGAATTTTCCA | |
| | AATAGGAAAGAACAGAAGGAGACCAGAAAC | |
| | TCTAGGGGGGAGAAAGAGAATGAGAGAAAG | |
| | AGAATGAGAGAGAGAGAAACACAAACACAG | |
| | TGACACAGTGAGAGCTTAGTCTCCAAGAGC | |
| | CTATTCATTGATTCAAACACCCAAGCCACA | |
| | GGATACCTCAGATGGCCCTCTTGCCAGCTG | |
| | GAAGCTCTTTCTCCAATGAGCAAAGTTACA | |
| | GTGACCTGGCTGGAGTTACCTGGTGCACAT | |
| | AGGACCTTAGGGGAAAGTTCAGCGTGGACT | |
| | ACACTTGCTCTGGGATCTGCTTTTCCACAT | |
| | GTGTGTATGGCACGCCTTTTTCTGCTGGAT | |
| | TGGGAAGGACAAGATTTTGCTGTGCTAGGG | |
| | AGAAATGAAAACGGGGTGAGCTGAGTAGCT | |
| | GGGTTTCTGGAGGATAGAACATCAGATGGG | |
| | GAGGCTTTCCGAGGTGAAGAATGAGAGGGA | |
| | ACCACTTACTAGAGAGAAAAGAGCTCCAGG | |
| | CCTGGGGAACAGCACGTGCGAAGGCCAGGA | |
| | GAGAAGAACTGTTGAAACAACGAGAAGGGT | |
| | GGCACGGCTGGAGCTGAGCCAGCAAGGGGG | |
| | ATCGTGAGGAGCCTTGGGGTTGGGGAGATC | |
| | TGCAGAAGCATCAGACCAGGCAGGGCCTCG | |
| | TACGCAGTCCTGAGGAGTTTTACTTTTATT | |
| | CTAAGACAGTTGGGGAGCTCCAGGAGCTGT | |
| | TTTAAGTTGGGGAGAGACTGGATTCCAGCC | |
| | TGCAAAAGCTGTTTTGTGAAGACTAAAACC | |
| | AGTGAGGAGAGGTGGAGGTGCTTTGGGGAC | |
| | ACTGAAATGGATTCTTGGAAAGATTCTGAA | |
| | GGCTGTGTTGAAAAGACACCTATAGCTGTG | |
| | GGGACATGACTATAATCCCAGCATTTGGGG | |
| | AGACCGAGGCTGGCAGATCACTTAAGGTCA | |
| | GGAGTTTGAGACCAGCCTGGCCAACATGGC | |
| | GAAACCCCATCTCTGCTAAAAATACAAAAA | |
| | TTAGCTGGGTGCAGTGGTGCATGCCTGTAG | |
| | TCCCAGCTACTCAGGAGACTGAGGCGGGAG | |
| | AATTGTTTGAACCCTGGAGGCAGAGGTTGT | |
| | AGTGAGTCGTGATCACACAACTGCACTCCA | |
| | GCCTGGGCAACAGAACAATACTCCATTCCC | |
| | TCCCCTCTACCCCACCAAAAAAAAAAAAAA | |
| | ATCCTGCCCTTAGATGAGCTCTAGGGCTGC | |
| | TGAGTACAGTTGTCCCAGTTGCACAGTGCC | |
| | CAAGGGTTTGGCATTGCTAAGAAGGCCACG | |
| | TGCAAATCCTAGATATTGAGTGTTGTATGT | |
| | TTGTGACGTTGGTTTCCCGACATGTGAATG | |
| | GCCCAAGTGTCTGGAAGAAGTGGCGCCACT | |
| | TTCTAATTTGCTTGGAGATGTTGCATGTCC | |
| | CTTAAATTCAGACAGGTGCAGGTAACTGGA | |
| | GGTTCTGAACCAAAGGTTAAAATGCAAATT | |
| | CTCATACAGGGTTGGGAAGTTGTAGCCAGG | |
| | GATAAGCTTATGTGACTGTTATATGGACTG | |
| | AGGAGCAGATGTGAATTTCGAACCATGACA | |
| | TGGCTGAGGGTAGGGGTCGGGTGGATGGAT | |
| | GATTCAGGGTTGTAACCCATAGAGCCCAAA | |
| | GGGGAAGTGATCTGTGACCTGGGGTGAGGG | |
| | TGATCTGGAAGATTTTTGGATGGCTGGAAA | |
| | GAAATGGGGAAGTCGAGCTGCCTGAGAGAG | |
| | CCAAGTTATTTCCCAAAAGATTCCTTAGGA | |
| | GTCTTTCTGTTCAAGACCTCCGTGTGTGTG | |
| | TGTGTGTGTTTAGGGTTCCCCAGCAATGGC | |
| | CCAGGCATGTGAAGGAAACAAGCTTCTTCA | |
| | GGGAATATTTGTTGAATGAGTTTTCCTGAC | |
| | TCCCAGGCTAGAACTGTTTTTGCAATTTCC | |
| | ACCCTCTTTTCTTTCCCCCAGAGAACTCCT | |
| | ATTCGTCCTTCAAAACCCATCACGGAAACC | |
| | CCTCTTGGAGAAAACCCTCCTTCCTTCCCC | |
| | TCAGGACTTTCCCAGCCACCGTCTCTCCTC | |
| | CAGTCCAGCCTGATGCCATGGGACTGGGGG | |
| | TTTCTCTGTCCAGCTCTGTTTCTCCCAGAC | |
| | TGGGGTCTGAGGACTCTCAGGACCCCCAAC | |
| | TTTACCTAGCACAGGCTGGGCACAAGTGGG | |
| | TGACAGGGAGTCTACGCCTAGTGGAATTAT | |
| | GTATTGGGGCAGGGTCAGTGTGAGAATACA | |
| | CATCCGCATGCATGTCTGTCCATGTCTGTC | |
| | CGTACCAACCTTCCCCTTCCACACGGACCT | |
| | GGGCACATAGGAGGTGTCTGAGCCTGACAC | |
| | ATGGGACAGAGAGTGGACATGGCTGAGACA | |
| | CGGACAGAGAAAAGACAAGGAGTCCAGGGG | |
| | GCTGAAAGCCTTTTGAAATCAGGAAGTTCC | |
| | TGTATTGGCAGAACAAAGCCCAGAGAGGAG | |
| | CAGGGCTTTCCTCAACGCCACCCAGCAAGT | |
| | GGACACAGAGCCCGGCCTTGGATGACACCT | |
| | CCAGGGTTCTGAACCCTGGACCTCGCTTTA | |
| | TGCAAGGAGCTGGCCCCACATTTCCATGAA | |
| | TCGGGGAAACAGCACAAGAAGGTTGGCCTG | |
| | TGGCAGGGCAAGGGTTAAAGGGGTGACATT | |
| | GAGGGATGCCTCAGAGTCAAAGTCCCCTGA | |
| | CCAAGAGGAATAGAGTAGAAAACACAGAGA | |
| | CAGAGGGTGAGATCACGCCCCGATGAGGAC | |
| | GGAGAGAGACAGAGATGGAGAGAGACATAG | |
| | AGGTGGAAATATACAGAGAAAGATAAATGC | |
| | AGAGACCAAGGCAGGGAGTGTCGGGGGAAG | |
| | TAAAGAGGGTGTCCTGAAGAAAGAAGGATC | |
| | TGTTCACTCTTACCAGTCTGTCCTCGAATG | |
| | ATTTGCATAAAATGAGGAGGTGCCTGTCCA | |
| | CACCCCCAATTCCTCTCTCAGGCCCCAGAG | |
| | CCTGAGACCTCACCATGCCCCCATCAGAGA | |
| | TGCAAAAAACTAAACACCCAACTAGAAATC | |
| | CTTGGGACCTCTCTCGGCTGGGATCTCAGA | |
| | GCCTTTCTGTCCCCTACCCCTACCCCATGT | |
| | GCTGTCGATTTTGCAGATGGGGACAACCTG | |
| | GGGCCTCCCGGAACTCTGCCACCCTGGGGA | |
| | AGTTGGGGGAGGGCCTTAGTCCCGGATCAC | |
| | AACCCCGTCTGCTCCCCAGAATCCTTTCCT | |
| | AAGAATCGTTGAGGACCAAAGTTGTCTTTG | |
| | CTGACACGTGTTGCTTTTCTCTTTGCCTTT | |
| | TATTGTTTCAGAGAAAAATCAAGTTGACTG | |
| | TGTCAAGTAACACCCCACCCCTTACCCCCG | |
| | TCCAGCCATAGTGGCTCTCTGGAGACACAG | |
| | GTCACAGGCGGAGGGTCCCCTGATCATCCC | |
| | CAACCACACAGCCAGGGGGACTTGACCCCT | |
| | GTCCACCCCTGTCTCGTGCTCCCTCAGACC | |
| | CCCACAAACCGGCCAAGCAGTCCGGGGAGG | |
| | CTTCCCCTCCACACAACTCTTAGCATGTGA | |
| | TTGCAGATGTGAAATCAAAACGTTGTTTGT | |
| | TTTTTGTTTTGTTTTGATTCTACCCCGTCG | |
| | GTCCAGTGTCTGCACAGACGCCTTCATTTC | |
| | TCTGTAAATATGTGACTTGGAACAAATGTT | |
| | TAACACAAACGAGAAGTGGTCATGAATGCA | |
| | TGGTGTTGAGATGTTTTGCACTATTCTGAC | |
| | TTTTTGGTCTCTGTAAAAATATTTTATTAA | |
| | CAGCAGACATTAAAAAAAGAAAAACCACAC | |
| | ACA | |
| Cryptic | MSLLCVGVKKAKFDGAQEKENTYVTLKVQN | 8 |
| Exon | VKSTTIAVRGSQPSWEQDFMFEINRLDLGL | |
| Splice | TVEVWNKGLIWDTMVGTVWIPLRTIRQSNE | |
| Variant | EGPGEWLTLDSQVIMADSEICGTKDPTFHR | |
| 1 | ILLDTRFELPLDIPEEEARYWAKKLEQLNA | |
| | MRDQDEYSFQDEQDKPLPVPSNQCCNWNYF | |
| | GWGEQHNDDPDSAVDDRDSDYRSETSNSIP | |
| | PPYYTTSQPNASVHQYSVRPPPLGSRESYS | |
| | DSMHSYEEFSEPQALSPTGSSRYASSGELS | |
| | QGSSQLSEDFDPDEHSLQGSDMEDERDRDS | |
| | YHSCHSSVSYHKDSPRWDQDEEELEEDLED | |
| | FLEEEELPEDEEELEEEEEEVPDDLGSYAQ | |
| | REDVAVAEPKDFKRISLPPAAPGKEDKAPV | |
| | APTEAPDMAKVAPKPATPDKVPAAEQIPEA | |
| | EPPKDEESFRPREDEEGQEGQDSMSRAKAN | |
| | WLRAFNKVRMQLQEARGEGEMSKSLWFKGG | |
| | PGGGLIIIDSMPDIRKRKPIPLVSDLAMSL | |
| | VQSRKAGITSALASSTLNNEELKNHVYKKT | |
| | LQALIYPISCTTPHNFEVWTATTPTYCYEC | |
| | EGLLWGIARQGMRCTECGVKCHEKCQDLLN | |
| | ADCLQRAAEKSSKHGAEDRTQNIIMVLKDR | |
| | MKIRERNKPEIFELIQEIFAVTKTAHTQQM | |
| | KAVKQSVLDGTSKWSAKISITVVCAQGLQA | |
| | KDKTGSSDPYVTVQVGKTKKRTKTIYGNLN | |
| | PVWEENFHFECHNSSDRIKVRVWDEDDDIK | |
| | SRVKQRFKRESDDELGQTIIEVRTLSGEMD | |
| | VWYNLDKRTDKSAVSGAIRLHISVEIKGEE | |
| | KVAPYHVQYTCLHELPGFPGKNSYPQELVC | |
| | * | |
| Cryptic Exon | GCCCCCGGTGCTGAACCAAGATGGCCGGTG | 9 |
| Splice Variant | GCGGCCGGGCCCCGGCGTGAGCCAAGCGCG | |
| 2 | GGCTGCAGCCGGGAGATGCCCCAGCCCAGC | |
| | GGCCGCTGAGCCCGACCCGACAGAGCCGGC | |
| | CCGGCCGCCTCCGGCCCACCTGCGAGCTCG | |
| | GAGACATGTCTCTGCTTTGCGTTGGAGTCA | |
| | AAAAAGCCAAGTTTGATGGTGCCCAAGAGA | |
| | AATTCAACACGTACGTGACCCTGAAAGTGC | |
| | AGAATGTCAAGAGCACGACCATCGCGGTGC | |
| | GGGGCAGCCAGCCCAGCTGGGAGCAGGATT | |
| | TCATGTTCGAGATTAACCGTCTGGATTTGG | |
| | GACTGACGGTGGAGGTGTGGAATAAGGGTC | |
| | TCATCTGGGACACAATGGTGGGCACTGTGT | |
| | GGATCCCACTGAGGACCATCCGCCAGTCCA | |
| | ATGAGGAGGGCCCTGGAGAGTGGCTGACGC | |
| | TGGACTCCCAGGTCATCATGGCAGACAGTG | |
| | AGATCTGTGGCACCAAGGACCCCACCTTCC | |
| | ACCGCATCCTCCTGGACACGCGCTTTGAGC | |
| | TACCCTTAGACATTCCTGAAGAGGAGGCTC | |
| | GCTACTGGGCCAAGAAGCTGGAGCAGCTCA | |
| | ATGCTATGCGGGACCAGGATGAATATTCGT | |
| | TCCAAGATGAGCAAGACAAGCCTCTGCCTG | |
| | TCCCCAGCAACCAGTGCTGCAACTGGAATT | |
| | ATTTTGGCTGGGGTGAGCAGCACAACGATG | |
| | ACCCCGACAGTGCAGTGGATGATCGTGACA | |
| | GTGACTACCGCAGTGAAACGAGCAACAGCA | |
| | TCCCGCCGCCCTATTATACTACGTCACAAC | |
| | CCAACGCCTCAGTCCACCAATATTCTGTTC | |
| | GCCCACCACCCCTGGGCTCCCGGGAGTCCT | |
| | ACAGTGACTCCATGCACAGTTACGAGGAGT | |
| | TCTCTGAGCCACAAGCCCTCAGCCCCACGG | |
| | GTAGCAGCCGCTATGCCTCTTCCGGGGAGC | |
| | TGAGCCAGGGAAGCTCTCAGCTGAGCGAGG | |
| | ACTTCGACCCTGACGAGCACAGCCTGCAGG | |
| | GCTCCGACATGGAGGATGAGCGGGACCGGG | |
| | ACTCCTACCACTCCTGCCACAGCTCGGTCA | |
| | GCTACCACAAAGACTCGCCTCGCTGGGACC | |
| | AGGATGAGGAAGAGCTGGAGGAGGACCTGG | |
| | AGGACTTCCTGGAGGAGGAGGAGCTGCCTG | |
| | AAGATGAGGAGGAGCTGGAGGAGGAGGAGG | |
| | AGGAGGTGCCTGACGATTTGGGCAGCTATG | |
| | CCCAGCGTGAAGACGTAGCTGTGGCTGAGC | |
| | CCAAAGACTTCAAACGCATCAGCCTCCCGC | |
| | CAGCTGCCCCAGGGAAGGAGGACAAGGCCC | |
| | CAGTGGCACCCACCGAGGCCCCCGACATGG | |
| | CCAAGGTGGCCCCCAAGCCAGCCACGCCCG | |
| | ACAAGGTGCCTGCAGCTGAGCAGATCCCTG | |
| | AGGCTGAGCCACCCAAGGACGAGGAGAGTT | |
| | TCAGGCCGAGAGAGGATGAGGAAGGCCAGG | |
| | AGGGGCAGGACTCCATGTCCAGGGCCAAGG | |
| | CCAACTGGCTGCGTGCCTTCAACAAGGTGC | |
| | GGATGCAGCTGCAGGAGGCCCGGGGAGAAG | |
| | GAGAGATGTCTAAATCCCTATGGTTCAAAG | |
| | GCGGCCCAGGGGGCGGTCTCATCATCATCG | |
| | ACAGCATGCCAGACATCCGCAAGAGGAAAC | |
| | CTATCCCACTCGTGAGCGACTTGGCCATGT | |
| | CCCTGGTCCAGTCCAGGAAAGCGGGCATCA | |
| | CCTCGGCCTTGGCCTCCAGCACGTTGAACA | |
| | ACGAGGAGCTGAAAAACCACGTTTACAAGA | |
| | AGACCCTGCAAGCCTTAATCTACCCCATCT | |
| | CGTGCACGACGCCACACAACTTCGAAGTGT | |
| | GGACGGCCACCACGCCCACCTACTGCTACG | |
| | AGTGCGAGGGGCTGCTGTGGGGCATCGCGA | |
| | GGCAGGGCATGCGCTGCACCGAGTGCGGTG | |
| | TCAAGTGCCACGAGAAGTGCCAGGACCTGC | |
| | TCAACGCCGACTGCCTGCAGCGGGCTGCGG | |
| | AGAAGAGCTCCAAGCACGGGGCGGAGGACC | |
| | GGACACAGAACATCATCATGGTGCTCAAGG | |
| | ACCGCATGAAGATCCGGGAGCGCAACAAGC | |
| | CCGAGATCTTCGAGCTCATCCAGGAGATCT | |
| | TCGCGGTGACCAAGACGGCGCACACGCAGC | |
| | AGATGAAGGCGGTCAAGCAGAGCGTGCTGG | |
| | ACGGCACGTCCAAGTGGTCCGCCAAGATCA | |
| | GCATCACCGTGGTCTGCGCCCAGGGCTTGC | |
| | AGGCAAAGGACAAGACAGGATCCAGTGACC | |
| | CCTATGTCACCGTCCAGGTCGGGAAGACCA | |
| | AGAAACGGACAAAAACCATCTATGGGAACC | |
| | TCAACCCGGTGTGGGAGGAGAATTTCCACT | |
| | TTGAATGTCACAATTCCTCCGACCGCATCA | |
| | AGGTGCGCGTCTGGGACGAGGATGACGACA | |
| | TCAAATCCCGCGTGAAACAGAGGTTCAAGA | |
| | GGGAATCTGACGATTTCCTGGGGCAGACGA | |
| | TCATTGAGGTGCGGACGCTCAGCGGCGAGA | |
| | TGGACGTGTGGTACAACCTGGACAAGCGAA | |
| | CTGACAAATCTGCCGTGTCGGGTGCCATCC | |
| | GGCTCCACATCAGTGTGGAGATCAAAGGCG | |
| | AGGAGAAGGTGGCCCCGTACCATGTCCAGT | |
| | ACACCTGTCTGCATGAGCCCTAACCACTCA | |
| | GGATTGGGCCGTTTGTGTCTGGGTATGTCT | |
| | CTTCCAGCTGCCTGGGTTTCCTGGAAAGAA | |
| | CTCTTATCCCCAGGAACTAGTTTGTTGAAT | |
| | AAATGCTGGTGAATGAATGAATGATTGAAC | |
| | AGATGAATGAGTGATGAGTAGATAAAAGGA | |
| | TGGATGGAGAGATGGGAACCTGTTCCACTT | |
| | CGTGACCGACGTGCAGAACAATGGGGTCGT | |
| | GAAGATCCCAGATGCCAAGGGTGACGATGC | |
| | CTGGAAGGTTTACTACGATGAGACAGCCCA | |
| | GGAGATTGTGGACGAGTTTGCCATGCGCTA | |
| | CGGCGTCGAGTCCATCTACCAAGCCATGAC | |
| | CCACTTTGCCTGCCTCTCCTCCAAGTATAT | |
| | GTGCCCAGGGGTGCCTGCCGTCATGAGCAC | |
| | CCTGCTCGCCAACATCAATGCCTACTACGC | |
| | ACACACCACCGCCTCCACCAACGTGTCTGC | |
| | CTCCGACCGCTTCGCCGCCTCCAACTTTGG | |
| | GAAAGAGCGCTTCGTGAAACTCCTGGACCA | |
| | GCTGCATAACTCCCTGCGGATTGACCTCTC | |
| | CATGTACCGGAATAACTTCCCAGCCAGCAG | |
| | CCCGGAGAGACTCCAGGACCTCAAATCCAC | |
| | TGTGGACCTTCTCACCAGCATCACCTTCTT | |
| | TCGGATGAAGGTACAAGAACTCCAGAGCCC | |
| | GCCCCGAGCCAGCCAGGTGGTAAAGGACTG | |
| | TGTGAAAGCCTGCCTTAATTCTACCTACGA | |
| | GTACATCTTCAATAACTGCCATGAACTGTA | |
| | CAGCCGGGAGTACCAGACAGACCCGGCCAA | |
| | GAAGGGGGAAGTTCTCCCAGAGGAACAGGG | |
| | GCCCAGCATCAAGAACCTCGACTTCTGGTC | |
| | CAAGCTGATTACCCTCATAGTGTCCATCAT | |
| | TGAGGAAGACAAGAATTCCTACACTCCCTG | |
| | CCTCAACCAGTTTCCCCAGGAGCTGAATGT | |
| | GGGTAAAATCAGCGCTGAAGTGATGTGGAA | |
| | TCTGTTTGCCCAAGACATGAAGTACGCCAT | |
| | GGAGGAGCACGACAAGCATCGTCTATGCAA | |
| | GAGTGCCGACTACATGAACCTCCACTTCAA | |
| | GGTGAAATGGCTCTACAATGAGTATGTGAC | |
| | GGAACTTCCCGCCTTCAAGGACCGCGTGCC | |
| | TGAGTACCCTGCATGGTTTGAACCCTTCGT | |
| | CATCCAGTGGCTGGATGAGAATGAGGAGGT | |
| | GTCCCGGGATTTCCTGCACGGTGCCCTGGA | |
| | GCGAGACAAGAAGGATGGGTTCCAGCAGAC | |
| | CTCAGAGCATGCCCTATTCTCCTGCTCCGT | |
| | GGTGGATGTTTTCTCCCAACTCAACCAGAG | |
| | CTTTGAAATCATCAAGAAACTCGAGTGTCC | |
| | CGACCCTCAGATCGTGGGGCACTACATGAG | |
| | GCGCTTTGCCAAGACCATCAGTAATGTGCT | |
| | CCTCCAGTATGCAGACATCATCTCCAAGGA | |
| | CTTTGCCTCCTACTGCTCCAAGGAGAAGGA | |
| | GAAAGTGCCCTGCATTCTCATGAATAACAC | |
| | TCAACAGCTACGAGTTCAGCTGGAGAAGAT | |
| | GTTCGAAGCCATGGGAGGAAAGGAGCTGGA | |
| | TGCTGAAGCCAGTGACATCCTGAAGGAGCT | |
| | TCAGGTGAAACTCAATAACGTCTTGGATGA | |
| | GCTCAGCCGGGTGTTTGCTACCAGCTTCCA | |
| | GCCGCACATTGAAGAGTGTGTCAAACAGAT | |
| | GGGTGACATCCTTAGCCAGGTTAAGGGCAC | |
| | AGGCAATGTGCCAGCCAGTGCCTGCAGCAG | |
| | CGTGGCCCAGGACGCGGACAATGTGTTGCA | |
| | GCCCATCATGGACCTGCTGGACAGCAACCT | |
| | GACCCTCTTTGCCAAAATCTGTGAGAAGAC | |
| | TGTGCTGAAGCGAGTGCTGAAGGAGCTGTG | |
| | GAAGCTGGTTATGAACACCATGGAGAAAAC | |
| | CATCGTCCTGCCGCCCCTCACTGACCAGAC | |
| | GATGATCGGGAACCTCTTGAGAAAACATGG | |
| | CAAGGGATTAGAAAAGGGCAGGGTGAAATT | |
| | GCCAAGCCACTCAGACGGAACCCAGATGAT | |
| | CTTCAATGCAGCCAAGGAGCTGGGTCAGCT | |
| | GTCCAAACTCAAGGATCACATGGTACGAGA | |
| | AGAAGCCAAGAGCTTGACCCCAAAGCAGTG | |
| | CGCGGTTGTTGAGTTGGCCCTGGACACCAT | |
| | CAAGCAATATTTCCACGCGGGTGGCGTGGG | |
| | CCTCAAGAAGACCTTCCTGGAGAAGAGCCC | |
| | GGACCTGCAATCCTTGCGCTATGCCCTGTC | |
| | GCTCTACACGCAGGCCACCGACCTGCTAAT | |
| | CAAGACCTTTGTACAGACGCAATCGGCCCA | |
| | GGGCTTGGGTGTAGAAGACCCTGTGGGTGA | |
| | AGTCTCTGTCCATGTTGAGCTGTTCACTCA | |
| | TCCAGGAACTGGGGAACACAAGGTCACAGT | |
| | GAAAGTGGTGGCTGCCAATGACCTCAAGTG | |
| | GCAGACTTCTGGCATCTTCCGGCCGTTCAT | |
| | CGAGGTCAACATCATTGGGCCCCAGCTCAG | |
| | CGACAAGAAACGCAAGTTTGCGACCAAATC | |
| | CAAGAACAATAGCTGGGCTCCCAAGTACAA | |
| | TGAGAGCTTCCAGTTCACGCTGAGCGCCGA | |
| | CGCGGGTCCCGAGTGCTATGAGCTGCAGGT | |
| | GTGCGTCAAGGACTACTGCTTCGCGCGCGA | |
| | GGACCGCACGGTGGGGCTGGCCGTGCTGCA | |
| | GCTGCGTGAGCTGGCCCAGCGCGGGAGCGC | |
| | CGCCTGCTGGCTGCCGCTCGGCCGCCGCAT | |
| | CCACATGGACGACACGGGCCTCACGGTGCT | |
| | GCGAATCCTCTCGCAGCGCAGCAACGACGA | |
| | GGTGGCCAAGGAGTTCGTGAAGCTCAAGTC | |
| | GGACACGCGCTCCGCCGAGGAGGGCGGTGC | |
| | CGCGCCTGCGCCTTAGCGCGGGCGGTCGGC | |
| | CGAGCGGCACTGCGCCTGCGCGGAGGGCGC | |
| | TGGGCGGGGAGGGACGGGGCTTGCGCCTTG | |
| | GTGGGACCTCCCCAGGGGCGGGGCTCGGGG | |
| | GGCTCCACGCCAAGGGTGGGCTGCGCCTAC | |
| | GCCCTTGACTCAGCTTTCCCTTTTGGGGAA | |
| | TTAGGAATGGAGGATGCCCCGCCCTCTCGG | |
| | GAGGCCACGCCCAAGGGCGCGACGAAGGAA | |
| | GGAGCCACATCCCCAACTTGAGGCCACGCC | |
| | CCCAGCACCTAGGGGGCATTTTGAGCTGGG | |
| | ATGGGGGAAACCTCGTCCCTATGGAGGAGG | |
| | CCACATCCCGGGGCTCTGGTACCGGGAGGC | |
| | ACCACCTCATGTCCCCTGGAAAAGCCATAA | |
| | GATGGGACCCAGACCCCTGGGACCCCAGAC | |
| | CAATTGCCAAGTATGGAAATCTCAGCTCCC | |
| | TCGAGGGGGGGCCCTGGGCAAGGGGTAGGG | |
| | CTCTCTGGAGCGCCCCTCTAGGTGGCCTGG | |
| | GGACTGGAGGGACCAGGATGCTGGTTGGAG | |
| | GGCCCCGGAATACCGGAGTCCCTTTAGATA | |
| | TTTGTGCAAAAAATAAATGGGGGGAGGGGG | |
| | GAGGATGGGATTTCAAAAGCACATGCGCCC | |
| | TTGGGCGCCCAAACCCTGGGGGCCGAGGGG | |
| | ACGGCTCTGGTTCCCCACGCTGCCCCTACT | |
| | TCCCTTTGGGAGTTTGCCTCTCCCTCTCCC | |
| | CCAACAAACCCAGTCCTCATATCATAGAGT | |
| | TCAACACACCCATTTGACAGATGGCAAAAC | |
| | TGAGGCTTAAAGAGCTGCTTGAGACTTGGC | |
| | CAAGGTTCCAGGTGCCATACCCTCTGTGCC | |
| | CCTCCCTTAGGCCTGTGTGCCCCATGGAAG | |
| | GGTGGGCTGAGATCGGGATGACCTGACACA | |
| | GCTCCCTATTGCTGCTAATTCCCCCTCGGC | |
| | CTCCTCCAAGGGGTGGGAATTCCAGGCCAA | |
| | GACCCCTACTTCGCCTTTCCTTCTCCGGCT | |
| | GCCAAGCAGGACCTTTGCCCTCAGCCCTTT | |
| | CTCCTGGGATCTCCATGGGGGATGCCATGA | |
| | GGGCCTCCCACCACAAAAGAGAATTTGGGA | |
| | TCCCCTGGTCCCAGGTTTCTCCATCCCTTC | |
| | TTCCTTTTCCAGAATTTTCCAAATAGGAAA | |
| | GAACAGAAGGAGACCAGAAACTCTAGGGGG | |
| | GAGAAAGAGAATGAGAGAAAGAGAATGAGA | |
| | GAGAGAGAAACACAAACACAGTGACACAGT | |
| | GAGAGCTTAGTCTCCAAGAGCCTATTCATT | |
| | GATTCAAACACCCAAGCCACAGGATACCTC | |
| | AGATGGCCCTCTTGCCAGCTGGAAGCTCTT | |
| | TCTCCAATGAGCAAAGTTACAGTGACCTGG | |
| | CTGGAGTTACCTGGTGCACATAGGACCTTA | |
| | GGGGAAAGTTCAGCGTGGACTACACTTGCT | |
| | CTGGGATCTGCTTTTCCACATGTGTGTATG | |
| | GCACGCCTTTTTCTGCTGGATTGGGAAGGA | |
| | CAAGATTTTGCTGTGCTAGGGAGAAATGAA | |
| | AACGGGGTGAGCTGAGTAGCTGGGTTTCTG | |
| | GAGGATAGAACATCAGATGGGGAGGCTTTC | |
| | CGAGGTGAAGAATGAGAGGGAACCACTTAC | |
| | TAGAGAGAAAAGAGCTCCAGGCCTGGGGAA | |
| | CAGCACGTGCGAAGGCCAGGAGAGAAGAAC | |
| | TGTTGAAACAACGAGAAGGGTGGCACGGCT | |
| | GGAGCTGAGCCAGCAAGGGGGATCGTGAGG | |
| | AGCCTTGGGGTTGGGGAGATCTGCAGAAGC | |
| | ATCAGACCAGGCAGGGCCTCGTACGCAGTC | |
| | CTGAGGAGTTTTACTTTTATTCTAAGACAG | |
| | TTGGGGAGCTCCAGGAGCTGTTTTAAGTTG | |
| | GGGAGAGACTGGATTCCAGCCTGCAAAAGC | |
| | TGTTTTGTGAAGACTAAAACCAGTGAGGAG | |
| | AGGTGGAGGTGCTTTGGGGACACTGAAATG | |
| | GATTCTTGGAAAGATTCTGAAGGCTGTGTT | |
| | GAAAAGACACCTATAGCTGTGGGGACATGA | |
| | CTATAATCCCAGCATTTGGGGAGACCGAGG | |
| | CTGGCAGATCACTTAAGGTCAGGAGTTTGA | |
| | GACCAGCCTGGCCAACATGGCGAAACCCCA | |
| | TCTCTGCTAAAAATACAAAAATTAGCTGGG | |
| | TGCAGTGGTGCATGCCTGTAGTCCCAGCTA | |
| | CTCAGGAGACTGAGGCGGGAGAATTGTTTG | |
| | AACCCTGGAGGCAGAGGTTGTAGTGAGTCG | |
| | TGATCACACAACTGCACTCCAGCCTGGGCA | |
| | ACAGAACAATACTCCATTCCCTCCCCTCTA | |
| | CCCCACCAAAAAAAAAAAAAAATCCTGCCC | |
| | TTAGATGAGCTCTAGGGCTGCTGAGTACAG | |
| | TTGTCCCAGTTGCACAGTGCCCAAGGGTTT | |
| | GGCATTGCTAAGAAGGCCACGTGCAAATCC | |
| | TAGATATTGAGTGTTGTATGTTTGTGACGT | |
| | TGGTTTCCCGACATGTGAATGGCCCAAGTG | |
| | TCTGGAAGAAGTGGCGCCACTTTCTAATTT | |
| | GCTTGGAGATGTTGCATGTCCCTTAAATTC | |
| | AGACAGGTGCAGGTAACTGGAGGTTCTGAA | |
| | CCAAAGGTTAAAATGCAAATTCTCATACAG | |
| | GGTTGGGAAGTTGTAGCCAGGGATAAGCTT | |
| | ATGTGACTGTTATATGGACTGAGGAGCAGA | |
| | TGTGAATTTCGAACCATGACATGGCTGAGG | |
| | GTAGGGGTCGGGTGGATGGATGATTCAGGG | |
| | TTGTAACCCATAGAGCCCAAAGGGGAAGTG | |
| | ATCTGTGACCTGGGGTGAGGGTGATCTGGA | |
| | AGATTTTTGGATGGCTGGAAAGAAATGGGG | |
| | AAGTCGAGCTGCCTGAGAGAGCCAAGTTAT | |
| | TTCCCAAAAGATTCCTTAGGAGTCTTTCTG | |
| | TTCAAGACCTCCGTGTGTGTGTGTGTGTGT | |
| | TTAGGGTTCCCCAGCAATGGCCCAGGCATG | |
| | TGAAGGAAACAAGCTTCTTCAGGGAATATT | |
| | TGTTGAATGAGTTTTCCTGACTCCCAGGCT | |
| | AGAACTGTTTTTGCAATTTCCACCCTCTTT | |
| | TCTTTCCCCCAGAGAACTCCTATTCGTCCT | |
| | TCAAAACCCATCACGGAAACCCCTCTTGGA | |
| | GAAAACCCTCCTTCCTTCCCCTCAGGACTT | |
| | TCCCAGCCACCGTCTCTCCTCCAGTCCAGC | |
| | CTGATGCCATGGGACTGGGGGTTTCTCTGT | |
| | CCAGCTCTGTTTCTCCCAGACTGGGGTCTG | |
| | AGGACTCTCAGGACCCCCAACTTTACCTAG | |
| | CACAGGCTGGGCACAAGTGGGTGACAGGGA | |
| | GTCTACGCCTAGTGGAATTATGTATTGGGG | |
| | CAGGGTCAGTGTGAGAATACACATCCGCAT | |
| | GCATGTCTGTCCATGTCTGTCCGTACCAAC | |
| | CTTCCCCTTCCACACGGACCTGGGCACATA | |
| | GGAGGTGTCTGAGCCTGACACATGGGACAG | |
| | AGAGTGGACATGGCTGAGACACGGACAGAG | |
| | AAAAGACAAGGAGTCCAGGGGGCTGAAAGC | |
| | CTTTTGAAATCAGGAAGTTCCTGTATTGGC | |
| | AGAACAAAGCCCAGAGAGGAGCAGGGCTTT | |
| | CCTCAACGCCACCCAGCAAGTGGACACAGA | |
| | GCCCGGCCTTGGATGACACCTCCAGGGTTC | |
| | TGAACCCTGGACCTCGCTTTATGCAAGGAG | |
| | CTGGCCCCACATTTCCATGAATCGGGGAAA | |
| | CAGCACAAGAAGGTTGGCCTGTGGCAGGGC | |
| | AAGGGTTAAAGGGGTGACATTGAGGGATGC | |
| | CTCAGAGTCAAAGTCCCCTGACCAAGAGGA | |
| | ATAGAGTAGAAAACACAGAGACAGAGGGTG | |
| | AGATCACGCCCCGATGAGGACGGAGAGAGA | |
| | CAGAGATGGAGAGAGACATAGAGGTGGAAA | |
| | TATACAGAGAAAGATAAATGCAGAGACCAA | |
| | GGCAGGGAGTGTCGGGGGAAGTAAAGAGGG | |
| | TGTCCTGAAGAAAGAAGGATCTGTTCACTC | |
| | TTACCAGTCTGTCCTCGAATGATTTGCATA | |
| | AAATGAGGAGGTGCCTGTCCACACCCCCAA | |
| | TTCCTCTCTCAGGCCCCAGAGCCTGAGACC | |
| | TCACCATGCCCCCATCAGAGATGCAAAAAA | |
| | CTAAACACCCAACTAGAAATCCTTGGGACC | |
| | TCTCTCGGCTGGGATCTCAGAGCCTTTCTG | |
| | TCCCCTACCCCTACCCCATGTGCTGTCGAT | |
| | TTTGCAGATGGGGACAACCTGGGGCCTCCC | |
| | GGAACTCTGCCACCCTGGGGAAGTTGGGGG | |
| | AGGGCCTTAGTCCCGGATCACAACCCCGTC | |
| | TGCTCCCCAGAATCCTTTCCTAAGAATCGT | |
| | TGAGGACCAAAGTTGTCTTTGCTGACACGT | |
| | GTTGCTTTTCTCTTTGCCTTTTATTGTTTC | |
| | AGAGAAAAATCAAGTTGACTGTGTCAAGTA | |
| | ACACCCCACCCCTTACCCCCGTCCAGCCAT | |
| | AGTGGCTCTCTGGAGACACAGGTCACAGGC | |
| | GGAGGGTCCCCTGATCATCCCCAACCACAC | |
| | AGCCAGGGGGACTTGACCCCTGTCCACCCC | |
| | TGTCTCGTGCTCCCTCAGACCCCCACAAAC | |
| | CGGCCAAGCAGTCCGGGGAGGCTTCCCCTC | |
| | CACACAACTCTTAGCATGTGATTGCAGATG | |
| | TGAAATCAAAACGTTGTTTGTTTTTTGTTT | |
| | TGTTTTGATTCTACCCCGTCGGTCCAGTGT | |
| | CTGCACAGACGCCTTCATTTCTCTGTAAAT | |
| | ATGTGACTTGGAACAAATGTTTAACACAAA | |
| | CGAGAAGTGGTCATGAATGCATGGTGTTGA | |
| | GATGTTTTGCACTATTCTGACTTTTTGGTC | |
| | TCTGTAAAAATATTTTATTAACAGCAGACA | |
| | TTAAAAAAAGAAAAACCACACACA | |
| Cryptic | MSLLCVGVKKAKFDGAQEKFNTYVTLKVQN | 10 |
| Exon | VKSTTIAVRGSQPSWEQDFMFEINRLDLGL | |
| Splice | TVEVWNKGLIWDTMVGTVWIPLRTIRQSNE | |
| Variant | EGPGEWLTLDSQVIMADSEICGTKDPTFHR | |
| 2 | ILLDTRFELPLDIPEEEARYWAKKLEQLNA | |
| | MRDQDEYSFQDEQDKPLPVPSNQCCNWNYF | |
| | GWGEQHNDDPDSAVDDRDSDYRSETSNSIP | |
| | PPYYTTSQPNASVHQYSVRPPPLGSRESYS | |
| | DSMHSYEEFSEPQALSPTGSSRYASSGELS | |
| | QGSSQLSEDFDPDEHSLQGSDMEDERDRDS | |
| | YHSCHSSVSYHKDSPRWDQDEEELEEDLED | |
| | FLEEEELPEDEEELEEEEEEVPDDLGSYAQ | |
| | REDVAVAEPKDFKRISLPPAAPGKEDKAPV | |
| | APTEAPDMAKVAPKPATPDKVPAAEQIPEA | |
| | EPPKDEESFRPREDEEGQEGQDSMSRAKAN | |
| | WLRAFNKVRMQLQEARGEGEMSKSLWFKGG | |
| | PGGGLIIIDSMPDIRKRKPIPLVSDLAMSL | |
| | VQSRKAGITSALASSTLNNEELKNHVYKKT | |
| | LQALIYPISCTTPHNFEVWTATTPTYCYEC | |
| | EGLLWGIARQGMRCTECGVKCHEKCQDLLN | |
| | ADCLQRAAEKSSKHGAEDRTQNIIMVLKDR | |
| | MKIRERNKPEIFELIQEIFAVTKTAHTQQM | |
| | KAVKQSVLDGTSKWSAKISITVVCAQGLQA | |
| | KDKTGSSDPYVTVQVGKTKKRTKTIYGNLN | |
| | PVWEENFHFECHNSSDRIKVRVWDEDDDIK | |
| | SRVKQRFKRESDDFLGQTIIEVRTLSGEMD | |
| | VWYNLDKRTDKSAVSGAIRLHISVEIKGEE | |
| | KVAPYHVQYTCLHEP* | |
| TDP-43 | MSEYIRVTEDENDEPIEIPSEDDGTVLLST | 378 |
| | VTAQFPGACGLRYRNPVSQCMRGVRLVEGI | |
| | LHAPDAGWGNLVYVVNYPKDNKRKMDETDA | |
| | SSAVKVKRAVQKTSDLIVLGLPWKTTEQDL | |
| | KEYFSTFGEVLMVQVKKDLKTGHSKGFGFV | |
| | RFTEYETQVKVMSQRHMIDGRWCDCKLPNS | |
| | KQSQDEPLRSRKVFVGRCTEDMTEDELREF | |
| | FSQYGDVMDVFIPKPFRAFAFVTFADDQIA | |
| | QSLCGEDLIIKGISVHISNAEPKHNSNRQL | |
| | ERSGREGGNPGGFGNQGGFGNSRGGGAGLG | |
| | NNQGSNMGGGMNFGAFSINPAMMAAAQAAL | |
| | QSSWGMMGMLASQQNQSGPSGNNQNQGNMQ | |
| | REPNQAFGSGNNSYSGSNSGAAIGWGSASN | |
| | AGSGSGENGGFGSSMDSKSSGWGM | |

### UNC13A Cryptic Exon Splice Variant Specific Inhibitors

The present disclosure also provides UNC13A cryptic exon splice variant specific inhibitors, which may be used for research and therapeutic methods described herein. In embodiments, an UNC13A cryptic exon splice variant specific inhibitor selectively binds to or reduces or inhibits the expression or activity of UNC13A cryptic exon splice variant over full length UNC13A or other variants thereof (i.e., variants that do not contain a cryptic exon from intron 20-21 such as SEQ ID NO:5 or SEQ ID NO:6). In embodiments, an UNC13A cryptic exon splice variant specific inhibitor selectively binds to or reduces or inhibits the activity of UNC13A cryptic exon splice variant #1, UNC13A cryptic exon splice variant #2, or both UNC13A cryptic exon splice variant #1 and UNC13A cryptic exon splice variant #2 over full length UNC13A or other variants thereof. In embodiments, an UNC13A cryptic exon splice variant specific inhibitor specifically targets the cryptic exon from intron 20-21, e.g., SEQ ID NO:5 or SEQ ID NO:6, or the peptide region encoded therefrom. In embodiments, an UNC13A cryptic exon splice variant specific inhibitor exhibits about 90%, 80%, 70%, 60%, 50%, 40%, 30%, 20%, 10%, or 5% or less of the activity for full length UNC13A or variants that do not contain a cryptic exon from intron 20-21 as compared to an UNC13A cryptic exon splice variant.

UNC13A cryptic exon splice variant specific inhibitors include, but are not limited to inhibitory nucleic acids (e.g., RNA interference agents, antisense oligonucleotides), peptides, antibodies, binding proteins, small molecules, ribozymes, and aptamers.

In embodiments, the UNC13A cryptic exon splice variant specific inhibitor comprises a small molecule. A small molecule is a compound that is less than 2000 Daltons in mass. The molecular mass of the small molecule is preferably less than 1000 Daltons, more preferably less than 600 Daltons, e.g., the compound is less than 500 Daltons, less than 400 Daltons, less than 300 Daltons, less than 200 Daltons, or less than 100 Daltons.

Small molecules may be organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically small molecules are less than one kilodalton.

In embodiments, the UNC13A cryptic exon splice variant specific inhibitor comprises an antibody or binding fragment thereof. The term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

A monoclonal antibody or antigen-binding portion thereof may be non-human, chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

The terms “VL” and “VH” refer to the variable binding region from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be determined according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300).

In embodiments, the UNC13A cryptic exon splice variant specific antibody or antigen binding fragment thereof binds to a peptide encoded by SEQ ID NO:5 or SEQ ID NO:6.

In embodiments, the UNC13A cryptic exon splice variant specific inhibitor comprises an inhibitory nucleic acid. An “inhibitory nucleic acid” refers to a short, single stranded or double stranded nucleic acid molecule that has sequence complementary to a target gene or mRNA transcript and is capable of reducing expression of the target gene or mRNA transcript. Reduced expression may be accomplished via a variety of processes, including blocking of transcription or translation (e.g., steric hindrance), degradation of the target mRNA transcript, blocking of pre-mRNA splicing sites, blocking mRNA processing (e.g., capping, polyadenylation). Inhibitory nucleic acids may be single stranded or double stranded. Inhibitory nucleic acids may be composed of DNA, RNA, or both. Inhibitory nucleic acids may contain unmodified nucleotides or may contain modified nucleotides, non-natural nucleotides, or analog nucleotides. Inhibitory nucleic acids include but are not limited to antisense oligonucleotides, siRNAs, shRNAs, miRNAs, double-stranded RNAs (dsRNAs), and endoribonuclease-prepared siRNAs (esiRNAs).

As used herein, the terms “siRNA” or “short interfering RNA” refer to a short, double-stranded polynucleotide sequence (e.g., 17-30 subunits) that mediates a process of sequence-specific post-transcriptional gene silencing, translational inhibition, transcriptional inhibition, or epigenetic RNAi in animals (Zamore et al., Cell 101:25-33, 2000; Fire et al., Nature 391:806, 1998; Hamilton et al., Science 286:950-951, 1999; Lin et al., Nature 402:128-129, 1999; Sharp, Genes Dev. 13:139-141, 1999; and Strauss, Science 286:886, 1999).

In embodiments, a siRNA comprises a first strand and a second strand that have the same number of nucleosides; however, the first and second strands are offset such that the two terminal nucleosides on the first and second strands are left overhanging. In embodiments, the two overhanging nucleosides are thymidine resides. The antisense (or guide) strand of the siRNA includes a region which is at least partially complementary to the target RNA. In embodiments, there is 100% complementarity between the antisense strand of the siRNA and the target RNA. In embodiments where there is partial complementarity of the antisense strand of the siRNA, the complementarity must be sufficient to enable the siRNA, or a cleavage product thereof, to direct sequence specific silencing, such as by RNAi cleavage of the target RNA. In some embodiments, an antisense strand of a siRNA comprises one or more, such as 10, 8, 6, 5, 4, 3, 2 or fewer, mismatches with respect to the target RNA. The mismatches are most tolerated in the terminal regions, and if present are preferably in a terminal region or regions, e.g., within 6, 5, 4, or 3 nucleotides of the 5′ or 3′ terminus. The sense (or passenger) strand of the siRNA need only be sufficiently complementary to the antisense strand to maintain the overall double-strand character of the molecule RNA-induced silencing complex (RISC).

In embodiments, a siRNA may be modified or include nucleoside analogs. Single stranded regions of a siRNA may be modified or include nucleoside analogs, e.g., the unpaired region or regions of a hairpin structure or a region that links two complementary regions. In embodiments, a siRNA may be modified to stabilize the 3′-terminus, the 5′-terminus, or both, of the siRNA. For example, modifications can stabilize the siRNA against degradation by exonucleases, or to favor the antisense strand to enter into a RNA-induced silencing complex (RISC). In embodiments, each strand of a siRNA can be equal to or less than 30, 25, 24, 23, 22, 21, or 20 nucleotides in length. In further embodiments, each strand is at least 19 nucleotides in length. For example, each strand can be from 21 to 25 nucleotides in length such that the siRNA has a duplex region of at leastl7, 18, 19, 29, 21, 22, 23, 24, or 25 nucleotide pairs, and one or more overhangs of 2-3 nucleotides, such as overhangs one or both 3′-ends.

Endoribonuclease-prepared siRNAs (esiRNAs) are siRNAs resulting from cleavage of long double stranded RNA with an endoribonuclease such as RNAse III or dicer. The esiRNA product is a heterogenous mixture of siRNAs that target the same mRNA sequence.

As used herein, the terms “miRNA” or “microRNA” refer to small non-coding RNAs of about 20-22 nucleotides, which is generated from longer RNA hairpin loop precursor structures known as pri-miRNAs. The pri-miRNA undergoes a two-step cleavage process into a microRNA duplex, which is incorporated into RISC. The level of complementarity between the miRNA guide strand and the target RNA determines which silencing mechanism is employed. miRNAs that bind with perfect or extensive complementarity to RNA target sequences, typically in the 3′-UTR, induce cleavage of the target via RNA-mediated interference (RNAi) pathway. miRNAs with limited complementarity to the target RNA, repress target gene expression at the level of translation.

As used herein, the terms “shRNA” or “short hairpin RNA” refer to double-stranded structure formed two complementary (19-22 bp) RNA sequences linked by a short loop (4-11 nt). shRNAs are usually encoded by a vector that is introduced into cells, and the shRNA is processed in the cytosol by Dicer into siRNA duplexes, which are incorporated into the RISC complex, where complementarity between the guide strand and RNA target mediates RNA target specific cleavage and degradation.

As used herein, the term “ribozyme” refers to a catalytically active RNA molecule capable of site-specific cleavage of target mRNA. In certain embodiments, a ribozyme is a Varkud satellite ribozyme, a hairpin ribozyme, a hammerhead ribozyme, or a hepatitis delta ribozyme.

In embodiments, antisense oligonucleotides of the present disclosure target intron 20-21 and/or adjacent sequence in exon 20 or exon 21. Aberrant splicing can be corrected using splice-switching antisense oligonucleotides. Splice-switching antisense oligonucleotides block aberrant splicing sites by hybridizing at or near the splicing sites thereby preventing recognition by the cellular splicing machinery. In embodiments, splice-switching antisense oligonucleotides are modified to be resistant to nucleases, and the resulting target nucleic acid:oligonucleotide heteroduplex is not cleaved by by RNase H. Splice-switching antisense oligonucleotides may comprise nucleotides that do not form RNase H substrates when paired with RNA or a mixture of nucleotide chemistries such that runs of consecutive DNA-like bases are avoided. Thus, in embodiments, splice-switching antisense oligonucleotides may modify UNC13A splicing without altering the abundance of the UNC13A mRNA transcript.

In embodiments, the antisense oligonucleotide is complementary to: the exon 20 splice donor site region in a preprocessed mRNA encoding UNC13A; the cryptic exon splice acceptor site region in a preprocessed mRNA encoding UNC13A; the cryptic exon splice donor site region in a preprocessed mRNA encoding UNC13A; or the exon 21 splice acceptor site region in a preprocessed mRNA encoding UNC13A. In embodiments, the exon 20 splice donor site region in the preprocessed mRNA encoding UNC13A comprises or consists of SEQ ID NO:12. In embodiments, the cryptic exon splice acceptor site region in the preprocessed mRNA encoding UNC13A comprises or consists of SEQ ID NO:91. In embodiments, the cryptic exon splice donor site region in the preprocessed mRNA encoding UNC13A comprises or consists of SEQ ID NO:220. In embodiments, the exon 21 splice acceptor site region in the preprocessed mRNA encoding UNC13A comprises or consists of SEQ ID NO:299.

In embodiments, the inhibitory nucleic acid, e.g., an antisense oligonucleotide, comprises a sequence that is complementary to the 5′ end of the cryptic exon having a sequence set forth in SEQ ID NO:641. In embodiments, the inhibitory nucleic acid, e.g., an antisense oligonucleotide, comprises a sequence that is complementary to the 3′ end of the cryptic exon having a sequence set forth in SEQ ID NO:642.

In embodiments, the inhibitory nucleic acid, e.g., an antisense oligonucleotide, comprises a sequence that is complementary to the 5′ end of the cryptic exon having a sequence set forth in SEQ ID NO:643. In embodiments, the inhibitory nucleic acid, e.g., an antisense oligonucleotide, comprises a sequence that is complementary to the 3′ end of the cryptic exon having a sequence set forth in SEQ ID NO:644.In embodiments, the UNC13A cryptic exon splice variant specific antisense oligonucleotide has about 15-40 bases, e.g., about 15, 16, 17, 18, 19, 20, 21, 22, 23, 24, 25, 26, 27, 28, 29, 30, 31, 32, 33, 34, 35, 36, 37, 38, 39, or 40 bases in length. In embodiments, the UNC13A cryptic exon splice variant specific antisense oligonucleotide has about 18-30 bases, 18-bases, 18-22 bases, or 20-30 bases.

In embodiments, the UNC13A cryptic exon splice variant specific antisense oligonucleotide has a base sequence that has at least 80%, 85%, 90%, 95%, or 100% identity to any one of the sequences in Tables 2-7 (e.g., SEQ ID NOS:13-90, 92-219, 221-298, 300-377, and 423-640). In embodiments, the UNC13A cryptic exon splice variant specific antisense oligonucleotide comprises or consists of any one of the sequences in Tables 2-5 (e.g., SEQ ID NOS: 13-90, 92-219, 221-298, 300-377, and 423-640). In embodiments, the UNC13A cryptic exon splice variant specific antisense oligonucleotide comprises or consists of any one of the sequences set forth in SEQ ID NOS:423-432, 439-443, 491-498, 502-507, and 513-514.

In embodiments, the UNC13A cryptic exon splice variant specific antisense oligonucleotide has 18-30 bases, 18-25 bases, or 18-22 bases that are complementary to SEQ ID NO:650. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:650.

In embodiments, the UNC13A cryptic exon splice variant specific antisense oligonucleotide has 18-30 bases, 18-25 bases, or 18-22 bases that are complementary to SEQ ID NO: 651. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO: 651.

In embodiments, the UNC13A cryptic exon splice variant specific antisense oligonucleotide has 18-30 bases, 18-25 bases, or 18-22 bases that are complementary to SEQ ID NO:652. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:652.

In embodiments, the UNC13A cryptic exon splice variant specific antisense oligonucleotide has 18-30 bases, 18-25 bases, or 18-22 bases that are complementary to SEQ ID NO:653. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:653.

In embodiments, the UNC13A cryptic exon splice variant specific antisense oligonucleotide has 18-21 bases that are complementary to SEQ ID NO:654. In embodiments, the UNC13A cryptic exon splice variant specific antisense oligonucleotide has 18, 19, 20, or 21 bases that are complementary to SEQ ID NO:654.

In embodiments, the UNC13A cryptic exon splice variant specific antisense oligonucleotide is a modified antisense oligonucleotide. A modified antisense oligonucleotide may comprise at least one backbone modification, nucleobase modification, 2′-ribose substitution, or bridged nucleic acid, Examples of modified oligonucleotide chemistries include, without limitation, phosphoramidate morpholino oligonucleotides and phosphorodiamidate morpholino oligonucleotides (PMO), phosphorothioate modified oligonucleotides, 2′ O-methyl (2′ O-Me) modified oligonucleotides, peptide nucleic acid (PNA), locked nucleic acid (LNA), phosphorodithioate oligonucleotides, 2′ O-Methoxyethyl (2′-MOE) modified oligonucleotides, 2′-fluoro-modified oligonucleotides, 2′0,4′C-ethylene-bridged nucleic acids (ENAs), tricyclo-DNAs, tricyclo-DNA phosphorothioate nucleotides, constrained ethyl bridged nucleic acids, 2′-O-[2-(N-methylcarbamoyl)ethyl] modified oligonucleotides, morpholino oligonucleotides, and peptide-conjugated phosphoramidate morpholino oligonucleotides (PPMO). In embodiments, the UNC13A cryptic exon splice variant specific antisense oligonucleotide comprises 2′0-Me modified nucleotides and phosphorothioate linkages.

In some embodiments, the compositions provided herein may be assembled into pharmaceutical or research kits to facilitate their use in therapeutic or research use. A kit may include one or more containers comprising: (a) UNC13A cryptic exon splice variant specific antisense oligonucleotide(s) described herein; and (b) instructions for use. In some embodiments, the kit component (a) may be in a pharmaceutical formulation and dosage suitable for a particular use and mode of administration. For example, the kit component (a) may be presented in unit-dose or multi-dose containers, such as sealed ampoules or vials. The components of the kit may require mixing one or more components prior to use or may be prepared in a premixed state. The components of the kit may be in liquid or solid form, and may require addition of a solvent or further dilution. The components of the kit may be sterile. The instructions may be in written or electronic form and may be associated with the kit (e.g., written insert, CD, DVD) or provided via internet or web-based communication. The kit may be shipped and stored at a refrigerated or frozen temperature.

### Pharmaceutical Compositions

In some aspects, the disclosure provides pharmaceutical compositions comprising an UNC13A cryptic exon splice variant specific inhibitor as described herein and a pharmaceutically acceptable carrier. As used herein, the term “pharmaceutically acceptable” refers to those compounds, materials, compositions, and/or dosage forms which are, within the scope of sound medical judgment, suitable for use in contact with cells and/or tissues without excessive toxicity, irritation, allergic response, or other problem or complication, commensurate with a reasonable benefit/risk ratio.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Each carrier must be “acceptable” in the sense of being compatible with the other ingredients of the formulation and not injurious to the cell or tissue being contacted. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As is well known in the medical arts, the dosage for any one patient depends upon many factors, including the patient's size, weight, body surface area, age, the level of UNC13A cryptic exon splice variant specific inhibitor required to achieve a therapeutic effect, stability of the UNC13A cryptic exon splice variant specific inhibitor, specific disease being treated, stage of disease, sex, time and route of administration, general health, and other drugs being administered concurrently.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions (e.g., pharmaceutical compositions) may be administered by any route, including enteral (e.g., oral), parenteral, intravenous, intramuscular, intra-arterial, intramedullary, intrathecal, subpial, intraparenchymal, intrastriatal, intracranial, intracisternal, intra-cerebral, intracerebral ventricular, intraocular, intraventricular, intralumbar, subcutaneous, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors including the nature of the agent (e.g., its stability in the environment of the gastrointestinal tract), and/or the condition of the subject. In some embodiments, compositions are directly injected into the CNS of the subject. In some embodiments, direct injection into the CNS is intracerebral injection, intraparenchymal injection, intrathecal injection, subpial injection, or any combination thereof. In some embodiments, direct injection into the CNS is direct injection into the cerebrospinal fluid (CSF) of the subject, optionally wherein the direct injection is intracisternal injection, intraventricular injection, and/or intralumbar injection.

### Methods of Using UNC13A Cryptic Splice Variant Inhibitors

The present disclosure provides methods of using UNC13A cryptic exon splice variant specific inhibitors disclosed herein for various research and therapeutics uses. In one aspect, the present disclosure provides a method of reducing expression of a UNC13A cryptic exon splice variant in a cell comprising administering a UNC13A cryptic exon splice variant specific inhibitor, wherein the UNC13A cryptic exon splice variant comprises a cryptic exon between exon 20 and exon 21 of the UNC13A cryptic exon splice variant mature mRNA transcript. In embodiments, the UNC13A cryptic exon splice variant specific inhibitor selectively inhibits the expression or activity of the UNC13A cryptic exon splice variant over full length UNC13A (wildtype) or other variants thereof (i.e., variants that do not contain a cryptic exon from intron 20-21 such as SEQ ID NO:5 or SEQ ID NO:6).

In embodiments, the cryptic exon is obtained from intron 20-21 of the UNC13A gene. In embodiments, the cryptic exon comprises SEQ ID NO:5 or SEQ ID NO:6. In embodiments, the UNC13 cryptic exon splice variant comprises a polynucleotide sequence of SEQ ID NO:7 or SEQ ID NO:9. In embodiments, the UNC13 cryptic exon splice variant comprises the amino acid sequence of SEQ ID NO:8 or SEQ ID NO:10.

In embodiments, the UNC13 cryptic splice variant specific inhibitor comprises an inhibitory nucleic acid, peptides, antibody, binding protein, small molecule, ribozyme, or aptamer.

In embodiments, the UNC13 cryptic splice variant specific inhibitor comprises an inhibitory nucleic acid. The inhibitory nucleic acid may be an antisense oligonucleotide, siRNA, shRNA, miRNA, double-stranded RNA (dsRNAs), or esiRNA.

In embodiments, the inhibitory nucleic acid comprises an antisense oligonucleotide that is complementary to: the exon 20 splice donor site region in a preprocessed mRNA encoding UNC13A; the cryptic exon splice acceptor site region in a preprocessed mRNA encoding UNC13A; the cryptic exon splice donor site region in a preprocessed mRNA encoding UNC13A; or the exon 21 splice acceptor site region in a preprocessed mRNA encoding UNC13A. In embodiments, the exon 20 splice donor site region comprises or consists of SEQ ID NO:12. In embodiments, the cryptic exon splice acceptor site region comprises or consists of SEQ ID NO:91. In embodiments, the cryptic exon splice donor site region comprises or consists of SEQ ID NO:220. In embodiments, the exon 21 splice acceptor site comprises or consists of SEQ ID NO:299.

In embodiments, the inhibitory nucleic acid, e.g., an antisense oligonucleotide, comprises a sequence that is complementary to the 5′ end of the cryptic exon having a sequence set forth in SEQ ID NO:641. In embodiments, the inhibitory nucleic acid, e.g., an antisense oligonucleotide, comprises a sequence that is complementary to the 3′ end of the cryptic exon having a sequence set forth in SEQ ID NO:642.

In embodiments, the inhibitory nucleic acid, e.g., an antisense oligonucleotide, comprises a sequence that is complementary to the 5′ end of the cryptic exon having a sequence set forth in SEQ ID NO:643. In embodiments, the inhibitory nucleic acid, e.g., an antisense oligonucleotide, comprises a sequence that is complementary to the 3′ end of the cryptic exon having a sequence set forth in SEQ ID NO:644.

In embodiments, the UNC13 cryptic splice variant specific antisense oligonucleotide has about 15-40 bases in length, preferably about 18-30 bases, 18-25 bases, 18-22 bases, or 20-30 bases in length.

In embodiments, the UNC13 cryptic splice variant specific antisense oligonucleotide has a base sequence that is at least 80%, 85%, 90%, 95%, 97%, or 100% identical to any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640). In embodiments, the UNC13 cryptic splice variant specific antisense oligonucleotide has a base sequence comprising or consisting of any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640).

In embodiments, the UNC13A cryptic exon splice variant specific antisense oligonucleotide has 18-30 bases, 18-25 bases, or 18-22 bases that are complementary to SEQ ID NO:650. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:650.

In embodiments, the UNC13A cryptic exon splice variant specific antisense oligonucleotide has 18-30 bases, 18-25 bases, or 18-22 bases that are complementary to SEQ ID NO: 651. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO: 651.

In embodiments, the UNC13A cryptic exon splice variant specific antisense oligonucleotide has 18-30 bases, 18-25 bases, or 18-22 bases that are complementary to SEQ ID NO:652. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:652.

In embodiments, the UNC13A cryptic exon splice variant specific antisense oligonucleotide has 18-30 bases, 18-25 bases, or 18-22 bases that are complementary to SEQ ID NO:653. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:653.

In embodiments, the UNC13A cryptic exon splice variant specific antisense oligonucleotide has 18-21 bases that are complementary to SEQ ID NO:654. In embodiments, the UNC13A cryptic exon splice variant specific antisense oligonucleotide has 18, 19, 20, or 21 bases that are complementary to SEQ ID NO:654.In embodiments, the UNC13 cryptic splice variant specific antisense oligonucleotide is a modified antisense oligonucleotide. In embodiments, the modified antisense oligonucleotide comprises a phosphoramidate morpholino oligonucleotide, phosphorodiamidate morpholino oligonucleotide, phosphorothioate modified oligonucleotide, 2′ O-methyl (2′ O-Me) modified oligonucleotide, peptide nucleic acid (PNA), locked nucleic acid (LNA), phosphorodithioate oligonucleotide, 2′ 0-Methoxyethyl (2′-MOE) modified oligonucleotide, 2′-fluoro-modified oligonucleotide, 2′0,4′C-ethylene-bridged nucleic acid (ENAs), tricyclo-DNA, tricyclo-DNA phosphorothioate nucleotide, constrained ethyl bridged nucleic acid, 2′-O-[2-(N-methylcarbamoyl)ethyl] modified oligonucleotide, morpholino oligonucleotide, and peptide-conjugated phosphoramidate morpholino oligonucleotide (PPMO), or any combination thereof.

In embodiments, the cell is within a subject. As used here, a “patient” or “subject” includes an animal, such as a human, cow, horse, sheep, lamb, pig, chicken, turkey, quail, cat, dog, mouse, rat, rabbit or guinea pig. The animal can be a mammal, such as a non-primate and a primate (e.g., monkey and human). In embodiments, a patient is a human, such as a human infant, child, adolescent or adult.

In embodiments, the subject has been identified as having a UNC13A gene mutation in intron 20-21. In embodiments, the UNC13 gene mutation comprises rs12608932 (hg38 chrl9:17.641,880 A→C), rs12973192 (hg38 chrl9: 17,642,430 C→G), rs56041637 (hg38 chrl9:17,642,033-17,642,056 0-2 CATC repeats →3-5 CATC repeats), and rs62121687 (hg38 chrl9:17,642,351 C→A), or any combination thereof.

In another aspect, the present disclosure provides a method of reducing phosphorylated TAR-DNA binding protein-43 (TDP-43) in a cell comprising administering a UNC13A cryptic exon splice variant specific inhibitor, wherein the UNC13A cryptic exon splice variant comprises a cryptic exon between exon 20 and exon 21 of the UNC13A cryptic exon splice variant mature mRNA transcript.

In embodiments, the UNC13A cryptic exon splice variant specific inhibitor selectively inhibits the expression or activity of the UNC13A cryptic exon splice variant over full length UNC13A (wildtype) or other variants thereof (i.e., variants that do not contain a cryptic exon from intron 20-21 such as SEQ ID NO:5 or SEQ ID NO:6).

In embodiments, the cryptic exon is obtained from intron 20-21 of the UNC13A gene. In embodiments, the cryptic exon comprises SEQ ID NO:5 or SEQ ID NO:6. In embodiments, the UNC13 cryptic exon splice variant comprises a polynucleotide sequence of SEQ ID NO:7 or SEQ ID NO:9. In embodiments, the UNC13 cryptic exon splice variant comprises the amino acid sequence of SEQ ID NO:8 or SEQ ID NO:10.

In embodiments, the UNC13 cryptic splice variant specific inhibitor comprises an inhibitory nucleic acid, peptides, antibody, binding protein, small molecule, ribozyme, or aptamer.

In embodiments, the UNC13 cryptic splice variant specific inhibitor comprises an inhibitory nucleic acid. The inhibitory nucleic acid may be an antisense oligonucleotide, siRNA, shRNA, miRNA, double-stranded RNA (dsRNAs), or esiRNA.

In embodiments, the inhibitory nucleic acid comprises an antisense oligonucleotide that is complementary to: the exon 20 splice donor site region in a preprocessed mRNA encoding UNC13A; the cryptic exon splice acceptor site region in a preprocessed mRNA encoding UNC13A; the cryptic exon splice donor site region in a preprocessed mRNA encoding UNC13A; or the exon 21 splice acceptor site region in a preprocessed mRNA encoding UNC13A. In embodiments, the exon 20 splice donor site region comprises or consists of SEQ ID NO:12. In embodiments, the cryptic exon splice acceptor site region comprises or consists of SEQ ID NO:91. In embodiments, the cryptic exon splice donor site region comprises or consists of SEQ ID NO:220. In embodiments, the exon 21 splice acceptor site comprises or consists of SEQ ID NO:299.

In embodiments, the inhibitory nucleic acid, e.g., an antisense oligonucleotide, comprises a sequence that is complementary to the 5′ end of the cryptic exon having a sequence set forth in SEQ ID NO:641. In embodiments, the inhibitory nucleic acid, e.g., an antisense oligonucleotide, comprises a sequence that is complementary to the 3′ end of the cryptic exon having a sequence set forth in SEQ ID NO:642.

In embodiments, the inhibitory nucleic acid, e.g., an antisense oligonucleotide, comprises a sequence that is complementary to the 5′ end of the cryptic exon having a sequence set forth in SEQ ID NO:643. In embodiments, the inhibitory nucleic acid, e.g., an antisense oligonucleotide, comprises a sequence that is complementary to the 3′ end of the cryptic exon having a sequence set forth in SEQ ID NO:644.

In embodiments, the UNC13 cryptic splice variant specific antisense oligonucleotide has about 15-40 bases in length, preferably about 18-30 bases, 18-25 bases, 18-22 bases, or 20-30 bases in length.

In embodiments, the UNC13 cryptic splice variant specific antisense oligonucleotide has a base sequence that is at least 80%, 85%, 90%, 95%, 97%, or 100% identical to any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640). In embodiments, the UNC13 cryptic splice variant specific antisense oligonucleotide has a base sequence comprising or consisting of any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640).

In embodiments, the UNC13A cryptic exon splice variant specific antisense oligonucleotide has 18-30 bases, 18-25 bases, or 18-22 bases that are complementary to SEQ ID NO:650. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:650.

In embodiments, the UNC13A cryptic exon splice variant specific antisense oligonucleotide has 18-30 bases, 18-25 bases, or 18-22 bases that are complementary to SEQ ID NO: 651. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO: 651.

In embodiments, the UNC13A cryptic exon splice variant specific antisense oligonucleotide has 18-30 bases, 18-25 bases, or 18-22 bases that are complementary to SEQ ID NO:652. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:652.

In embodiments, the UNC13A cryptic exon splice variant specific antisense oligonucleotide has 18-30 bases, 18-25 bases, or 18-22 bases that are complementary to SEQ ID NO:653. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:653.

In embodiments, the UNC13A cryptic exon splice variant specific antisense oligonucleotide has 18-21 bases that are complementary to SEQ ID NO:654. In embodiments, the UNC13A cryptic exon splice variant specific antisense oligonucleotide has 18, 19, 20, or 21 bases that are complementary to SEQ ID NO:654.

In embodiments, the UNC13 cryptic splice variant specific antisense oligonucleotide is a modified antisense oligonucleotide. In embodiments, the modified antisense oligonucleotide comprises a phosphoramidate morpholino oligonucleotide, phosphorodiamidate morpholino oligonucleotide, phosphorothioate modified oligonucleotide, 2′ O-methyl (2′ O-Me) modified oligonucleotide, peptide nucleic acid (PNA), locked nucleic acid (LNA), phosphorodithioate oligonucleotide, 2′ 0-Methoxyethyl (2′-MOE) modified oligonucleotide, 2′-fluoro-modified oligonucleotide, 2′O,4′C-ethylene-bridged nucleic acid (ENAs), tricyclo-DNA, tricyclo-DNA phosphorothioate nucleotide, constrained ethyl bridged nucleic acid, 2′-O-[2-(N-methylcarbamoyl)ethyl] modified oligonucleotide, morpholino oligonucleotide, and peptide-conjugated phosphoramidate morpholino oligonucleotide (PPMO), or any combination thereof.

In embodiments, the cell is within a subject. In embodiments, the subject has been identified as having a UNC13A gene mutation in intron 20-21. In embodiments, the UNC13 gene mutation comprises rs12608932 (hg38 chrl9:17.641,880 A→C), rs12973192 (hg38 chrl9: 17,642,430 C→G), rs56041637 (hg38 chrl9:17,642,033-17,642,056 0-2 CATC repeats →3-5 CATC repeats), and rs62121687 (hg38 chrl9:17,642,351 C→A), or any combination thereof.

In another aspect, the present disclosure provides a method of treating TAR-DNA binding protein-43 (TDP-43) proteinopathy in a subject comprising administering a UNC13A cryptic exon splice variant specific inhibitor to the subject, wherein the UNC13A cryptic exon splice variant comprises a cryptic exon between exon 20 and exon 21 of the UNC13A cryptic exon splice variant mature mRNA transcript.

In embodiments, the UNC13A cryptic exon splice variant specific inhibitor selectively inhibits the expression or activity of the UNC13A cryptic exon splice variant over full length UNC13A (wildtype) or other variants thereof (i.e., variants that do not contain a cryptic exon from intron 20-21 such as SEQ ID NO:5 or SEQ ID NO:6).

In embodiments, the cryptic exon is obtained from intron 20-21 of the UNC13A gene. In embodiments, the cryptic exon comprises SEQ ID NO:5 or SEQ ID NO:6. In embodiments, the UNC13 cryptic exon splice variant comprises a polynucleotide sequence of SEQ ID NO:7 or SEQ ID NO:9. In embodiments, the UNC13 cryptic exon splice variant comprises the amino acid sequence of SEQ ID NO:8 or SEQ ID NO:10.

In embodiments, the UNC13 cryptic splice variant specific inhibitor comprises an inhibitory nucleic acid, peptides, antibody, binding protein, small molecule, ribozyme, or aptamer.

In embodiments, the UNC13 cryptic splice variant specific inhibitor comprises an inhibitory nucleic acid. The inhibitory nucleic acid may be an antisense oligonucleotide, siRNA, shRNA, miRNA, double-stranded RNA (dsRNAs), or esiRNA.

In embodiments, the inhibitory nucleic acid comprises an antisense oligonucleotide that is complementary to: the exon 20 splice donor site region in a preprocessed mRNA encoding UNC13A; the cryptic exon splice acceptor site region in a preprocessed mRNA encoding UNC13A; the cryptic exon splice donor site region in a preprocessed mRNA encoding UNC13A; or the exon 21 splice acceptor site region in a preprocessed mRNA encoding UNC13A. In embodiments, the exon 20 splice donor site region comprises or consists of SEQ ID NO:12. In embodiments, the cryptic exon splice acceptor site region comprises or consists of SEQ ID NO:91. In embodiments, the cryptic exon splice donor site region comprises or consists of SEQ ID NO:220. In embodiments, the exon 21 splice acceptor site comprises or consists of SEQ ID NO:299.

In embodiments, the inhibitory nucleic acid, e.g., an antisense oligonucleotide, comprises a sequence that is complementary to the 5′ end of the cryptic exon having a sequence set forth in SEQ ID NO:641. In embodiments, the inhibitory nucleic acid, e.g., an antisense oligonucleotide, comprises a sequence that is complementary to the 3′ end of the cryptic exon having a sequence set forth in SEQ ID NO:642.

In embodiments, the inhibitory nucleic acid, e.g., an antisense oligonucleotide, comprises a sequence that is complementary to the 5′ end of the cryptic exon having a sequence set forth in SEQ ID NO:643. In embodiments, the inhibitory nucleic acid, e.g., an antisense oligonucleotide, comprises a sequence that is complementary to the 3′ end of the cryptic exon having a sequence set forth in SEQ ID NO:644.

In embodiments, the UNC13 cryptic splice variant specific antisense oligonucleotide has about 15-40 bases in length, preferably about 18-30 bases, 18-25 bases, 18-22 bases, or 20-30 bases in length.

In embodiments, the UNC13 cryptic splice variant specific antisense oligonucleotide has a base sequence that is at least 80%, 85%, 90%, 95%, 97%, or 100% identical to any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640). In embodiments, the UNC13 cryptic splice variant specific antisense oligonucleotide has a base sequence comprising or consisting of any one of the sequences listed in Table 2 (e.g., SEQ ID NOS:13-90), Table 3 (SEQ ID NOS:92-219), Table 4 (SEQ ID NOS:221-298), and Table 5 (SEQ ID NOS:300-377), Table 7B (SEQ ID NOS:423-522), and Table 8B (SEQ ID NOS:523-640).

In embodiments, the UNC13A cryptic exon splice variant specific antisense oligonucleotide has 18-30 bases, 18-25 bases, or 18-22 bases that are complementary to SEQ ID NO:650. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:650.

In embodiments, the UNC13A cryptic exon splice variant specific antisense oligonucleotide has 18-30 bases, 18-25 bases, or 18-22 bases that are complementary to SEQ ID NO: 651. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO: 651.

In embodiments, the UNC13A cryptic exon splice variant specific antisense oligonucleotide has 18-30 bases, 18-25 bases, or 18-22 bases that are complementary to SEQ ID NO:652. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:652.

In embodiments, the UNC13A cryptic exon splice variant specific antisense oligonucleotide has 18-30 bases, 18-25 bases, or 18-22 bases that are complementary to SEQ ID NO:653. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:653.

In embodiments, the UNC13A cryptic exon splice variant specific antisense oligonucleotide has 18-21 bases that are complementary to SEQ ID NO:654. In embodiments, the UNC13A cryptic exon splice variant specific antisense oligonucleotide has 18, 19, 20, or 21 bases that are complementary to SEQ ID NO:654.

In embodiments, the UNC13 cryptic splice variant specific antisense oligonucleotide is a modified antisense oligonucleotide. In embodiments, the modified antisense oligonucleotide comprises a phosphoramidate morpholino oligonucleotide, phosphorodiamidate morpholino oligonucleotide, phosphorothioate modified oligonucleotide, 2′ O-methyl (2′ O-Me) modified oligonucleotide, peptide nucleic acid (PNA), locked nucleic acid (LNA), phosphorodithioate oligonucleotide, 2′ 0-Methoxyethyl (2′-MOE) modified oligonucleotide, 2′-fluoro-modified oligonucleotide, 2′O,4′C-ethylene-bridged nucleic acid (ENAs), tricyclo-DNA, tricyclo-DNA phosphorothioate nucleotide, constrained ethyl bridged nucleic acid, 2′-O-[2-(N-methylcarbamoyl)ethyl] modified oligonucleotide, morpholino oligonucleotide, and peptide-conjugated phosphoramidate morpholino oligonucleotide (PPMO), or any combination thereof.

In embodiments, the cell is within a subject. In embodiments, the subject has been identified as having a UNC13A gene mutation in intron 20-21. In embodiments, the UNC13 gene mutation comprises rs12608932 (hg38 chrl9:17.641,880 A→C), rs12973192 (hg38 chrl9: 17,642,430 C→G), rs56041637 (hg38 chrl9:17,642,033-17,642,056 0-2 CATC repeats →3-5 CATC repeats), and rs62121687 (hg38 chrl9:17,642,351 C→A), or any combination thereof.

In embodiments, the TDP-43 proteinopathy comprises amyotrophic lateral sclerosis (ALS), frontotemporal lobar degeneration (FTLD), primary lateral sclerosis (PLS), progressive muscular atrophy (PMA), facial onset sensory and motor neuronopathy (FOSMN), hippocampal sclerosis (HS), limbic-predominant age-related TDP-43 encephalopathy (LATE), cerebral age-related TDP-43 with sclerosis (CARTS), Guam Parkinson-dementia complex (G-PDC), Guan ALS (G-ALS), Multisystem proteinopathy (MSP), Perry disease, Alzheimer's disease (AD), and chronic traumatic encephalopathy (CTE), or any combination thereof.

In another aspect, the present disclosure provides a method of treating a subject has been identified as having an UNC13A gene mutation in intron 20-21 comprising administering an UNC13A cryptic exon splice variant specific inhibitor to the subject, wherein the UNC13A cryptic exon splice variant comprises a cryptic exon between exon 20 and exon 21 of the UNC13A cryptic exon splice variant mature mRNA transcript. In embodiments, the UNC13 gene mutation comprises rs12608932 (hg38 chrl9:17.641,880 A→C), rs12973192 (hg38 chrl9: 17,642,430 C→G), rs56041637 (hg38 chrl9:17,642,033-17,642,056 0-2 CATC repeats→3-5 CATC repeats), and rs62121687 (hg38 chrl9:17,642,351 C→A), or any combination thereof.

In embodiments, the subject has decreased expression of TDP-43. In embodiments, the subject exhibits decreased nuclear TDP-43.

In embodiments, the UNC13A cryptic exon splice variant specific inhibitor selectively inhibits the expression or activity of the UNC13A cryptic exon splice variant over full length UNC13A (wildtype) or other variants thereof (i.e., variants that do not contain a cryptic exon from intron 20-21 such as SEQ ID NO:5 or SEQ ID NO:6).

In embodiments, the cryptic exon is obtained from intron 20-21 of the UNC13A gene. In embodiments, the cryptic exon comprises SEQ ID NO:5 or SEQ ID NO:6. In embodiments, the UNC13 cryptic exon splice variant comprises a polynucleotide sequence of SEQ ID NO:7 or SEQ ID NO:9. In embodiments, the UNC13 cryptic exon splice variant comprises the amino acid sequence of SEQ ID NO:8 or SEQ ID NO:10.

In embodiments, the UNC13 cryptic splice variant specific inhibitor comprises an inhibitory nucleic acid, peptides, antibody, binding protein, small molecule, ribozyme, or aptamer.

In embodiments, the UNC13 cryptic splice variant specific inhibitor comprises an inhibitory nucleic acid. The inhibitory nucleic acid may be an antisense oligonucleotide, siRNA, shRNA, miRNA, double-stranded RNA (dsRNAs), or esiRNA.

In embodiments, the inhibitory nucleic acid comprises an antisense oligonucleotide that is complementary to: the exon 20 splice donor site region in a preprocessed mRNA encoding UNC13A; the cryptic exon splice acceptor site region in a preprocessed mRNA encoding UNC13A; the cryptic exon splice donor site region in a preprocessed mRNA encoding UNC13A; or the exon 21 splice acceptor site region in a preprocessed mRNA encoding UNC13A. In embodiments, the exon 20 splice donor site region comprises or consists of SEQ ID NO:12. In embodiments, the cryptic exon splice acceptor site region comprises or consists of SEQ ID NO:91. In embodiments, the cryptic exon splice donor site region comprises or consists of SEQ ID NO:220. In embodiments, the exon 21 splice acceptor site comprises or consists of SEQ ID NO:299.

In embodiments, the inhibitory nucleic acid, e.g., an antisense oligonucleotide, comprises a sequence that is complementary to the 5′ end of the cryptic exon having a sequence set forth in SEQ ID NO:641. In embodiments, the inhibitory nucleic acid, e.g., an antisense oligonucleotide, comprises a sequence that is complementary to the 3′ end of the cryptic exon having a sequence set forth in SEQ ID NO:642.

In embodiments, the inhibitory nucleic acid, e.g., an antisense oligonucleotide, comprises a sequence that is complementary to the 5′ end of the cryptic exon having a sequence set forth in SEQ ID NO:643. In embodiments, the inhibitory nucleic acid, e.g., an antisense oligonucleotide, comprises a sequence that is complementary to the 3′ end of the cryptic exon having a sequence set forth in SEQ ID NO:644.

In embodiments, the UNC13 cryptic splice variant specific antisense oligonucleotide has about 15-40 bases in length, preferably about 18-30 bases, 18-25 bases, 18-22 bases, or 20-30 bases in length.

In embodiments, the UNC13 cryptic splice variant specific antisense oligonucleotide has a base sequence that is at least 80%, 85%, 90%, 95%, 97%, or 100% identical to any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640). In embodiments, the UNC13 cryptic splice variant specific antisense oligonucleotide has a base sequence comprising or consisting of any one of the sequences listed in Table 2 (e.g., SEQ ID NOS:13-90), Table 3 (SEQ ID NOS:92-219), Table 4 (SEQ ID NOS:221-298), Table 5 (SEQ ID NOS:300-377), Table 7B (SEQ ID NOS:423-522), and Table 8B (SEQ ID NOS:523-640).

In embodiments, the UNC13A cryptic exon splice variant specific antisense oligonucleotide has 18-30 bases, 18-25 bases, or 18-22 bases that are complementary to SEQ ID NO:650. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:650.

In embodiments, the UNC13A cryptic exon splice variant specific antisense oligonucleotide has 18-30 bases, 18-25 bases, or 18-22 bases that are complementary to SEQ ID NO: 651. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO: 651.

In embodiments, the UNC13A cryptic exon splice variant specific antisense oligonucleotide has 18-30 bases, 18-25 bases, or 18-22 bases that are complementary to SEQ ID NO:652. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:652.

In embodiments, the UNC13A cryptic exon splice variant specific antisense oligonucleotide has 18-30 bases, 18-25 bases, or 18-22 bases that are complementary to SEQ ID NO:653. In embodiments, the UNC13A cryptic exon splice variant specific antisense oligonucleotide has 18, 19, 20, 21, 22, 23, 24, 25, 26, 27, 28, 29, or 30 bases that are complementary to SEQ ID NO:653.

In embodiments, the UNC13A cryptic exon splice variant specific antisense oligonucleotide has 18-21 bases that are complementary to SEQ ID NO:654. In embodiments, the UNC13A cryptic exon splice variant specific antisense oligonucleotide has 18, 19, 20, or 21 bases that are complementary to SEQ ID NO:654.

In embodiments, the UNC13 cryptic splice variant specific antisense oligonucleotide is a modified antisense oligonucleotide. In embodiments, the modified antisense oligonucleotide comprises a phosphoramidate morpholino oligonucleotide, phosphorodiamidate morpholino oligonucleotide, phosphorothioate modified oligonucleotide, 2′ O-methyl (2′ O-Me) modified oligonucleotide, peptide nucleic acid (PNA), locked nucleic acid (LNA), phosphorodithioate oligonucleotide, 2′ 0-Methoxyethyl (2′-MOE) modified oligonucleotide, 2′-fluoro-modified oligonucleotide, 2′0,4′C-ethylene-bridged nucleic acid (ENAs), tricyclo-DNA, tricyclo-DNA phosphorothioate nucleotide, constrained ethyl bridged nucleic acid, 2′-O-[2-(N-methylcarbamoyl)ethyl] modified oligonucleotide, morpholino oligonucleotide, and peptide-conjugated phosphoramidate morpholino oligonucleotide (PPMO), or any combination thereof.

In embodiments, the subject has a TDP-43 proteinopathy. In embodiments, the TDP-43 proteinopathy comprises amyotrophic lateral sclerosis (ALS), frontotemporal lobar degeneration (FTLD), primary lateral sclerosis (PLS), progressive muscular atrophy (PMA), facial onset sensory and motor neuronopathy (FOSMN), hippocampal sclerosis (HS), limbic-predominant age-related TDP-43 encephalopathy (LATE), cerebral age-related TDP-43 with sclerosis (CARTS), Guam Parkinson-dementia complex (G-PDC), Guan ALS (G-ALS), Multisystem proteinopathy (MSP), Perry disease, Alzheimer's disease (AD), and chronic traumatic encephalopathy (CTE), or a combination thereof.

In embodiments, the methods for treatment of the present disclosure reduces, prevents, or slows development or progression of one or more symptom characteristic of a TDP-43 proteinopathy. Examples of symptoms characteristic of TDP-43 proteinopathy include motor dysfunction, cognitive dysfunction, emotional/behavioral dysfunction, paralysis, shaking, unsteadiness, rigidity, twitching, muscle weakness, muscle cramping, muscle stiffness, muscle atrophy, difficulty swallowing, difficulty breathing, speech and language difficulties (e.g., slurred speech), slowness of movement, difficulty with walking, dementia, depression, anxiety, or any combination thereof.

In embodiments, the methods for treatment of the present disclosure comprise administration of the UNC13A cryptic splice variant specific inhibitor as a monotherapy or in combination with one or more additional therapies for the treatment of the TDP-43 proteinopathy. Combination therapy may mean administration of the compositions of the present disclosure (e.g., antisense oligonucleotide) to the subject concurrently, prior to, subsequent to one or more additional therapies. Concurrent administration of combination therapy may mean that the compositions of the present disclosure (e.g., antisense oligonucleotide) and additional therapy are formulated for administration in the same dosage form or administered in separate dosage forms.

In embodiments, the one or additional therapies that may be used in combination with the UNC13A cryptic splice variant specific inhibitors of the present disclosure include: inhibitory nucleic acids or antisense oligonucleotides that target neurodegenerative disease related genes or transcripts (e.g., C90RF72), gene editing agents (e.g., CRISPR, TALEN, ZFN based systems) that target neurodegenerative related genes (e.g., C90RF72), agents that reduce oxidative stress, such as free radical scavengers (e.g., Radicava (edaravone), bromocriptine); antiglutamate agents (e.g., Riluzole, Topiramate, Lamotrigine, Dextromethorphan, Gabapentin and AMPA receptor antagonist (e.g., Talampanel)); anti-apoptosis agents (e.g., Minocycline, Sodium phenylbutyrate and Arimoclomol); anti-inflammatory agents (e.g., ganglioside, Celecoxib, Cyclosporine, Nimesulide, Azathioprine, Cyclophosphamide, Plasmapheresis, Glatiramer acetate and thalidomide); Beta-lactam antibiotics (penicillin and its derivatives, ceftriaxone, and cephalosporin); Dopamine agonists (Pramipexole, Dexpramipexole); and neurotrophic factors (e.g., IGF-1, GDNF, BDNF, CTNF, VEGF, Colivelin, Xaliproden, Thyrotrophin-releasing hormone and ADNF).

In embodiments, an UNC13A cryptic splice variant specific inhibitor of the present disclosure is administered in combination with an additional therapy targeting C90RF72. In some embodiments, the additional therapy targeting C90RF72 comprises an inhibitory nucleic acid targeting C90RF72 transcript, a C90RF72 specific antisense oligonucleotide, or a C90RF72 specific gene editing agent.

Examples of C90RF72 specific therapies are described in U.S. Pat. No. 9,963,699 (antisense oligonucleotides); PCT Publication No. WO2019/032612 (antisense oligonucleotides); U.S. Pat. No. 10,221,414 (antisense oligonucleotides); U.S. Pat. No. 10,407,678 (antisense oligonucleotides); U.S. Pat. No. 9,963,699 (antisense oligonucleotides); US Patent Publication US2019/0316126 (inhibitory nucleic acids); US Patent Publication No. 2019/0167815 (gene editing); PCT Publication No. WO2017/109757 (gene editing), each of which is incorporated by reference in its entirety.

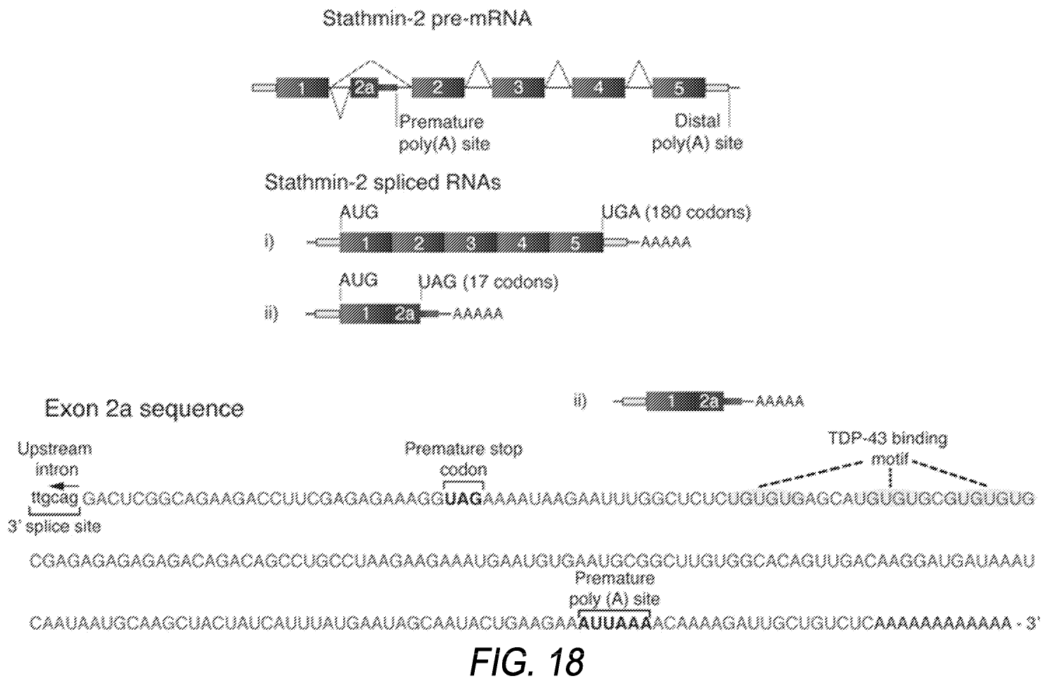

In embodiments, the methods for treatment of the present disclosure, including treating a TDP-43 proteinopathy such as ALS or FTD, may be used in combination with an STMN2 cryptic splice variant specific inhibitor. STMN2, which encodes a regulator of microtubule stability called Stathmin-2, is the gene whose expression is most significantly reduced when TDP-43 is depleted from neurons. The stathmin-2 gene is annotated to contain 5 constitutive exons plus a proposed alternative exon between exons 4 and 5 (see Table 10). STMN2 harbors a cryptic exon (exon 2a) contained in intron 1 that is normally excluded from the mature STMN2 mRNA (see, FIG. 18). The first intron of STMN2 (Table 10) contains a TDP-43 binding site. When TDP-43 is lost or its function is impaired, exon2a gets incorporated into the mature mRNA. Exon 2a harbors a stop codon and a polyadenylation signal (FIG. 18), resulting in truncated STMN2 mRNA and 8-fold reduction of Stathmin-2. Aberrant splicing and reduced Stathmin-2 levels seem to be a major feature of sporadic and familial ALS cases (except those with SOD] mutations) and in FTLD-TDP.

TABLE 10
STMN2 transcript sequence and intron 1 sequences
STMN2 transcript (NCBI Reference NM_001199214.1 Sequence)
AGCTCCTAGGAAGCTTCAGGGCTTAAAGCTCCACTCTACTTGGACTGTACTATCA
GGCCCCCAAAATGGGGGGAGCCGACAGGGAAGGACTGATTTCCATTTCAAACTG
CATTCTGGTACTTTGTACTCCAGCACCATTGGCCGATCAATATTTAATGCTTGGAG
ATTCTGACTCTGCGGGAGTCATGTCAGGGGACCTTGGGAGCCAATCTGCTTGAGC
TTCTGAGTGATAATTATTCATGGGCTCCTGCCTCTTGCTCTTTCTCTAGCACGGTC
CCACTCTGCAGACTCAGTGCCTTATTCAGTCTTCTCTCTCGCTCTCTCCGCTGCTG
TAGCCGGACCCTTTGCCTTCGCCACTGCTCAGCGTCTGCACATCCCTACAATGGCT
AAAACAGCAATGGCCTACAAGGAAAAAATGAAGGAGCTGTCCATGCTGTCACTG
ATCTGCTCTTGCTTTTACCCGGAACCTCGCAACATCAACATCTATACTTACGATGA
TATGGAAGTGAAGCAAATCAACAAACGTGCCTCTGGCCAGGCTTTTGAGCTGATC
TTGAAGCCACCATCTCCTATCTCAGAAGCCCCACGAACTTTAGCTTCTCCAAAGA
AGAAAGACCTGTCCCTGGAGGAGATCCAGAAGAAACTGGAGGCTGCAGAGGAA
AGAAGAAAGTCTCAGGAGGCCCAGGTGCTGAAACAATTGGCAGAGAAGAGGGA
ACACGAGCGAGAAGTCCTTCAGAAGGCTTTGGAGGAGAACAACAACTTCAGCAA
GATGGCGGAGGAAAAGCTGATCCTGAAAATGGAACAAATTAAGGAAAACCGTG
AGGCTAATCTAGCTGCTATTATTGAACGTCTGCAGGAAAAGCTGGTCAAGTTTAT
TTCTTCTGAACTAAAAGAATCTATAGAGTCTCAATTTCTGGAGCTTCAGAGGGAA
GGAGAGAAGCAATGAGAGGCATGCTGCGGAGGTGCGCAGGAACAAGGAACTCC
AGGTTGAACTGTCTGGCTGAAGCAAGGGAGGGTCTGGCACGCCCCACCAATAGT
AAATCCCCCTGCCTATATTATAATGGATCATGCGATATCAGGATGGGGAATGTAT
GACATGGTTTAAAAAGAACTCATTATAAAAAAAAAAAAACAAAAAAAATCAAA
AATTAAAAAAAATCAATGCGGTCTCTTTGCAGAATGTTTTGCTTGATGTTTAAAA
AATACCTTGGATCTTATTTTGTAAATACTTACATTTTTGTTAAAAAATACAAGTAT
TGCATTATGCAAGTTATTTCATAATCTTACATGTCCTGTAACAGGCTTTTGATGTT
GTGTCTTTCCACTCAAATGAATTTGCTAGGTCTGTTCTTTTTGAAGCTCCCCATGT
CTAACTCCATTCCAAAAGAAAAATGAGGTCAGTAGACAGTCTATGGTGCTAGAA
ACCCACCATTGCCTAATGACCTAGAAGGCTTTGTTGTCTCTGAGCTTGACTAAGA
CCATACCTAGATCACAGGTATTATGACTCCACATGAACCTTCACATTTGTTCGCTC
ATAATCTACTTACTGCCTAAAAACTACAAAACCAGGCTAAGAAATACCACCAGTC
ATAGCATTTACTTCTGCTTCTCCTGGATTATGTGCTACAAATGTGCTTTGGCTTTA
GAAAGGGATGGATGAGAAGACAGACCTGAGACCAATCTGGGTAGAAGCAAAAA
GTTGAACCTTTTAAAGTGCTGAACACAAATCCAAATTCGAATGGTTCAAGCAGCC
GTGAAATCGCTCTTCATAAAGTGGGCTTAATTCTCTAGTTTAAGTTCTTTTGATGG
AATGAATTAATTAATGTGTCAGGTGGCTTATTTGTGGATGCCATGATTGATGATG
TTCATTTTAAGCTCTTACCTATAGTACAAGTACATGATGCTACTGAATATTTTTCC
ACTTGGAAACTGTGAGCTGGTTGTTGCATTAAAACACACATACAAACAAAATCA
AAAACACTGCGGACTTTCACTCAAGCTGGTCTTTCTTCCCCAGTGTAAGGCAATC
CTGCCTACTAACAACACCAACAACAAAACACTCCATCTGTGAAGCTGACGCAGTT
AAGGGGGCTAGGCAGGGCATTTGTGCCAACTAAGAATCACCAGATACCCACCAT
AAGTACCTATCGCAGTTTTGAAGTCGTTTCTCCCCAACTCCCAACTCCTGAAGGTT
GCTGCCTGCATATTTACTCTTCATTAGTGCTATTTTCCTGTATGTCATTGTGAGCA
AGCTGTGATTAATAAAGAATTGGAGTTCTGTGAACTAATAAAGGTTTGGTCTGTT
AAAAAAAAA (SEQ ID NO: 390)
STMN2 Intron 1 Sequence
gtaaggcactgcgcctcgttctccgtcggctctacctggagcccacctctcacctcctctcttgagctctagaagcattcagagatatttta
taaagaaaaagatgttaatggtaacacaggaccaggaaggacagggcagttctgggggaggtgggagggcagagaagaggtctat
ggaaatctaaagcgaagaatttcttttaaaaggtagaagcgggtaagttgccctcctatgggtagagaatttattctgtttccatatttaaaat
taggactcaatcgtgaggggaggaagctaccttaactgtttgccttaaatgggcttaagggacattttggaaagtgctttataacgaccttt
tttttttttatttcttctctagtttaagaagaaaataggaaaggggtaaagggaaggtgggagaaaggaaaaagaaaattgcaaagtcaaa
gcggtcccatcccgctgtttgaaagatgggtggagacggggggaggggatggagagaactgggcacattttacggtattgtctcgtcg
aagaaaccgctagtcctggggtgcggtgcagggaggtaagacggcgggggacaggggggggtaggacctccgctcctttgtttta
gggcaagggaggggaaggagagaggaagtcgcggagggcgtggagggcgcggggggcagctgcaggggggggaagcgc
gcggcagggaggggtggagggacagcggcttcgaaggcgctggggggggtttctttgtgtgcggaccagcggtcccgggggga
ggcacctgcagcgctggggcacaatgcggacagccccacccagtgcggaaccgcgcagccccgcccccccgcccggtgctgc
atcttcattcgaaagggggtcgggtggggagcgcagcgtgacacccaggagcccaaccctgcggggacagcggcgccacgcccc
gcgctccccgctcccgactccccgccgcggcttccaagagagacctgaccactgaccccgccctccccacgctggcctcattgttctg
cttttaagagagatgggaaaagtgggttaacatttttcttttcggaagcaaattacatagagtgtttagacatagacacagataaagggttct
ttgaagacctttgatcgtttgcgggaaaagcttctagaacctagacatgtgtatgtataataatagagatgacatgaaatcgtatataaagc
aaaagaggtcaaagtcttaagttaagccacgcgaaatttccgttttgtgggtcagacagtgccaaatatcggcaatttcataagctcaga
gagacaagacagtggagacacaggatgaccggaaaagattctggattcagggccttcatccgcaattggtcttgtgccttgagtgccc
acggttctggcgctcagtggccccggggtgaaaaggcagggtggggcctggggtcctgtggcagctggaagcacgtgtcccccgg
gacttggttgcaggatgcggagacagggaaagctgccgaaaggactccatctgcgcggctccgccctgccctaccctccccgcgga
gccggggagacctcaggctccgagactggcggggaagaggaatatgggaggggcagttgagctgtatgcagtcctggaacctctttt
ttcagccccgcagtccacaacggcccgagcaccccttgatgtgcgcagacccccggcgtggctctcagccccagcaccgagcccct
cccagccaagcgggtggctctgcagaaaagctggctcgagccccgcccggccacacaaaggcgcggccccacccagcccgggc
gcgagaccgcagaggtgacccccttcccagggattcagggagggctgtctcttctcgcccacccacggtccgcggagctcggggct
ttttttcccccagcccaagccccccgcccaccctctgttctctatgattttccagaatggagaccccgcgaggggcttctctaagggaga
ccctcgctcctccagcggggcgcggctcggccccacccctcccagctgaggcccagagccgcctaccgctggccggggggggc
gcacgtggcgactgggtgtgtggagcgcagccagccctgcagagccccgcgccgcgccctgcgctcccctccccggagttgggcg
ctcgcccccgcggtgcagccggggagaccggtttctgcgcagtgtcctgagctacccccgctttccacaattcgcagttcactcgcac
gtccagaaaggttctgagaatgggtggtgggggcgatctcgcctcgctttctgcacccctcagaaaggtttccgctgcaggctagtggc
tgcaaactcatcgtcatcatcagtattattatcatttcaaatcgttgttattatttaatgattcagtagccttgtttgttctcatttgttcaaaaggg
acgtggattgctcttggttaaggattaacccttgttgcgttcgctttgcttcctcctaattgccctcatccctttcccccacaaaaaggtaaatt
tgtctccagttgttcattttaagttataaagcaaatatatttttgcttcctgccaggattatgtatgttcatgtggctaagatacatgtgcaagtg
cttgctaagagcagggtttgtgtgccaacgattgctggaaaattctctgcaaagaattgtttgtggctgcaatgggtgagaatacacatat
ataattgagatgatcttcaacataaggttatatctataaatatataaatatagtttatgcacaaaattttaagttttttcccctgaaactgttcttcc
aactgctgattcttgatacagcctcaatcctacacagatacatggatcgtgaaatggtagccgccatccaaataaaaatcccaccccaaa
tatgacaaacgcaagcatcctttctggccataatttaactgcatttgcaaatcatgaaaaaaacactacttctgcagtattaaaataatagatt
ttgaaattaattccaatttcaaagataattaattatcagggcgagtgcttttttcctgattcattaaacaattatgtattcagcatgattgtaaga
ggtgcatataatattccccattatcttttctaatgaagtgggcaccttctgaatggatatataagtaactagaaatgaaaagctgaggatttg
gtcagaatttcaggataaaactgaaagaaatggcagtagtttatcaattaatctcatgtatttagtttataccaggtgagtaagctgagcctg
caataaacactctctgtcccagtgtaacacgtcgcaggtagctagaatgataggataaattaatagaccttgtggtgtttgtctatgcacgt
taaaattctctgagagaaagtatattttaaaatgataattaagattggacatttgtgctattaaaatctacaactttagtcaaaattcacaatggt
ttttttttacaataatgtgacttacagatttgtagtaaattattctattctaaaagagaaatgagtgtttttattgttacagctattacctcattaat
atttttagcaaacttttatttgttgcattgaaagcagttttaattactttgggtttttatttttcaaattactaatggatagatggtggaataagcat
ttaatcatttggcacaatatgacttccatcaaatagctcattctcagtgattaaaaaatgctacaagaggctacaatttactcagattcaggaaat
gtcctttcagagtgccataaggctgattcatataataaaatagttttcttccctataatttaagatcaaatagttacttagttctgtgaataccta
gcagtagctatcaaacagaattttaaagttaaatctgtacaactaacaatgaagtggaggatgaatcgatacatattgaatggaagacttt
gtcattgataaattcaggccatctttaggaaaattccggatttatcaatcaccattattttttacttcaactgagtgtgactgatcacatgctca
ggctaccttggtagctcattgctcacaggaggctgaaaaaagctggcctccgagcaggaggaagctcagagcacaaacctaggcct
gggcgtggccactgggagctgctgatagcgaaccccagctcacaccagtttcttttttggtcgtgggaagaaaaacacatattatcctgt
tgtcacaagatctgtgaccttatatgaaaaaatgctagaattttttcattaaaaaagaaaatactgaactagccagtgacccagatgttttca
gaacctagactggttctgtccattggaaaacctcggtgtctgcattaacttttcaccacactagagggcaatcatgttctctaaaaaagcag
atgattgatgtaaacctagttccaaatattaactgtttaataaaatcttttcttttaccaggaacattcaagtgtttattcaataagctgatgccat
gctttaccctagtggatgaacagagcttgtacaattttcaaggagacaggatgaaatgagtggtcataatctgaaagtagatacacgccc
tggttaattattccctgatggttttacttctcagttttattacattgttattataataccatttatgttacttctgagattttgtagtggataaatag
tagaaaaatgtcagtagtaatagcaaagttatttagcagccgaatattttaatgcttaaaaataaaggaataaattaaagaaaatcattgtttact
tcttcatcgattgaaatgtgccccctgttcagagcacatctgaatatcagagtctccacctgcagagaacatgcagcttagcgagtaaaa
caggcaggtatgtgatactgaggaggtgtaccaaaaactgactgctgttatttttcccatcttctaagtctgtctttcttttccatttaaagata
cctttttaaatctaatccaatgtgatttcaatctagttttatcagatttcaacaattattgagcatctccttgtagtggttttctgtttattagaaaa
tcgatgttaattttaacgaagtaagaagaaatatataagtataaactaattttgggtatcatcaaaagtggattttttaaatatgcattgatagaa
ttattttttgattacattttatgtaattctaatccagctataaaatatttaatagtgtcatattactgtgttcctcaaactttgatgtgcatatgaat
tacctttgattttcattaaaatgcaaattctgattcaatacatctggcttgaggcagacattctgtcttccgaacaagctcccagatgatgctgat
tctgaccactaaacacatcagttttagggatattaacttgtaatatacaggtatccctcctggtaagctctggtattatgtcttaacatttttaaa
tctatggtaatctttacaaaatattttacttccgaactcatatacctggggattttattactctgggaattatgtgttctgccccatcactctctctt
aattggatttttaaaattatattcatattgcaggactcggcagaagaccttcgagagaaaggtagaaaataagaatttggctctctgtgtga
gcatgtgtgcgtgtgtgcgagagagagagacagacagcctgcctaagaagaaatgaatgtgaatgcggcttgtggcacagttgacaa
ggatgataaatcaataatgcaagcttactatcatttatgaatagcaatactgaagaaattaaaacaaaagattgctgtctcaatatatcttata
tttattatttaccaaattattctaagagtatttcttcctgaataccatgtgagaaaattcttaagaatttattgagtatgactgtatatttgaaaaga
gtgttttcttctgcttatctaagccaataaaggatcttcattattcaattctaactttctaaggaagtcaacctacagatcagaaagaggatctt
caaggaatagcatcaaagacatagtcaggtctcccatgcagtgactggctgaccatgcagccattaccacctttctggaaatattatgct
gcaaaaatgatacaatacacgaaatatctcaaattaaaaaatataacatttcccaaatagggcactaaaaacatgatcccaaataaaacta
gcttcagggtttgcagaatatactgttactcaacacaaagttggactaagtctcaaagttagccattcagttgttgttaacagttcatttcagg
gtctctcagaagctgggaaactttccatttttgcaatttcttgtacattgaaggaaaggaagacacacttaagacagcattacaaaagtaat
tcatgttttaaatgtttaattctggcagtcgggcagggctctctgtataacctcatttggagatgacaaaaatctaaacttgagggcctcgag
ccaataagtcttcctatttctttactcaaacattttcccgcaatggtgctttctttcaactgtttttctggtgtattcataaattccagattctctat
gggaagtaacttttattgattgatttaacccttgtatagcacatataacatgcaaggcattgttctaagaactttccacatattaactgtgttaatc
acttaataatcctaagtaggttctattacagatatggaaactgaggcacagaaagttgaagtatcttactcaaggtcacacagttagtcaga
tccagaatttgggcccaggccatctggcttcggaatccatctttcaccgattgctgctagtctcatatctgttccatgttagaggtgagctcc
cattgcagaggtcacacctgtgatatcaccattttatttaaacagaccagagatggtcttctcctttctgatcacagactcaccttgaagaga
aaatacttccaaattgatgcctagttttaatagcttacctggggcttattcaaataattgccatgatttaggctttgggagaaagagagctatg
aggccgtgtgggttgtaacgtatgagacacatggcgttctgcaggctcagcacagcatcgatttctggtgggaacacactctgatgacc
agttccagaaataacattgacttaatctcctcagtcccatcatggttagcacatttcaaaatgcctccttaactacttccataggccagagat
atttagttttaacattttgttgaataaaataaatttacacattcacatttaatataactattagatgttatttcaagattctcttcatattaccatca
aagcaggcaggcaggcaggagagaactgtaggaaggttttgaatcccttgtgaaacatttttaattatcttttaataaaggaatcaggccctg
tcatttgtcaaggagacatttgcagtagtaaagcttgtgtttataatatccatttttattagtcatgattaaagataacatttgtgtacatttgttct
cacaaaacacttttatatgagtgtaaaggttaattaatgcatttcagccatcattttgctggtcatgtggaaatatagcttctttaggaattgta
cttagagtaggagccacatattatactataaaaccataacaaaaatattttaagtttgttctcacttgttgttgacctccagagtaaaatattta
atactctggaaagttatgggtttcaaaatttattttatggcaagaaatagataattacagttctcatagagcacatttaaaataatttatttttata
gggcaaaaatattgcctaggactgaatgatttttttttttttacaaagattgtaaagcaacgcctgcaagagtgcccatttagcagttattcttc
tggaataattgtattttggatgttggagttcgcacattaaccattagtacaagtacccaatataacaatagatcatcaggataataaatctgtc
catcttttagttgtatgtctttatatcaggataaagagaattgagtgaaatttatctaaacctagtcccacaaatacttttacaagagagcatgt
taaagtgtaaattaaatttttattagcattctactctgtctttggaagttttttttccttatgaaatgcagccataaagtttaacttccattaacaaa
gctgctcacagtaaacctattataataatagtttcccagtttgggcttcctagtgaggagcaacctaactcacacgaaacaaccccaactt
ataatatattgactgttacaaaactgagaccagaaaatcccatcaagatggtactgttatcatttccagactctcgggaagaacattaatca
tctcaggcacttttaggatagacttattgcagcctccctgggaactctgcttcagaacataattatttttattaatgcagagttactttttatttcc
aacaaaaatatctattgttattatttaagtcttacagctttatctgagaaattccaattagcacccttctcataataaatattcaaacacatgaaa
aattaccaaagttgttctagtcttttaatgacatattacatgatcctgcactcttgtcactttaaaaattatctttttattatatttctgatgatttt
tttcttatatagttttttaaaaggagcaggcaagcatagaagactaaaaaatgttcaaaagaaaaattaaatcgcatgatctatctatatgggacc
ttgtcatttttagaaaacattcacctgcttcatccttttgaatcttcatataatccctctgagatgggcatactatacaagttgtcttatttaaagat
tggtaaatttaagctcaaataatttattcagtggcaagcctcagaggcagactcggaacacaggtctaatatatattatatatatattataac
atataatatatatattacatataataaagttgtgtatattatttacctatcaaaatatttatatgtaatatataaatatgttatatatcatgtatgtg
cctatttcatacatatatacacattcatgcaaaataaggtttagcactccctccactgtcctgtaataaaacatgcacagtgagaatagtcatac
acgaggcatatttgtcttcagtttaaagtcattgatagtcagtgtcactaactaaagtaaaatagattggagcaccaactttgttctgaagcc
tgtgccaggtattatgagaacaaaaataaaaatgttcctcacccttggtggatttagtcttttgcagaaaaaaagatcctgtacatgtcaga
aagttcaatagtaataatggtaatttataactataaatggaagtcaccatctcacaatttcaccatcttaacaattttgttaaactgccctacaa
tattacaagatagtacataatgatacactagtaacatcaactaggaagtaccaagatccaccaaaaggctgaaaaatttaaatatttaatga
gtccatcaaccaatctggccagagaattctttaattaaaatgcttcccaaattttactgagaatcagcagcgtttgaggagctagcctccac
ccccagaggttctcactctattaggtctgaagcaggtcccatggatttgcatttctaacaagctcccaggtggtgctgatgaggctgattc
agaaccacacttggagtagacctaaaacagcagtgacctgtagggtccccaagcagcaggccaggacagcatgtgagttacgtcctc
tgtggagctctgcaacaaggcgtcaagaggtcagagtctaagtccccatcagctctgcccttctccaccagtgctgctggtgctgcatg
gaaggaagagcccagaagggattctgagtttcagtctttactcttgctgacgcaccttggtcaggtcaattttcctgtttgttcctctaattca
gcatctgtaaaatagccatgtgaactgccttgtccatatcagagggtctttttcagactcaaggaaaaaaacgtgaaagtgattagtgtctg
tcaagtagtatataaatgcaagaagttgagtttttaaattgtcattagatataaatacccatgtgcatgcatttagaatgagtaaagagggaa
caaggagcgcaatcaaaaactgcgtcatttgctttttgaaaaatactttctatgtaatgaaaagtgaaataaaatgttaattgagtccctctg
acaacagcatcagacgttttgcagttcttgtgattagaacccacctggccagcccttcttcctcctaaagaagagccttcttcttcttaaatg
aaggttggctcagaagaagcaattaactcattcaacgttttgttacagtcaatccacatccaacttttccccaactcaatctgctttaaggga
aggatggtaagtggtggcccaagatggcaaccatcaagcttagagaatctctagaagcaggggtgtccccagcaagtagacactgaa
aatatgagagggctgataagccagagataaaactcagtacttactttgcttctagtccatgtctacccctttcttggcaccaccttgacacta
ccctctgagtccaccttcctgagatggtacaaactctgcttagacaaagcagcccatgtccaaaggtgttagggctcagtttaaagctgc
cttcaaaagttaaaacagaagtgtaaagttctgtgcaattaaaaataatcagcttgtcttggaactcaaacgaatgtaaaatcctatgaaaa
ttaaaaagcagtaccacaagttaccccaaaagtccttaggtcagtaactgttcctgttacaggtaagagagagcatggattagaggtg
ggcgtgggtatccagtggacatggttttgaaccatgctccactactactcactatctgagaattcttaaatttattaatcatttctatattataat
tttctcagttatgaaatgggaaaacaatacctaaatcacatggttgttaagtaagcaattgattgttaagcatttggtcatcaaaaatattaatc
cccttccctgattccctagataaatgatgaaaatactaaataaaaataataaaaatttaaagtgaacatctcaattcttatactttgttaatttct
acatgtattacaaatctactagaaattacttggaattgaggaaatgattactgcttaataattctttgtggtagagggagagttggtatcatatt
tatgagacagcagccaatatagtatatctcaaaggaaaaaatccattctacataatgccagaatttaatagttaagcattttatctaggtcac
agcacaataagcaagatggataattaaaataaaagtatatttctcttgcatatatttctcatttcatgtttccctatcatattttatatcttacctta
cttcaaatacatatataccttcaataaaactgagccttcttgcttacccaggaagtttcatcattcagtagaaataaaagatgactttagaaat
attaaaatacaaaaatctacactgaggtcttttgaatgcaggaaaaagaattatatcacacacacacgtacacgcacgcatgcatacaca
cacacagaacctctcgttctttcttaacatcttatcaatccatcagtttcactcccactccgtatcacctgactgtgcacaatatctcattgcca
cctcccagtcttctccctgcctggcaccctcctgctctcctgcttccactttaaacacccttccttcagctaggtcttttctttcagggatcctc
ccgttgctttcttatctggatcaatttagccttcctcttctccacccattagtggataagcacgacaaagacactagagtcaaataatacaaa
cagaatataccttagatgagtatggtgatgaaaaggatatggatacttagagtttagcactattctctcagccactcaggaaagcaacgcc
tttacaatcaatagtgtttcaggtaccaatcaataatctgttattgctatttttaaaatctataaggtatcagtaaaatgtaattactagagcaac
aaagatatcttgtgaaatcaaattagtattcatccagcaactgagtacaaaggtttaagggaggataactaccaataccaaaacattttaag
cattttgttttgcctcctaaatatcaaatcatgtaaatgtgtggtacataaattaggaattatatttatgacatagctgcagacatattaagaga
aatatgtgcttatatttacaagtatagtacagttctttttcatattagatactgttgatgataatctgcatataaaaatgctcaatattttttcacat
ttataagccataaaatacagctaataaaatgtgtttctactttctcataaacatggaatagtgacaaacaaggagctttatatgaaagcaccatt
acaatttaaactctcacaaggtcataatatattgcactaagcaggagagttcagcttatttaaaaaaaaaaataaactctaatgaggttctg
gaatgcagagccaaagcataaagatggaaataaaagaattgcatgtcttctgaactgacttggttgatgatttttttaaaaaaggttttgtgt
cttctgacttggttgatgattttttaaaaaaacgttttgtggtagaacaaataaggtaaatgaaattcagtatttaggatgaaaagtttttctaat
ttcaggaacaacattgaagaaatattgaactaagcagctttgaaagaatcagattccatttgttgaaatttttctgagaatgaatttttttaaga
cagtgtacacagttgcagtgtgtattggttatggattgtggcaagctatattacaacttacccaagaaataaggaggctgggcgtggtgg
ctcacacctgtaatcccagcactttgggtggccgaggcgggcggatcacgaggtcaggagatcgagaccatcctggctaacacggtg
aaaccccgtctctactaaaagtacaaaaaattagccgggtgtggtggcgggtgcctgtagtcccagctactcgggaggctgaggcag
gagaatggcgtgaatccgggagggggagtttgcagtgagccgagattgtaccactgcactccagcctgggcgacagagcgagactc
cgtctcaaaaaaaaaaaaaaaaaaaaaaaaagaaagaaagaaagaaggaaaaaagtcacttgaaaagaatactggactttgtgtcca
gcttgcatagctgaaaagaataaaaacctgtccacttaaactcattgcaaaaagaagatgtcactcctacaaatagcaaagagtcatgaa
attattctatccagaaaagtatacatttcatccctttggataaattttagaagtgaactatgaatacatacggtgaggatagccagctaagaa
gtcaagaaggatttctcaaatttgctgctcagaaagatcatactctccacaaaacaaataatagcaggctttccaagtcaaccttgaatcc
agctttcctttatctttccttcttgtgaactttcactagtttactatctaacaatgaatttgacgatagccacataccatcttatagcaatatttgtt
atcatatcccttgttatttatcattcacctgctctgcttgagccagctacaagtcacatgtcccacgcactttttcctgtttgattttttacagcact
ttgagacatgtctcattattcctacttgacaggaaagaagccatggaaagttgagtgacttgctcctgatcacaaatgctggccaaggaag
agtcgagtttcaaatctaatgatctttccactgcactctagattcctcattttgaactatttttttattttttgcactatagacttttttccacattt
tgaactgttttttattttttgcactatagacttttctcttatacccaactatattgatgacttcttttaggctagaaacttgtttcacttactttccc
tttcttcagattgctgcaatattggccaacatgtattgggtacttactgagtcaagtactgtgattgtgccaagtatcttataggaggattatcatcc
tcatttttacaggtgagaaaggaaaggaggtaaagtcacacacagccaacaaaaatggtagcaccaggatttgaaacaaatcagtctgac
ccaagttgactttgttaaccactgtatgcacagtcttcttagacatagtaagagctctaattgtgtttggtgatttgattattatgacaaagtaa
gtaagggaagcagggagaattataagaaataaggctccacaacacttggctatagcaaagccccttaaaacttcaaaaggtcacccaa
agaataaagatcaggctgggagcagtggctcacgcctgtaatcccagcactttgggaggccgaggtgggtggatcacctgagttcag
gagttcgagaccagcctggacaacatggtgaaaccctgtctctactaaaaatacaaaaattagctggatgtggtggttgccgcctgtaat
cccagctacttgggaggctgaggcagggagaatcgcttgaacccaggaggtggaggttgcagtgagccgagatcatgccactgcac
tccagcctgggcaacaagagcaaaaaactctgactcaaaaaaataaataaatcaatcaataaaataaagatcaatttggagaaattaat
gcttattaataagcaatgtcttgcacagcacttcagtttctcaatacattacctaactcaatccttacaacaacaccctatccccattttgtgga
taaataaactcatgttcagaaggttgaataaattatctaaggttaatagttcctgacctagagctcaaatcttcagtttctatcatattcttgccc
ttaccctggggtagctaacattcactcactagtattggagctaaaataagggagagaacatataaatgaatacaaaggagacattcacct
gccttctctttctccttacatagagaaggttgattatctgctattgtgaagtttgccttttgaaggatagaaatgagaagactttcttaaattttg
cctctacgccaagaaattagagtggtaccaccagtagttccattttcaaactatcactgtagctaaagctatgtggtaagggccaaggaa
aagaagtattcttgcacttcaaaatgcactgaaataccagtcagtagcataatataaaggaatttagtggagagaagagttgacctcaatc
tggctccaacatctcggctcttaacccctaccctacacttgttcttcatggggaagctaattgggccactggaagattcagcagctaccatt
tgcagctgagggacagcccctccctgcttagcaaccaatggatatgcatttatggaacacctgctaactgcgacacacactcctatgtat
gagggaaaatacaaaaaatgttaaaggagatgccttcccttgccctcaggaaacttaagtatagttgcaaagaaatgattagcagcaaa
cgaaaccatggagaagtaagggctaaggtctgtgaaacaagcctagaaaataaccttgtccttgaaaaacacaaaaagaaagaaaga
aagaaaagaaactccaaggcccttgtgaaggaaaccattaagtttgcttcacttctgtgtttaggaagacacaaacccagtcttaatgaac
ctcaaggccacaactactggagacatttaggaattgtcaccacattctaatgtatatatcctctgtttggcccttcctattaatattttgtaa
aatttttgaagatatgagcaatgtttaaaaccatgaatccccctttttttataagtaatatttaggctgaataaacaagagaaaataggacata
aaggggagccaacgtgtgccttcatttataatgtattcccaagttgtgagtttggtttatcagcaatttatcatgccaaattccaagtcatattt
atctatgcagatcaaacacttgattctatttttgccttaatttttttattgggtatgtttatgaccaagtcatatggtattttctgtgacagataaaa
tgcacaggttattccaatctggctcagccagtcatagcaacatgtagtccttctcatgtcttaagaatgagtatcaagaattcaaagggagtt
ccagatggcatccaaaaagcttacagtttatgcatcacttattctaacagtagaaaaagaatatttgaagccaaaaatagaccttgcatgta
gcatgtggaagagtagaaattgccctgatagttaaacaatttgaaattcaagacattaatttctttatgaagcatttgtcacatcataggtaat
attttatgcctatcatatatatacttattatgaaatacaaagaaattattcattctatctaagactttgtatcctttaccaatatctctccattctcc
cacctccaccctagcccctggaaaccacccttctactctctgcttctatgagttcttttttagtgagatcatgcagtatttgtctttctgttcctgtc
ttatttcacttgacataatgtccttcaggcttatccatgttgtcacaaatgacagaatttccttcttaaggctgaatagtattccattgtgtgtatg
tagcacattttctttattaattcatttgttgatggatactcatattgattccatatcttgggtcttgtgaataatgatgcagtgaacataggagtgc
agatatctttttgacatactgattccactttgatgggatatatacccagtagtgggactgctggatcatctagtagttttatttttttttatttttta
ttttttttattttgagacagagccttgctatgtcgcccaggctggagtacagtggtgccatctaggctcactgcaatctctgcctcctgggttca
agcaattttcctgcctcagcctcctgagtagctgggattacaggcacgcaccaccatgcccggctaatttttgtatgtttagtagagacgg
ggtttcaccatgtctcgaactcctgtcttcaagtgatccgtccacctcagactcccaaagtgctgcgattacaggtgtgagccaccacgc
ctggcctagtagttctgtttttaattttttgaggagcctccatactgctttccataatggctctaggaatttacattccaccagcagtgcacaag
gattgcttttctccacattctggctaaccagtctcctgtctttttgagaacagacatttcaacacgtgtgagataatatctcattgtggttttgatt
tgcatttccctgatgattagtgatcttgtgccttttttcatataactgctggacattaatatgccttcctttgagaactgtgtatacaggagaaaa
taatcacttctcagaggagctttcatttcaaaatatccgggaaaaaaatagaaaaaatggaaaatttatcctagagtaagttgtcttttatattt
tgaccctgtttgtgacataaactggatgatacaaaactggaatgcaaaggctttaggaggattacttacttacttgtatattgctttaggttgtt
tgcagaaaattatactaattgaagttcaggctatgatgtgataaaatctatgtcaggagatgagtctacatgcaaagtttgaggaagtgac
atttgagtttcaaaacaaaaaagcaattttcaatgtcatatctaggttaacccaaaagatttctttcaccctatttagctgcctctaagatggat
gctgaggataattacactgtagaacaataggacgatgcttcacactcacctcacaggctctgttattcccacatactgccagagatactcc
aaaataaaatcactgcaacatcaggcagttataaacctcaacggtattattttctatttatatacagtatattttatattttacaagtataaaata
gaatatatttattctattctctttgacacaaagtgaccataagacatattacttaagtatgactagcaaagtcatggggcttgtcattcaggag
gaaactcttaactaactgttcagtttttgttcactgcaccatttacataagccaaactaatgcttcacactgtgcaaaacaatgcacagtgttg
tgaatgaatggctaaaataaaactctaatgagtggggtttgaaaaatgcaactttagaaaactgttgagaaaatgttgcacactgcgcattt
tacaaaatttcgttgaaggacactggatattctttttaggattatggagggaagcaaaattttggctcctacatgcagtttttgtggcctttgc
ctgaaatagtcatctcccattaattatttagatatcattcatttcctaagacaacatttagggagactgccttaagtacaatttgtacactaccc
agataagaattctttttggtgaaacatcgataaatattacttggcagtaacaccaagttaaaatatttgtttcacagtcgacgttaataactatt
atagataaagtgaattttataagacatactcagatctaaaacagcaatatggagctcttcaaatccattgaaacttcataccagcctacgga
agtagaggtttttatgcaaactcttcaagaaatatgctctgaacttttaattccttagattgatagaggaattaaatcatgatataactaatagg
tttgtggtacaaattgctgctgcttaatctgactctgtgtcttcccagtgttctatatgaattagatattccattatctaaagacaatcaacccca
tcccacggtgatagctctaggactccctttgagttcattaaatctgtattctcagtctccaaacttctggttaattcaaacagaaaagtcaact
ggcccatgaactaaaataaagtcatctgaattttttttttattttgcagtgtgataaaagtctcgcactttttatttctgaaagtttctgctttcact
gagagcataataggctatccacccttatgcaatcttacatacaaagtcatagtcaggctaaattcaaaaacacatgtgagatagaagtca
acgtttattttctggagaaaagccacacattacaacaaagtgaacaatgaagctggcatccttatcactggtgaccaaaacatttgtgac
tctggacattggccccacaaatgcgataaacattctgcataggaagtgagttttgctaattaaaaatggatccaaaatactttctactcttca
gccaagaattaaaaagtaatagggaggaattgaaatcacttgggtgctacattgagccattctggagaagcaattcagagaatgtcatg
gcagcctcaaattgctgctcaggagcatcccagcttagaagattgcaggaaaggaagagcaaagtcattcttacatgagaactgtcctt
aaccagatgaatagactctccattttttaccctggctttgtctcatttaagtcccaaccaatctagctatcattttaggttttactacctgctagt
atttaggagcttagggggataaaaaaatccctcaatactcagaattagacttggtgataaaaatcttgacacataaacagaataaagcgct
ttcattactcctctaaaccacagtgtcatttggtctctatcaaggactgtaagaatttctttcatcaggggaaagaaaaaaaggacaagagc
ctgcaagatgtagcggaactctcattaaacacagcaggagctttaactggaatccagagtaaggtgaggtaccaggttacaacaattta
ctgcttttattacaattttgatcacaaggactgattcatgtcatctagtttcttttccttgtcactatcactggtgctaagaatacatcaaattgaa
atttaagagcctcatatgtttctgtataacccagtgatgggttgtactgctttgaccttcttaaatgtccctttatttcatttgatatccattcccat
agaaaaactataatgctttggttggtcaaaatattaatctttcaaaacctccctggcttagaaaaccaaatttttgtagagagagatgggtag
aatctaattttattctaaagcaattagcattacatcatcacagcagaaatatctagaatattacctcatgtcagtgatcttctgatatgttaaaaa
gggtattttaaaatctgagttatttctttttctttttaaagttacatcattaattacatactcatcaaccaaaatattttatgctccaaatttgaacc
gatatagtatgtaagaagtgttcaaaatgaaattattttggtctattttgtctttgaagaagatcacagggatggacctcccaaaaggattttta
aatgggattacatatctgacttttaaaaaaaattatctgaccttgagttatagtgccccaaagtaagcaaagttccaaacacacagtatcatc
agaattgagttaaaattatcaccaggggcttaatttctgaaattaaaaaggaaatgttatttccttatgaaaagaaaaggaaccaaaaatg
aacttcaaggtagctgatttctgtctatgttaagacttaggtaatgggagaaagggaaaaggaaggacagaattaggagaggagcagt
gtttaacaattgcgggtgcaagactcaagttttttagaatccattagcagagaaccctatttctcccattaactgctgtccttttaaatcctgg
caccagctctgaggactgcagggtccatagctagtgccccactctacccagtttaaagacaccactgcctggaaatgacaggggtttttt
tcttaaggaaagaggtgctttctgccacgtatatataaattggtaagcttcaaataaagtgcttttgtcctttctgtctatcagaaactgtgcaa
atcgaattgctgtaaaaccaagggcaagagacatcaatcctgcattctatagcatctgattttatcctttatccccaggcacatttcaaaag
gaaaaaaatgaggttgcatttaaattgagtatttgggacttgccaggaaaacctcccgctagactaatatgattgcagggaaaacaagag
aaaggaaaagtggagagggagtgtgctaacagatcctgggcctcgtcagcagagccgtcctgagcacaaggccatggtcagacatc
tggtcccgcgaatgacgttttctttatggtcattaagaacaccagtgtgtcgggacacaaacaagtattcctttcagggattatgacacattt
tctcccaaagtagtatattaatgacatttccagagcattctttactatcttttatatgtgatcaggaagactaatacatatcactacttcttttaca
cacagcattagccaaaactaaagtgtcaaatacaattttgcctaggatgaataaacagaagaaatttttatgatactgcactatcaattcca
aattaaataacaacaaaatgataagtgttaaaattcatattaatgattgttcccacacaagccggaaaaaatctttctaagaagtctttcatga
gttaatcccatctttcaaagtgttcagtggctccgaattcagttactgtttcctatcagttcttctttcattaagtctcttcccttttttttctcttt
gcactatttcccttagccgggtacataatctgctgtgctttattcatttgtgtcttaagtttgtttcccgatgacatacctttccagcaacgccatct
ggggagtttgggcaactgtaccacgttaggaggaaacccttcttcacaggagagtgtgcctttgctgcagggaaggaattaggatttgctt
ggactgtggttgcagctggcttttaaggatctccttagaatgcaagcaactcatcaatgagaatctctgcaatggttgtcactgggtagag
tcatgctatgtggggtcatagcctttgaaacaaataacagtaaagataaaaatgctattaaaggaatcaccacccacagaggttaactgg
gttttgtccccagaccacctcgaacaagaaagaacatttttatcagtcattttcttagttttagctgataaaacaaagtaccatagactaggt
ggcttataaacaacagaaatttatttttcacagctttggaaactggaagtctgagatcaggccgccagaatgatcagattctagttagggc
ctactttgcttttgcagactgccaacttctagctgcattttcatgtggcaaaaggagattgagctagctctctggtctcttcttataaggacac
taatcccattcatgaaggcttcaccttcatcatctaattactctccaaagaccccacctccaaatactatcacattgggaattagatttcaaat
acaaattttgcggggacacaaatattcagtccataatagtaatgattactcattatacatagggctctaaatgtgctagcttctgatagttttta
cactcacttctctttattagcttgtcaagcataattagggcagtggccttactgaaaattattgaatttagtttcctaaggacagatattgagga
gttttttcttcactaaaaattcacgttccgatacagctttcatctgttactactttgtgagatggaaaatcttttattttatttttatgtttggattg
acccttcttaataaagtcggcatgtaatatgcttcatgtgtttctaatatgtgcttaattttgcaaaatgttttgcataccagaatgcatttctcttc
caaaaaaggtaccagcctacaaaaccttgctgttactgttttcaattagttcatggaattaaatgtattaaatgttttatgctctggcagaaattat
gattctcacttaactccatataaatctggatctgcctgggcctttataagtgacacaatttcattaactgaataaacaaatgatacaaagaaa
tttggtttagccttctaaaattccaaaggcgttcaacaaaatatctcagaatggatgttccaggacttttatggcacaggacaacatgtattg
cttattttaagaaaataagctaaatagtgaggggattcttttagcagatcctcaggatgtgttaggttgaatcataggcaaatgatatttgatc
attgcacctgttaacacattgaacctcatcctaaaattgtagagctagaagaaagccttctggcagtttttaaatagattgatttactgcaattt
atccagaagcttcaccgttgtcactggctacatgtgactttggcctctgtggggctatatcctcatttgtaaaattggtggtgaggtaggtg
gacagttgactaaataatctcttagaataattctagtatctgtggatctaaagcatccaggggttgaatatgtttctttctggccaagaaaag
atgcacctgtcaataatgcccaaactcatcttctgagaatcctctttcccaagatacccactctcccttgggttatattatagtaatgatcaga
agcccctgccaagaagaaactgttaacctgggaggtctatattttatttcacagccatctgtttatactttctcacaagttagtgcacagtata
cccatcattttctaccattttccttaatttattaattttactaattgcataattaacaaaagtaagaagattttacctccttatccccatctggtagt
ttgcagatacttggcctgatgacaactgacagtgatgagatactcaccaagtttaccagggcaggaggcttcctagagaaaaaatgaga
aaatgaaatggggaaggggagtgaaggattgaggaggtgacaatctggactcttgcaactgcatggcaaggttggcacacaagctg
ggttgcaacggagggaaggagatccttatcagatgtaatcagagctcagatcgagggctttggtgtgtgtagaaagagggagagaca
aagaacttaaaacagagctgccatttgaccttgcaatcccattacttggtgtatacccaaaggagaataaatcattctattaaaaagacac
atgtgcttgtatgttcatggcagcactattcacaatagctaagacatggaatcaaactaggtgtccatctatggcagattggataaagaaa
atggggtaaatataaagcatgcaatacaacatggccataagaaaaaatgaaatcatgtcctttgctgcaacatggatgcagttgggacc
cataatcctaagtgaattaacacaggaacagaaaaccaaatacagcatgttctcacttataagtgggagctaaacactgagcacacatg
gacataaatatgagaacaataaacactgtggactactagaggggggaaggagagaggtttgtaaaactacctatcaggtgctatgctca
atacctgggtgatgggatttacaccccaaacatcagcatcatttaatattcccatgtaaaaagactgcacatataccccttgtatctaaaata
aaacttgaaattaaaaaaaaaagaaagaaagaaagaggctggaaatagaggctcacacctgtaatcccagcactttgggtggccaag
gtgggtggattgcttgagcccgggaattcaagaccagcctgagaaacctggtgaaactctgtctgtacaaaaaatacaaaaattatcca
ggcatggtggagcgcacctgtagtcccagctaatggggaggctgaggggggaacatcacttgagcccaggaggtggaggttgcagt
gagctgggatcacaccactgcactacagcctgggtaacagagcaactctgtctcaaagagagagaggaaagaaaaaagaaaagatg
gacagataagaaaatgcacttggagattaagagaaagcagcaacataggaccctggataatgtgtttgcttaataactatcctgatgagt
tatctgactattcccaaatgagtacgtggcaattcaggctgaaccatcagagtagccctccggaatcttacttatgtacaatagacctgcat
gcacatttactagaatgagcctctctctctggtaatcatgtctgcttccactaattccatctgtttcctctctctccctcctatcctgctagatctt
aattccttcgaccttcctttgtttttctaactccctttctttctcttgttatttaacctgctatactatgcaattgatctcctctgcactaaggaaca
tgcacttcagaattctgttgacatcttgcattcctttatatttagtgaaagaatgcaaaggagtctacctggcaatattcactctgcaggaggc
aataattattattcaaattaaaggaagcagtaaagagaaattcagaaaaaatgaaatatactaatcttcagottttcatttcag (SEQ ID
NO: 393)

In embodiments, the STMN2 cryptic exon splice variant specific inhibitor selectively inhibits the expression or activity of the STMN2 cryptic exon splice variant over full length STMN2 (wildtype) or other variants thereof (i.e., variants that do not contain a cryptic exon 2a contained in intron 1.

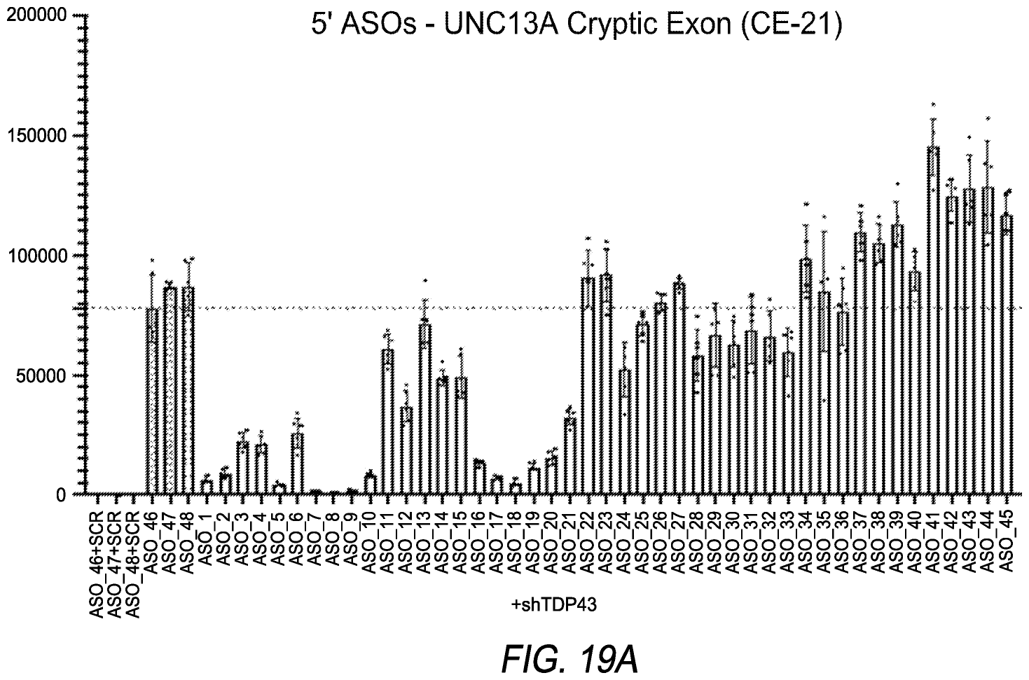

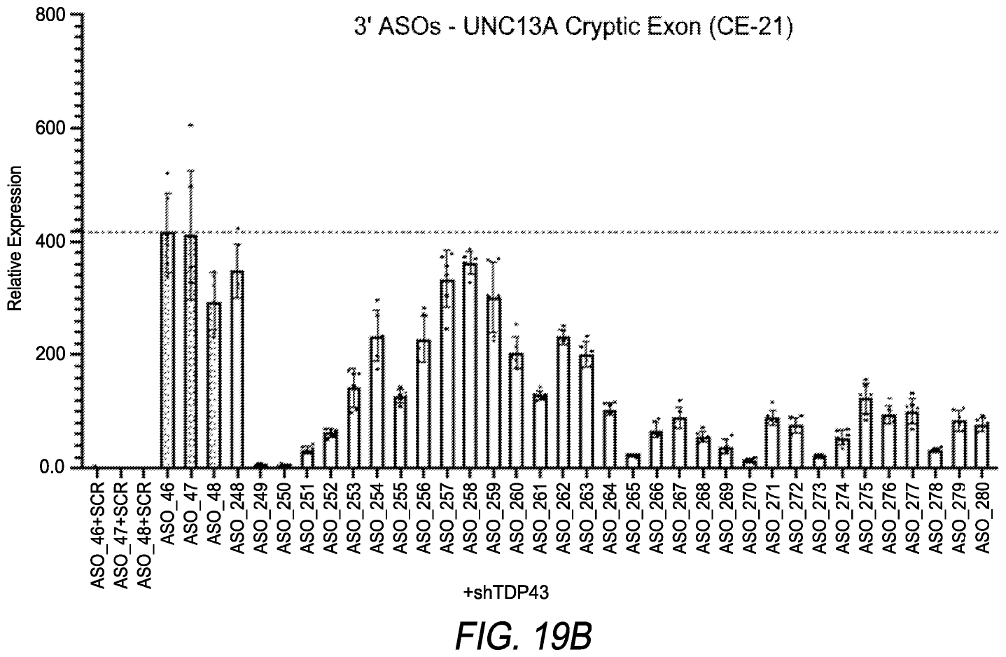

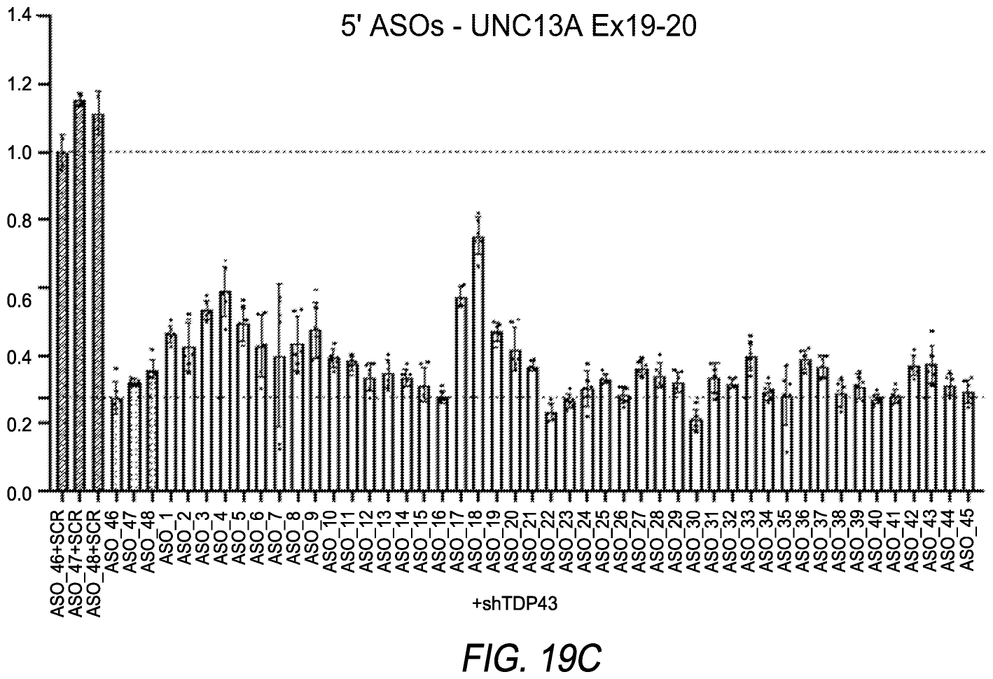

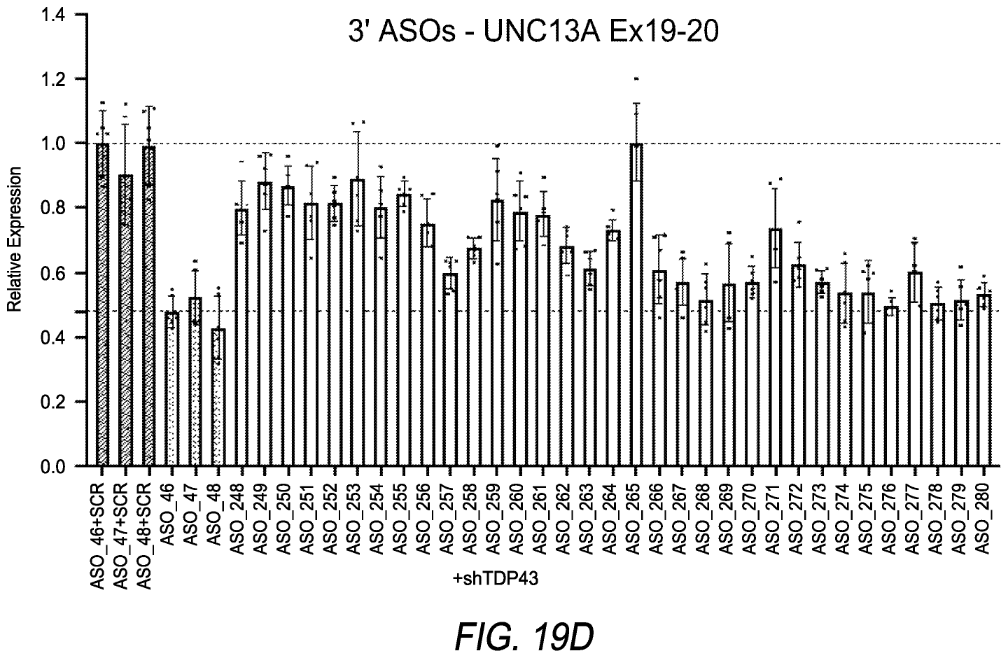

In embodiments, the STMN2 cryptic exon is obtained from intron 1 of the STMN2 gene. In embodiments, the cryptic exon 2a comprises the red sequence shown in FIG. 19.

In embodiments, the STMN2 cryptic splice variant specific inhibitor comprises an inhibitory nucleic acid, peptides, antibody, binding protein, small molecule, ribozyme, or aptamer.

In embodiments, the STMN2 cryptic splice variant specific inhibitor targets the cryptic exon 2a.

In embodiments, the STMN2 cryptic splice variant specific inhibitor comprises an inhibitory nucleic acid. The inhibitory nucleic acid may be an antisense oligonucleotide, siRNA, shRNA, miRNA, double-stranded RNA (dsRNAs), or esiRNA.

In embodiments, the inhibitory nucleic acid comprises an antisense oligonucleotide that is complementary to: the exon 1 splice donor site region in a preprocessed mRNA encoding STMN2; the cryptic exon 2a splice acceptor site region in a preprocessed mRNA encoding STMN2.

In embodiments, the STMN2 cryptic splice variant specific antisense oligonucleotide has about 15-40 bases in length, preferably about 18-30 bases, 18-25 bases, 18-22 bases, or 20-30 bases in length.

In embodiments, the STMN2 cryptic splice variant specific antisense oligonucleotide is a modified antisense oligonucleotide. In embodiments, the modified antisense oligonucleotide comprises a phosphoramidate morpholino oligonucleotide, phosphorodiamidate morpholino oligonucleotide, phosphorothioate modified oligonucleotide, 2′ O-methyl (2′ O-Me) modified oligonucleotide, peptide nucleic acid (PNA), locked nucleic acid (LNA), phosphorodithioate oligonucleotide, 2′ 0-Methoxyethyl (2′-MOE) modified oligonucleotide, 2′-fluoro-modified oligonucleotide, 2′0,4′C-ethylene-bridged nucleic acid (ENAs), tricyclo-DNA, tricyclo-DNA phosphorothioate nucleotide, constrained ethyl bridged nucleic acid, 2′-O-[2-(N-methylcarbamoyl)ethyl] modified oligonucleotide, morpholino oligonucleotide, and peptide-conjugated phosphoramidate morpholino oligonucleotide (PPMO), or any combination thereof.

UNC13A cryptic splice variant specific inhibitors of the present disclosure may be administered to a subject by any route, including enteral (e.g., oral), parenteral, intravenous, intramuscular, intra-arterial, intramedullary, intrathecal, subpial, intraparenchymal, intrastriatal, intracranial, intracisternal, intra-cerebral, intracerebral ventricular, intraocular, intraventricular, intralumbar, subcutaneous, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol Preferably, UNC13A cryptic splice variant specific inhibitors of the present disclosure (e.g., antisense oligonucleotide) are administered directly to the CNS of the subject, e.g., by intrathecal, subpial, intraparenchymal, intrastriatal, intracranial, intracisternal, intra-cerebral, intracerebral ventricular, intraocular, intraventricular, intralumbar administration, or any combination thereof.

In embodiments, the methods of the present disclosure reduces UNC13A cryptic splice variant expression or activity in a cell by at least 10%, at least 15%, at least 20%, at least 25%, at least 30%, at least 35%, at least 40%, at least 45%, at least 50%, at least 60%, at least 70%, at least 80%, at least 90% at least 95% or more in a cell compared to the expression level of UNC13A cryptic splice variant in a cell that has not been contacted with the UNC13A cryptic splice variant specific inhibitor. In some embodiments, the methods of the present disclosure reduces UNC13A cryptic splice variant expression or activity in a cell by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of UNC13A cryptic splice variant in a cell that has not been contacted with the inhibitory nucleic acid.

In embodiments, the methods of the present disclosure reduces UNC13A cryptic splice variant expression or activity in the CNS of a subject by at least 10%, at least 15%, at least 20%, at least 25%, at least 30%, at least 35%, at least 40%, at least 45%, at least 50%, at least 60%, at least 70%, at least 80%, at least 90% at least 95% or more in the CNS compared to the expression level of UNC13A cryptic splice variant in the CNS of an untreated subject. In embodiments, the methods of the present disclosure reduces UNC13A cryptic splice variant expression or activity in the CNS of a subject by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of UNC13A cryptic splice variant in the CNS of an untreated subject.

### EXAMPLES

### Example 1: TDP-43 Represses Cryptic Exon Inclusion in FTD/ALS Gene UNC13A

### Materials and Methods

RNA-Seq alinment and splicing analysis

Detailed pipeline v2.0.I for RNA-Seq alignment and splicing analysis is available on https://github.com/eme/2cube/Bioinformatics/sh_RNAseq.sh. FASTQ files were downloaded from the Gene Expression Omnibus (GEO) database as GSE126543. Adaptors in FASTQ files were removed using trimmomatic (0.39) (ILLUMINACLIP:TruSeq3-PE.fa:2:30:10 LEADING:3 TRAILING:3 SLIDINGWINDOW:4:15 MINLEN:36). The quality of the resulting files was then evaluated using FastQC (v0.11.9). RNA-Seq reads were then mapped to the human (hg38) using STAR v2.7.3a.

Splicing analysis

MAJIQ: Alternative splicing events were analyzed using MAJIQ (2.2) and VOILA (12). Briefly, uniquely mapped, junction-spannin^{g} reads were used by MAJIQ with the following parameters “majiq build -c config -min-intronic-cov 1-simplify” to construct splice graphs for transcripts by using the UCSC transcriptome annotation (release 82) supplemented with de novo detected junctions. Here, de novo refers to junctions that were not in the UCSC transcriptome annotation, but had sufficient evidence in the RNA-Seq data (-min-intronic-cov 1). Distinct local splice variations (LSVs) were identified in gene splice graphs and the MA JIQ quantifier (majiq psi) estimated the fraction of each junction in each LSV, denoted as percent spliced in (PSI or Ψ), in each RNA-Seq samples. The changes in each junction's PSI (ΔPSI or ΔΨ) between the two conditions (TDP-43-positive neuronal nuclei vs. TDP-43-negative neuronal nuclei) were calculated by using the command “majiq deltapsi”. The gene splice graphs, the posterior distribution of PSI and ΔPSI were visualized using VOILA.

LeafCutter (commit 249fc26 on https://github.coin/davidaknowles/ieafcutter): Using the already aligned RNA-Seq reads as previously described, reads that span exon-exon junction and map with a minimum of 6 np into each exon were extracted from the alignment (bam) files using filter_cs.py with the default settings. Intron clustering was performed using the default settings in leafcutter_cluster.py. Differential excision of the introns between the two conditions (TDP-43-positive neuronal nuclei vs. TDP-43-negative neuronal nuclei) were calculated using leafcutter_ds.R

Cell culture

SH-SY5Y (ATCC) cells were grown in DMEMI/F12 media supplemented with Glutamax (Thermo Scientific), 10% Fetal Bovine Serum and 10% penicillin-streptomycin at 37° C., 5% CO2. For shRNA treatments, cells were plated on Day 0, transduced with shRNA on Day 2 followed by media refresh on Day 3, and harvested for readout (RT-qPCR, immunoblotting) on Day 6. HEK293T TDP-43 knock-out cells and parent I-EK-293T cells were generated as described in (37). The cells were cultured in DMEM medium (Gibco 10564011) supplemented with 10% Fetal Bovine Serum (Invitrogen 16000-044), 1% penicillin-streptomycin, 2 mM L-glutamine (Gemini Biosciences), 1× MEM non-essential amino acids solution (Gibco) at 37° C., 5% CO2.

### Immunoblotting

SH-SY5Y cells and iPSC derived motor neurons (iPSCs-MNs) were transfected and treated as above before lysis. Cells were lysed in ice-cold RIPA buffer (Sigma-Aldrich R0278) supplemented with a protease inhibitor cocktail (Thermo Fisher 78429) and phosphatase inhibitor (Thermo Fisher 78426). After pelleting lysates at maximum speed on a table-top centrifuge for 15 min at 4° C., bicinchoninic acid (Invitrogen 23225) assays were conducted to determine protein concentrations. 60 μg (SH-SY5Y) and 30 prg (iPSCs-M Ns) protein of each sample was denatured for 10 min at 70° C. in LDS sample buffer (Invitrogen NP0008) containing 2.5% 2-mercaptoethanol (Sigma-Aldrich). These samples were loaded onto 4-12% Bis-Tris gels (Thermo Fisher NP0335BOX) for gel electrophoresis, then transferred onto 0.45-nm nitrocellulose membranes (Bio-Rad 162-0115) at 100 V for 2 h using the wet transfer method (Bio-Rad Mini Trans-Blot Electrophoretic Cell 170-3930). Membranes were blocked in Odyssey Blocking Buffer (LiCOr 927-40010) for ih then incubated overnight at room temperature in blocking buffer containing antibodies against UNC13A (1:500, Proteintech 55053-1-AlP), TDP-43 (1:1,000, Abnova H00023435-M01), or GAPDH (Cell Signaling Technologies 5174S). Membranes were subsequently incubated in blocking buffer containing HRP-conjugated anti-mouse IgG (H+L) (1:2000, Fisher 62-6520) or HRP-conjugated anti-rabbit IgG (H+L) (1:2000, Life Technologies 31462) for one hour. ECL Prime kit (Invitrogen) was used for development of blots, which were imaged using ChemiDox XRS+System (BIO-RAD). The intensity of bands was quantified using Fiji, and then normalized to the corresponding controls.

RNA Extraction, cDNA Synthesis, and RT-qPCR/RT-PCR for detecting the IJNC13A splice variant

Total RNA was extracted using RNeasy Micro kit (Qiagen) per manufacturer's instructions, with lysate passed through a QlAshredder column (Qiagen) to maximize yield. RNA was quantified by Nanodrop (Thermo Scientific), with 75 ng used for cDNA synthesis with SuperScript IV VILO Master Mix (Thermo Scientific). qPCR was run with 6 ng c[)NA input in a 20u1 reaction using PowerTrack SYBR Green Master Mix (Thermo Scientific) with readout on a QuantStudio 6 Flex using standard cycling parameters (95° C. for 2 minutes, 40 cycles of 95° C. for 15s/60° C. for 60s), followed by standard dissociation (95° C. for 15s at 1.6° C./second, 60° C. for 60s at 1.6° C./second, 95° C. for l 5s at 0.075° C./second). AACt was calculated with RPLPO as housekeeper and relevant shScramble as reference; measured Ct values greater than 40 were set to 40 for visualizations. The following primer pairs were used:

| | | SEQ |
|---|---|---|
| | | ID |
| Primer Name | Sequence | NO: |
| UNC13A_CE FWD 5′-3′ | TGGATGGAGAGATGGAACCT | 379 |
| UNC13A_CE RVS 5′-3′ | GGGCTGTCTCATCGTAGTAAAC | 380 |
| UNC13A FWD 5′-3′ | GGACGTGTGGTACAACCTGG | 381 |
| UNC13A RVS 5′-3′ | GTGTACTGGACATGGTACGGG | 382 |
| TARDBP_1 FWD 5′-3′ | AATTCTGCATGCCCCAGA | 383 |
| TARDBP_1 RVS 5′-3′ | GAAGCATCTGTCTCATCCATTTT | 384 |
| RPLP0_1 FWD 5′-3′ | TCTACAACCCTGAAGTGCTTGAT | 385 |
| RPLP0_1 RVS 5′-3′ | CAATCTGCAGACAGACACTGG | 386 |

RT-PCR was conducted with 15 ng cDNA input in a 100 ul reaction using NEBNext Ultra I1 Q5 Master Mix (New England Biolabs), with resulting products visualized on a 1.5% TAE gel. The following primer pairs were used:

| | | SEQ |
|---|---|---|
| | | ID |
| Primer Name | Sequence | NO: |
| UNC13A_19_21 FWD 5′-′3 | CAACCTGGACAAGCGAACTG | 387 |
| UNC13A_19_21 RVS 5′-3′ | GGGCTGTCTCATCGTAGTAAAC | 388 |
| UNC13A_CE FWD 5′-3′ | TGGATGGAGAGATGGAACCT | 379 |
| UNC13A_CE RVS 5′-3′ | GGGCTGTCTCATCGTAGTAAAC | 380 |

shRNA cloning lentiviral packaging, and cellular transduction

shRNA sequences originated from the Broad GPP Portal (TDP-43: AGA TCTT AAGACTGGTCATTC (SEQ I) NO:391), scramble: GATATCGCTTCTACTAGTAAG (SEQ ID NO:392)). To clone, complementary oligos were synthesized to generate 4 nt overhangs, annealed, and ligated into pRSITCH (Tet inducible 16) or pRS116 (constitutive U6) (Cellecta). Ligations were transformed into Stb13 chemically competent cells (Thermo Scientific) and grown at 30° C. Large scale plasmid generation was performed using Maxiprep columns (Promega), with purified plasmid used as input for lentiviral packaging with second generation packaging plasmids psPAX2 and pMD2.G (Cellecta), transduced with Lipofectamine 2000 (Invitrogen) in Lenti-X 293T cells (Takara). Viral supernatant was collected at 48 and 72 hours post transfection and concentrated using Lenti-X Concentrator (Takara). Viral titer was established by serial dilution in relevant cell lines and readout of % BFP+by flow cytometry, with a dilution achieving a minimum of 80% BFP+cells selected for experiments.

Variant validation

Variants in iPSC-derived motor neuron cells were established by PCR amplification from UNCI3A exon 19 to exon 21 (UNCI3A 19_21 FWD 5′-3′ CAACCTGGACAAGCGAACTG (SEQ ID NO:387), UNC13A_19_21 RVS 5′-3′=GGGCTGTCTCATCGTAGTAAAC (SEQ ID NO:388)). Resulting products were purified using Wizard SV Gel and PCR Clean-Up columns (Promega) and submitted for Sanger and NGS (Amplicon EZ) (Genewiz).

iPSC maintenance and differentiation into motor neurons (iPSC-MNs)

iPSC lines were obtained from public biobanks (GM25256-Corriell Institute; NDS00262, NDS00209-NINDS) and maintained in mTeSR1 media (StemCell Technologies) on matrigel (Corning). iPSCs were fed daily and split every 4-7 days using ReLeSR (StemCell Technologies) according to manufacturer's instructions. Differentiation of iPSCs into motor neurons was carried out as previously described (41). Briefly, iPSCs were dissociated and placed in ultra-low adhesion flasks (Corning) to form 3D spheroids in media containing DNMEIF12/Neurobasal (Thermo Fisher), N2 supplement (Thermo Fisher), and B-27 supplement-Xeno free (Thermo Fisher). Small molecules were added to induce neuronal progenitor patterning of the spheroids, (LDN193189, SB-431542, Chir99021), followed by motor neuron induction (RA, SAG, DAPT). After 14 days, neuronal spheroids were dissociated with Papain and DNAse (Worthington Biochemical) and plated on Poly-D-Lysine/Laminin coated plates in Neurobasal medium (Thermo Fisher) containing neurotrophic factors (BDNF, GDNF, CNTF; R&D Systems). For viral transductions, neuronal cultures were incubated for 18 hr with media containing lentivirus particles for shScramble, or shTDP-43. Infection efficiency of over 90% was assessed by RFP expression. Neuronal cultures were analyzed for RNA and protein 7 days post transduction.

| Cell line name | Sex | Age | Disease Mutation | Source |
|---|---|---|---|---|
| GM25256 | M | 30 | N/A | Coriell Institute |
| NDS00209 | M | 64 | TDP43 G298S | NINDS |
| NDS00262 | M | N/A | N/A | NINDS |

Human iPSC-neurons for detecting UNC13A splice variant PGP25,T1

Complementary cDNA was available from CRISPRi-i³Neuron iPSCs (i³N) generated from our previous publication (10), in which TDP-43 is downregulated to about 50%. Quantitative real-time PCR (RT-qPCR) was performed using SYBR GreenER qPCR SuperMix (Invitrogen). Samples were run in triplicate, and RT-qPCRs were run on a QuantStudio^{T}M 7 Flex Real-Time PCR System (Applied Biosystems). The following primer pairs were used: UNC13A_CE FWD 5′-3′=TGGATGGAGAGATGGAACCT (SEQ ID NO:379). UNC13A CE RVS 5′-3′=GGGCTGTCTCATCGTAGTAAAC (SEQ I) NO:380) Relative quantification was determined using the AACt method and normalized to the endogenous controls RPLPO and GAPDH (GA^{I}DH FWD 5-3′ GTTCGACAGTCAGCCGCATC (SEQ [D NO:397), GAPDHRVS 5′-3′=GGAATTTGCCATGGGTGGA (SEQ ID NO:398); RPLP0_2 FWD 5′-3′=TCTACAACCCTG-AAGTGCTTGAT (SEQ ID NO:399), RPLP0_2R VS 5′-3′=CAA TCTGCAGACAGCACTGG (SEQ ID NO:400)). Relative transcript levels for wild-type UNC13A were normalized to that of the healthy controls (mean set to 1).

Post-mortem brain tissues for detecting UNC13A splice variant

Post-mortem brain tissues from patients with FTLD-TDP and cognitively normal control individuals were obtained from the Mayo Clinic Florida Brain Bank. Diagnosis was independently ascertained by trained neurologists and neuropathologists upon neurological and pathological examinations, respectively. Written informed consent was given by all participants or authorized family members and all protocols were approved by the Mayo Clinic Institution Review Board and Ethics Committee. Complementary DNA (cDNA) obtained from 500 ng of RNA (RIN >7.0) from medial frontal cortex was available from a previous study, as well as matching pTDP-43 data from the same samples (42). Following standard protocols, quantitative real-time PCRs (RT-qPCR) were conducted using SYBR GreenER qPCR SuperMix (Invitrogen, Carlsbad, CA, USA) for all samples in triplicates. Primer pair used for detecting UNC13A splice variant were UNC13A_CE FWD 5′-3′=TGGATGGAGAGATGGAACCT (SEQ ID NO:379), UNC13A_CE RVS 5′-3′=GGGCTGTCTCATCGTAGTAAAC (SEQ ID NO:380). RT-qPCRs were run in a QuantStudio™ 7 Flex Real-Time PCR System (Applied Biosystems). Relative quantification was deternined using the ΔΔCt method and normalized to the endogenous controls RPLPO and GAPDH (GAPDH FWD 5′-3′=GTTCGACAGTCAGCCGCATC (SEQ ID NO:397), GAPDU RVS 5′-3′=GGAA TTTGCCA TGGGTGGA (SEQ ID NO:398); RPLP0 2 FWD 5′-3′=TCTACAACCCTGAAGTGCTTGAT (SEQ ID NO:399), RPLPO 2 RVS 5′-3′ 5′ CAA TCTGCAGACAGACACTGG (SEQ ID NO:400)). Relative transcript levels were normalized to that of the healthy controls (mean set to 1).

Quantification of UNC13A splice variants

RNA-Seq data generated by NYGC ALS Consortium cohort were downloaded from the NCBI's Gene Expression Omnibus (GEO) database (GSE137810, GSE124439, GSE116622, and GSE153960). The 1658 available and quality-controlled samples classified as described in (10) was used. After pre-processing and aligning the reads to human (hg38) as described previously, the expression of the full-length UNC3A was estimated using RSEM (v1.3.2). The average TPM of UNC13A across all the tissue samples from all the individuals was 10.5 on average. PCR duplicates were removed using MarkDuplicates from Picard Tools (2.23.0) using the command “MarkDuplicates REMOVE_DUPLICATES=true CREATE INDEX=true”. Reads that span either “Exon 19-Exon 20” junction, “Exon 20-CE” junction, “CE-Exon 21” junction, or “Exon 20-exon 21“junction were quantified using bedtools (2.27.1) using the command “bedtools intersect -split”. Because of the relatively low level of expression of U_NC13A in post-mortem tissues and the heterogeneity of the tissues, it is possible that not all tissues have enough detectable UNC13A for us to detect the splice variants. Since UNC13A contains more than 40 exons and RNA-Seq coverages of mRNA transcripts are often not uniformly distributed (43), reads spanning “Exon 19-Exon 20” junction, which is included in both the canonical isoform and the splice variant, were examined and there is a strong correlation (Pearson's r:=0.99) between the numbers of reads mapped to “Exon 19-Exon 20” junction and “Exon 20-Exon 21” junction. Samples that have at least 2 reads spanning either “Exon 20-CE” junction or “CE-Exon 21” junction were observed to have at least either UNC13A TPM=1.55 or 20 reads spanning “Exon 19-Exon 20” junction. Therefore, the 1151 samples that had a TPM >1.55, or at least 20 reads mapped to the “Exon 19-Exon 20” junction were selected as samples suitable for UNC/13A splice variant analysis.

Determination of rs12608932 and rs12973192 SNP genotvpe in human postmortem brain

Genomic DNA (gDNA) was extracted from human frontal cortex using Wizard Genomic DNA Purification Kit (Promega), according to the manufacturer's instructions. TaqMan SNP genotyping assays were performed on 20 ng ofgDNA per assay, using a commercial pre-mixture consisted of a primer pair and VIC/FAM labeled probes specific for each SNP (Cat #4351379, assay ID “43881386_10” for rs12608932 and “11514504_10” for rs12973192, Thermo Fisher Scientific), and run on a QuantStudio™ 7 Flex Real-Time PCR system (Applied Biosystems), according to the manufacturer's instructions. The PCR-programs were 60° C. for 30 s, 95° C. for 10 min, 40 cycles of 95° C. for 15s and, 60° C. (rs12973192) or 62.5° C. for 1 min (rs12608932), and 60° C. for 30s.

### Splicing Reporter Assay

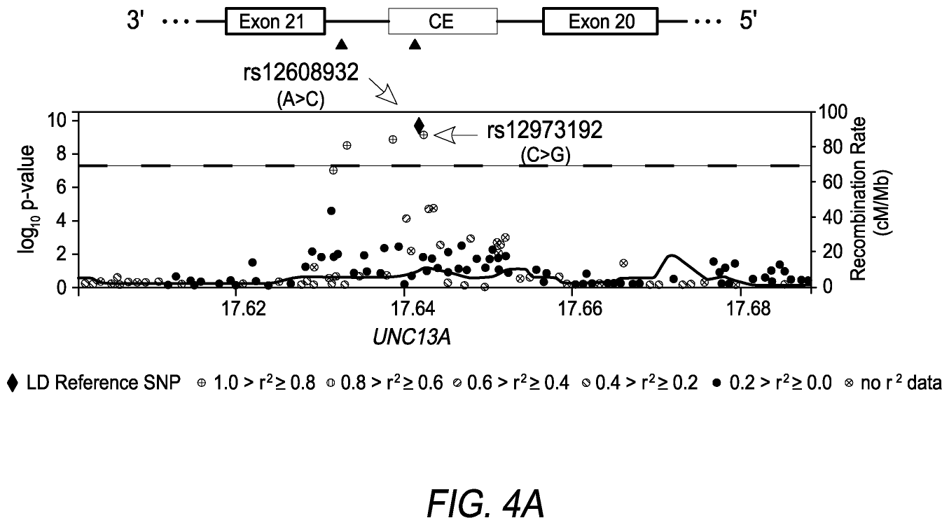

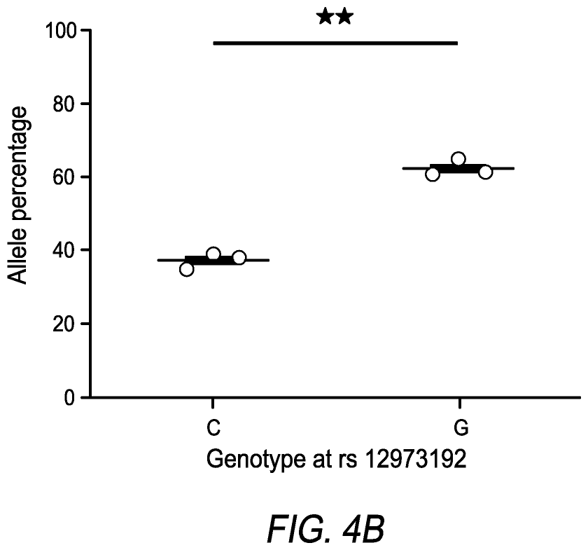

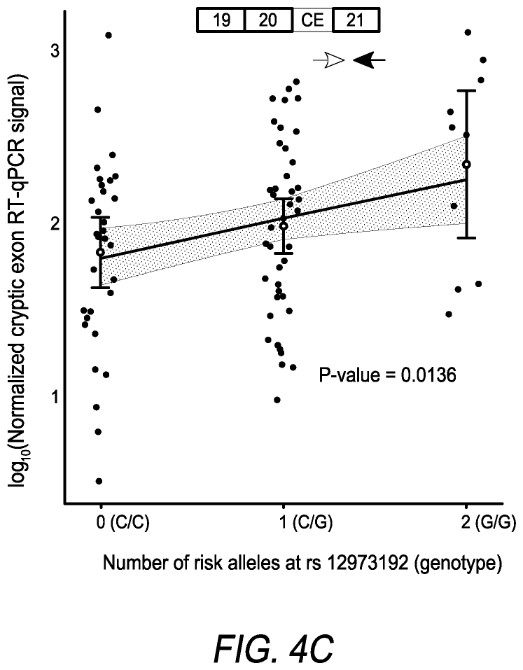

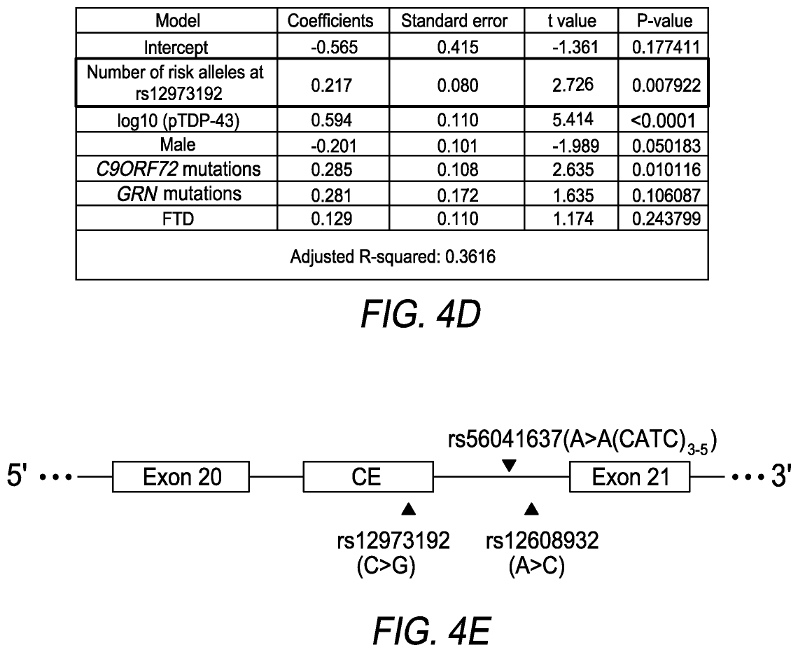

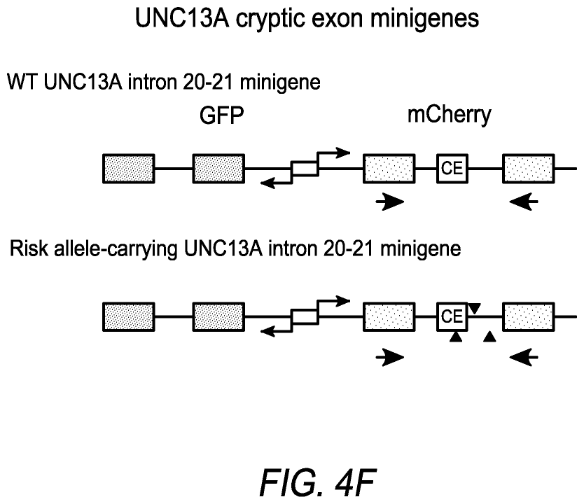

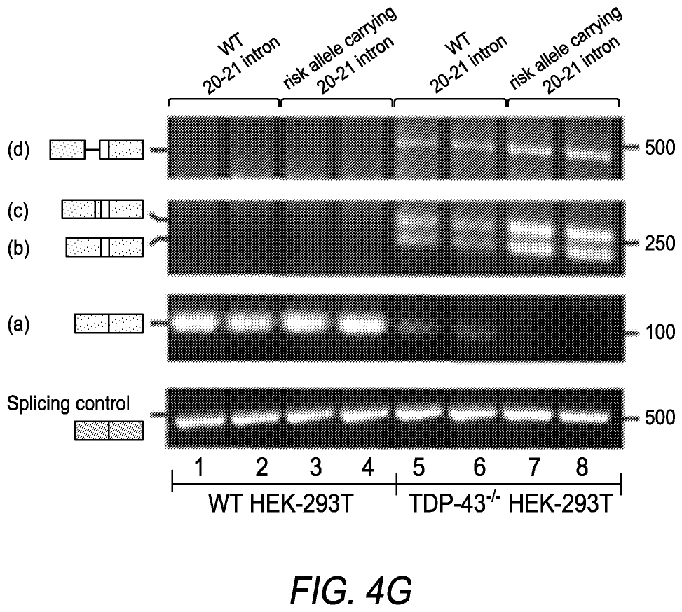

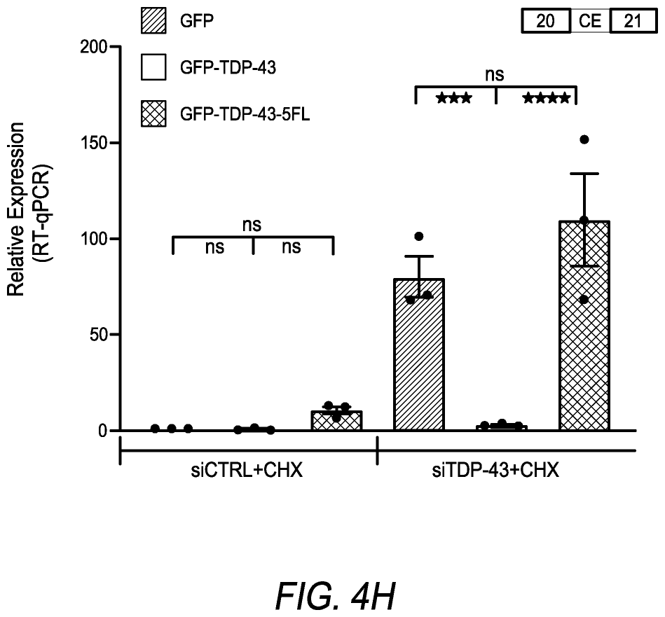

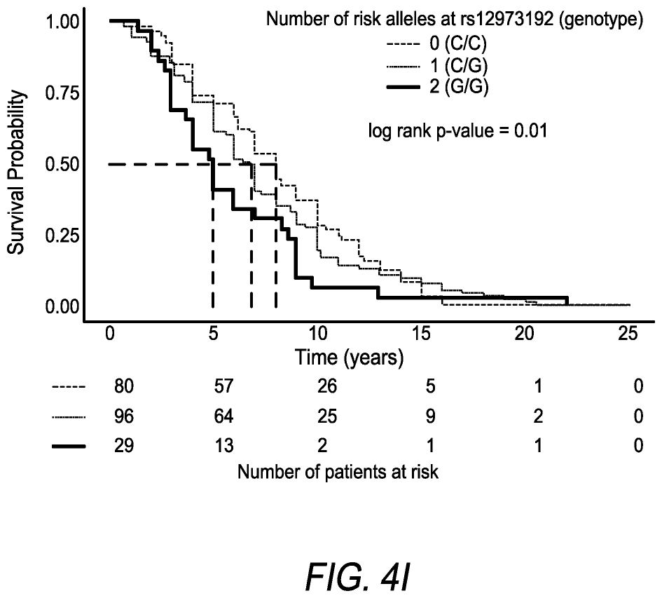

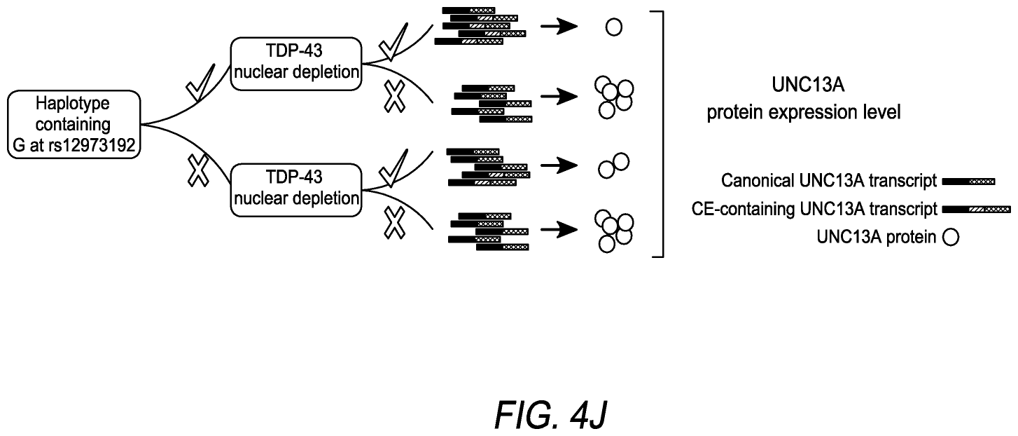

Minigene constructs were designed in silico, synthesized by GeneScript and sub-cloned into a vector with the GFP splicing control. HEK293T TDP-43 knock-out cells and the parent HEK-293T cells were seeded into standard P12 tissue culture plates (at 1.6×10⁵ cells/well), allowed to adhere overnight and transfected with the indicated splicing reporter constructs (400 ng/well) using Lipofectamine 3000 Transfection Reagent (Invitrogen). Each reporter comprised one of the splicing modules (shown in FIG. 4E), which is expressed from a bidirectional promoter. Twenty-four hours after transfection, RNA was extracted from these cells using PureLink RNA Mini Kit (Life Technologies) according to the manufacturer's protocol, with on-column PureLink DNase treatment. The RNA was reverse transcribed into cDNA using the High Capacity cDNA Reverse Transcription Kit (Invitrogen) according to the manufacturers' instructions. PCRs were performed using OneTag 2X Master Mix with Standard Buffer (NEB) using the following primers: mCherry FWD 5′-3′=GTTCATGCGCTTCAAGGTG (SEQ ID NO:407), mCherry RVS 5′-3′=TTGGTCACCTTCAGCTTGG (SEQ ID NO:408); EGFP FWD 5′-3′=ACAGGTACTGTGCCTATCAAAG (SEQ ID NO:409); EGFP RVS 5′-3′=TGTGGCGGATCTTGAAGTTAG (SEQ ID NO:410) on a Mastercycler Pro (Eppendorf) thermocycler PCR machine. PCR products were separated by electrophoresis on a 1.5% TAE gel and imaged ChemiDox XRS+System (BIO-RAD).

Generation of pTB UNC 13A minigene construct

The pTB U.,NC13A minigene construct containing the human UNVC13A cryptic exon sequence and the nucleotide flanking sequences upstream (50 bp at the of end of intron 19, the entire exon 20, the entire intron 20 sequence upstream of the cryptic exon) and downstream (˜300 bp intron 20) of the cryptic exon were amplified from human genomic DNA using the following primers: FWD 5′-3′=AGGTCTAGCACGTATAGGGGAAGTTC (SEQ 11) NO:411) and RVS 5′-3′=CTTACATATGTAATAACTCAACCACACTTCCATC SEQ ID NO:412); and subcloned into the NdeI site of the pTB vector. Note a similar approach to study TDP-43 splicing regulation of other TDP-43 targets was previously used (44).

Rescue of UVC13A splicing using the pTB minigene and TDP-43 overexpression constructs

HeLa cells were grown in Opti-M-EM I Reduced Serum Medium, GlutaMAX Supplement (Gibco) plus 10% fetal bovine serum (Sigma) and 1% penicillin/streptomycin (Gibco). For double-transfection and knockdown experiments, cells were first transfected with 1.0 μg of pTB UNC13A minigene construct and 1.0 Ig of one of the following plasmids: GFP, GFP-TDP-43 or GFP-TDP-43 5FL (constructs to express GFP-tagged TDP-43 proteins have been previously described (40, 44), in serum-free media and using Lipofectamine 2000 following manufacturer's instructions (Invitrogen). Four hours following transfection, media was replaced with complete media containing siLentfect (Bio-Rad) and siRNA complexes (AllStars Neg. Control siRNA or siRNA against ARDBP 3′UTR, a region not included in the TDP-43 overexpression constructs) (Qiagen) following the manufacturer's protocol Cycloheximide (Sigma) was added at a final concentration of 100 μg/ml at six hours prior harvesting the cells. Then cells were harvested and RNA extracted using TRIzol Reagent (Zymo Research), following manufacturer's instructions. Approximately 1 μg of RNA was converted into cDNA using the High Capacity cDNA Reverse Transcription Kit with RNA inhibitor (Applied Biosystems). The RT-qPCR assay was performed on cDNA (diluted 1:40) with SYBR GreenER qPCR SuperMix (Invitrogen) using QuantStudio7^{T}rm Flex Real-Time PCR System (Applied Biosystems). All samples were analyzed in triplicates. The RT-qPCR program was as follows: 50° C. for 2 min, 95° C. for 10 min, and 40 cycles of 95° C. for 15 s and 60° C. for 1 min. For dissociation curves, a dissociation stage of 95° C. for 15 s, 60° C. for i min and 95° C. for 15 s was added at the end of the program. Relative quantification was determined using the AACt method and normalized to the endogenous controls RPLP0 and GAPDI. Relative transcript levels for wild-type [INfC13 1 and GFP were normalized to that of the control siRNA condition (mean set to 1).

The following primer pairs were used:

| | | SEQ |
|---|---|---|
| | | ID |
| Primer Name | Sequence | NO: |
| UNC13A_CE_minigene | GATTGAACAGATGAATGAGTGATGA | 413 |
| FWD 5′-3′ | | |
| UNC13A_CE_minigene | TGTCTGGACCAATGTTGGTG | 414 |
| RVS 5′-3′ | | |
| GFP_OE FWD 5′-3′ | GAAGCGCGATCACATGGT | 415 |
| GFP_OE RVS 5′-3′ | CCATGCCGAGAGTGATCC | 416 |
| GAPDH FWD 5′-3′ | GTTCGACAGTCAGCCGCATC | 397 |
| GAPDH RVS 5′-3′ | GGAATTTGCCATGGGTGGA | 398 |
| RPLP0_2 FWD 5′-3′ | TCTACAACCCTGAAGTGCTTGAT | 399 |
| RPLP0_2 RVS 5′-3′ | CAATCTGCAGACAGACACTGG | 400 |
| TARDBP_2 FWD 5′-3′ | TGGACGATGGTGTGACTGCAA | 421 |
| TARDBP_2 RVS 5′-3′ | AGAGAAGAACTCCCGCAGCTCA | 422 |

In situ hybridization UNC1 3A cryptic exon analvsis in postmortem brain samples Patients and dignostic neuropathological assessment Postmortem brain tissue samples used for this study were obtained from the

University of California San Francisco (UCSF) Neurodegenerative Disease Brain Bank. Table 6 provides demographic, clinical, and neuropathological information. Consent for brain donation was obtained from subjects or their surrogate decision makers in accordance to the Declaration of Helsinki, and following a procedure approved by the UCSF Committee on Human Research. Brains were cut fresh into 1 cm thick coronal slabs and alternate slices were fixed in 10% neutral buffered formalin for 72 h. Blocks from medial frontal pole were dissected from the fixed coronal slabs, cryoprotected in graded sucrose solutions, frozen, and cut into 50 μm thick sections as described previously (45). Clinical and neuropathological diagnosis were performed as described previously (44). Subjects were selected based on clinical and neuropathological assessment. Patients selected had a primary clinical diagnosis of behavioral variant frontotemporal dementia (bv1FTD) with or without amyotrophic lateral sclerosis (ALS)/motor neuron disease (MND) and 2) a neuropathological diagnosis of frontotemporal lobar degeneration (FTLD)-TDP, Type B. We excluded subjects if they had a known disease-causing mutation, post-mortem interval>24 h, Alzheimer's disease neuropathologic change>low, Thal amyloid phase>2, Braak neurofibrillary tangle stage>4, CERAD neuritic plaque density>sparse, and Lewy body disease>brainstem predominant (45).

TABLE 6
Post-mortem brain tissue samples

| | | | | | Primary | |
|---|---|---|---|---|---|---|
| | | | | | neuro- | |
| Case | Age | | PMI | Clinical | pathological | |
| Number | (years) | Sex | (hrs) | diagnosis | diagnosis | ADNC |
| FTD- | 72 | M | 6.7 | bvFTD-MND | FTLD-TDP-B | Not |
| MND 1 | | | | | | |
| FTD- | 57 | M | 7.6 | bvFTD-ALS | FTLD-TDP-B, | Not |
| MND 2 | | | | | MND | |
| FTD- | 66 | M | 12.1 | bvFTD/nfvPPA, | FTLD-TDP-B, | Low |
| MND 3 | | | | MND | ALS | |
| FTD- | 65 | F | 8.5 | bvFTD | FTLD-TDP-B, | Low |
| MND 4 | | | | | MND | |
| Control 1 | 76 | M | 8.2 | N/A | None | Low |
| Control 2 | 67 | F | 19.4 | N/A | None | Not |
| Control 3 | 60 | F | 20.5 | N/A | None | Low |

### In Situ Hybridization (ISH) and Immunofluorescence

To detect single RNA molecules, a BaseScope Red Assay kit (ACDBIO, USA) was used. One 50 μm thick fixed frozen tissue section from each subject was used for staining. Experiments were performed under RNase free conditions as appropriate. Probes that target the transcript of interest, UNC13A, specific to either the mRNA (exon20/21 junction) or the cryptic exon containing spliced target (exon20/cryptic exon junction) were used. Positive (Homo sapiens PPI13) and negative (Escherichia coli DapB) control probes were also included. In situ hybridization was performed based on vendor specifications for the BaseScope Red Assay kit. Briefly, frozen tissue sections were washed in PBS and placed under an LED grow light (1-JTG Supply, LED-6B²40) chamber for 48 h at 4° C. to quench tissue autofluorescence. Sections were quickly rinsed in PBS and blocked for endogenous peroxidase activity. Sections were transferred on to slides and dried overnight. Slides were subjected to target retrieval and protease treatment and advanced to ISI. Probes were detected with TSA Plus-Cy3 (Akoya Biosciences) and subjected to immunofluorescence staining with antibodies to TDP-43 (rabbit polyclonal, Proteintech, RRID: AB_615042) and NeuN (Guinea pig polyclonal, Synaptic systems) and counterstained with DAPI (Life Technologies) for nuclei.

Image acquisition and analysis

Z-stack images were captured using a Leica SP8 confocal microscope with an 63× oil immersion objective (1 0.4 NA). For RNA probes, image capture settings were established during initial acquisition based on PPIB and DAPB signal and remained constant across UNC13A probes and subjects. TDP-43 and NeuN image capture settings were modified based on staining intensity differences between cases. For each case, 6 non-overlapping Z-stack images were captured across cortical layers 2-3. RNA puncta for the UNC13A cryptic exon were quantified using the “analyze particle” plugin in ImageJ. Briefly, all images were adjusted for brightness using similar parameters and converted to maximum intensity Z-projections, images were adjusted for auto-threshold (intermodes), and puncta were counted (size: 6-infinity, circularity—0-1).

Linkage Disequilibrium analysis

Recalibrated VCF files generated by GATK HlaplotypeCallers were downloaded from Answer ALS in July 2020. VCFtools (0.1.16) were used to filter for sites that are in intron 20-21. The filtered VCF files were merged using BCFtools (1.8). Since there are sites that contain more than 2 alleles, we tested for genotype independence using the chi-squared statistics by using the command “vcftools --geno-chisq --min-alleles 2--max-alleles 8” (4.0.0).

Statistical methods

Survival curves were compared using the coxph function in the survival (3 1.12) R package, which fits a multivariable Cox proportional hazards model that contains sex, reported genetic mutations and age at onset, and performs a Score (log-rank) test. Effect sizes are reported as the hazard ratios. Proportional Hazards assumptions were tested using cox.zph( ) function. The survival curves were plotted using ggsurvplot( ) in suvminer (v.0.4.8) R package.

Correlations between the cryptic exon signal and phosphorylation levels of TDP-43 or number of risk haplotypes were done after filtering out all the samples that do not have the cryptic exon signal (n=4). Linear mixed effects models were analyzed using lmerTest R package (3.1.3).

Statistical analyses were performed using R (version 4.0.0), or Prism 8 (GraphPad), which were also used to generate graphs.

### Results

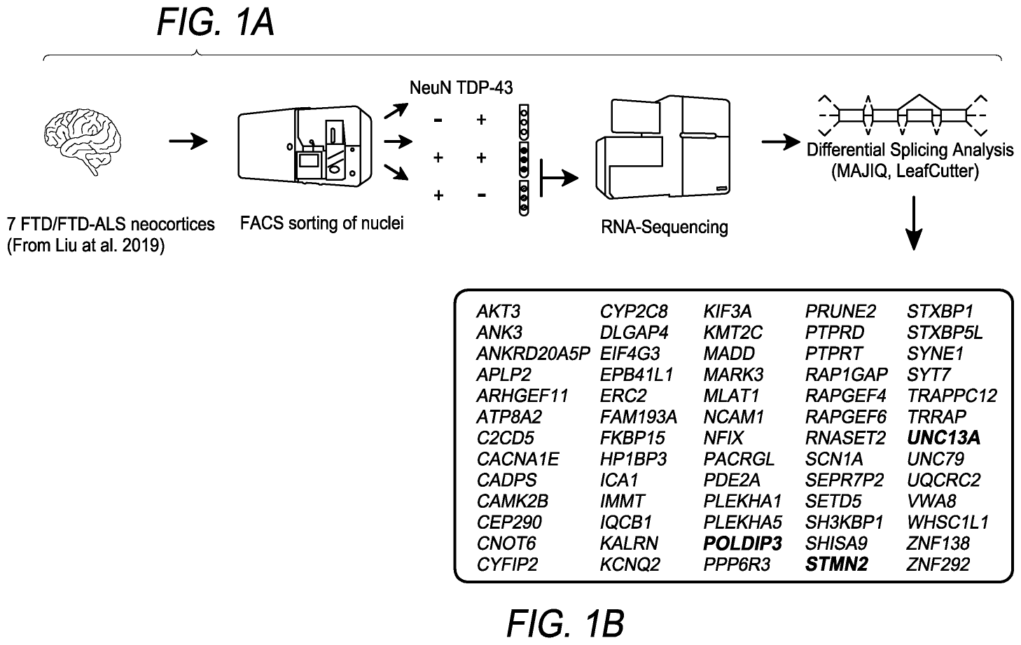

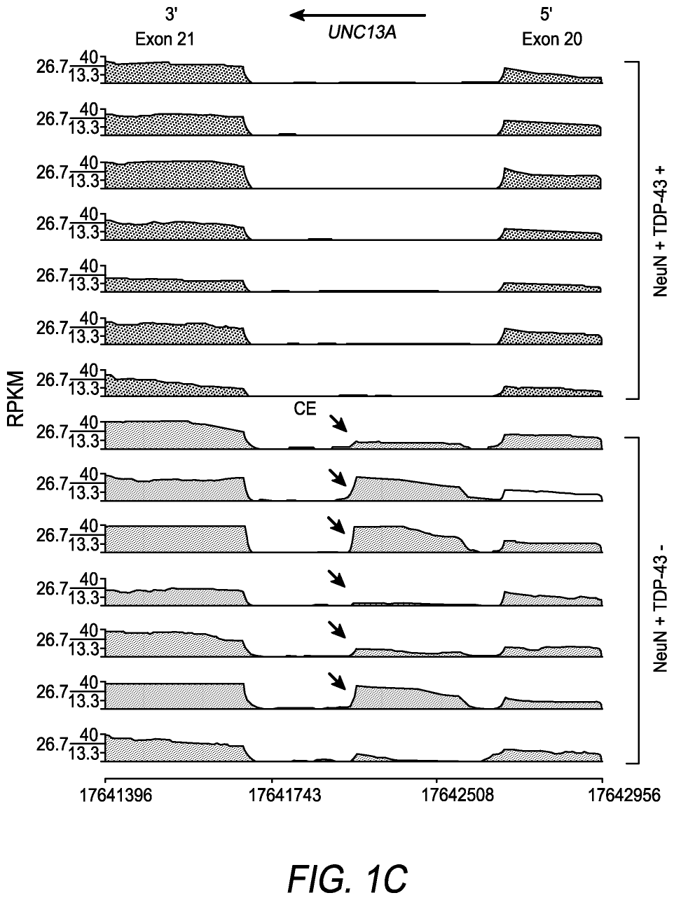

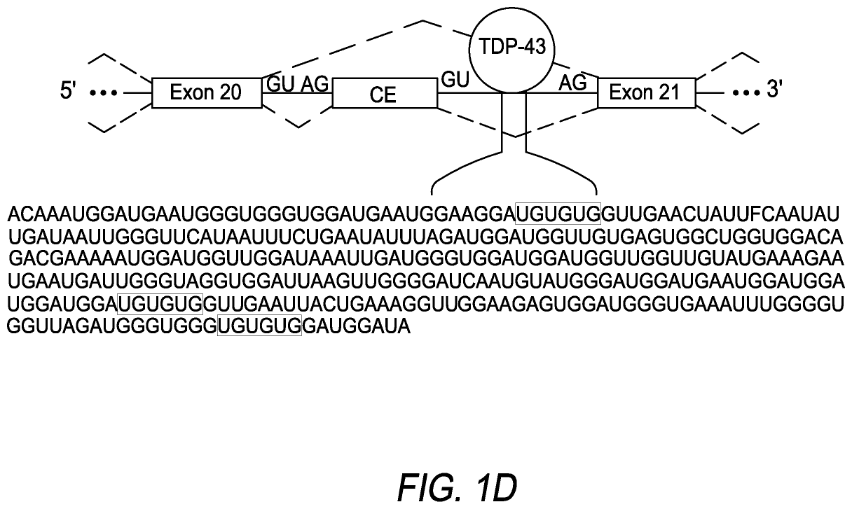

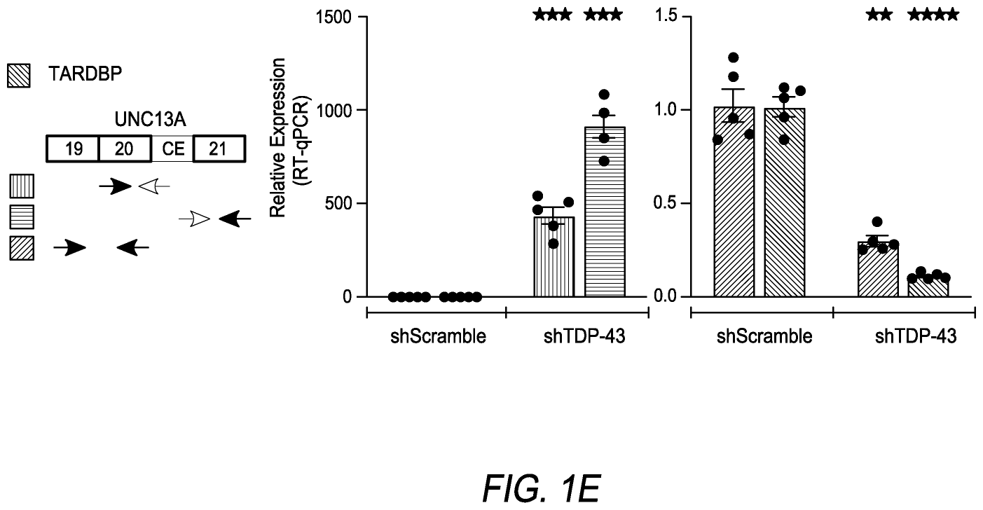

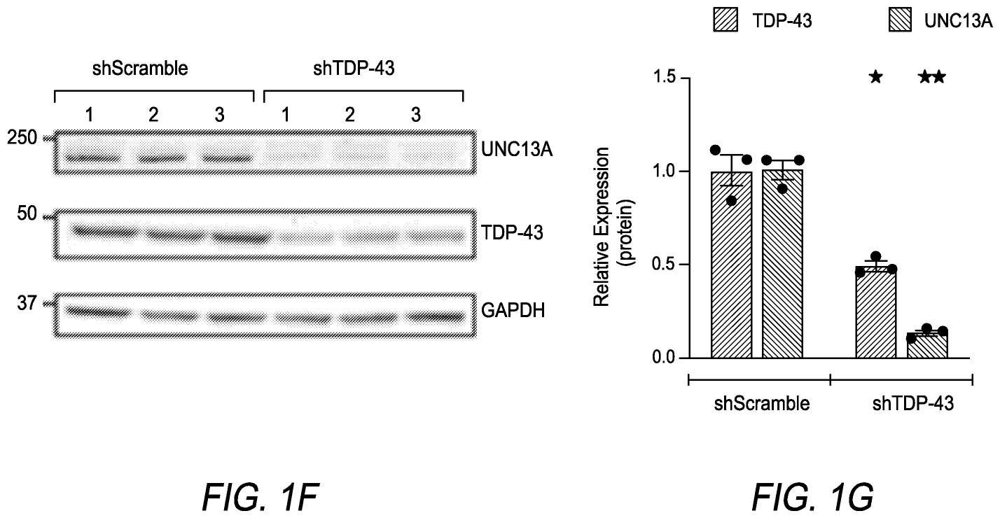

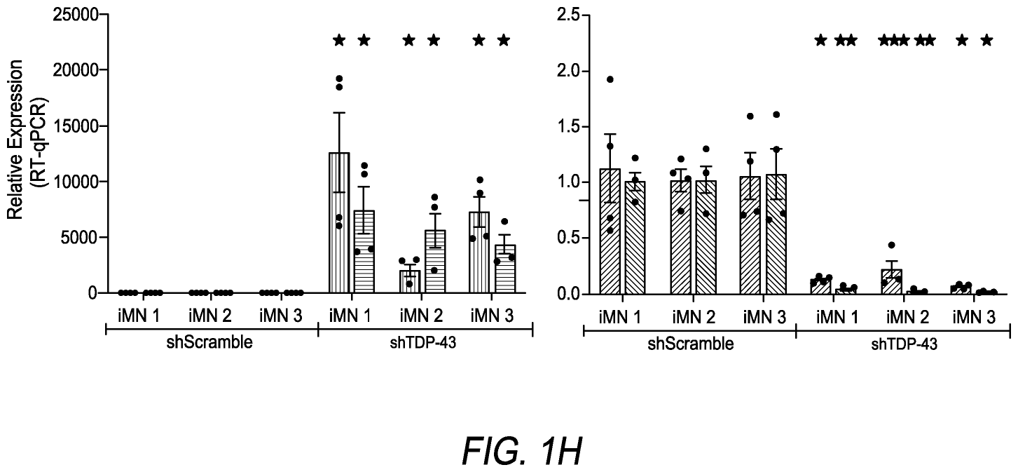

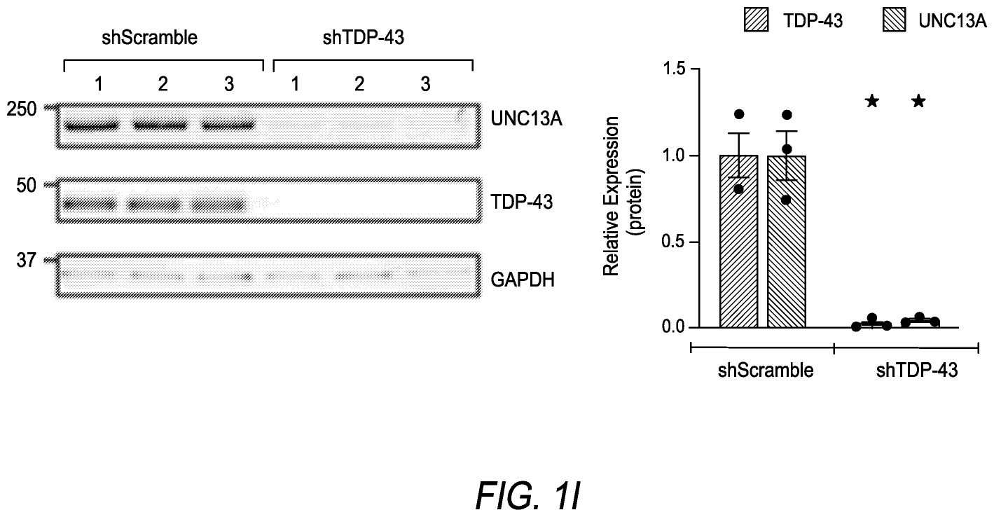

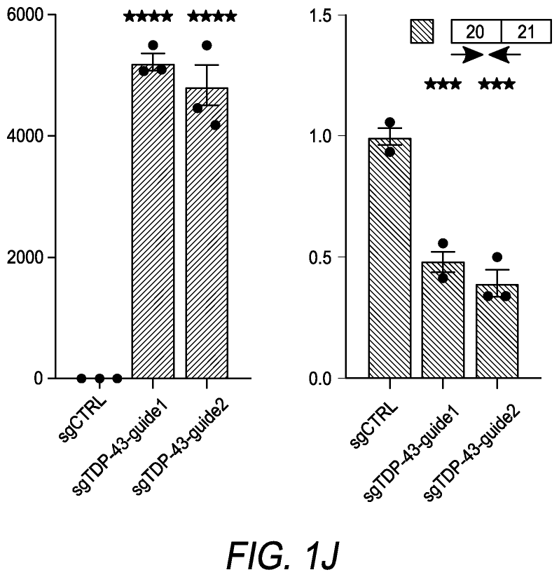

To discover cryptic splicing targets that are regulated by TDP-43 that may also play a role in disease pathogenesis, a recently generated RNA sequencing (RNA-seq) dataset was utilitzed (11). To identify changes associated with loss of TDP-43 from the nucleus, Liu et al. cleverly realized that they could use fluorescence-activated cell sorting (FACS) to enrich neuronal nuclei that either contained TDP-43 or did not and then perform RNA-seq to compare the transcriptomes between TDP-43-positive and TDP-43-negative neuronal nuclei from 7 frozen neocortices of postmortem brains from FTD/ALS patients. They identified a multitude of interesting differentially expressed genes (11). The present study re-analyzed the data in a different way—not looking for differentially expressed genes like Liu et al. did but instead searching for novel alternative splicing events impacted by the loss of TDP-43. Splicing analyses using two pipelines, MAJIQ (12) and LeafCutter (13) was performed, designed to detect novel splicing events (FIG. 1A). Each RNA-seq library contains approximately 50M paired-end reads with a length of 125 bp, greater read length and coverage facilitating discovery of splicing changes caused by the loss of TDP-43. 197 alternative splicing events (P(ΔΨ>0.1) >0.95)(ΔΨ, changes of local splicing variations between two conditions; P: probability) were identified with MAJIQ and 152 with LeafCutter (P<0.05). There were 65 alternatively spliced genes in common between both analyses (FIG. 1B), likely because each tool uses different definitions for transcript variations and different criteria to control for false positives. Notably, among the alternatively spliced genes identified by both tools were STMN2 and POLDIP3, both of which have been extensively validated as bonafide TDP-43 splicing targets (8-10, 14).

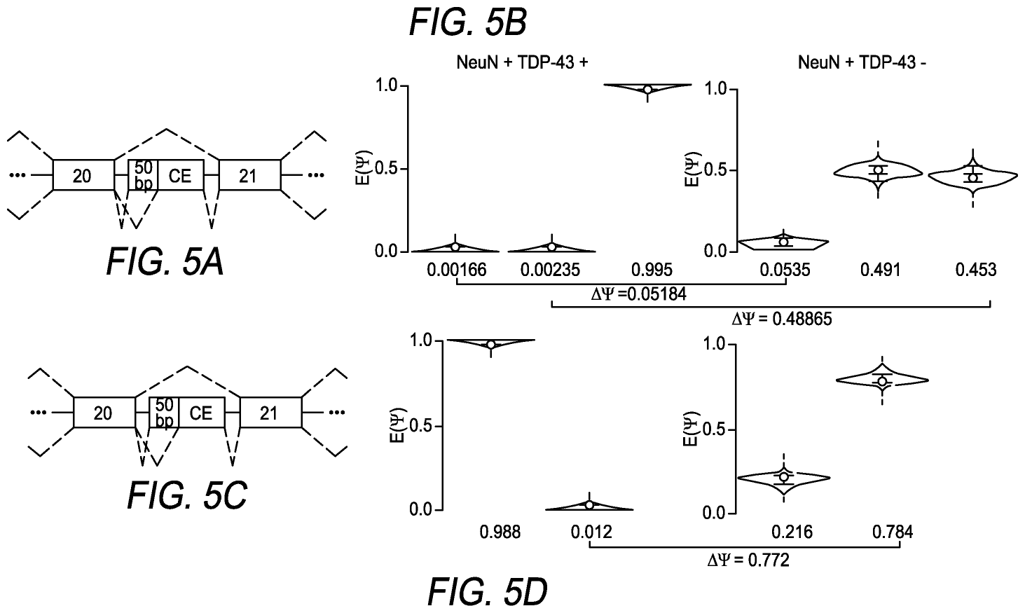

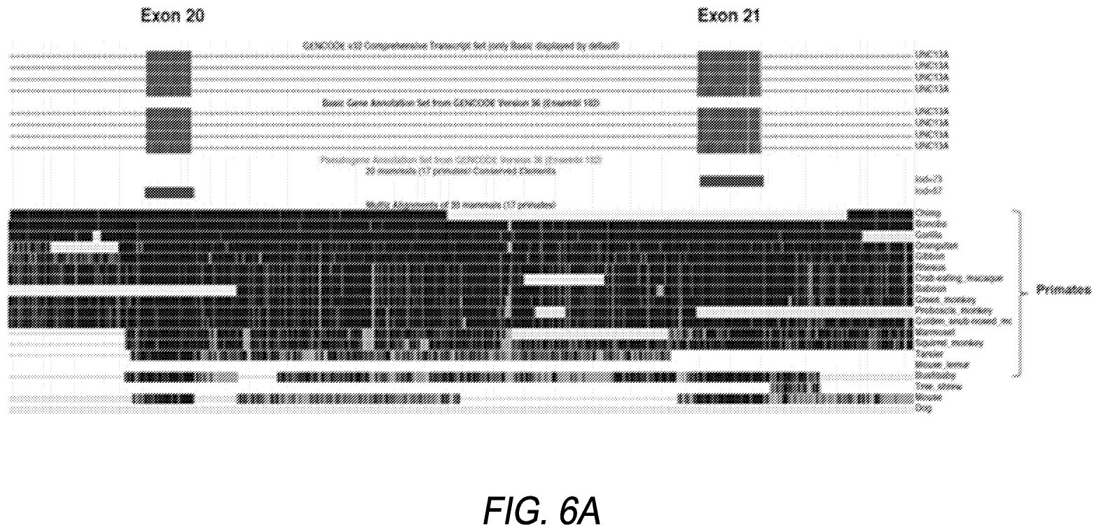

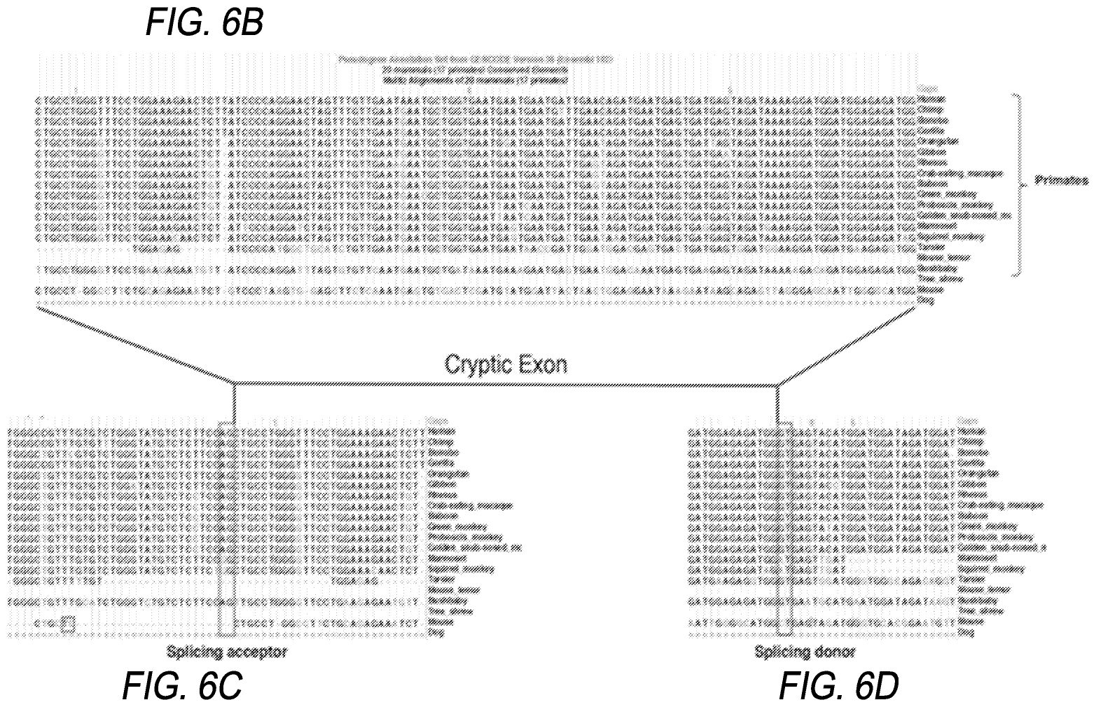

Unexpectedly, UNC13A was found to be one of the most significantly alternatively spliced genes in neurons with TDP-43 depleted from the nucleus (FIG. 1B and FIGS. 5A-5D). Depletion of TDP-43 resulted in the inclusion of a 128 bp cryptic exon #1 between the canonical exons 20 and 21 (hg38; chrl9: 17642414-17642541) (FIGS. 1C and 1D) or a ###bp cryptic exon #2 between exons 20 and 21 (hg38; chrl9: 17642414-1764 2591). Since higher usage of the chr19:1764254i 1 3′ splicing acceptor was observed, the focus of the study is on the 128 bp cryptic exon #1. Hereinafter, in this example, if not specified, reference to cryptic exon refers to the 128 bp cryptic exon #1. This new exon, referred to as CE #1 (for cryptic exon), was absent in wild type neuronal nuclei (FIG. 1C) and is not present in any of the known human isoforms of UNC13A (15). Furthermore, analysis of ultraviolet cross-linking and immunoprecipitation (iCLIP) data for TDP-43 in SH-SY5Y cells (3) provides evidence that TDP-43 directly binds to the intron harboring this cryptic exon (FIG. 1D). Insertion of the 128 bp cryptic exon sequence into the mature transcript was confirmed by direct sequencing. Intron 20-21 of UNC13A and the CE sequence are conserved among most primates (FIGS. 6A and 6B) but not conserved in mouse, similar to STWN2 and other cryptic splicing targets of TDP-43 (4, 8, 9). Together, these results suggest that TDP-43 functions to repress the inclusion of a cryptic exon in the UNC13A mRNA transcript.

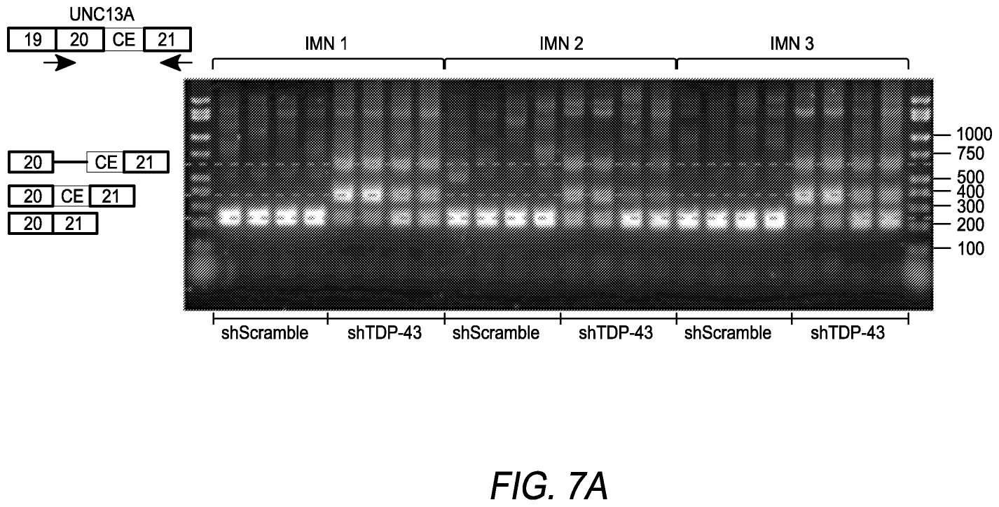

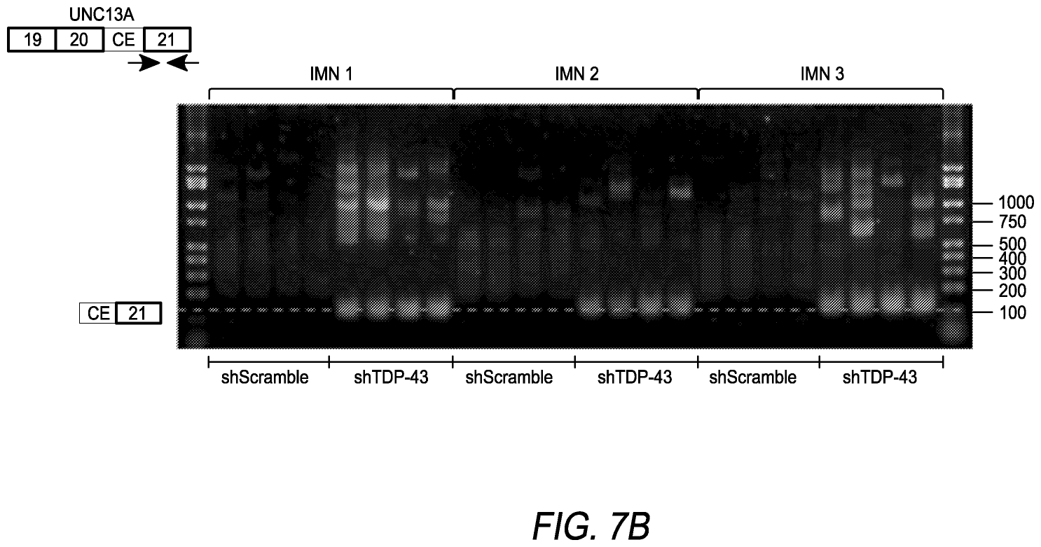

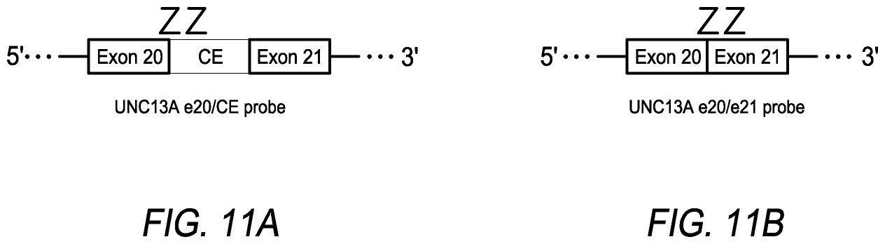

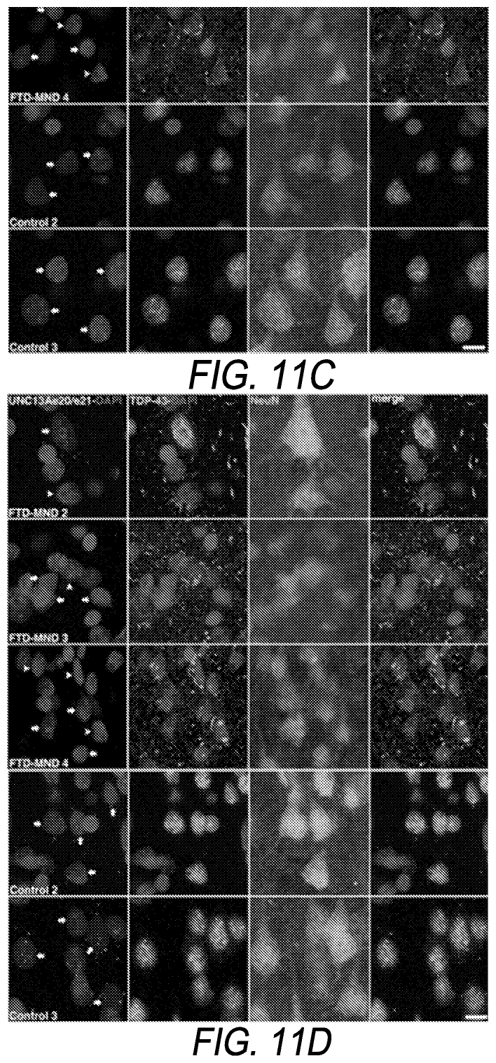

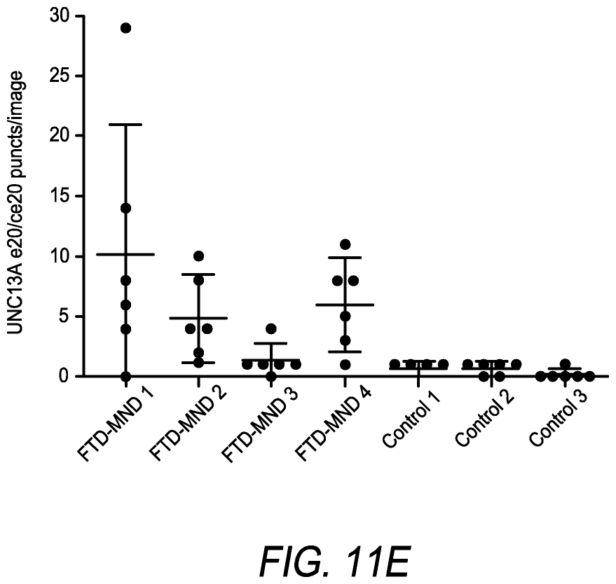

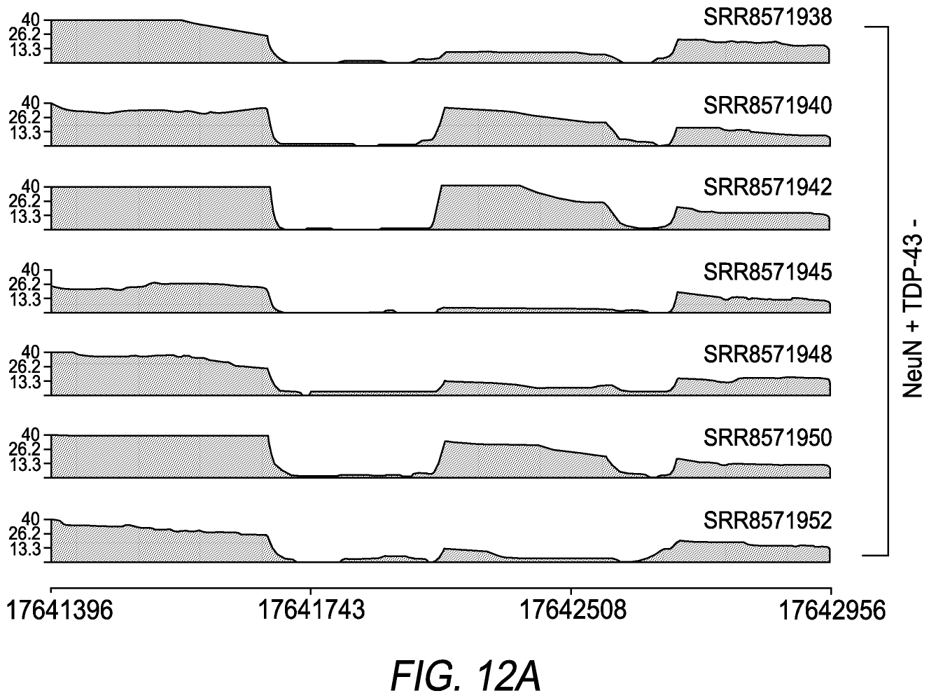

To test if TDP-43 directly regulates this UNC13A cryptic splicing event, doxycycline-inducible shRNA was used to reduce TDP-43 levels in SH-SY5Y cells. Quantitative reverse transcription PCR (qRT-PCR) was used to detect cryptic exon inclusion, which was present in cells with TDP-43 depleted (by treatment with shTARDBP) but not in control shRNA treated cells (FIG. 1E). Along with the increase in cryptic exon levels, there was a corresponding decrease in levels of the canonical UNC13A transcript upon TDP-43 depletion (FIG. 1E). By immunoblotting, a marked reduction in UNC13A protein in TDP-43-depleted cells was also observed (FIGS. 1F, 1G). TDP-43 levels were reduced in induced motor neurons (iMNs) (FIGS. 111, 11; FIGS. 7A and 7B) and excitatory neurons (i³Ns) derived from human iPS cells (FIG. 1J). TDP-43 depletion resulted in cryptic exon inclusion in UNC13A and a reduction in UNC13A mRNA and protein. Thus, lowering levels of TDP-43 in human cells and neurons causes inclusion of a cryptic exon in the UNC13A transcript, resulting in decreased UNC13A protein.

UNC13A belongs to a family of genes originally discovered in C. elegans based on the uncoordinated (unc) movements exhibited by animals with mutations in these genes (16), owing to deficits in neurotransmitter release. UNC13A encodes a large multidomain protein expressed in the nervous system, where it localizes to neuromuscular junctions and plays an essential role in the vesicle priming step, prior to synaptic vesicle fusion (17-20). In vitro studies demonstrate that the cryptic exon splicing event upon TDP-43 depletion causes marked reduction in UNC13A expression (FIG. 1F). Mice lacking Uncl3a (also called Muncl3-1) show morphological defects in spinal cord motor neurons and functional deficits at the neuromuscular junction. These data suggest that depletion of TDP-43 leads to a loss of UNC13A function (21).

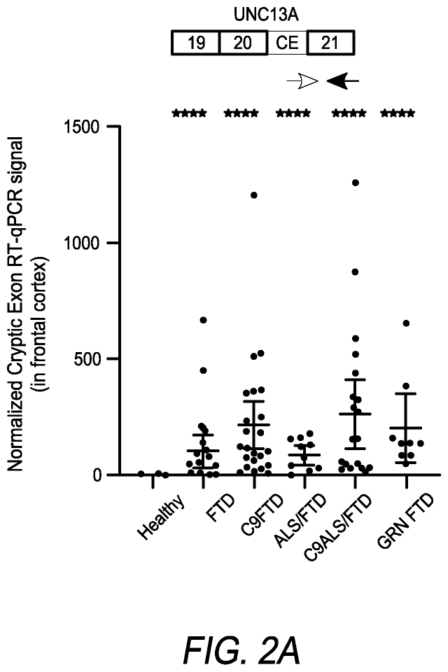

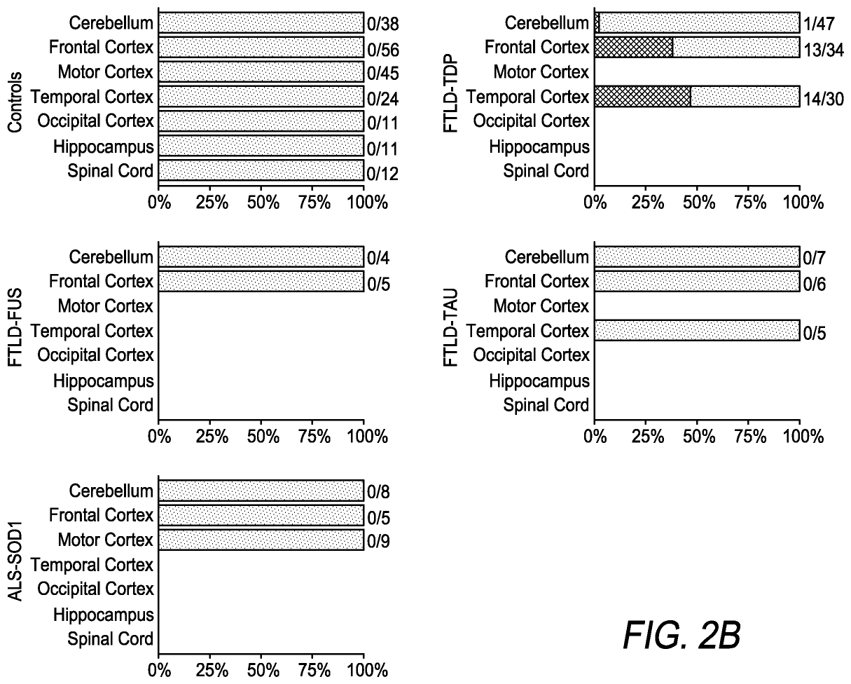

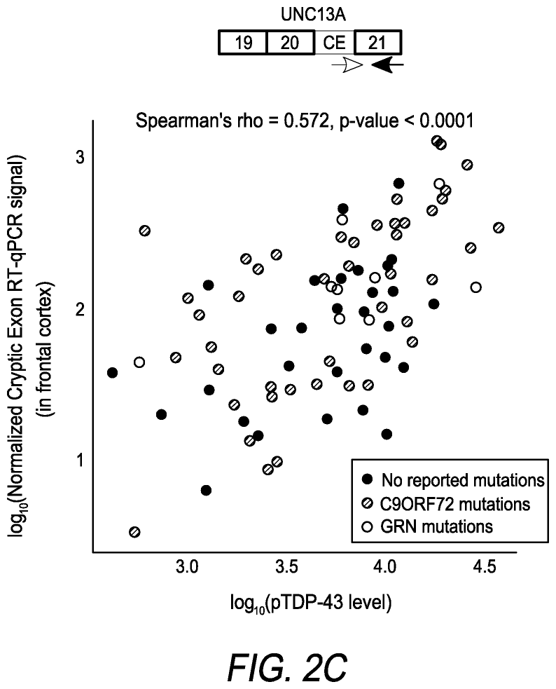

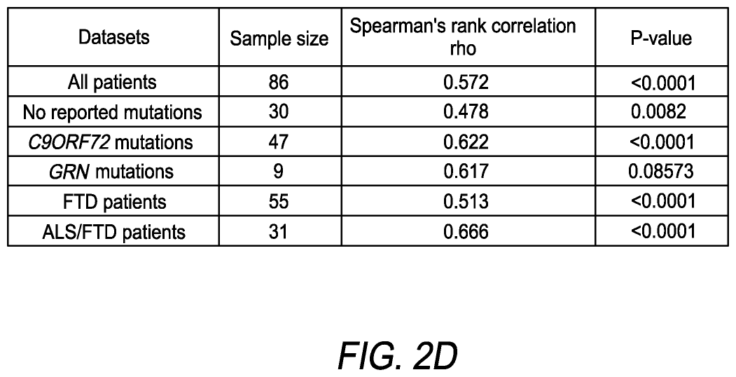

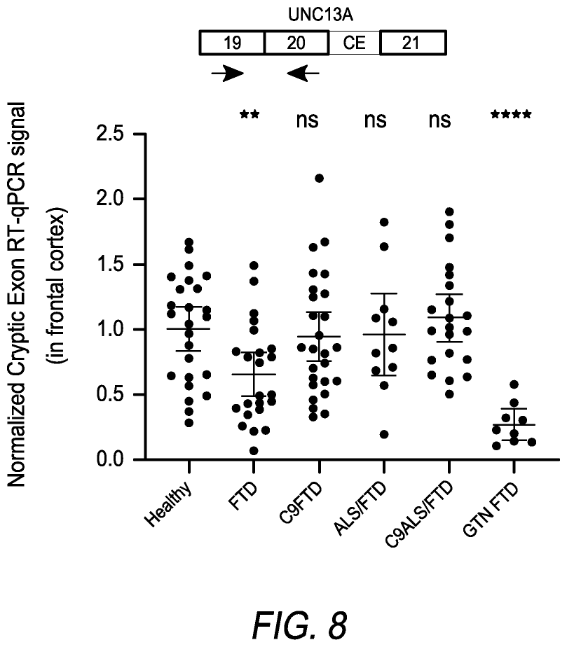

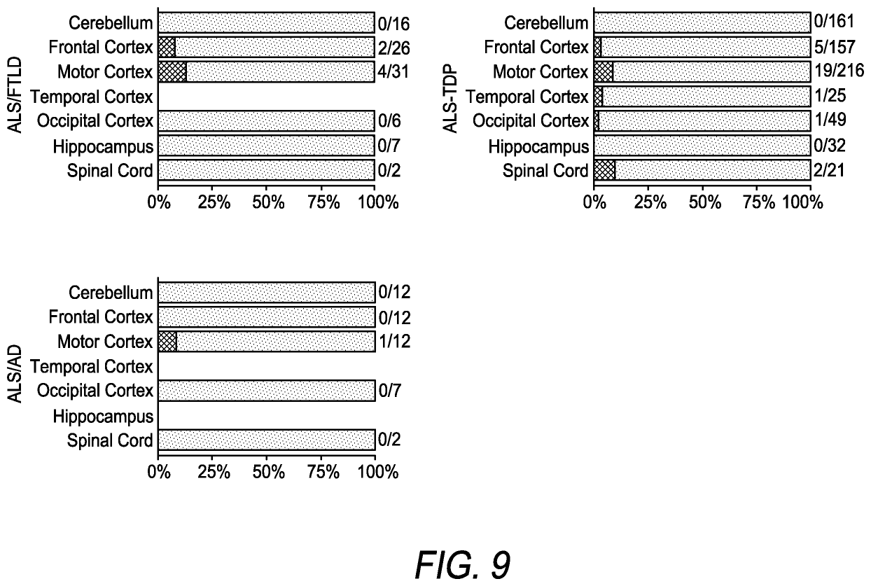

To extend this analysis of UNC13A cryptic exon inclusion to a larger collection of patient samples, a series of 115 frontal cortex brain samples from the Mayo Clinic brain bank were first analyzed and a significant increase in UNC13A cryptic exon (CE) levels was found in FTLD-TDP patients compared to healthy controls (FIG. 2A). A decrease in total UNC13A transcripts in frontal cortex of some subtypes of FTD patients was also observed (FIG. 8). Next, brain samples from the New York Genome Center (NYGC) were analyzed. After filtering for relatively high-quality data (Methods), this data set includes RNA-seq data from 1151 samples from 413 individuals (more than one tissue per individual), 330 of which are ALS or FTD patients. Because FACS analysis by Liu et al. (11) indicates that pathological neuronal nuclei with loss of TDP-43 represent only ˜7% of all neuronal nuclei and less than 2% of all cortical cells (11) it was expected that splicing analysis algorithms would struggle to detect differentially spliced genes in RNA-seq data generated from bulk RNA sequencing. To overcome this problem, reads that spanned the exon 20-CE and CE-exon 21 junctions were specifically looked for. Owing to noise generated from bulk sequencing, the UNC13A splice variant was scored as present if there were more than two reads spanning at least one of the exon-exon junctions. 63 samples, from 49 patients, were identified which met the above criteria. Notably, UNC13A splice variant was detected in close to 50% of the frontal cortical and temporal cortical tissues donated by neuropathologically confirmed FTLD-TDP patients. The splice variants were also detected in some of the ALS patients whose pathology has not been confirmed (FIG. 9). Notably, UNC13A CE was not observed in any of the samples from FTLD-FUS (n=9), FTLD-TAU (n=18) and ALS-SOD1 (n=22) patients, nor in any of the control samples (n=197). Thus, UNC13A cryptic exon inclusion is a robust and specific facet of pathology in TDP-43 proteinopathies (FIG. 2B).

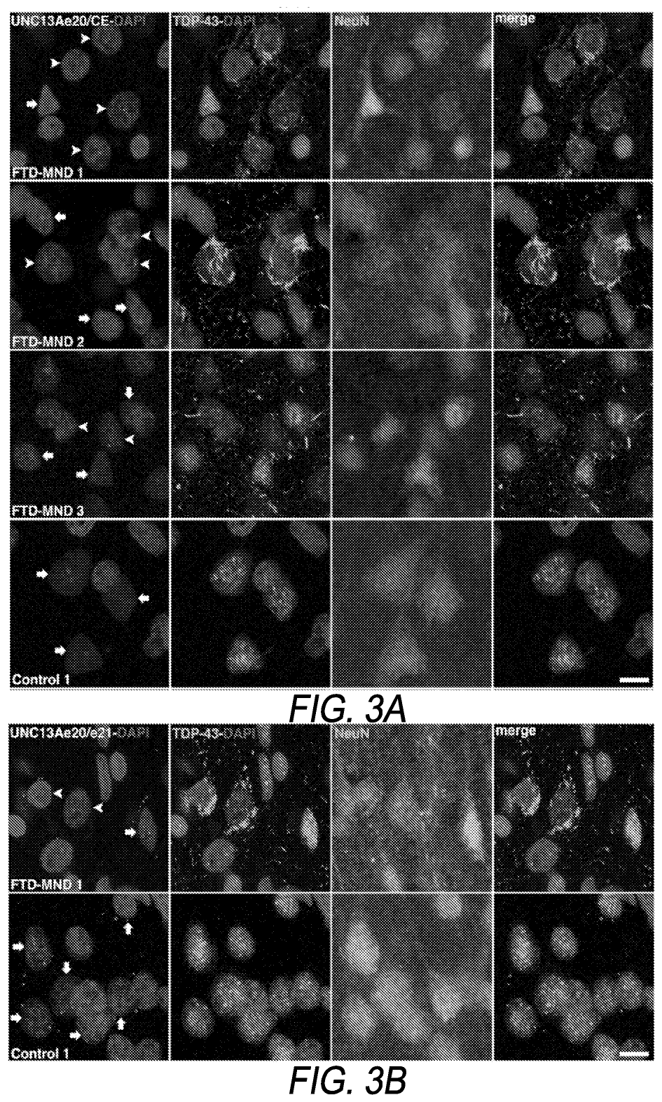

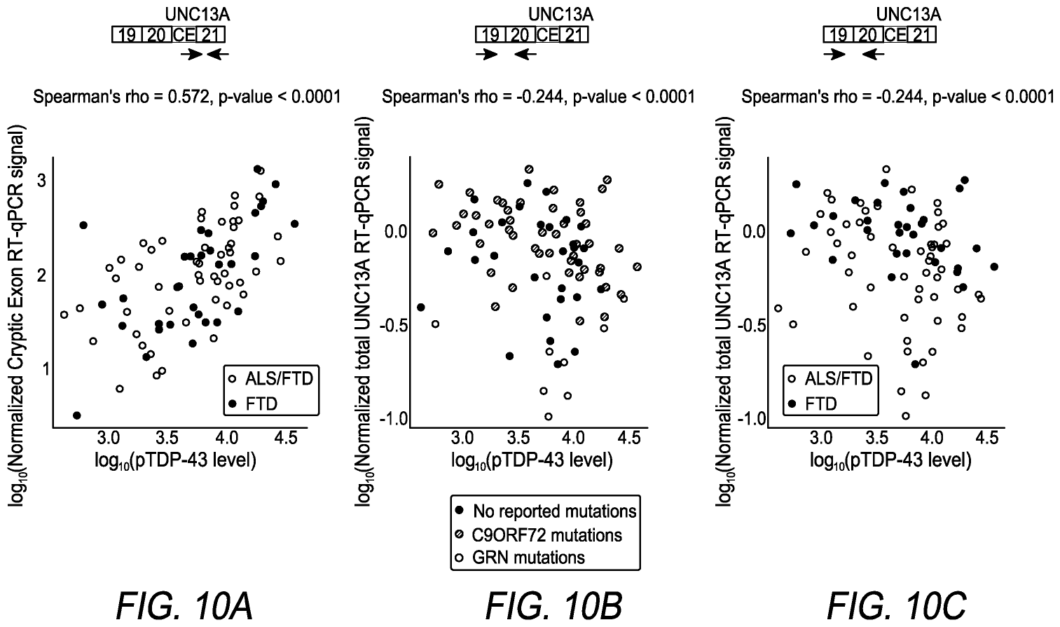

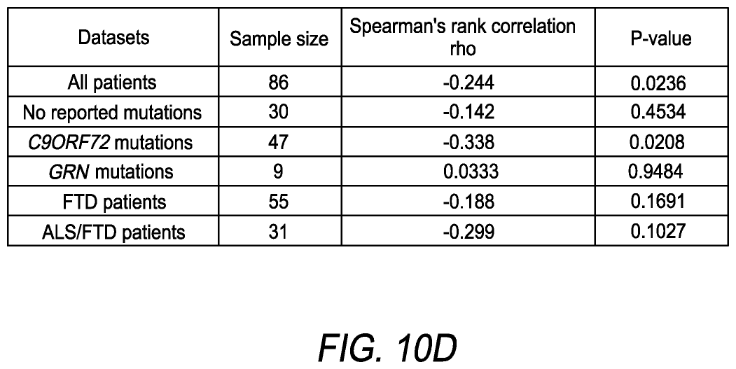

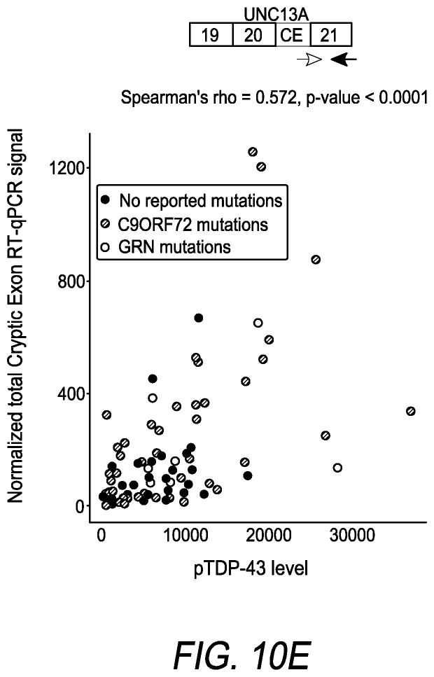

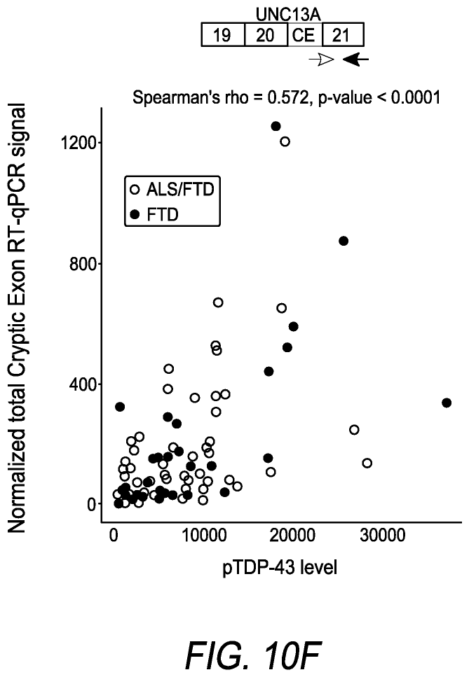

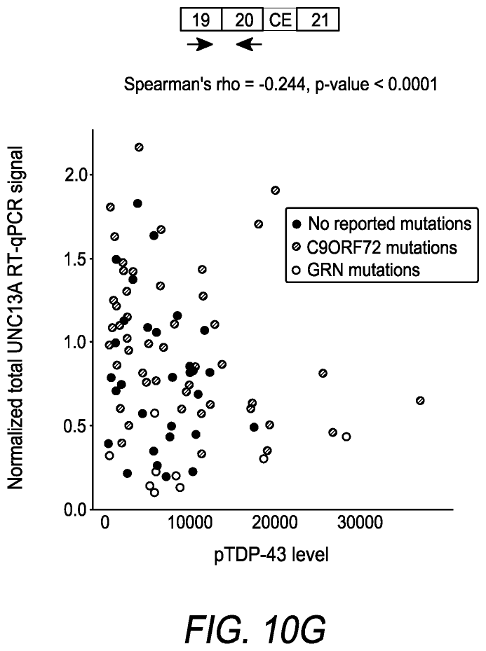

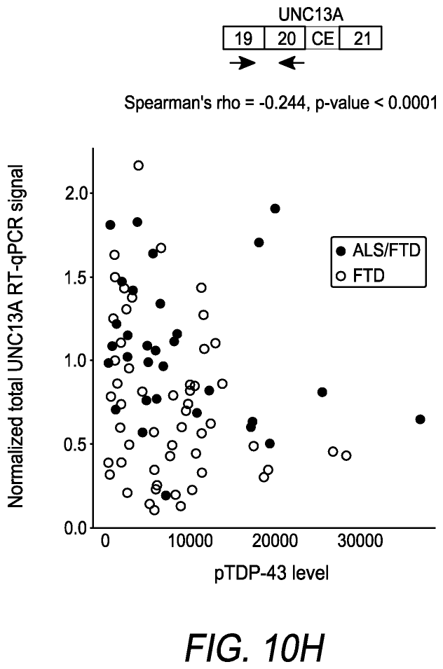

Once TDP-43 becomes depleted from the nucleus and accumulates in the cytoplasm, it becomes phosphorylated. Hyperphosphorylated TDP-43 (pTDP-43) is a key feature of pathology (22). To determine the relationship between pTDP-43 levels and UNC13A cryptic exon inclusion, a set of 86 FTD patients from the Mayo Clinic brain bank, for which RNA-seq and pTDP-43 levels from frontal cortices was obtained, was analyzed. A striking association between higher pTDP-43 levels and higher levels of UNC13A cryptic exon inclusion was found in patients from all disease subtypes (Spearman's rho=0.564, P<0.0001) (FIGS. 3C and 3D, and FIG. 10A; figures using untransformed data: FIGS. 10E and 10F). The levels of total UNC13A transcripts were also negatively correlatedly with pTDP-43 levels (FIGS. 10B, 10C, 10G and 10H). Thus, UNC13A cryptic exon inclusion and decreased full-length transcript level seem to be a common feature of multiple TDP-43 proteinopathies and to strongly correlate with the burden of TDP-43 pathology.

To visualize the UNC13A CE at single cell sensitivity with spatial resolution, custom BaseScope™ in situ hybridization probes were designed that specifically bind to the exon 20-exon 21 (FIG. 11A) or the exon 21-CE junction (FIG. 11B). The probes were designed to span exon-exon junctions in order to minimize the possibility of binding to pre-mRNA. These probes were used for in situ hybridization along with immunofluorescence for NeuN (to detect neurons) and TDP-43 (to detect nuclear or cytoplasmic TDP-43). Sections from the medial frontal pole of 4 FTLD-TDP patients and 3 controls were stained. Using the exon21-CE probe robust UNC13A CE inclusion was detected in nearly every neuron with TDP-43 depleted from the nucleus but not in ones with nuclear TDP-43 (FIG. 3A, FIGS. 11C and 11E). UNC13A mRNA was detected using the exon20/21 probe in neurons of both cases and controls (FIG. 3B, FIG. 11D). UNC13A cryptic exon inclusion now seems to be a robust facet of FTLD-TDP pathology.

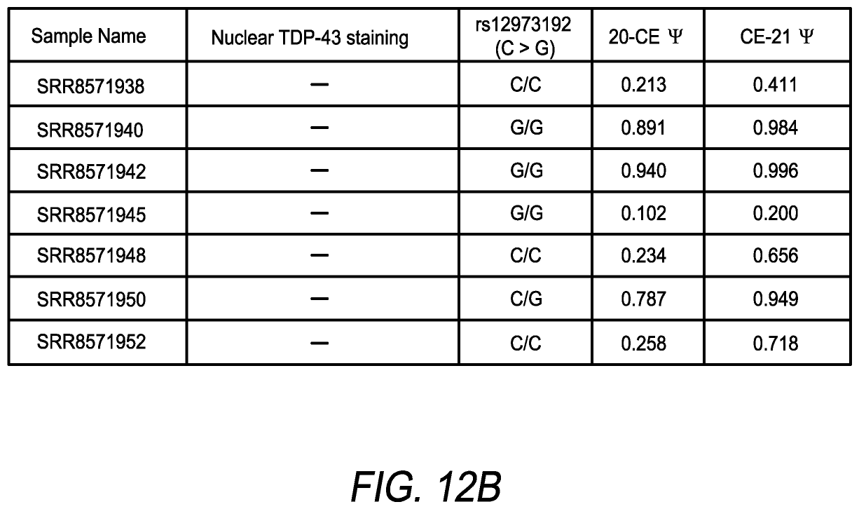

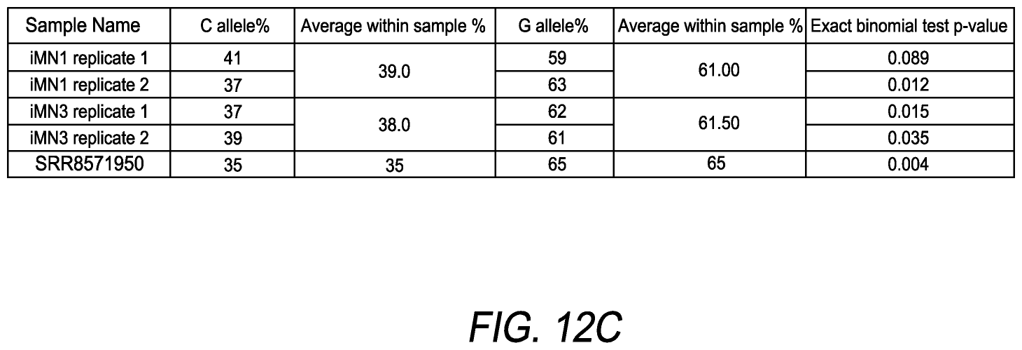

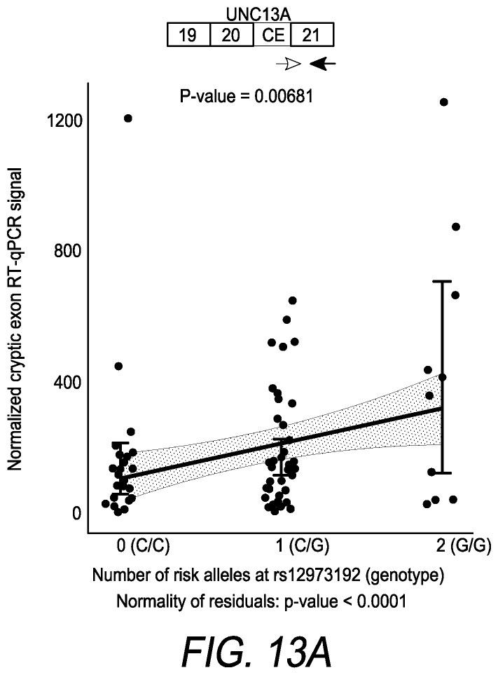

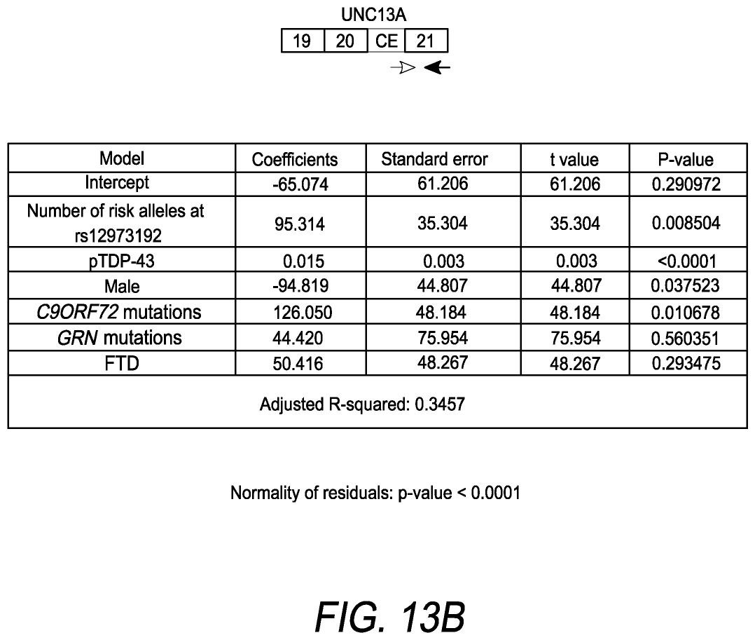

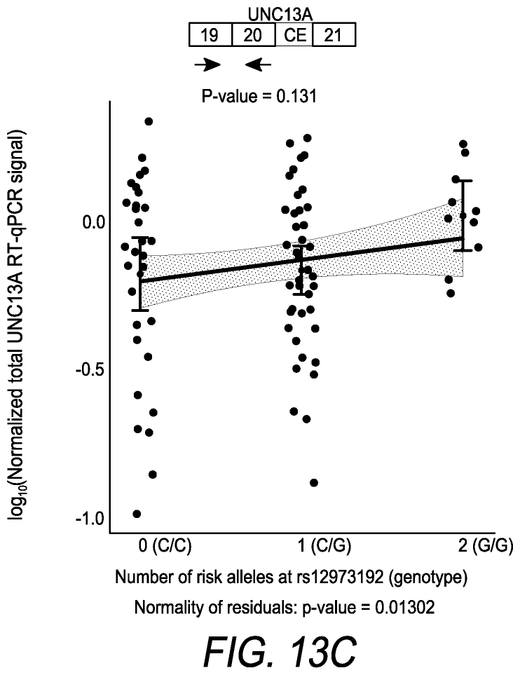

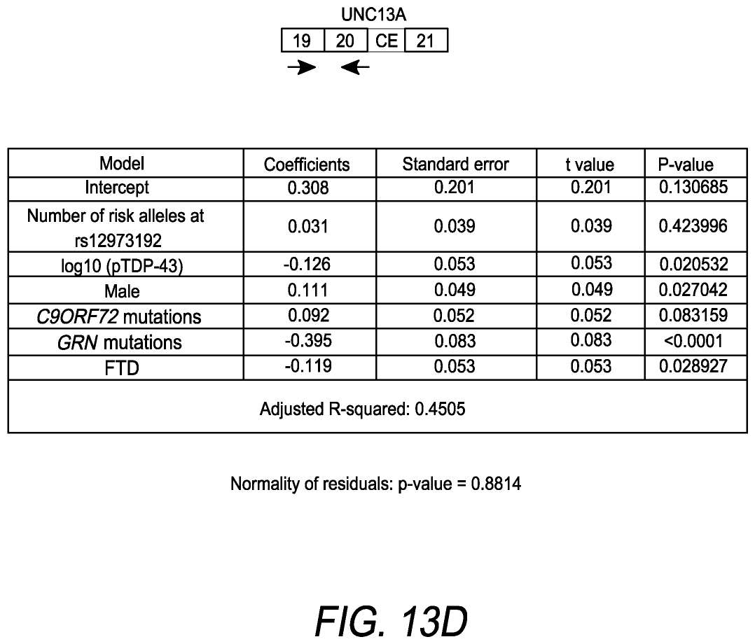

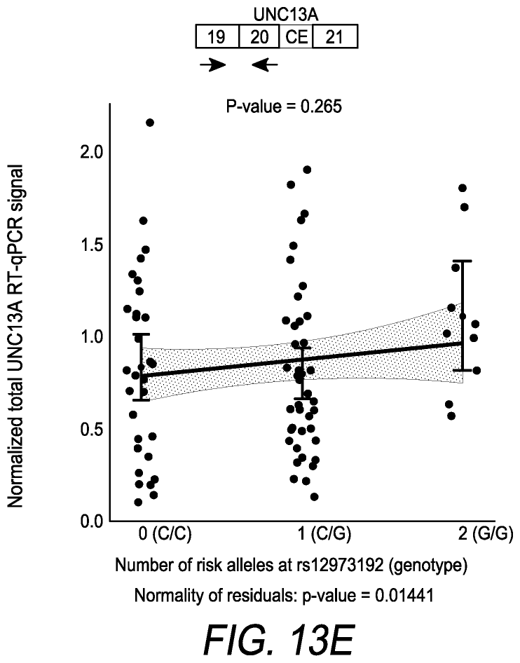

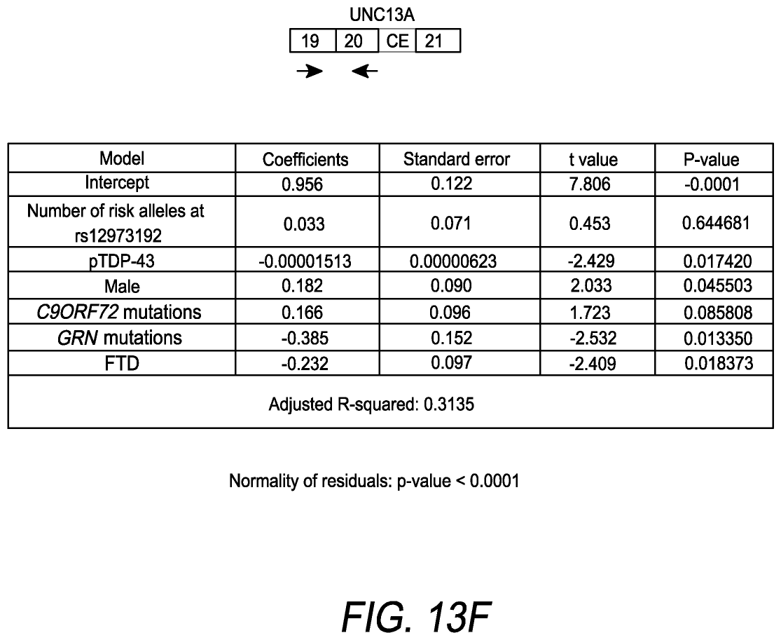

UNC13A is one of the top GWAS hits for ALS and FTD-ALS, replicated across multiple studies (23-28). SNPs in UNC13A are associated with increased risk of sporadic ALS (24) and sporadic FTD with TDP-43 pathology (23). In addition to increasing susceptibility to ALS, SNPs in UNC13A are also associated with shorter survival in ALS patients (29-32). But the mechanism by which genetic variation in UNC13A increase risk for ALS and FTD is unknown. Remarkably, the two most significantly associated SNPs, rs12608932 (A>C) and rs12973192 (C>G), are both located in the same intron that we found harbors the cryptic exon, with rs12973192 located right in the cryptic exon itself (FIG. 4A). This immediately suggested the hypothesis that these SNPs (or other genetic variation nearby tagged by these SNPs) might make UNC13A more vulnerable to cryptic exon inclusion upon TDP-43 depletion. To test this hypothesis, the percentage of RNA-seq reads (FIGS. 12A and 12B) that span intron 20-21 that support the inclusion of the cryptic exon was analyzed. Among the 7 RNA-Seq libraries from TDP-43 depleted neuronal nuclei that were included in the initial splicing analysis, 2 out of 3 patients that were homozygous (G/G) and the one patient that was heterozygous (C/G) for the risk allele at rs12973192 showed inclusion of the cryptic exon in almost every UNC13A mRNA that was mapped to intron 20-21. In contrast, the patients who were homozygous for the reference allele (C/C) showed much less inclusion of the cryptic exon. Another way to directly assess the impact of the UNC13A risk alleles on cryptic exon inclusion is to measure potential allele imbalance in RNAs from individuals who happen to be heterozygous for the risk allele. In other words, is there an equal number of RNAs with cryptic exon inclusion produced from the risk allele as the protective allele? Or are there more from the risk allele? Two of the iMN lines that were used to detect cryptic exon inclusion upon TDP-43 knockdown (FIG. 1G, iMN1 and iMN2) are heterozygous (C/G) at rs12973192. The RT-PCR product that spans the cryptic exon was sequenced and the allele distribution from these two samples was analyzed as well as the one patient sample from the original RNA-seq dataset (FIG. 1A) that is heterozygous (C/G) at rs12973192 (FIG. 12B). A significant difference between the percentage of C and G alleles was found in the spliced variant, with higher inclusion of the risk allele (p-value=0.01, two-tailed paired t-test; FIG. 4B and FIG. 12C). Given this evidence for an effect of the risk allele on cryptic exon inclusion, analysis was extended by genotyping FTD-TDP patients (n=86) in the Mayo Clinic brain bank dataset for the UNC13A risk alleles at rs12973192 and rs12608932. One patient who is homozygous for the reference allele (C/C) at the rs12973192 but heterozygous (A/C) at rs12608932 was excluded. The rest of the patients (n=85) have exactly the same number of risk alleles at both loci. The correlation between the level of cryptic exon inclusion (from RNA-seq of frontal cortex) and the number of risk alleles at rs12973192 was first modeled as a simple linear regression—a strong correlation (P=0.0136) between the number of risk alleles and the abundance of UNC13A cryptic exon inclusion was found (FIG. 4C). After including other known variables such as TDP-43 phosphorylation levels, sex, genetic mutations and disease types as predictors of the abundance of UNC13A cryptic exon in a multiple linear regression model (adjusted R²=0.3616, figure and statistics from untransformed data FIG. 13A), it was found that the number of risk alleles is one of the strongest predictors of cryptic exon inclusion (p-value=0.00792, figure from untransformed data FIG. 13B), but not of overall UNC13A expression level (FIGS. 13C and 13D, untransformed data FIGS. 13E and 13F). Taken together, these data suggest that genetic variation in UNC13A that increases risk for ALS and FTD in humans promote cryptic exon inclusion upon TDP-43 nuclear depletion.

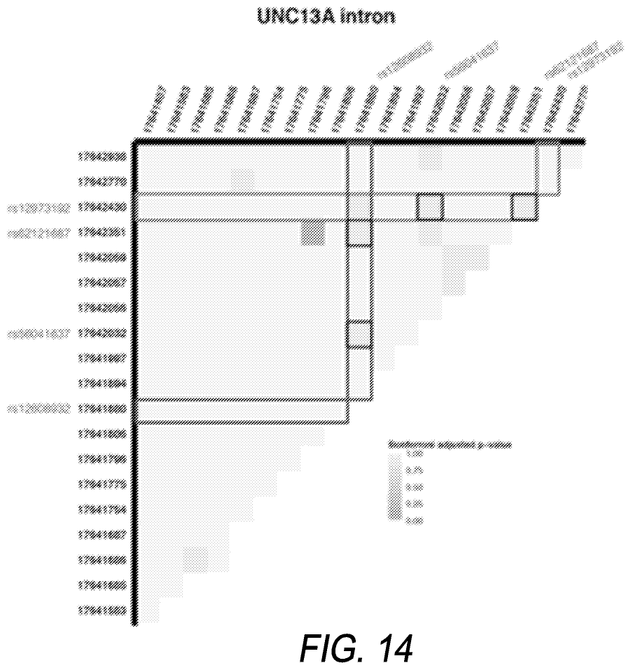

GWAS SNPs typically do not cause the trait but rather “tag” other neighboring genetic variation (33). Thus, a major challenge in human genetics is to go from GWAS hit to identifying the causative genetic variation that increases risk for disease (34). A LocusZoom (35) plot (FIG. 4A) generated using a linear mixed model analysis of ALS GWAS results (36) suggests that the strongest association signal on UNC13A is indeed in the region surrounding the two lead SNPs (rs12973192 and rs12608932). To look for other genetic variants in intron 20-21 that might also cause risk for disease by influencing cryptic exon inclusion but were not included in the original GWASs, genetic variants identified in whole genome sequencing data of ALS patients (Answer ALS) were analyzed. This dataset includes 297 ALS patients of European descent. Novel genetic variants that could be tagged by the two SNPs were searched for by looking for other loci in intron 20-21 that are in linkage disequilibrium with both rs12608932 and rs12973192. One was found that fit these criteria—rs56041637 (FDR-corrected P-value <0.0001 with rs12608932, P-value <0.0001 with rs12973192) (FIG. 14). rs56041637 is a CATC-repeat insertion. In the patient dataset, it was observed that patients who are homozygous for the risk alleles at both rs12608932 and rs12973192 tend to have 3 to 5 CATC-repeats at rs56041637; patients who are homozygous for reference alleles at both rs12608932 and rs12973192 tend to have shorter (0 to 2) repeats at rs56041637. Thus, in addition to the two lead GWAS SNPs (rs12608932 and rs12973192), now another one, rs56041637, is nominated as potentially contributing to risk for disease by making UNC13A more vulnerable to cryptic exon inclusion when TDP-43 is depleted from the nucleus.

To directly test if these three variants in UNC13A, which are part of the FTD/ALS risk haplotype, increase cryptic exon inclusion upon TDP-43 depletion, we synthesized minigene reporter constructs, containing either the risk haplotype or the protective haplotype (FIG. 4F). The reporter uses a bidirectional reporter to co-express full-length EGFP and an mCherry construct interrupted by UNC13A intron 20-21 with either the reference sequence (control) or the ALS/FTD risk alleles at rs12608932 (C), rs56041637 ((CATC)₄) and rs12973192 (G). WT and TDP-43-deficient HEK-293T cells (37), which do not express UNC13A endogenously, were transfected with each minigene reporter construct. Using RT-PCR, both versions of intron 20-21 were found to be efficiently spliced out in WT cells (FIG. 4G, lane 1-4). However, in TDP43−/− cells there was a decrease in splicing products that completely excise intron 20-21. Instead, splicing products that contain the cryptic exon, the longer variant of the cryptic exon (cryptic exon #2) (FIG. 5A) or both CE and intron 20-CE (FIG. 4G, lane 5-6). Strikingly, in TDP-43−/− cells transfected with the minigene construct harboring the risk haplotype in the intron, there was an even greater decrease in complete intron 20-21 splicing, and a concomitant increase in cryptic splicing products (FIG. 4G, lane 7-8). The expression of the splicing reporter and the efficiency of the splicing machinery independent of TDP-43 is shown by the expression level of EGFP, which is not TDP-43-dependent. A different minigene reporter construct, this one with the UNC13A intron embedded in the context of the CFTR gene, was also tested. Knockdown of TDP-43 in HeLa cells transfected with this construct resulted in mis-splicing defects. Demonstrating a direct role of TDP-43 in regulating this splicing event, expressing WT TDP-43 (but not an RNA-binding deficient mutant version with five phenylalanine residues mutated to leucine (5FL)) rescued mis-splicing (FIG. 411). Together, these two assays provide direct functional evidence that 1) TDP-43 regulates splicing of UNC13A intron 20-21 and 2) genetic variants associated with ALS and FTD susceptibility potentiate cryptic exon inclusion when TDP-43 is dysfunctional.

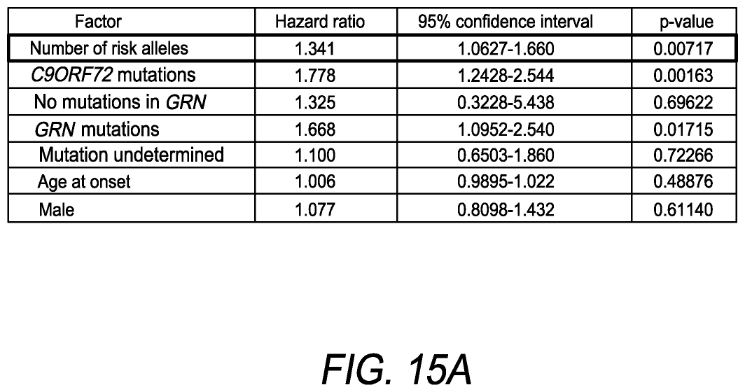

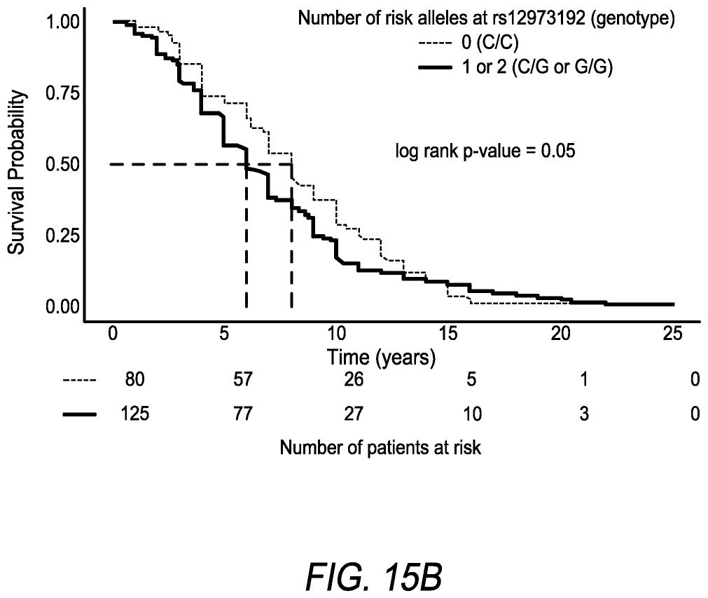

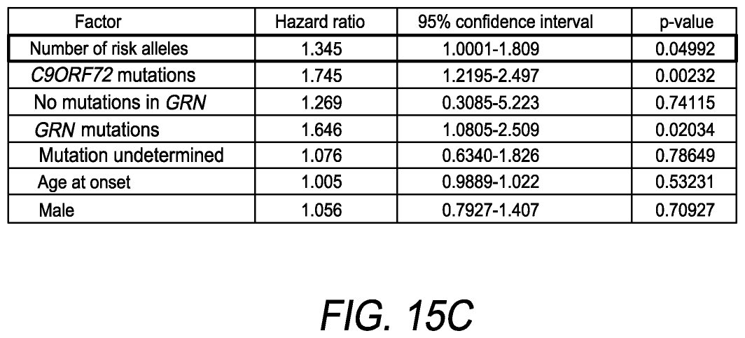

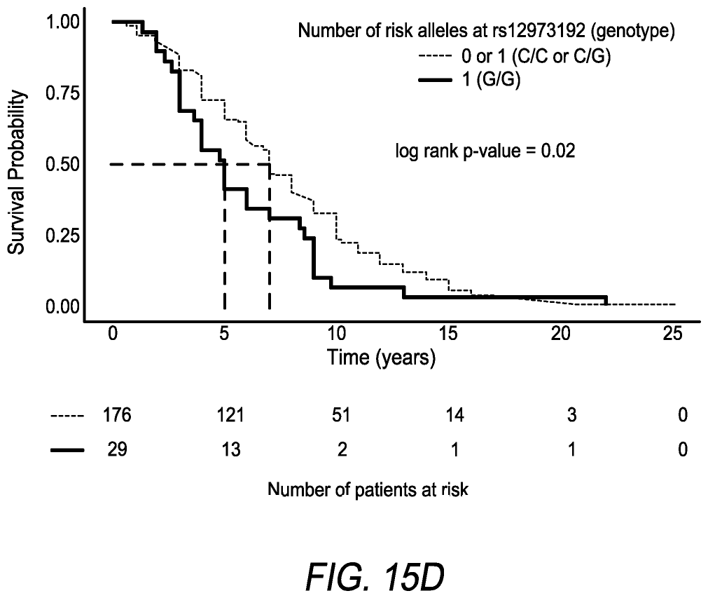

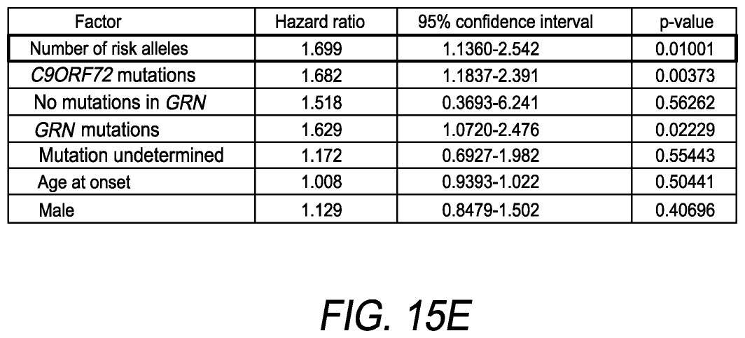

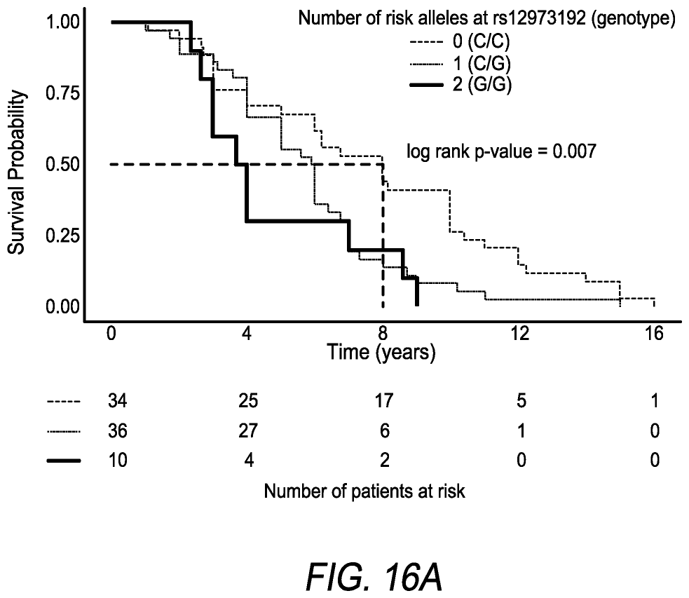

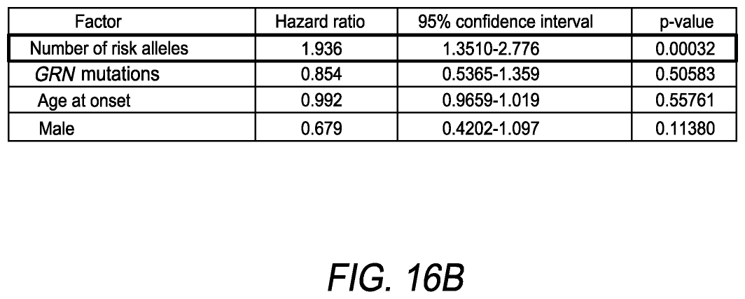

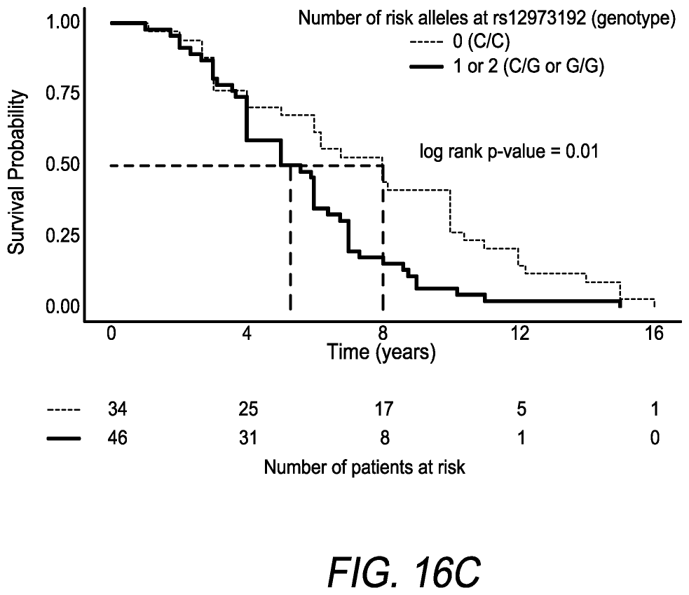

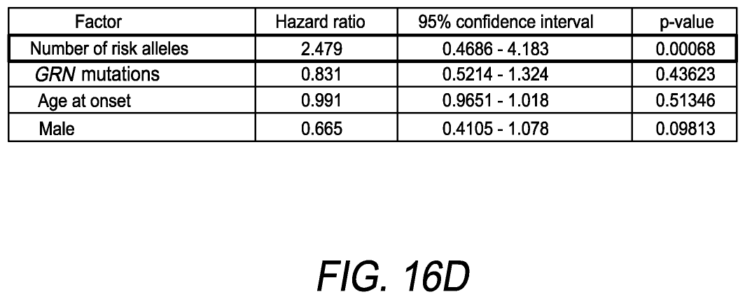

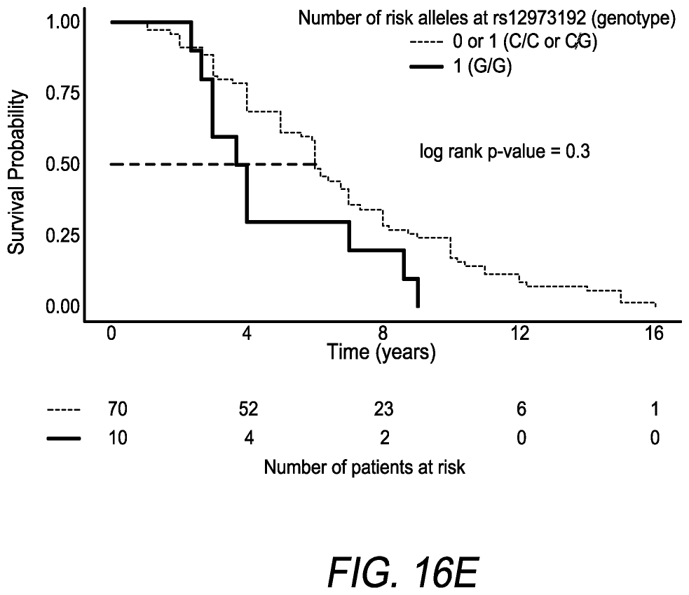

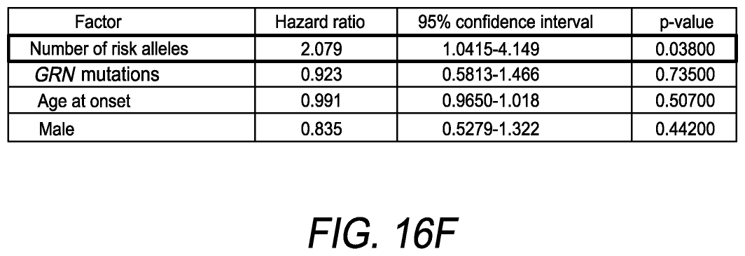

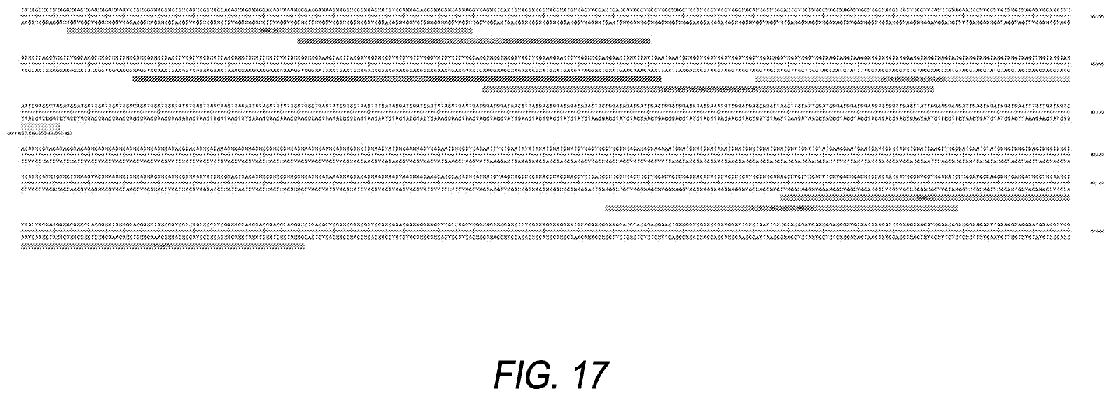

To define if these SNPs affect survival of the FTD-ALS patients (n=205) in the Mayo Clinic Brain bank, the association of the risk haplotype with survival time after disease onset was evaluated. Using Cox multivariable analysis adjusting for other factors (genetic mutations, sex, age at onset) known to influence survival, the risk haplotype was associated with survival time under an additive model (log-rank p-value=0.01) ((FIG. 4I). The number of risk haplotypes an individual carries was a strong prognostic factor (hazard ratio (HR)=1.733, p-value=0.00717) (FIG. 15A). The association remained significant under a dominant model (log-rank p-value=0.05, FIGS. 15B and 15C) and a recessive model (log-rank p-value=0.02 FIGS. 15D and 15E), indicating that carrying the risk haplotype reduces patient survival time after disease onset. The effect was more significant when only including patients carrying either the C90RF72 hexanucleotide repeat expansion or GRNmutations (FIGS. 16A-16F). Thus, genetic variants in UNC13A that increase cryptic exon inclusion are associated with decreased survival in patients.

Here, it was found that TDP-43 regulates a cryptic splicing event in the FTD/ALS gene UNC13A. The most significant genetic variants associated with disease risk, including a new one that we have nominated here, are located right in the intron harboring the cryptic exon itself. Brain samples from FTLD-TDP patients carrying these SNPs exhibited more UNC13A cryptic exon inclusion than did samples from FTLD-TDP patients that did not contain the risk alleles. It does not seem that these risk alleles are sufficient to cause cryptic exon inclusion because we do not detect them in RNA-seq data from healthy control samples (e.g., GTEx). Instead, the risk alleles in UNC13A are genuine genetic risk factors or modifiers and that the cryptic splicing event is TDP-43-loss dependent. In that way, the UNC13A risk alleles is proposed to act as a kind of Achilles' heel—lurking under the surface, not causing problems up until TDP-43 starts becoming dysfunctional (FIG. 4J). Severe loss of function mutations in the UNC13A coding region is not expected to be observed because these would result in early lethality, like in mouse. The SNPs that promote cryptic exon inclusion seem to be innocuous on their own and only become deleterious when TDP-43 function is compromised (e.g., by mutation or nuclear depletion). The discovery of a novel TDP-43-dependent cryptic splicing event in a bonafide FTD-ALS risk gene opens up a multitude of new directions for validating UNC13A as a biomarker and therapeutic target in ALS and FTD. It still remains a mystery why TDP-43 pathology is associated with ALS or FTD or FTD/ALS, or even other aging-related neuropathological changes (38). TDP-43 dysfunction-related cryptic splicing plays out across the diverse regional and neuronal landscape of the human brain. It is tempting to speculate that in addition to STMN2, and now UNC13A, there could be disease subtype specific portfolios of other important cryptic exon splicing events (and genetic variations that increase or decrease susceptibility to some of these events) that contribute to heterogeneity in clinical manifestation of TDP-43 dysfunction.

### Example 2: Inhibition of Unc13a Cryptic Exon Splice Variant Using Antisense Oligonucleotides

Antisense oligonucleotides (ASOs) targeting the UNC13A transcript are synthesized (Tables 2-5) and delivered to cultured iPSC-derived motor neurons (MNs) either by lipid transfection or gymnotic (free) uptake. iMNs are cultured in the presence of ASOs for 2-3 days followed by introduction of lentivirus delivering either a scrambled or TDP-43 targeting shRNA. The cells are cultured for an additional 4-5 days post-lentiviral infection, followed by mRNA and protein isolation. mRNA are reverse transcribed into cDNA and subjected to qPCR with primers/probes specific for UNC13A cryptic exon inclusion, in addition to primers/probes targeting properly spliced (WT) UNC13A and housekeeping genes. Protein lystates are processed for UNC13A detection by Western blot.

TABLE 2
Antisense Oligonucleotides Targeting Exon 20
Splice Donor Region of UNC13A

| | | | SEQ |
|---|---|---|---|
| | | | ID |
| Name | Position | Nucleotide Sequence | NO: |
| Exon | chr19: | GCGAGGAGAAGGTGGCCCCGT | 12 |
| 20 | 17,642,794- | ACCATGTCCAGTACACCTGTC | |
| splice | 17,642,894 | TGCATGAGGTGAGGGTCATTG | |
| donor | | CTCGGCCCCTCCCATGCCACT | |
| | | TCCACTCACCATTCCTG | |
| Exon | Reverse | CAGGAATGGTGAGTGGAAGTGGCAT | 13 |
| 20 | complement | GGGAGGGGCCGAGCAATGACCCTCA | |
| splice | | CCTCATGCAGACAGGTGTACTGGAC | |
| donor | | ATGGTACGGGGCCACCTTCTCCTCG | |
| | | C | |
| MTx_ | 1 | CAGGAATGGTGAGTGGAAGTGGCAT | 14 |
| ASO_ | | | |
| 0001 | | | |
| MTx_ | 2 | AGGAATGGTGAGTGGAAGTGGCATG | 15 |
| ASO_ | | | |
| 0002 | | | |
| MTx_ | 3 | GGAATGGTGAGTGGAAGTGGCATGG | 16 |
| ASO_ | | | |
| 0003 | | | |
| MTx_ | 4 | GAATGGTGAGTGGAAGTGGCATGGG | 17 |
| ASO_ | | | |
| 0004 | | | |
| MTx_ | 5 | AATGGTGAGTGGAAGTGGCATGGGA | 18 |
| ASO_ | | | |
| 0005 | | | |
| MTx_ | 6 | ATGGTGAGTGGAAGTGGCATGGGAG | 19 |
| ASO_ | | | |
| 0006 | | | |
| MTx_ | 7 | TGGTGAGTGGAAGTGGCATGGGAGG | 20 |
| ASO_ | | | |
| 0007 | | | |
| MTx_ | 8 | GGTGAGTGGAAGTGGCATGGGAGGG | 21 |
| ASO_ | | | |
| 0008 | | | |
| MTx_ | 9 | GTGAGTGGAAGTGGCATGGGAGGGG | 22 |
| ASO_ | | | |
| 0009 | | | |
| MTx_ | 10 | TGAGTGGAAGTGGCATGGGAGGGGC | 23 |
| ASO_ | | | |
| 0010 | | | |
| MTx_ | 11 | GAGTGGAAGTGGCATGGGAGGGGCC | 24 |
| ASO_ | | | |
| 0011 | | | |
| MTx_ | 12 | AGTGGAAGTGGCATGGGAGGGGCCG | 25 |
| ASO_ | | | |
| 0012 | | | |
| MTx_ | 13 | GTGGAAGTGGCATGGGAGGGGCCGA | 26 |
| ASO_ | | | |
| 0013 | | | |
| MTx_ | 14 | TGGAAGTGGCATGGGAGGGGCCGAG | 27 |
| ASO_ | | | |
| 0014 | | | |
| MTx_ | 15 | GGAAGTGGCATGGGAGGGGCCGAGC | 28 |
| ASO_ | | | |
| 0015 | | | |
| MTx_ | 16 | GAAGTGGCATGGGAGGGGCCGAGCA | 29 |
| ASO_ | | | |
| 0016 | | | |
| MTx_ | 17 | AAGTGGCATGGGAGGGGCCGAGCAA | 30 |
| ASO_ | | | |
| 0017 | | | |
| MTx_ | 18 | AGTGGCATGGGAGGGGCCGAGCAAT | 31 |
| ASO_ | | | |
| 0018 | | | |
| MTx_ | 19 | GTGGCATGGGAGGGGCCGAGCAATG | 32 |
| ASO_ | | | |
| 0019 | | | |
| MTx_ | 20 | TGGCATGGGAGGGGCCGAGCAATGA | 33 |
| ASO_ | | | |
| 0020 | | | |
| MTx_ | 21 | GGCATGGGAGGGGCCGAGCAATGAC | 34 |
| ASO_ | | | |
| 0021 | | | |
| MTx_ | 22 | GCATGGGAGGGGCCGAGCAATGACC | 35 |
| ASO_ | | | |
| 0022 | | | |
| MTx_ | 23 | CATGGGAGGGGCCGAGCAATGACCC | 36 |
| ASO_ | | | |
| 0023 | | | |
| MTx_ | 24 | ATGGGAGGGGCCGAGCAATGACCCT | 37 |
| ASO_ | | | |
| 0024 | | | |
| MTx_ | 25 | TGGGAGGGGCCGAGCAATGACCCTC | 38 |
| ASO_ | | | |
| 0025 | | | |
| MTx_ | 26 | GGGAGGGGCCGAGCAATGACCCTCA | 39 |
| ASO_ | | | |
| 0026 | | | |
| MTx_ | 27 | GGAGGGGCCGAGCAATGACCCTCAC | 40 |
| ASO_ | | | |
| 0027 | | | |
| MTx_ | 28 | GAGGGGCCGAGCAATGACCCTCACC | 41 |
| ASO_ | | | |
| 0028 | | | |
| MTx_ | 29 | AGGGGCCGAGCAATGACCCTCACCT | 42 |
| ASO_ | | | |
| 0029 | | | |
| MTx_ | 30 | GGGGCCGAGCAATGACCCTCACCTC | 43 |
| ASO_ | | | |
| 0030 | | | |
| MTx_ | 31 | GGGCCGAGCAATGACCCTCACCTCA | 44 |
| ASO_ | | | |
| 0031 | | | |
| MTx_ | 32 | GGCCGAGCAATGACCCTCACCTCAT | 45 |
| ASO_ | | | |
| 0032 | | | |
| MTx_ | 33 | GCCGAGCAATGACCCTCACCTCATG | 46 |
| ASO_ | | | |
| 0033 | | | |
| MTx_ | 34 | CCGAGCAATGACCCTCACCTCATGC | 47 |
| ASO_ | | | |
| 0034 | | | |
| MTx_ | 35 | CGAGCAATGACCCTCACCTCATGCA | 48 |
| ASO_ | | | |
| 0035 | | | |
| MTx_ | 36 | GAGCAATGACCCTCACCTCATGCAG | 49 |
| ASO_ | | | |
| 0036 | | | |
| MTx_ | 37 | AGCAATGACCCTCACCTCATGCAGA | 50 |
| ASO_ | | | |
| 0037 | | | |
| MTx_ | 38 | GCAATGACCCTCACCTCATGCAGAC | 51 |
| ASO_ | | | |
| 0038 | | | |
| MTx_ | 39 | CAATGACCCTCACCTCATGCAGACA | 52 |
| ASO_ | | | |
| 0039 | | | |
| MTx_ | 40 | AATGACCCTCACCTCATGCAGACAG | 53 |
| ASO_ | | | |
| 0040 | | | |
| MTx_ | 41 | ATGACCCTCACCTCATGCAGACAGG | 54 |
| ASO_ | | | |
| 0041 | | | |
| MTx_ | 42 | TGACCCTCACCTCATGCAGACAGGT | 55 |
| ASO_ | | | |
| 0042 | | | |
| MTx_ | 43 | GACCCTCACCTCATGCAGACAGGTG | 56 |
| ASO_ | | | |
| 0043 | | | |
| MTx_ | 44 | ACCCTCACCTCATGCAGACAGGTGT | 57 |
| ASO_ | | | |
| 0044 | | | |
| MTx_ | 45 | CCCTCACCTCATGCAGACAGGTGTA | 58 |
| ASO_ | | | |
| 0045 | | | |
| MTx_ | 46 | CCTCACCTCATGCAGACAGGTGTAC | 59 |
| ASO_ | | | |
| 0046 | | | |
| MTx_ | 47 | CTCACCTCATGCAGACAGGTGTACT | 60 |
| ASO_ | | | |
| 0047 | | | |
| MTx_ | 48 | TCACCTCATGCAGACAGGTGTACTG | 61 |
| ASO_ | | | |
| 0048 | | | |
| MTx_ | 49 | CACCTCATGCAGACAGGTGTACTGG | 62 |
| ASO_ | | | |
| 0049 | | | |
| MTx_ | 50 | ACCTCATGCAGACAGGTGTACTGGA | 63 |
| ASO_ | | | |
| 0050 | | | |
| MTx_ | 51 | CCTCATGCAGACAGGTGTACTGGAC | 64 |
| ASO_ | | | |
| 0051 | | | |
| MTx_ | 52 | CTCATGCAGACAGGTGTACTGGACA | 65 |
| ASO_ | | | |
| 0052 | | | |
| MTx_ | 53 | TCATGCAGACAGGTGTACTGGACAT | 66 |
| ASO_ | | | |
| 0053 | | | |
| MTx_ | 54 | CATGCAGACAGGTGTACTGGACATG | 67 |
| ASO_ | | | |
| 0054 | | | |
| MTx_ | 55 | ATGCAGACAGGTGTACTGGACATGG | 68 |
| ASO_ | | | |
| 0055 | | | |
| MTx_ | 56 | TGCAGACAGGTGTACTGGACATGGT | 69 |
| ASO_ | | | |
| 0056 | | | |
| MTx_ | 57 | GCAGACAGGTGTACTGGACATGGTA | 70 |
| ASO_ | | | |
| 0057 | | | |
| MTx_ | 58 | CAGACAGGTGTACTGGACATGGTAC | 71 |
| ASO_ | | | |
| 0058 | | | |
| MTx_ | 59 | AGACAGGTGTACTGGACATGGTACG | 72 |
| ASO_ | | | |
| 0059 | | | |
| MTx_ | 60 | GACAGGTGTACTGGACATGGTACGG | 73 |
| ASO_ | | | |
| 0060 | | | |
| MTx_ | 61 | ACAGGTGTACTGGACATGGTACGGG | 74 |
| ASO_ | | | |
| 0061 | | | |
| MTx_ | 62 | CAGGTGTACTGGACATGGTACGGGG | 75 |
| ASO_ | | | |
| 0062 | | | |
| MTx_ | 63 | AGGTGTACTGGACATGGTACGGGGC | 76 |
| ASO_ | | | |
| 0063 | | | |
| MTx_ | 64 | GGTGTACTGGACATGGTACGGGGCC | 77 |
| ASO_ | | | |
| 0064 | | | |
| MTx_ | 65 | GTGTACTGGACATGGTACGGGGCCA | 78 |
| ASO_ | | | |
| 0065 | | | |
| MTx_ | 66 | TGTACTGGACATGGTACGGGGCCAC | 79 |
| ASO_ | | | |
| 0066 | | | |
| MTx_ | 67 | GTACTGGACATGGTACGGGGCCACC | 80 |
| ASO_ | | | |
| 0067 | | | |
| MTx_ | 68 | TACTGGACATGGTACGGGGCCACCT | 81 |
| ASO_ | | | |
| 0068 | | | |
| MTx_ | 69 | ACTGGACATGGTACGGGGCCACCTT | 82 |
| ASO_ | | | |
| 0069 | | | |
| MTx_ | 70 | CTGGACATGGTACGGGGCCACCTTC | 83 |
| ASO_ | | | |
| 0070 | | | |
| MTx_ | 71 | TGGACATGGTACGGGGCCACCTTCT | 84 |
| ASO_ | | | |
| 0071 | | | |
| MTx_ | 72 | GGACATGGTACGGGGCCACCTTCTC | 85 |
| ASO_ | | | |
| 0072 | | | |
| MTx_ | 73 | GACATGGTACGGGGCCACCTTCTCC | 86 |
| ASO_ | | | |
| 0073 | | | |
| MTx_ | 74 | ACATGGTACGGGGCCACCTTCTCCT | 87 |
| ASO_ | | | |
| 0074 | | | |
| MTx_ | 75 | CATGGTACGGGGCCACCTTCTCCTC | 88 |
| ASO_ | | | |
| 0075 | | | |
| MTx_ | 76 | ATGGTACGGGGCCACCTTCTCCTCG | 89 |
| ASO_ | | | |
| 0076 | | | |
| MTx_ | 77 | TGGTACGGGGCCACCTTCTCCTCGC | 90 |
| ASO_ | | | |
| 0077 | | | |

TABLE 3
Antisense Oligonucleotides Targeting Cryptic Exon Splice
Acceptor Region of UNC13A

| Name | Position | Nucleotide Sequence | SEQ ID |
|---|---|---|---|
| | | | NO: |
| Cryptic exon | chr19: | CTCCAGGTTGACTCTCACTACTCATCATC | 91 |
| splice | 17,642,491- | AGGTTCTTCCTTCTATTCCAGCCCTAACC | |
| acceptor | 17,642,641 | ACTCAGGATTGGGCCGTTTGTGTCTGGGT | |
| | | ATGTCTCTTCCAGCTGCCTGGGTTTCCTG | |
| | | GAAAGAACTCTTATCCCCAGGAACTAGTT | |
| | | TGTTGA | |
| Cryptic exon | Reverse | TCAACAAACTAGTTCCTGGGGATAAGAGT | 92 |
| splice | complement | TCTTTCCAGGAAACCCAGGCAGCTGGAAG | |
| acceptor | | AGACATACCCAGACACAAACGGCCCAATC | |
| | | CTGAGTGGTTAGGGCTGGAATAGAAGGAA | |
| | | GAACCTGATGATGAGTAGTGAGAGTCAAC | |
| | | CTGGAG | |
| MTx_ASO_0078 | 1 | TCAACAAACTAGTTCCTGGGGATAA | 93 |
| MTx_ASO_0079 | 2 | CAACAAACTAGTTCCTGGGGATAAG | 94 |
| MTx_ASO_0080 | 3 | AACAAACTAGTTCCTGGGGATAAGA | 95 |
| MTx_ASO_0081 | 4 | ACAAACTAGTTCCTGGGGATAAGAG | 96 |
| MTx_ASO_0082 | 5 | CAAACTAGTTCCTGGGGATAAGAGT | 97 |
| MTx_ASO_0083 | 6 | AAACTAGTTCCTGGGGATAAGAGTT | 98 |
| MTx_ASO_0084 | 7 | AACTAGTTCCTGGGGATAAGAGTTC | 99 |
| MTx_ASO_0085 | 8 | ACTAGTTCCTGGGGATAAGAGTTCT | 100 |
| MTx_ASO_0086 | 9 | CTAGTTCCTGGGGATAAGAGTTCTT | 101 |
| MTx_ASO_0087 | 10 | TAGTTCCTGGGGATAAGAGTTCTTT | 102 |
| MTx_ASO_0088 | 11 | AGTTCCTGGGGATAAGAGTTCTTTC | 103 |
| MTx_ASO_0089 | 12 | GTTCCTGGGGATAAGAGTTCTTTCC | 104 |
| MTx_ASO_0090 | 13 | TTCCTGGGGATAAGAGTTCTTTCCA | 105 |
| MTx_ASO_0091 | 14 | TCCTGGGGATAAGAGTTCTTTCCAG | 106 |
| MTx_ASO_0092 | 15 | CCTGGGGATAAGAGTTCTTTCCAGG | 107 |
| MTx_ASO_0093 | 16 | CTGGGGATAAGAGTTCTTTCCAGGA | 108 |
| MTx_ASO_0094 | 17 | TGGGGATAAGAGTTCTTTCCAGGAA | 109 |
| MTx_ASO_0095 | 18 | GGGGATAAGAGTTCTTTCCAGGAAA | 110 |
| MTx_ASO_0096 | 19 | GGGATAAGAGTTCTTTCCAGGAAAC | 111 |
| MTx_ASO_0097 | 20 | GGATAAGAGTTCTTTCCAGGAAACC | 112 |
| MTx_ASO_0098 | 21 | GATAAGAGTTCTTTCCAGGAAACCC | 113 |
| MTx_ASO_0099 | 22 | ATAAGAGTTCTTTCCAGGAAACCCA | 114 |
| MTx_ASO_0100 | 23 | TAAGAGTTCTTTCCAGGAAACCCAG | 115 |
| MTx_ASO_0101 | 24 | AAGAGTTCTTTCCAGGAAACCCAGG | 116 |
| MTx_ASO_0102 | 25 | AGAGTTCTTTCCAGGAAACCCAGGC | 117 |
| MTx_ASO_0103 | 26 | GAGTTCTTTCCAGGAAACCCAGGCA | 118 |
| MTx_ASO_0104 | 27 | AGTTCTTTCCAGGAAACCCAGGCAG | 119 |
| MTx_ASO_0105 | 28 | GTTCTTTCCAGGAAACCCAGGCAGC | 120 |
| MTx_ASO_0106 | 29 | TTCTTTCCAGGAAACCCAGGCAGCT | 121 |
| MTx_ASO_0107 | 30 | TCTTTCCAGGAAACCCAGGCAGCTG | 122 |
| MTx_ASO_0108 | 31 | CTTTCCAGGAAACCCAGGCAGCTGG | 123 |
| MTx_ASO_0109 | 32 | TTTCCAGGAAACCCAGGCAGCTGGA | 124 |
| MTx_ASO_0110 | 33 | TTCCAGGAAACCCAGGCAGCTGGAA | 125 |
| MTx_ASO_0111 | 34 | TCCAGGAAACCCAGGCAGCTGGAAG | 126 |
| MTx_ASO_0112 | 35 | CCAGGAAACCCAGGCAGCTGGAAGA | 127 |
| MTx_ASO_0113 | 36 | CAGGAAACCCAGGCAGCTGGAAGAG | 128 |
| MTx_ASO_0114 | 37 | AGGAAACCCAGGCAGCTGGAAGAGA | 129 |
| MTx_ASO_0115 | 38 | GGAAACCCAGGCAGCTGGAAGAGAC | 130 |
| MTx_ASO_0116 | 39 | GAAACCCAGGCAGCTGGAAGAGACA | 131 |
| MTx_ASO_0117 | 40 | AAACCCAGGCAGCTGGAAGAGACAT | 132 |
| MTx_ASO_0118 | 41 | AACCCAGGCAGCTGGAAGAGACATA | 133 |
| MTx_ASO_0119 | 42 | ACCCAGGCAGCTGGAAGAGACATAC | 134 |
| MTx_ASO_0120 | 43 | CCCAGGCAGCTGGAAGAGACATACC | 135 |
| MTx_ASO_0121 | 44 | CCAGGCAGCTGGAAGAGACATACCC | 136 |
| MTx_ASO_0122 | 45 | CAGGCAGCTGGAAGAGACATACCCA | 137 |
| MTx_ASO_0123 | 46 | AGGCAGCTGGAAGAGACATACCCAG | 138 |
| MTx_ASO_0124 | 47 | GGCAGCTGGAAGAGACATACCCAGA | 139 |
| MTx_ASO_0125 | 48 | GCAGCTGGAAGAGACATACCCAGAC | 140 |
| MTx_ASO_0126 | 49 | CAGCTGGAAGAGACATACCCAGACA | 141 |
| MTx_ASO_0127 | 50 | AGCTGGAAGAGACATACCCAGACAC | 142 |
| MTx_ASO_0128 | 51 | GCTGGAAGAGACATACCCAGACACA | 143 |
| MTx_ASO_0129 | 52 | CTGGAAGAGACATACCCAGACACAA | 144 |
| MTx_ASO_0130 | 53 | TGGAAGAGACATACCCAGACACAAA | 145 |
| MTx_ASO_0131 | 54 | GGAAGAGACATACCCAGACACAAAC | 146 |
| MTx_ASO_0132 | 55 | GAAGAGACATACCCAGACACAAACG | 147 |
| MTx_ASO_0133 | 56 | AAGAGACATACCCAGACACAAACGG | 148 |
| MTx_ASO_0134 | 57 | AGAGACATACCCAGACACAAACGGC | 149 |
| MTx_ASO_0135 | 58 | GAGACATACCCAGACACAAACGGCC | 150 |
| MTx_ASO_0136 | 59 | AGACATACCCAGACACAAACGGCCC | 151 |
| MTx_ASO_0137 | 60 | GACATACCCAGACACAAACGGCCCA | 152 |
| MTx_ASO_0138 | 61 | ACATACCCAGACACAAACGGCCCAA | 153 |
| MTx_ASO_0139 | 62 | CATACCCAGACACAAACGGCCCAAT | 154 |
| MTx_ASO_0140 | 63 | ATACCCAGACACAAACGGCCCAATC | 155 |
| MTx_ASO_0141 | 64 | TACCCAGACACAAACGGCCCAATCC | 156 |
| MTx_ASO_0142 | 65 | ACCCAGACACAAACGGCCCAATCCT | 157 |
| MTx_ASO_0143 | 66 | CCCAGACACAAACGGCCCAATCCTG | 158 |
| MTx_ASO_0144 | 67 | CCAGACACAAACGGCCCAATCCTGA | 159 |
| MTx_ASO_0145 | 68 | CAGACACAAACGGCCCAATCCTGAG | 160 |
| MTx_ASO_0146 | 69 | AGACACAAACGGCCCAATCCTGAGT | 161 |
| MTx_ASO_0147 | 70 | GACACAAACGGCCCAATCCTGAGTG | 162 |
| MTx_ASO_0148 | 71 | ACACAAACGGCCCAATCCTGAGTGG | 163 |
| MTx_ASO_0149 | 72 | CACAAACGGCCCAATCCTGAGTGGT | 164 |
| MTx_ASO_0150 | 73 | ACAAACGGCCCAATCCTGAGTGGTT | 165 |
| MTx_ASO_0151 | 74 | CAAACGGCCCAATCCTGAGTGGTTA | 166 |
| MTx_ASO_0152 | 75 | AAACGGCCCAATCCTGAGTGGTTAG | 167 |
| MTx_ASO_0153 | 76 | AACGGCCCAATCCTGAGTGGTTAGG | 168 |
| MTx_ASO_0154 | 77 | ACGGCCCAATCCTGAGTGGTTAGGG | 169 |
| MTx_ASO_0155 | 78 | CGGCCCAATCCTGAGTGGTTAGGGC | 170 |
| MTx_ASO_0156 | 79 | GGCCCAATCCTGAGTGGTTAGGGCT | 171 |
| MTx_ASO_0157 | 80 | GCCCAATCCTGAGTGGTTAGGGCTG | 172 |
| MTx_ASO_0158 | 81 | CCCAATCCTGAGTGGTTAGGGCTGG | 173 |
| MTx_ASO_0159 | 82 | CCAATCCTGAGTGGTTAGGGCTGGA | 174 |
| MTx_ASO_0160 | 83 | CAATCCTGAGTGGTTAGGGCTGGAA | 175 |
| MTx_ASO_0161 | 84 | AATCCTGAGTGGTTAGGGCTGGAAT | 176 |
| MTx_ASO_0162 | 85 | ATCCTGAGTGGTTAGGGCTGGAATA | 177 |
| MTx_ASO_0163 | 86 | TCCTGAGTGGTTAGGGCTGGAATAG | 178 |
| MTx_ASO_0164 | 87 | CCTGAGTGGTTAGGGCTGGAATAGA | 179 |
| MTx_ASO_0165 | 88 | CTGAGTGGTTAGGGCTGGAATAGAA | 180 |
| MTx_ASO_0166 | 89 | TGAGTGGTTAGGGCTGGAATAGAAG | 181 |
| MTx_ASO_0167 | 90 | GAGTGGTTAGGGCTGGAATAGAAGG | 182 |
| MTx_ASO_0168 | 91 | AGTGGTTAGGGCTGGAATAGAAGGA | 183 |
| MTx_ASO_0169 | 92 | GTGGTTAGGGCTGGAATAGAAGGAA | 184 |
| MTx_ASO_0170 | 93 | TGGTTAGGGCTGGAATAGAAGGAAG | 185 |
| MTx_ASO_0171 | 94 | GGTTAGGGCTGGAATAGAAGGAAGA | 186 |
| MTx_ASO_0172 | 95 | GTTAGGGCTGGAATAGAAGGAAGAA | 187 |
| MTx_ASO_0173 | 96 | TTAGGGCTGGAATAGAAGGAAGAAC | 188 |
| MTx_ASO_0174 | 97 | TAGGGCTGGAATAGAAGGAAGAACC | 189 |
| MTx_ASO_0175 | 98 | AGGGCTGGAATAGAAGGAAGAACCT | 190 |
| MTx_ASO_0176 | 99 | GGGCTGGAATAGAAGGAAGAACCTG | 191 |
| MTx_ASO_0177 | 100 | GGCTGGAATAGAAGGAAGAACCTGA | 192 |
| MTx_ASO_0178 | 101 | GCTGGAATAGAAGGAAGAACCTGAT | 193 |
| MTx_ASO_0179 | 102 | CTGGAATAGAAGGAAGAACCTGATG | 194 |
| MTx_ASO_0180 | 103 | TGGAATAGAAGGAAGAACCTGATGA | 195 |
| MTx_ASO_0181 | 104 | GGAATAGAAGGAAGAACCTGATGAT | 196 |
| MTx_ASO_0182 | 105 | GAATAGAAGGAAGAACCTGATGATG | 197 |
| MTx_ASO_0183 | 106 | AATAGAAGGAAGAACCTGATGATGA | 198 |
| MTx_ASO_0184 | 107 | ATAGAAGGAAGAACCTGATGATGAG | 199 |
| MTx_ASO_0185 | 108 | TAGAAGGAAGAACCTGATGATGAGT | 200 |
| MTx_ASO_0186 | 109 | AGAAGGAAGAACCTGATGATGAGTA | 201 |
| MTx_ASO_0187 | 110 | GAAGGAAGAACCTGATGATGAGTAG | 202 |
| MTx_ASO_0188 | 111 | AAGGAAGAACCTGATGATGAGTAGT | 203 |
| MTx_ASO_0189 | 112 | AGGAAGAACCTGATGATGAGTAGTG | 204 |
| MTx_ASO_0190 | 113 | GGAAGAACCTGATGATGAGTAGTGA | 205 |
| MTx_ASO_0191 | 114 | GAAGAACCTGATGATGAGTAGTGAG | 206 |
| MTx_ASO_0192 | 115 | AAGAACCTGATGATGAGTAGTGAGA | 207 |
| MTx_ASO_0193 | 116 | AGAACCTGATGATGAGTAGTGAGAG | 208 |
| MTx_ASO_0194 | 117 | GAACCTGATGATGAGTAGTGAGAGT | 209 |
| MTx_ASO_0195 | 118 | AACCTGATGATGAGTAGTGAGAGTC | 210 |
| MTx_ASO_0196 | 119 | ACCTGATGATGAGTAGTGAGAGTCA | 211 |
| MTx_ASO_0197 | 120 | CCTGATGATGAGTAGTGAGAGTCAA | 212 |
| MTx_ASO_0198 | 121 | CTGATGATGAGTAGTGAGAGTCAAC | 213 |
| MTx_ASO_0199 | 122 | TGATGATGAGTAGTGAGAGTCAACC | 214 |
| MTx_ASO_0200 | 123 | GATGATGAGTAGTGAGAGTCAACCT | 215 |
| MTx_ASO_0201 | 124 | ATGATGAGTAGTGAGAGTCAACCTG | 216 |
| MTx_ASO_0202 | 125 | TGATGAGTAGTGAGAGTCAACCTGG | 217 |
| MTx_ASO_0203 | 126 | GATGAGTAGTGAGAGTCAACCTGGA | 218 |
| MTx_ASO_0204 | 127 | ATGAGTAGTGAGAGTCAACCTGGAG | 219 |

TABLE 4
Antisense Oligonucleotides Targeting Cryptic Exon Splice
Donor Region of UNC13A

| | | | SEQ ID |
|---|---|---|---|
| Name | Position | Nucleotide Sequence | NO: |
| Cryptic exon | chr19: | TGAACAGATGAATGAGTGATGAGTAGATA | 220 |
| splice donor | 17,642,363- | AAAGGATGGATGGAGAGATGGGTGAGTAC | |
| | 17,642,463 | ATGGATGGATAGATGGATGAGTTGGTGGG | |
| | | TAGATTCGTGGCTA | |
| Cryptic exon | Reverse | TAGCCACGAATCTACCCACCAACTCATCC | 221 |
| splice donor | Complement | ATCTATCCATCCATGTACTCACCCATCTC | |
| | | TCCATCCATCCTTTTATCTACTCATCACT | |
| | | CATTCATCTGTTCA | |
| MTx_ASO_0205 | 1 | TAGCCACGAATCTACCCACCAACTC | 222 |
| MTx_ASO_0206 | 2 | AGCCACGAATCTACCCACCAACTCA | 223 |
| MTx_ASO_0207 | 3 | GCCACGAATCTACCCACCAACTCAT | 224 |
| MTX_ASO_0208 | 4 | CCACGAATCTACCCACCAACTCATC | 225 |
| MTx_ASO_0209 | 5 | CACGAATCTACCCACCAACTCATCC | 226 |
| MTx_ASO_0210 | 6 | ACGAATCTACCCACCAACTCATCCA | 227 |
| MTx_ASO_0211 | 7 | CGAATCTACCCACCAACTCATCCAT | 228 |
| MTx_ASO_0212 | 8 | GAATCTACCCACCAACTCATCCATC | 229 |
| MTx_ASO_0213 | 9 | AATCTACCCACCAACTCATCCATCT | 230 |
| MTx_ASO_0214 | 10 | ATCTACCCACCAACTCATCCATCTA | 231 |
| MTx_ASO_0215 | 11 | TCTACCCACCAACTCATCCATCTAT | 232 |
| MTx_ASO_0216 | 12 | CTACCCACCAACTCATCCATCTATC | 233 |
| MTx_ASO_0217 | 13 | TACCCACCAACTCATCCATCTATCC | 234 |
| MTx_ASO_0218 | 14 | ACCCACCAACTCATCCATCTATCCA | 235 |
| MTx_ASO_0219 | 15 | CCCACCAACTCATCCATCTATCCAT | 236 |
| MTx_ASO_0220 | 16 | CCACCAACTCATCCATCTATCCATC | 237 |
| MTX_ASO_0221 | 17 | CACCAACTCATCCATCTATCCATCC | 238 |
| MTx_ASO_0222 | 18 | ACCAACTCATCCATCTATCCATCCA | 239 |
| MTx_ASO_0223 | 19 | CCAACTCATCCATCTATCCATCCAT | 240 |
| MTx_ASO_0224 | 20 | CAACTCATCCATCTATCCATCCATG | 241 |
| MTx_ASO_0225 | 21 | AACTCATCCATCTATCCATCCATGT | 242 |
| MTx_ASO_0226 | 22 | ACTCATCCATCTATCCATCCATGTA | 243 |
| MTx_ASO_0227 | 23 | CTCATCCATCTATCCATCCATGTAC | 244 |
| MTx_ASO_0228 | 24 | TCATCCATCTATCCATCCATGTACT | 245 |
| MTx_ASO_0229 | 25 | CATCCATCTATCCATCCATGTACTC | 246 |
| MTx_ASO_0230 | 26 | ATCCATCTATCCATCCATGTACTCA | 247 |
| MTx_ASO_0231 | 27 | TCCATCTATCCATCCATGTACTCAC | 248 |
| MTx_ASO_0232 | 28 | CCATCTATCCATCCATGTACTCACC | 249 |
| MTx_ASO_0233 | 29 | CATCTATCCATCCATGTACTCACCC | 250 |
| MTx_ASO_0234 | 30 | ATCTATCCATCCATGTACTCACCCA | 251 |
| MTx_ASO_0235 | 31 | TCTATCCATCCATGTACTCACCCAT | 252 |
| MTx_ASO_0236 | 32 | CTATCCATCCATGTACTCACCCATC | 253 |
| MTx_ASO_0237 | 33 | TATCCATCCATGTACTCACCCATCT | 254 |
| MTx_ASO_0238 | 34 | ATCCATCCATGTACTCACCCATCTC | 255 |
| MTx_ASO_0239 | 35 | TCCATCCATGTACTCACCCATCTCT | 256 |
| MTx_ASO_0240 | 36 | CCATCCATGTACTCACCCATCTCTC | 257 |
| MTx_ASO_0241 | 37 | CATCCATGTACTCACCCATCTCTCC | 258 |
| MTx_ASO_0242 | 38 | ATCCATGTACTCACCCATCTCTCCA | 259 |
| MTx_ASO_0243 | 39 | TCCATGTACTCACCCATCTCTCCAT | 260 |
| MTx_ASO_0244 | 40 | CCATGTACTCACCCATCTCTCCATC | 261 |
| MTx_ASO_0245 | 41 | CATGTACTCACCCATCTCTCCATCC | 262 |
| MTx_ASO_0246 | 42 | ATGTACTCACCCATCTCTCCATCCA | 263 |
| MTx_ASO_0247 | 43 | TGTACTCACCCATCTCTCCATCCAT | 264 |
| MTx_ASO_0248 | 44 | GTACTCACCCATCTCTCCATCCATC | 265 |
| MTx_ASO_0249 | 45 | TACTCACCCATCTCTCCATCCATCC | 266 |
| MTx_ASO_0250 | 46 | ACTCACCCATCTCTCCATCCATCCT | 267 |
| MTx_ASO_0251 | 47 | CTCACCCATCTCTCCATCCATCCTT | 268 |
| MTx_ASO_0252 | 48 | TCACCCATCTCTCCATCCATCCTTT | 269 |
| MTx_ASO_0253 | 49 | CACCCATCTCTCCATCCATCCTTTT | 270 |
| MTx_ASO_0254 | 50 | ACCCATCTCTCCATCCATCCTTTTA | 271 |
| MTx_ASO_0255 | 51 | CCCATCTCTCCATCCATCCTTTTAT | 272 |
| MTx_ASO_0256 | 52 | CCATCTCTCCATCCATCCTTTTATC | 273 |
| MTx_ASO_0257 | 53 | CATCTCTCCATCCATCCTTTTATCT | 274 |
| MIX_ASO_0258 | 54 | ATCTCTCCATCCATCCTTTTATCTA | 275 |
| MTx_ASO_0259 | 55 | TCTCTCCATCCATCCTTTTATCTAC | 276 |
| MTx_ASO_0260 | 56 | CTCTCCATCCATCCTTTTATCTACT | 277 |
| MTx_ASO_0261 | 57 | TCTCCATCCATCCTTTTATCTACTC | 278 |
| MTx_ASO_0262 | 58 | CTCCATCCATCCTTTTATCTACTCA | 279 |
| MTx_ASO_0263 | 59 | TCCATCCATCCTTTTATCTACTCAT | 280 |
| MTx_ASO_0264 | 60 | CCATCCATCCTTTTATCTACTCATC | 281 |
| MTx_ASO_0265 | 61 | CATCCATCCTTTTATCTACTCATCA | 282 |
| MTX_ASO_0266 | 62 | ATCCATCCTTTTATCTACTCATCAC | 283 |
| MTx_ASO_0267 | 63 | TCCATCCTTTTATCTACTCATCACT | 284 |
| MTx_ASO_0268 | 64 | CCATCCTTTTATCTACTCATCACTC | 285 |
| MTx_ASO_0269 | 65 | CATCCTTTTATCTACTCATCACTCA | 286 |
| MTx_ASO_0270 | 66 | ATCCTTTTATCTACTCATCACTCAT | 287 |
| MTx_ASO_0271 | 67 | TCCTTTTATCTACTCATCACTCATT | 288 |
| MTx_ASO_0272 | 68 | CCTTTTATCTACTCATCACTCATTC | 289 |
| MTx_ASO_0273 | 69 | CTTTTATCTACTCATCACTCATTCA | 290 |
| MTx_ASO_0274 | 70 | TTTTATCTACTCATCACTCATTCAT | 291 |
| MTx_ASO_0275 | 71 | TTTATCTACTCATCACTCATTCATC | 292 |
| MTx_ASO_0276 | 72 | TTATCTACTCATCACTCATTCATCT | 293 |
| MTx_ASO_0277 | 73 | TATCTACTCATCACTCATTCATCTG | 294 |
| MTx_ASO_0278 | 74 | ATCTACTCATCACTCATTCATCTGT | 295 |
| MTx_ASO_0279 | 75 | TCTACTCATCACTCATTCATCTGTT | 296 |
| MTx_ASO_0280 | 76 | CTACTCATCACTCATTCATCTGTTC | 297 |
| MTx_ASO_0281 | 77 | TACTCATCACTCATTCATCTGTTCA | 298 |

TABLE 5
Antisense Oligonucleotides Targeting Exon 21
Splice Acceptor Region of UNC13A

| | | | SEQ |
|---|---|---|---|
| | | | ID |
| Name | Position | Nucleotide Sequence | NO: |
| Exon 21 | chr19: | CCCGGCGACCCCTTGCACTCT | 299 |
| splice | 17,641,506- | CCATGACACTTTCTCTCCCAT | |
| accep- | 17,641,606 | GGTGGCAGAACCTGTTCCACT | |
| tor | | TCGTGACCGACGTGCAGAACA | |
| | | ATGGGGTCGTGAAGATC | |
| Exon 21 | Reverse | GATCTTCACGACCCCATTGTTCTGC | 300 |
| splice | complement | ACGTCGGTCACGAAGTGGAACAGGT | |
| accep- | | TCTGCCACCATGGGAGAGAAAGTGT | |
| tor | | CATGGAGAGTGCAAGGGGTCGCCGG | |
| | | G | |
| MTx_ | 1 | GATCTTCACGACCCCATTGTTCTGC | 301 |
| ASO_ | | | |
| 0282 | | | |
| MTx_ | 2 | ATCTTCACGACCCCATTGTTCTGCA | 302 |
| ASO_ | | | |
| 0283 | | | |
| MTx_ | 3 | TCTTCACGACCCCATTGTTCTGCAC | 303 |
| ASO_ | | | |
| 0284 | | | |
| MTx_ | 4 | CTTCACGACCCCATTGTTCTGCACG | 304 |
| ASO_ | | | |
| 0285 | | | |
| MTx_ | 5 | TTCACGACCCCATTGTTCTGCACGT | 305 |
| ASO_ | | | |
| 0286 | | | |
| MTx_ | 6 | TCACGACCCCATTGTTCTGCACGTC | 306 |
| ASO_ | | | |
| 0287 | | | |
| MTx_ | 7 | CACGACCCCATTGTTCTGCACGTCG | 307 |
| ASO_ | | | |
| 0288 | | | |
| MTx_ | 8 | ACGACCCCATTGTTCTGCACGTCGG | 308 |
| ASO_ | | | |
| 0289 | | | |
| MTx_ | 9 | CGACCCCATTGTTCTGCACGTCGGT | 309 |
| ASO_ | | | |
| 0290 | | | |
| MTx_ | 10 | GACCCCATTGTTCTGCACGTCGGTC | 310 |
| ASO_ | | | |
| 0291 | | | |
| MTx_ | 11 | ACCCCATTGTTCTGCACGTCGGTCA | 311 |
| ASO_ | | | |
| 0292 | | | |
| MTx_ | 12 | CCCCATTGTTCTGCACGTCGGTCAC | 312 |
| ASO_ | | | |
| 0293 | | | |
| MTx_ | 13 | CCCATTGTTCTGCACGTCGGTCACG | 313 |
| ASO_ | | | |
| 0294 | | | |
| MTx_ | 14 | CCATTGTTCTGCACGTCGGTCACGA | 314 |
| ASO_ | | | |
| 0295 | | | |
| MTx_ | 15 | CATTGTTCTGCACGTCGGTCACGAA | 315 |
| ASO_ | | | |
| 0296 | | | |
| MTx_ | 16 | ATTGTTCTGCACGTCGGTCACGAAG | 316 |
| ASO_ | | | |
| 0297 | | | |
| MTx_ | 17 | TTGTTCTGCACGTCGGTCACGAAGT | 317 |
| ASO_ | | | |
| 0298 | | | |
| MTx_ | 18 | TGTTCTGCACGTCGGTCACGAAGTG | 318 |
| ASO_ | | | |
| 0299 | | | |
| MTx_ | 19 | GTTCTGCACGTCGGTCACGAAGTGG | 319 |
| ASO_ | | | |
| 0300 | | | |
| MTx_ | 20 | TTCTGCACGTCGGTCACGAAGTGGA | 320 |
| ASO_ | | | |
| 0301 | | | |
| MTx_ | 21 | TCTGCACGTCGGTCACGAAGTGGAA | 321 |
| ASO_ | | | |
| 0302 | | | |
| MTx_ | 22 | CTGCACGTCGGTCACGAAGTGGAAC | 322 |
| ASO_ | | | |
| 0303 | | | |
| MTx_ | 23 | TGCACGTCGGTCACGAAGTGGAACA | 323 |
| ASO_ | | | |
| 0304 | | | |
| MTx_ | 24 | GCACGTCGGTCACGAAGTGGAACAG | 324 |
| ASO_ | | | |
| 0305 | | | |
| MTx_ | 25 | CACGTCGGTCACGAAGTGGAACAGG | 325 |
| ASO_ | | | |
| 0306 | | | |
| MTx_ | 26 | ACGTCGGTCACGAAGTGGAACAGGT | 326 |
| ASO_ | | | |
| 0307 | | | |
| MTx_ | 27 | CGTCGGTCACGAAGTGGAACAGGTT | 327 |
| ASO_ | | | |
| 0308 | | | |
| MTx_ | 28 | GTCGGTCACGAAGTGGAACAGGTTC | 328 |
| ASO_ | | | |
| 0309 | | | |
| MTx_ | 29 | TCGGTCACGAAGTGGAACAGGTTCT | 329 |
| ASO_ | | | |
| 0310 | | | |
| MTx_ | 30 | CGGTCACGAAGTGGAACAGGTTCTG | 330 |
| ASO_ | | | |
| 0311 | | | |
| MTx_ | 31 | GGTCACGAAGTGGAACAGGTTCTGC | 331 |
| ASO_ | | | |
| 0312 | | | |
| MTx_ | 32 | GTCACGAAGTGGAACAGGTTCTGCC | 332 |
| ASO_ | | | |
| 0313 | | | |
| MTx_ | 33 | TCACGAAGTGGAACAGGTTCTGCCA | 333 |
| ASO_ | | | |
| 0314 | | | |
| MTx_ | 34 | CACGAAGTGGAACAGGTTCTGCCAC | 334 |
| ASO_ | | | |
| 0315 | | | |
| MTx_ | 35 | ACGAAGTGGAACAGGTTCTGCCACC | 335 |
| ASO_ | | | |
| 0316 | | | |
| MTx_ | 36 | CGAAGTGGAACAGGTTCTGCCACCA | 336 |
| ASO_ | | | |
| 0317 | | | |
| MTx_ | 37 | GAAGTGGAACAGGTTCTGCCACCAT | 337 |
| ASO_ | | | |
| 0318 | | | |
| MTx_ | 38 | AAGTGGAACAGGTTCTGCCACCATG | 338 |
| ASO_ | | | |
| 0319 | | | |
| MTx_ | 39 | AGTGGAACAGGTTCTGCCACCATGG | 339 |
| ASO_ | | | |
| 0320 | | | |
| MTx_ | 40 | GTGGAACAGGTTCTGCCACCATGGG | 340 |
| ASO_ | | | |
| 0321 | | | |
| MTx_ | 41 | TGGAACAGGTTCTGCCACCATGGGA | 341 |
| ASO_ | | | |
| 0322 | | | |
| MTx_ | 42 | GGAACAGGTTCTGCCACCATGGGAG | 342 |
| ASO_ | | | |
| 0323 | | | |
| MTx_ | 43 | GAACAGGTTCTGCCACCATGGGAGA | 343 |
| ASO_ | | | |
| 0324 | | | |
| MTx_ | 44 | AACAGGTTCTGCCACCATGGGAGAG | 344 |
| ASO_ | | | |
| 0325 | | | |
| MTx_ | 45 | ACAGGTTCTGCCACCATGGGAGAGA | 345 |
| ASO_ | | | |
| 0326 | | | |
| MTx_ | 46 | CAGGTTCTGCCACCATGGGAGAGAA | 346 |
| ASO_ | | | |
| 0327 | | | |
| MTx_ | 47 | AGGTTCTGCCACCATGGGAGAGAAA | 347 |
| ASO_ | | | |
| 0328 | | | |
| MTx_ | 48 | GGTTCTGCCACCATGGGAGAGAAAG | 348 |
| ASO_ | | | |
| 0329 | | | |
| MTx_ | 49 | GTTCTGCCACCATGGGAGAGAAAGT | 349 |
| ASO_ | | | |
| 0330 | | | |
| MTx_ | 50 | TTCTGCCACCATGGGAGAGAAAGTG | 350 |
| ASO_ | | | |
| 0331 | | | |
| MTx_ | 51 | TCTGCCACCATGGGAGAGAAAGTGT | 351 |
| ASO_ | | | |
| 0332 | | | |
| MTx_ | 52 | CTGCCACCATGGGAGAGAAAGTGTC | 352 |
| ASO_ | | | |
| 0333 | | | |
| MTx_ | 53 | TGCCACCATGGGAGAGAAAGTGTCA | 353 |
| ASO_ | | | |
| 0334 | | | |
| MTx_ | 54 | GCCACCATGGGAGAGAAAGTGTCAT | 354 |
| ASO_ | | | |
| 0335 | | | |
| MTx_ | 55 | CCACCATGGGAGAGAAAGTGTCATG | 355 |
| ASO_ | | | |
| 0336 | | | |
| MTx_ | 56 | CACCATGGGAGAGAAAGTGTCATGG | 356 |
| ASO_ | | | |
| 0337 | | | |
| MTx_ | 57 | ACCATGGGAGAGAAAGTGTCATGGA | 357 |
| ASO_ | | | |
| 0338 | | | |
| MTx_ | 58 | CCATGGGAGAGAAAGTGTCATGGAG | 358 |
| ASO_ | | | |
| 0339 | | | |
| MTx_ | 59 | CATGGGAGAGAAAGTGTCATGGAGA | 359 |
| ASO_ | | | |
| 0340 | | | |
| MTx_ | 60 | ATGGGAGAGAAAGTGTCATGGAGAG | 360 |
| ASO_ | | | |
| 0341 | | | |
| MTx_ | 61 | TGGGAGAGAAAGTGTCATGGAGAGT | 361 |
| ASO_ | | | |
| 0342 | | | |
| MTx_ | 62 | GGGAGAGAAAGTGTCATGGAGAGTG | 362 |
| ASO_ | | | |
| 0343 | | | |
| MTx_ | 63 | GGAGAGAAAGTGTCATGGAGAGTGC | 363 |
| ASO_ | | | |
| 0344 | | | |
| MTx_ | 64 | GAGAGAAAGTGTCATGGAGAGTGCA | 364 |
| ASO_ | | | |
| 0345 | | | |
| MTx_ | 65 | AGAGAAAGTGTCATGGAGAGTGCAA | 365 |
| ASO_ | | | |
| 0346 | | | |
| MTx_ | 66 | GAGAAAGTGTCATGGAGAGTGCAAG | 366 |
| ASO_ | | | |
| 0347 | | | |
| MTx_ | 67 | AGAAAGTGTCATGGAGAGTGCAAGG | 367 |
| ASO_ | | | |
| 0348 | | | |
| MTx_ | 68 | GAAAGTGTCATGGAGAGTGCAAGGG | 368 |
| ASO_ | | | |
| 0349 | | | |
| MTx_ | 69 | AAAGTGTCATGGAGAGTGCAAGGGG | 369 |
| ASO | | | |
| _0350 | | | |
| MTx_ | 70 | AAGTGTCATGGAGAGTGCAAGGGGT | 370 |
| ASO_ | | | |
| 0351 | | | |
| MTx_ | 71 | AGTGTCATGGAGAGTGCAAGGGGTC | 371 |
| ASO_ | | | |
| 0352 | | | |
| MTx_ | 72 | GTGTCATGGAGAGTGCAAGGGGTCG | 372 |
| ASO_ | | | |
| 0353 | | | |
| MTx_ | 73 | TGTCATGGAGAGTGCAAGGGGTCGC | 373 |
| ASO_ | | | |
| 0354 | | | |
| MTx_ | 74 | GTCATGGAGAGTGCAAGGGGTCGCC | 374 |
| ASO_ | | | |
| 0355 | | | |
| MTx_ | 75 | TCATGGAGAGTGCAAGGGGTCGCCG | 375 |
| ASO_ | | | |
| 0356 | | | |
| MTx_ | 76 | CATGGAGAGTGCAAGGGGTCGCCGG | 376 |
| ASO_ | | | |
| 0357 | | | |
| MTx_ | 77 | ATGGAGAGTGCAAGGGGTCGCCGGG | 377 |
| ASO_ | | | |
| 0358 | | | |

### Example 3: Antisense Oligonucleotide Screening

Antisense oligonucleotides (ASOs) were designed to target the cryptic exon of UNC13A transcript (Table 7A). ASOs 1-45 (SEQ ID NOS:423-467) of Table 7B are 18mers tiling the 5′ end of the cryptic exon containing the splice acceptor region (SEQ ID NO:641) with 3 nucleotide spacing. ASOs 121-142 (SEQ ID NOS:468-489) of Table 7B are 18mers tiling the 5′ end of the cryptic exon with 1 nucleotide spacing. ASOs 248-280 (SEQ ID NOS:490-522) of Table 7B are 18mers tiling the 3′ end of the cryptic exon containing the splice donor region (SEQ ID NO:642) with 3 nucleotide spacing. The genomic coordinates of the ASOs are set forth as follows: 5′end of cryptic exon: chrl9:17,642,491-17,642,641; 3′end of cryptic exon: chrl9:17,642,363-17,642,470. ASOs with 2′MOE modifications targeting the cryptic exon of UNC13A transcript were synthesized (Table 7B) and delivered to cultured iPSC-derived motor neurons (MNs) at a concentration of 3 mM by free uptake. Motor neurons were cultured in the presence of UNC13A-specific ASOs as well as three non-targeting ASOs for two days followed by introduction of lentivirus delivering either a scrambled or TDP-43 targeting shRNA. The cells were cultured for an additional seven days post-lentiviral infection, followed by mRNA isolation. mRNA were reverse transcribed into cDNA and subjected to qPCR with primers/probes specific for UNC13A cryptic exon inclusion (FIGS. 19A-19B), in addition to primers/probes targeting properly spliced UNC13A (FIGS. 19C-19D). Regions where active ASOs reduced cryptic exon inclusion while increasing total UNC13A RNA levels were identified (ASOs in 5′ splice acceptor region: ASOs 1-10 and 17-21 corresponding to SEQ ID NOS:423-432 and 439-443; ASOs in 3′ splice donor region: ASOs 249-256, 260-265, and 271-272 corresponding to SEQ ID NOS: 491-498, 502-507, and 513-514, respectively. 21mer ASOs were designed to further tile these regions (Table 8B). ASOs 306-354 (SEQ ID NOS:523-571) of Table 8B are 21mers tiling the 5′ end of the cryptic exon (SEQ ID NO:643) with 1 nucleotide spacing. ASOs 355-423 (SEQ ID NOS:572-640) of Table 8B are 21mers tiling the 3′ end of the cryptic exon (SEQ TD NO:644) with 1 nucleotide spacing.

TABLE 7A
UNC13A Cryptic Exon Targeted Regions

| | Tiling | |
|---|---|---|
| Name | Coordinates | Target Sequence |
| Cryptic | hg38 chr19: | TCCAGGTTGACTCTCACTACTCATC |
| Exon | 17,642,640- | ATCAGGTTCTTCCTTCTATTCCAGC |
| Splice | 17,642,491 | CCTAACCACTCAGGATTGGGCCGTT |
| Acceptor | | TGTGTCTGGGTATGTCTCTTCCAGC |
| | | TGCCTGGGTTTCCTGGAAAGAACTC |
| | | TTATCCCCAGGAACTAGTTTGTTGA |
| | | [SEQ ID NO: 641] |
| Cryptic | hg38 chr19 | AACTAGTTTGTTGAATAAATGCTGG |
| Exon | 17,642,504- | TGAATGAATGAATGATTGAACAGA |
| Splice | 17,642,391 | TGAATGAGTGATGAGTAGATAAAA |
| Donor | | GGATGGATGGAGAGATGGGTGAGT |
| | | ACATGGATGGATAGATG |
| | | [SEQ ID NO: 642] |

TABLE 7B
18mer Antisense Oligonucleotides Targeting
UNC13A Cryptic Exon

| | | | SEQ |
|---|---|---|---|
| | | Nucleotide | ID |
| Name | Target | Sequence | NO: |
| MTx_ASO_1 | UNC13A | TCAACAAACTAGTTCCTG | 423 |
| MTx_ASO_2 | UNC13A | ACAAACTAGTTCCTGGGG | 424 |
| MTx_ASO_3 | UNC13A | AACTAGTTCCTGGGGATA | 425 |
| MTx_ASO_4 | UNC13A | TAGTTCCTGGGGATAAGA | 426 |
| MTx_ASO_5 | UNC13A | TTCCTGGGGATAAGAGTT | 427 |
| MTx_ASO_6 | UNC13A | CTGGGGATAAGAGTTCTT | 428 |
| MTx_ASO_7 | UNC13A | GGGATAAGAGTTCTTTCC | 429 |
| MTx_ASO_8 | UNC13A | ATAAGAGTTCTTTCCAGG | 430 |
| MTx_ASO_9 | UNC13A | AGAGTTCTTTCCAGGAAA | 431 |
| MTx_ASO_10 | UNC13A | GTTCTTTCCAGGAAACCC | 432 |
| MTx_ASO_11 | UNC13A | CTTTCCAGGAAACCCAGG | 433 |
| MTx_ASO_12 | UNC13A | TCCAGGAAACCCAGGCAG | 434 |
| MTx_ASO_13 | UNC13A | AGGAAACCCAGGCAGCTG | 435 |
| MTx_ASO_14 | UNC13A | AAACCCAGGCAGCTGGAA | 436 |
| MTx_ASO_15 | UNC13A | CCCAGGCAGCTGGAAGAG | 437 |
| MTx_ASO_16 | UNC13A | AGGCAGCTGGAAGAGACA | 438 |
| MTx_ASO_17 | UNC13A | CAGCTGGAAGAGACATAC | 439 |
| MTx_ASO_18 | UNC13A | CTGGAAGAGACATACCCA | 440 |
| MTx_ASO_19 | UNC13A | GAAGAGACATACCCAGAC | 441 |
| MTx_ASO_20 | UNC13A | GAGACATACCCAGACACA | 442 |
| MTx_ASO_21 | UNC13A | ACATACCCAGACACAAAC | 443 |
| MTx_ASO_22 | UNC13A | TACCCAGACACAAACGGC | 444 |
| MTx_ASO_23 | UNC13A | CCAGACACAAACGGCCCA | 445 |
| MTx_ASO_24 | UNC13A | GACACAAACGGCCCAATC | 446 |
| MTx_ASO_25 | UNC13A | ACAAACGGCCCAATCCTG | 447 |
| MTx_ASO_26 | UNC13A | AACGGCCCAATCCTGAGT | 448 |
| MTx_ASO_27 | UNC13A | GGCCCAATCCTGAGTGGT | 449 |
| MTx_ASO_28 | UNC13A | CCAATCCTGAGTGGTTAG | 450 |
| MTx_ASO_29 | UNC13A | ATCCTGAGTGGTTAGGGC | 451 |
| MTx_ASO_30 | UNC13A | CTGAGTGGTTAGGGCTGG | 452 |
| MTx_ASO_31 | UNC13A | AGTGGTTAGGGCTGGAAT | 453 |
| MTx_ASO_32 | UNC13A | GGTTAGGGCTGGAATAGA | 454 |
| MTx_ASO_33 | UNC13A | TAGGGCTGGAATAGAAGG | 455 |
| MTx_ASO_34 | UNC13A | GGCTGGAATAGAAGGAAG | 456 |
| MTx_ASO_35 | UNC13A | TGGAATAGAAGGAAGAAC | 457 |
| MTx_ASO_36 | UNC13A | AATAGAAGGAAGAACCTG | 458 |
| MTx_ASO_37 | UNC13A | AGAAGGAAGAACCTGATG | 459 |
| MTx_ASO_38 | UNC13A | AGGAAGAACCTGATGATG | 460 |
| MTx_ASO_39 | UNC13A | AAGAACCTGATGATGAGT | 461 |
| MTx_ASO_40 | UNC13A | AACCTGATGATGAGTAGT | 462 |
| MTx_ASO_41 | UNC13A | CTGATGATGAGTAGTGAG | 463 |
| MTX_ASO_42 | UNC13A | ATGATGAGTAGTGAGAGT | 464 |
| MTx_ASO_43 | UNC13A | ATGAGTAGTGAGAGTCAA | 465 |
| MTx_ASO_44 | UNC13A | AGTAGTGAGAGTCAACCT | 466 |
| MTx_ASO_45 | UNC13A | AGTGAGAGTCAACCTGGA | 467 |
| MTx_ASO_121 | UNC13A | GGCAGCTGGAAGAGACAT | 468 |
| MTx_ASO_122 | UNC13A | GCAGCTGGAAGAGACATA | 469 |
| MTx_ASO_123 | UNC13A | CAGCTGGAAGAGACATAC | 470 |
| MTx_ASO_124 | UNC13A | AGCTGGAAGAGACATACC | 471 |
| MTx_ASO_125 | UNC13A | GCTGGAAGAGACATACCC | 472 |
| MTx_ASO_126 | UNC13A | CTGGAAGAGACATACCCA | 473 |
| MTx_ASO_127 | UNC13A | TGGAAGAGACATACCCAG | 474 |
| MTx_ASO_128 | UNC13A | GGAAGAGACATACCCAGA | 475 |
| MTx_ASO_129 | UNC13A | GAAGAGACATACCCAGAC | 476 |
| MTx_ASO_130 | UNC13A | AAGAGACATACCCAGACA | 477 |
| MTx_ASO_131 | UNC13A | AGAGACATACCCAGACAC | 478 |
| MTx_ASO_132 | UNC13A | GGCAGCTGGAAGAGACAT | 479 |
| MTx_ASO_133 | UNC13A | GCAGCTGGAAGAGACATA | 480 |
| MTx_ASO_134 | UNC13A | CAGCTGGAAGAGACATAC | 481 |
| MTx_ASO_135 | UNC13A | AGCTGGAAGAGACATACC | 482 |
| MTx_ASO_136 | UNC13A | GCTGGAAGAGACATACCC | 483 |
| MTx_ASO_137 | UNC13A | CTGGAAGAGACATACCCA | 484 |
| MTx_ASO_138 | UNC13A | TGGAAGAGACATACCCAG | 485 |
| MTx_ASO_139 | UNC13A | GGAAGAGACATACCCAGA | 486 |
| MTx_ASO_140 | UNC13A | GAAGAGACATACCCAGAC | 487 |
| MTx_ASO_141 | UNC13A | AAGAGACATACCCAGACA | 488 |
| MTx_ASO_142 | UNC13A | AGAGACATACCCAGACAC | 489 |
| MTx_ASO_248 | UNC13A | CATCTATCCATCCATGTA | 490 |
| MTx_ASO_249 | UNC13A | CTATCCATCCATGTACTC | 491 |
| MTx_ASO_250 | UNC13A | TCCATCCATGTACTCACC | 492 |
| MTx_ASO_251 | UNC13A | ATCCATGTACTCACCCAT | 493 |
| MTx_ASO_252 | UNC13A | CATGTACTCACCCATCTC | 494 |
| MTx_ASO_253 | UNC13A | GTACTCACCCATCTCTCC | 495 |
| MTx_ASO_254 | UNC13A | CTCACCCATCTCTCCATC | 496 |
| MTx_ASO_255 | UNC13A | ACCCATCTCTCCATCCAT | 497 |
| MTx_ASO_256 | UNC13A | CATCTCTCCATCCATCCT | 498 |
| MTx_ASO_257 | UNC13A | CTCTCCATCCATCCTTTT | 499 |
| MTx_ASO_258 | UNC13A | TCCATCCATCCTTTTATC | 500 |
| MTx_ASO_259 | UNC13A | ATCCATCCTTTTATCTAC | 501 |
| MTx_ASO_260 | UNC13A | CATCCTTTTATCTACTCA | 502 |
| MTx_ASO_261 | UNC13A | CCTTTTATCTACTCATCA | 503 |
| MTx_ASO_262 | UNC13A | TTTATCTACTCATCACTC | 504 |
| MTx_ASO_263 | UNC13A | ATCTACTCATCACTCATT | 505 |
| MTx_ASO_264 | UNC13A | TACTCATCACTCATTCAT | 506 |
| MTx_ASO_265 | UNC13A | TCATCACTCATTCATCTG | 507 |
| MTx_ASO_266 | UNC13A | TCACTCATTCATCTGTTC | 508 |
| MTx_ASO_267 | UNC13A | CTCATTCATCTGTTCAAT | 509 |
| MTx_ASO_268 | UNC13A | ATTCATCTGTTCAATCAT | 510 |
| MTx_ASO_269 | UNC13A | CATCTGTTCAATCATTCA | 511 |
| MTx_ASO_270 | UNC13A | CTGTTCAATCATTCATTC | 512 |
| MTx_ASO_271 | UNC13A | TTCAATCATTCATTCATT | 513 |
| MTx_ASO_272 | UNC13A | AATCATTCATTCATTCAC | 514 |
| MTx_ASO_273 | UNC13A | CATTCATTCATTCACCAG | 515 |
| MTx_ASO_274 | UNC13A | TCATTCATTCACCAGCAT | 516 |
| MTx_ASO_275 | UNC13A | TTCATTCACCAGCATTTA | 517 |
| MTx_ASO_276 | UNC13A | ATTCACCAGCATTTATTC | 518 |
| MTx_ASO_277 | UNC13A | CACCAGCATTTATTCAAC | 519 |
| MTx_ASO_278 | UNC13A | CAGCATTTATTCAACAAA | 520 |
| MTx_ASO_279 | UNC13A | CATTTATTCAACAAACTA | 521 |
| MTx_ASO_280 | UNC13A | TTATTCAACAAACTAGTT | 522 |

TABLE 8A
UNC13A Cryptic Exon Targeted Regions

| | Tiling | |
|---|---|---|
| Name | Coordinates | Target Sequence |
| Cryptic | hg38 chr19 | GTCTGGGTATGTCTCTTCCAGCTGC |
| Exon | 17,642,562- | CTGGGTTTCCTGGAAAGAACTCTTA |
| Splice | 17,642,494 | TCCCCAGGAACTAGTTTGT |
| Acceptor | | [SEQ ID NO: 643] |
| Cryptic | hg38 chr19 | TGAATGAATGAATGATTGAACAGA |
| Exon | 17,642,479- | TGAATGAGTGATGAGTAGATAAAA |
| Splice | 17,642,391 | GGATGGATGGAGAGATGGGTGAGT |
| Donor | | ACATGGATGGATAGATG |
| | | [SEQ ID NO: 644] |

TABLE 8B
21mer Antisense Oligonucleotides Targeting
UNC13A Spaced 1bp Apart

| | | | SEQ |
|---|---|---|---|
| | | | ID |
| Name | Target | Nucleotide Sequence | NO: |
| MTx_ASO_306 | UNC13A | ACAAACTAGTTCCTGGGGATA | 523 |
| MTx_ASO_307 | UNC13A | CAAACTAGTTCCTGGGGATAA | 524 |
| MTx_ASO_308 | UNC13A | AAACTAGTTCCTGGGGATAAG | 525 |
| MTx_ASO_309 | UNC13A | AACTAGTTCCTGGGGATAAGA | 526 |
| MTx_ASO_310 | UNC13A | ACTAGTTCCTGGGGATAAGAG | 527 |
| MTx_ASO_311 | UNC13A | CTAGTTCCTGGGGATAAGAGT | 528 |
| MTx_ASO_312 | UNC13A | TAGTTCCTGGGGATAAGAGTT | 529 |
| MTx_ASO_313 | UNC13A | AGTTCCTGGGGATAAGAGTTC | 530 |
| MTx_ASO_314 | UNC13A | GTTCCTGGGGATAAGAGTTCT | 531 |
| MTx_ASO_315 | UNC13A | TTCCTGGGGATAAGAGTTCTT | 532 |
| MTx_ASO_316 | UNC13A | TCCTGGGGATAAGAGTTCTTT | 533 |
| MTx_ASO_317 | UNC13A | CCTGGGGATAAGAGTTCTTTC | 534 |
| MTx_ASO_318 | UNC13A | CTGGGGATAAGAGTTCTTTCC | 535 |
| MTx_ASO_319 | UNC13A | TGGGGATAAGAGTTCTTTCCA | 536 |
| MTx_ASO_320 | UNC13A | GGGGATAAGAGTTCTTTCCAG | 537 |
| MTx_ASO_321 | UNC13A | GGGATAAGAGTTCTTTCCAGG | 538 |
| MTx_ASO_322 | UNC13A | GGATAAGAGTTCTTTCCAGGA | 539 |
| MTx_ASO_323 | UNC13A | GATAAGAGTTCTTTCCAGGAA | 540 |
| MTx_ASO_324 | UNC13A | ATAAGAGTTCTTTCCAGGAAA | 541 |
| MTx_ASO_325 | UNC13A | TAAGAGTTCTTTCCAGGAAAC | 542 |
| MTx_ASO_326 | UNC13A | AAGAGTTCTTTCCAGGAAACC | 543 |
| MTx_ASO_327 | UNC13A | AGAGTTCTTTCCAGGAAACCC | 544 |
| MTx_ASO_328 | UNC13A | GAGTTCTTTCCAGGAAACCCA | 545 |
| MTx_ASO_329 | UNC13A | AGTTCTTTCCAGGAAACCCAG | 546 |
| MTx_ASO_330 | UNC13A | GTTCTTTCCAGGAAACCCAGG | 547 |
| MTx_ASO_331 | UNC13A | TTCTTTCCAGGAAACCCAGGC | 548 |
| MTx_ASO_332 | UNC13A | TCTTTCCAGGAAACCCAGGCA | 549 |
| MTx_ASO_333 | UNC13A | CTTTCCAGGAAACCCAGGCAG | 550 |
| MTx_ASO_334 | UNC13A | TTTCCAGGAAACCCAGGCAGC | 551 |
| MTx_ASO_335 | UNC13A | TTCCAGGAAACCCAGGCAGCT | 552 |
| MTx_ASO_336 | UNC13A | TCCAGGAAACCCAGGCAGCTG | 553 |
| MTx_ASO_337 | UNC13A | CCAGGAAACCCAGGCAGCTGG | 554 |
| MTx_ASO_338 | UNC13A | CAGGAAACCCAGGCAGCTGGA | 555 |
| MTx_ASO_339 | UNC13A | AGGAAACCCAGGCAGCTGGAA | 556 |
| MTx_ASO_340 | UNC13A | GGAAACCCAGGCAGCTGGAAG | 557 |
| MTx_ASO_341 | UNC13A | GAAACCCAGGCAGCTGGAAGA | 558 |
| MTx_ASO_342 | UNC13A | AAACCCAGGCAGCTGGAAGAG | 559 |
| MTx_ASO_343 | UNC13A | AACCCAGGCAGCTGGAAGAGA | 560 |
| MTx_ASO_344 | UNC13A | ACCCAGGCAGCTGGAAGAGAC | 561 |
| MTx_ASO_345 | UNC13A | CCCAGGCAGCTGGAAGAGACA | 562 |
| MTx_ASO_346 | UNC13A | CCAGGCAGCTGGAAGAGACAT | 563 |
| MTx_ASO_347 | UNC13A | CAGGCAGCTGGAAGAGACATA | 564 |
| MTx_ASO_348 | UNC13A | AGGCAGCTGGAAGAGACATAC | 565 |
| MTx_ASO_349 | UNC13A | GGCAGCTGGAAGAGACATACC | 566 |
| MTx_ASO_350 | UNC13A | GCAGCTGGAAGAGACATACCC | 567 |
| MTx_ASO_351 | UNC13A | CAGCTGGAAGAGACATACCCA | 568 |
| MTx_ASO_352 | UNC13A | AGCTGGAAGAGACATACCCAG | 569 |
| MTx_ASO_353 | UNC13A | GCTGGAAGAGACATACCCAGA | 570 |
| MTx_ASO_354 | UNC13A | CTGGAAGAGACATACCCAGAC | 571 |
| MTx_ASO_355 | UNC13A | CATCTATCCATCCATGTACTC | 572 |
| MTx_ASO_356 | UNC13A | ATCTATCCATCCATGTACTCA | 573 |
| MTx_ASO_357 | UNC13A | TCTATCCATCCATGTACTCAC | 574 |
| MTx_ASO_358 | UNC13A | CTATCCATCCATGTACTCACC | 575 |
| MTx_ASO_359 | UNC13A | TATCCATCCATGTACTCACCC | 576 |
| MTx_ASO_360 | UNC13A | ATCCATCCATGTACTCACCCA | 577 |
| MTx_ASO_361 | UNC13A | TCCATCCATGTACTCACCCAT | 578 |
| MTx_ASO_362 | UNC13A | CCATCCATGTACTCACCCATC | 579 |
| MTx_ASO_363 | UNC13A | CATCCATGTACTCACCCATCT | 580 |
| MTx_ASO_364 | UNC13A | ATCCATGTACTCACCCATCTC | 581 |
| MTx_ASO_365 | UNC13A | TCCATGTACTCACCCATCTCT | 582 |
| MTx_ASO_366 | UNC13A | CCATGTACTCACCCATCTCTC | 583 |
| MTx_ASO_367 | UNC13A | CATGTACTCACCCATCTCTCC | 584 |
| MTx_ASO_368 | UNC13A | ATGTACTCACCCATCTCTCCA | 585 |
| MTx_ASO_369 | UNC13A | TGTACTCACCCATCTCTCCAT | 586 |
| MTx_ASO_370 | UNC13A | GTACTCACCCATCTCTCCATC | 587 |
| MTx_ASO_371 | UNC13A | TACTCACCCATCTCTCCATCC | 588 |
| MTx_ASO_372 | UNC13A | ACTCACCCATCTCTCCATCCA | 589 |
| MTx_ASO_373 | UNC13A | CTCACCCATCTCTCCATCCAT | 590 |
| MTx_ASO_374 | UNC13A | TCACCCATCTCTCCATCCATC | 591 |
| MTx_ASO_375 | UNC13A | CACCCATCTCTCCATCCATCC | 592 |
| MTx_ASO_376 | UNC13A | ACCCATCTCTCCATCCATCCT | 593 |
| MTx_ASO_377 | UNC13A | CCCATCTCTCCATCCATCCTT | 594 |
| MTx_ASO_378 | UNC13A | CCATCTCTCCATCCATCCTTT | 595 |
| MTx_ASO_379 | UNC13A | CATCTCTCCATCCATCCTTTT | 596 |
| MTx_ASO_380 | UNC13A | ATCTCTCCATCCATCCTTTTA | 597 |
| MTx_ASO_381 | UNC13A | TCTCTCCATCCATCCTTTTAT | 598 |
| MTx_ASO_382 | UNC13A | CTCTCCATCCATCCTTTTATC | 599 |
| MTx_ASO_383 | UNC13A | TCTCCATCCATCCTTTTATCT | 600 |
| MTx_ASO_384 | UNC13A | CTCCATCCATCCTTTTATCTA | 601 |
| MTx_ASO_385 | UNC13A | TCCATCCATCCTTTTATCTAC | 602 |
| MTx_ASO_386 | UNC13A | CCATCCATCCTTTTATCTACT | 603 |
| MTx_ASO_387 | UNC13A | CATCCATCCTTTTATCTACTC | 604 |
| MTx_ASO_388 | UNC13A | ATCCATCCTTTTATCTACTCA | 605 |
| MTx_ASO_389 | UNC13A | TCCATCCTTTTATCTACTCAT | 606 |
| MTx_ASO_390 | UNC13A | CCATCCTTTTATCTACTCATC | 607 |
| MTx_ASO_391 | UNC13A | CATCCTTTTATCTACTCATCA | 608 |
| MTx_ASO_392 | UNC13A | ATCCTTTTATCTACTCATCAC | 609 |
| MTx_ASO_393 | UNC13A | TCCTTTTATCTACTCATCACT | 610 |
| MTx_ASO_394 | UNC13A | CCTTTTATCTACTCATCACTC | 611 |
| MTx_ASO_395 | UNC13A | CTTTTATCTACTCATCACTCA | 612 |
| MTx_ASO_396 | UNC13A | TTTTATCTACTCATCACTCAT | 613 |
| MTx_ASO_397 | UNC13A | TTTATCTACTCATCACTCATT | 614 |
| MTx_ASO_398 | UNC13A | TTATCTACTCATCACTCATTC | 615 |
| MTx_ASO_399 | UNC13A | TATCTACTCATCACTCATTCA | 616 |
| MTx_ASO_400 | UNC13A | ATCTACTCATCACTCATTCAT | 617 |
| MTx_ASO_401 | UNC13A | TCTACTCATCACTCATTCATC | 618 |
| MTx_ASO_402 | UNC13A | CTACTCATCACTCATTCATCT | 619 |
| MTx_ASO_403 | UNC13A | TACTCATCACTCATTCATCTG | 620 |
| MTx_ASO_404 | UNC13A | ACTCATCACTCATTCATCTGT | 621 |
| MTx_ASO_405 | UNC13A | CTCATCACTCATTCATCTGTT | 622 |
| MTx_ASO_406 | UNC13A | TCATCACTCATTCATCTGTTC | 623 |
| MTx_ASO_407 | UNC13A | CATCACTCATTCATCTGTTCA | 624 |
| MTx_ASO_408 | UNC13A | ATCACTCATTCATCTGTTCAA | 625 |
| MTx_ASO_409 | UNC13A | TCACTCATTCATCTGTTCAAT | 626 |
| MTx_ASO_410 | UNC13A | CACTCATTCATCTGTTCAATC | 627 |
| MTx_ASO_411 | UNC13A | ACTCATTCATCTGTTCAATCA | 628 |
| MTx_ASO_412 | UNC13A | CTCATTCATCTGTTCAATCAT | 629 |
| MTx_ASO_413 | UNC13A | TCATTCATCTGTTCAATCATT | 630 |
| MTx_ASO_414 | UNC13A | CATTCATCTGTTCAATCATTC | 631 |
| MTx_ASO_415 | UNC13A | ATTCATCTGTTCAATCATTCA | 632 |
| MTx_ASO_416 | UNC13A | TTCATCTGTTCAATCATTCAT | 633 |
| MTx_ASO_417 | UNC13A | TCATCTGTTCAATCATTCATT | 634 |
| MTx_ASO_418 | UNC13A | CATCTGTTCAATCATTCATTC | 635 |
| MTx_ASO_419 | UNC13A | ATCTGTTCAATCATTCATTCA | 636 |
| MTx_ASO_420 | UNC13A | TCTGTTCAATCATTCATTCAT | 637 |
| MTx_ASO_421 | UNC13A | CTGTTCAATCATTCATTCATT | 638 |
| MTx_ASO_422 | UNC13A | TGTTCAATCATTCATTCATTC | 639 |
| MTx_ASO_423 | UNC13A | GTTCAATCATTCATTCATTCA | 640 |

TABLE 9
Subregions of cryptic exon targeted by active
18mer ASOs that reduced cryptic exon inclusion
while increasing total UNC13A RNA levels

| | | SEQ |
|---|---|---|
| | | ID |
| Sequence | | NO: |
| Description | Nucleotide Sequence | # |
| ASOs 1-10 | TCAACAAACTAGTTCCTGGGGATAAGA | 645 |
| compiled | GTTCTTTCCAGGAAACCC | |
| sequence | | |
| ASOs 1-10 | GGGTTTCCTGGAAAGAACTCTTATCCC | 650 |
| compiled | CAGGAACTAGTTTGTTGA | |
| sequence | | |
| reverse | | |
| complement | | |
| ASOs 17-21 | CAGCTGGAAGAGACATACCCAGACACA | 646 |
| compiled | AAC | |
| sequence | | |
| ASOs 17-21 | GTTTGTGTCTGGGTATGTCTCTTCCAG | 651 |
| compiled | CTG | |
| sequence | | |
| reverse | | |
| complement | | |
| ASOs 249-256 | CTATCCATCCATGTACTCACCCATCTC | 647 |
| compiled | TCCATCCATCCT | |
| sequence | | |
| ASOs 249-256 | AGGATGGATGGAGAGATGGGTGAGTAC | 652 |
| compiled | ATGGATGGATAG | |
| sequence | | |
| reverse | | |
| complement | | |
| ASOs 260-265 | CATCCTTTTATCTACTCATCACTCATT | 648 |
| compiled | CATCTG | |
| sequence | | |
| ASOs 260-265 | CAGATGAATGAGTGATGAGTAGATAAA | 653 |
| compiled | AGGATG | |
| sequence | | |
| reverse | | |
| complement | | |
| ASOs 271-272 | TTCAATCATTCATTCATTCAC | 649 |
| compiled | | |
| sequence | | |
| ASOs 271-272 | GTGAATGAATGAATGATTGAA | 654 |
| compiled | | |
| sequence | | |
| reverse | | |
| complement | | |

### REFERENCES

1. C. Lagier-Tourenne, M. Polymenidou, D. W. Cleveland, TDP-43 and FUS/TLS: Emerging roles in RNA processing and neurodegeneration. Hum. Mol. Genet. (2010), doi:10.1093/hmg/ddql37.
2. M. Polymenidou, C. Lagier-Tourenne, K. R. Hutt, S. C. Huelga, J. Moran, T. Y. Liang, S. C. Ling, E. Sun, E. Wancewicz, C. Mazur, H. Kordasiewicz, Y. Sedaghat, J. P. Donohue, L. Shiue, C. F. Bennett, G. W. Yeo, D. W. Cleveland, Long pre-mRNA depletion and RNA missplicing contribute to neuronal vulnerability from loss of TDP-43. Nat. Neurosci. (2011), doi:10.1038/nn.2779.
3. J. R. Tollervey, T. Curk, B. Rogelj, M. Briese, M. Cereda, M. Kayikci, J. König, T. Hortobágyi, A. L. Nishimura, V. \tilde{Z}upunski, R. Patani, S. Chandran, G. Rot, B. Zupan, C. E. Shaw, J. Ule, Characterizing the RNA targets and position-dependent splicing regulation by TDP-43. Nat. Neurosci. (2011), doi:10.1038/nn.2778.
4. J. P. Ling, O. Pletnikova, J. C. Troncoso, P. C. Wong, TDP-43 repression of nonconserved cryptic exons is compromised in ALS-FTD. Science (80-.). (2015), doi:10.1126/science.aab0983.
5. A. Donde, M. Sun, J. P. Ling, K. E. Braunstein, B. Pang, X. Wen, X. Cheng, L. Chen, P. C. Wong, Splicing repression is a major function of TDP-43 in motor neurons. Acta Neuropathol. (2019), doi:10.1007/s00401-019-02042-8.
6. M. Sun, W. Bell, K. D. LaClair, J. P. Ling, H. Han, Y. Kageyama, O. Pletnikova, J. C. Troncoso, P. C. Wong, L. L. Chen, Cryptic exon incorporation occurs in Alzheimer's brain lacking TDP-43 inclusion but exhibiting nuclear clearance of TDP-43. Acta Neuropathol. (2017), doi:10.1007/s00401-017-1701-2.
7. Y. H. Jeong, J. P. Ling, S. Z. Lin, A. N. Donde, K. E. Braunstein, E. Majounie, B. J. Traynor, K. D. LaClair, T. E. Lloyd, P. C. Wong, Tdp-43 cryptic exons are highly variable between cell types. Mol. Neurodegener. (2017), doi:10.1 186/s13024-016-0144-x.
8. J. R. Klim, L. A. Williams, F. Limone, I. Guerra San Juan, B. N. Davis-Dusenbery, D. A. Mordes, A. Burberry, M. J. Steinbaugh, K. K. Gamage, R. Kirchner, R. Moccia, S. H. Cassel, K. Chen, B. J. Wainger, C. J. Woolf, K. Eggan, ALS-implicated protein TDP-43 sustains levels of STMN2, a mediator of motor neuron growth and repair. Nat. Neurosci. (2019), doi:10.1038/s41593-018-0300-4.
9. Z. Melamed, J. López-Erauskin, M. W. Baughn, O. Zhang, K. Drenner, Y. Sun, F. Freyermuth, M. A. McMahon, M. S. Beccari, J. W. Artates, T. Ohkubo, M. Rodriguez, N. Lin, D. Wu, C. F. Bennett, F. Rigo, S. Da Cruz, J. Ravits, C. Lagier-Tourenne, D. W. Cleveland, Premature polyadenylation-mediated loss of stathmin-2 is a hallmark of TDP-43-dependent neurodegeneration. Nat. Neurosci. (2019), doi:10.1038/s41593-018-0293-z.
10. M. Prudencio, J. Humphrey, S. Pickles, A. L. Brown, S. E. Hill, J. M. Kachergus, J. Shi, M. G. Heckman, M. R. Spiegel, C. Cook, Y. Song, M. Yue, L. M. Daughrity, Y. Carlomagno, K. Jansen-West, C. F. de Castro, M. DeTure, S. Koga, Y. C. Wang, P. Sivakumar, C. Bodo, A. Candalija, K. Talbot, B. T. Selvaraj, K. Burr, S. Chandran, J. Newcombe, T. Lashley, I. Hubbard, D. Catalano, D. Kim, N. Propp, S. Fennessey, D. Fagegaltier, H. Phatnani, M. Secrier, E. M. C. Fisher, B. Oskarsson, M. van Blitterswijk, R. Rademakers, N. R. Graff-Radford, B. F. Boeve, D. S. Knopman, R. C. Petersen, K. A. Josephs, E. Aubrey Thompson, T. Raj, M. Ward, D. W. Dickson, T. F. Gendron, P. Fratta, L. Petrucelli, Truncated stathmin-2 is a marker of TDP-43 pathology in frontotemporal dementia. J. Clin. Invest. (2020), doi:10.1172/JCI139741.
11. E. Y. Liu, J. Russ, C. P. Cali, J. M. Phan, A. Amlie-Wolf, E. B. Lee, Loss of Nuclear TDP-43 Is Associated with Decondensation of LINE Retrotransposons. Cell Rep. (2019), doi:10.1016/j.celrep.2019.04.003.
12. J. Vaquero-Garcia, A. Barrera, M. R. Gazzara, J. Gonzalez-Vallinas, N. F. Lahens, J. B. Hogenesch, K. W. Lynch, Y. Barash, A new view of transcriptome complexity and regulation through the lens of local splicing variations. Elife (2016), doi:10.7554/eLife.11752.
13. Y. I. Li, D. A. Knowles, J. Humphrey, A. N. Barbeira, S. P. Dickinson, H. K. Im, J. K. Pritchard, Annotation-free quantification of RNA splicing using LeafCutter. Nat. Genet. (2018), doi:10.1038/s41588-017-0004-9.
14. A. Shiga, T. Ishihara, A. Miyashita, M. Kuwabara, T. Kato, N. Watanabe, A. Yamahira, C. Kondo, A. Yokoseki, M. Takahashi, R. Kuwano, A. Kakita, M. Nishizawa, H. Takahashi, O. Onodera, Alteration of POLDIP3 splicing associated with loss of function of TDP-43 in tissues affected with ALS. PLoS One (2012), doi:10.1371/journal.pone.0043120.
15. L. J. Carithers, K. Ardlie, M. Barcus, P. A. Branton, A. Britton, S. A. Buia, C. C. Compton, D. S. Deluca, J. Peter-Demchok, E. T. Gelfand, P. Guan, G. E. Korzeniewski, N. C. Lockhart, C. A. Rabiner, A. K. Rao, K. L. Robinson, N. V. Roche, S. J. Sawyer, A. V. Segre, C. E. Shive, A. M. Smith, L. H. Sobin, A. H. Undale, K. M. Valentino, J. Vaught, T. R. Young, H. M. Moore, L. Barker, M. Basile, A. Battle, J. Boyer, D. Bradbury, J. P. Bridge, A. Brown, R. Burges, C. Choi, D. Colantuoni, N. Cox, E. T. Dermitzakis, L. K. Derr, M. J. Dinsmore, K. Erickson, J. Fleming, T. Flutre, B. A. Foster, E. R. Gamazon, G. Getz, B. M. Gillard, R. Guigó, K. W. Hambright, P. Hariharan, R. Hasz, H. K. Im, S. Jewell, E. Karasik, M. Kellis, P. Kheradpour, S. Koester, D. Koller, A. Konkashbaev, T. Lappalainen, R. Little, J. Liu, E. Lo, J. T. Lonsdale, C. Lu, D. G. MacArthur, H. Magazine, J. B. Maller, Y. Marcus, D. C. Mash, M. I McCarthy, J. McLean, B. Mestichelli, M. Miklos, J. Monlong, M. Mosavel, M. T. Moser, S. Mostafavi, D. L. Nicolae, J. Pritchard, L. Qi, K. Ramsey, M. A. Rivas, B. E. Robles, D. C. Rohrer, M. Salvatore, M. Sammeth, J. Seleski, S. Shad, L. A. Siminoff, M. Stephens, J. Struewing, T. Sullivan, S. Sullivan, J. Syron, D. Tabor, M. Taherian, J. Tejada, G. F. Temple, J. A. Thomas, A. W. Thomson, D. Tidwell, H. M. Traino, Z. Tu, D. R. Valley, S. Volpi, G. D. Walters, L. D. Ward, X. Wen, W. Winckler, S. Wu, J. Zhu, A. Abdallah, A. Addington, J. M. Anderson, P. K. Bender, M. Cosentino, N. Diaz-Mayoral, T. Engel, F. Garci, A. Green, T. Hammond, K. Jaffe, J. Keen, M. Kennedy, P. Kigonya, B. Lander, S. Nampally, C. Ny, J. Robb, V. Santhanum, N. Sharopova, S. Singh, C. Soria, A. Sturcke, S. Sukari, E. J. Thomson, M. Tomaszewski, C. Trowbridge, F. Udoye, D. Vanscoy, N. Vatanian, E. L. Wilder, P. Williams, A Novel Approach to High-Quality Postmortem Tissue Procurement: The GTEx Project. Biopreserv. Biobank. (2015), doi:10.1089/bio.2015.0032.
16. S. Brenner, The genetics of Caenorhabditis elegans. Genetics (1974), doi:10.1093/genetics/77.1.71.
17. N. Lipstein, N. M. Verhoeven-Duif, F. E. Michelassi, N. Calloway, P. M. Van Hasselt, K. Pienkowska, G. Van Haaften, M. M. Van Haelst, R. Van Empelen, I. Cuppen, H. C. Van Teeseling, A. M. V. Evelein, J. A. Vorstman, S. Thoms, O. Jahn, K. J. Duran, G. R. Monroe, T. A. Ryan, H. Taschenberger, J. S. Dittman, J. S. Rhee, G. Visser, J. J. Jans, N. Brose, Synaptic UNC13A protein variant causes increased neurotransmission and dyskinetic movement disorder. J. Clin. Invest. (2017), doi:10.1172/JC190259.
18. L. Deng, P. S. Kaeser, W. Xu, T. C. S0dhof, RIM proteins activate vesicle priming by reversing autoinhibitory homodimerization of munc13. Neuron (2011), doi:10.1016/j.neuron.2011.01.005.
19. I. Augustin, C. Rosenmund, T. C. Sudhof, N. Brose, Muncl3-1 is essential for fusion competence of glutamatergic synaptic vesicles. Nature (1999), doi:10.1038/22768.
20. M. A. Bohme, C. Beis, S. Reddy-Alla, E. Reynolds, M. M. Mampell, A. T. Grasskamp, J. Lutzkendorf, D. D. Bergeron, J. H. Driller, H. Babikir, F. Gottfert, I. M. Robinson, C. J. O'Kane, S. W. Hell, M. C. Wahl, U. Stelzl, B. Loll, A. M. Walter, S. J. Sigrist, Active zone scaffolds differentially accumulate Unc13 isoforms to tune Ca2+channel-vesicle coupling. Nat. Neurosci. (2016), doi:10.1038/nn.4364.
21. F. Varoqueaux, M. S. Sons, J. J. Plomp, N. Brose, Aberrant Morphology and Residual Transmitter Release at the Munc13-Deficient Mouse Neuromuscular Synapse. Mol. Cell. Biol. (2005), doi:10.1 128/mcb.25.14.5973-5984.2005.
22. M. Hasegawa, T. Arai, T. Nonaka, F. Kametani, M. Yoshida, Y. Hashizume, T. G. Beach, E. Buratti, F. Baralle, M. Morita, I. Nakano, T. Oda, K. Tsuchiya, H. Akiyama, Phosphorylated TDP-43 in frontotemporal lobar degeneration and amyotrophic lateral sclerosis. Ann. Neurol. (2008), doi:10.1002/ana.21425.
23. F. P. Diekstra, V. M. Van Deerlin, J. C. Van Swieten, A. Al-Chalabi, A. C. Ludolph, J. H. Weishaupt, O. Hardiman, J. E. Landers, R. H. Brown, M. A. Van Es, R. J. Pasterkamp, M. Koppers, P. M. Andersen, K. Estrada, F. Rivadeneira, A. Hofman, A. G. Uitterlinden, P. Van Damme, J. Melki, V. Meininger, A. Shatunov, C. E. Shaw, P. N. Leigh, P. J. Shaw, K. E. Morrison, I. Fogh, A. Chio, B. J. Traynor, D. Czell, M. Weber, P. Heutink, P. I W. De Bakker, V. Silani, W. Robberecht, L. H. Van Den Berg, J. H. Veldink, C9orf72 and UNC13A are shared risk loci for amyotrophic lateral sclerosis and frontotemporal dementia: A genome-wide meta-analysis. Ann. Neurol. (2014), doi:10.1002/ana.24198.
24. M. A. Van Es, J. H. Veldink, C. G. J. Saris, H. M. Blauw, P. W. J. Van Vught, A. Birve, R. Lemmens, H. J. Schelhaas, E. J. N. Groen, M. H. B. Huisman, A. J. Van Der Kooi, M. De Visser, C. Dahlberg, K. Estrada, F. Rivadeneira, A. Hofman, M. J. Zwarts, P. T. C. Van Doormaal, D. Rujescu, E. Strengman, I. Giegling, P. Muglia, B. Tomik, A. Slowik, A. G. Uitterlinden, C. Hendrich, S. Waibel, T. Meyer, A. C. Ludolph, J. D. Glass, S. Purcell, S. Cichon, M. M. Nöthen, H. E. Wichmann, S. Schreiber, S. H. H. M. Vermeulen, L. A. Kiemeney, J. H. J. Wokke, S. Cronin, R. L. McLaughlin, O. Hardiman, K. Fumoto, R. J. Pasterkamp, V. Meininger, J. Melki, P. N. Leigh, C. E. Shaw, J. E. Landers, A. Al-Chalabi, R. H. Brown, W. Robberecht, P. M. Andersen, R. A. Ophoff, L. H. Van Den Berg, Genome-wide association study identifies 19 pl3.3 (UNC13A) and 9p21.2 as susceptibility loci for sporadic amyotrophic lateral sclerosis. Nat. Genet. (2009), doi:10.1038/ng.442.
25. A. Nicolas, K. Kenna, A. E. Renton, N. Ticozzi, F. Faghri, R. Chia, J. A. Dominov, B. J. Kenna, M. A. Nalls, P. Keagle, A. M. Rivera, W. van Rheenen, N. A. Murphy, J. J. F. A. van Vugt, J. T. Geiger, R. van der Spek, H. A. Pliner, Shankaracharya, B. N. Smith, G. Marangi, S. D. Topp, Y. Abramzon, A. S. Gkazi, J. D. Eicher, A. Kenna, F. O. Logullo, I. Simone, G. Logroscino, F. Salvi, I. Bartolomei, G. Borghero, M. R. Murru, E. Costantino, C. Pani, R. Puddu, C. Caredda, V. Piras, S. Tranquilli, S. Cuccu, D. Corongiu, M. Melis, A. Milia, F. Marrosu, M. G. Marrosu, G. Floris, A. Cannas, M. Capasso, C. Caponnetto, G. Mancardi, P. Origone, P. Mandich, F. L. Conforti, S. Cavallaro, G. Mora, K. Marinou, R. Sideri, S. Penco, L. Mosca, C. Lunetta, G. L. Pinter, M. Corbo, N. Riva, P. Carrera, P. Volanti, J. Mandrioli, N. Fini, A. Fasano, L. Tremolizzo, A. Arosio, C. Ferrarese, F. Trojsi, G. Tedeschi, M. R. Monsurro, G. Piccirillo, C. Femiano, A. Ticca, E. Ortu, V. La Bella, R. Spataro, T. Colletti, M. Sabatelli, M. Zollino, A. Conte, M. Luigetti, S. Lattante, M. Santarelli, A. Petrucci, M. Pugliatti, A. Pirisi, L. D. Parish, P. Occhineri, F. Giannini, S. Battistini, C. Ricci, M. Benigni, T. B. Cau, D. Loi, A. Calvo, C. Moglia, M. Brunetti, M. Barberis, G. Restagno, F. Casale, G. Marrali, G. Fuda, I. Ossola, S. Cammarosano, A. Canosa, A. Ilardi, U. Manera, M. Grassano, R. Tanel, F. Pisano, L. Mazzini, S. Messina, S. D′Alfonso, L. Corrado, L. Ferrucci, M. B. Harms, D. B. Goldstein, N. A. Shneider, S. Goutman, Z. Simmons, T. M. Miller, S. Chandran, S. Pal, G. Manousakis, S. Appel, E. Simpson, L. Wang, R. H. Baloh, S. Gibson, R. S. Bedlack, D. Lacomis, D. Sareen, A. Sherman, L. Bruijn, M. Penny, C. de A. M. Moreno, S. Kamalakaran, A. S. Allen, B. E. Boone, R. Brown, J. P. Carulli, A. Chesi, W. K. Chung, E. T. Cirulli, G. M. Cooper, J. Couthouis, A. G. Day-Williams, P. A. Dion, A. D. Gitler, J. Glass, Y. Han, T. Harris, S. D. Hayes, A. L. Jones, J. Keebler, B. J. Krueger, B. N. Lasseigne, S. E. Levy, Y. F. Lu, T. Maniatis, D. McKenna-Yasek, R. M. Myers, S. Petrovski, S. M. Pulst, A. R. Raphael, J. Ravits, Z. Ren, G. A. Rouleau, P. C. Sapp, K. B. Sims, J. F. Staropoli, L. L. Waite, Q. Wang, J. R. Wimbish, W. W. Xin, H. Phatnani, J. Kwan, J. R. Broach, X. Arcila-Londono, E. B. Lee, V. M. Van Deerlin, E. Fraenkel, L. W. Ostrow, F. Baas, N. Zaitlen, J. D. Berry, A. Malaspina, P. Fratta, G. A. Cox, L. M. Thompson, S. Finkbeiner, E. Dardiotis, E. Hornstein, D. J. MacGowan, T. Heiman-Patterson, M. G. Hammell, N. A. Patsopoulos, J. Dubnau, A. Nath, R. L. Musunuri, U. S. Evani, A. Abhyankar, M. C. Zody, J. Kaye, S. Wyman, A. LeNail, L. Lima, J. D. Rothstein, C. N. Svendsen, J. Van Eyk, N. J. Maragakis, S. J. Kolb, M. Cudkowicz, E. Baxi, M. Benatar, J. P. Taylor, G. Wu, E. Rampersaud, J. Wuu, R. Rademakers, S. Zuchner, R. Schule, J. McCauley, S. Hussain, A. Cooley, M. Wallace, C. Clayman, R. Barohn, J. Statland, A. Swenson, C. Jackson, J. Trivedi, S. Khan, J. Katz, L. Jenkins, T. Bums, K. Gwathmey, J. Caress, C. McMillan, L. Elman, E. Pioro, J. Heckmann, Y. So, D. Walk, S. Maiser, J. Zhang, V. Silani, C. Gellera, A. Ratti, F. Taroni, G. Lauria, F. Verde, I. Fogh, C. Tiloca, G. P. Comi, G. Sorarú, C. Cereda, F. De Marchi, S. Corti, M. Ceroni, G. Siciliano, M. Filosto, M. Inghilleri, S. Peverelli, C. Colombrita, B. Poletti, L. Maderna, R. Del Bo, S. Gagliardi, G. Querin, C. Bertolin, V. Pensato, B. Castellotti, W. Camu, K. Mouzat, S. Lumbroso, P. Corcia, V. Meininger, G. Besson, E. Lagrange, P. Clavelou, N. Guy, P. Couratier, P. Vourch, V. Danel, E. Bernard, G. Lemasson, H. Laaksovirta, L. Myllykangas, L. Jansson, M. Valori, J. Ealing, H. Hamdalla, S. Rollinson, S. Pickering-Brown, R. W. Orrell, K. C. Sidle, J. Hardy, A. B. Singleton, J. O. Johnson, S. Arepalli, M. Polak, S. Asress, S. Al-Sarraj, A. King, C. Troakes, C. Vance, J. de Belleroche, A. L. M. A. ten Asbroek, J. L. Munoz-Blanco, D. G. Hernandez, J. Ding, J. R. Gibbs, S. W. Scholz, M. K. Floeter, R. H. Campbell, F. Landi, R. Bowser, J. Kirby, R. Pamphlett, G. Gerhard, T. L. Dunckley, C. B. Brady, N. W. Kowall, J. C. Troncoso, I. Le Ber, T. D. Heiman-Patterson, F. Kamel, L. Van Den Bosch, T. M. Strom, T. Meitinger, A. Shatunov, K. van Eijk, M. de Carvalho, M. Kooyman, B. Middelkoop, M. Moisse, R. McLaughlin, M. van Es, M. Weber, K. B. Boylan, M. Van Blitterswijk, K. Morrison, A. N. Basak, J. S. Mora, V. Drory, P. Shaw, M. R. Turner, K. Talbot, O. Hardiman, K. L. Williams, J. A. Fifita, G. A. Nicholson, I. P. Blair, J. Esteban-Pérez, A. Garcia-Redondo, A. Al-Chalabi, A. Al Kheifat, P. Andersen, A. Chio, J. Cooper-Knock, A. Dekker, A. G. Redondo, M. Gotkine, W. Hide, A. Iacoangeli, M. Kiernan, J. Landers, J. Mill, M. M. Neto, J. M. Pardina, S. Newhouse, S. Pinto, S. Pulit, W. Robberecht, C. Shaw, W. Sproviero, G. Tazelaar, P. van Damme, L. van den Berg, J. van Vugt, J. Veldink, M. Zatz, D. C. Bauer, N. A. Twine, E. Rogaeva, L. Zinman, A. Brice, E. L. Feldman, A. C. Ludolph, J. H. Weishaupt, J. Q. Trojanowski, D. J. Stone, P. Tienari, A. Chió, C. E. Shaw, B. J. Traynor, Genome-wide Analyses Identify KIF5A as a Novel ALS Gene. Neuron (2018), doi:10.1016/j.neuron.2018.02.027.
26. K. Placek, G. M. Baer, L. Elman, L. McCluskey, L. Hennessy, P. M. Ferraro, E. B. Lee, V. M. Y. Lee, J. Q. Trojanowski, V. M. Van Deerlin, M. Grossman, D. J. Irwin, C. T. McMillan, UNC13A polymorphism contributes to frontotemporal disease in sporadic amyotrophic lateral sclerosis. Neurobiol. Aging (2019), doi:10.1016/j.neurobiolaging.2018.09.031.
27. C. Pottier, Y. Ren, R. B. Perkerson, M. Baker, G. D. Jenkins, M. van Blitterswijk, M. DeJesus-Hernandez, J. G. J. van Rooij, M. E. Murray, E. Christopher, S. K. McDonnell, Z. Fogarty, A. Batzler, S. Tian, C. T. Vicente, B. Matchett, A. M. Karydas, G. Y. R. Hsiung, H. Seelaar, M. 0. Mol, E. C. Finger, C. Graff, L. Oijerstedt, M. Neumann, P. Heutink, M. Synofzik, C. Wilke, J. Prudlo, P. Rizzu, J. Simon-Sanchez, D. Edbauer, S. Roeber, J. Diehl-Schmid, B. M. Evers, A. King, M. M. Mesulam, S. Weintraub, C. Geula, K. F. Bieniek, L. Petrucelli, G. L. Ahern, E. M. Reiman, B. K. Woodruff, R. J. Caselli, E. D. Huey, M. R. Farlow, J. Grafman, S. Mead, L. T. Grinberg, S. Spina, M. Grossman, D. J. Irwin, E. B. Lee, E. R. Suh, J. Snowden, D. Mann, N. Ertekin-Taner, R. J. Uitti, Z. K. Wszolek, K. A. Josephs, J. E. Parisi, D. S. Knopman, R. C. Petersen, J. R. Hodges, O. Piguet, E. G. Geier, J. S. Yokoyama, R. A. Rissman, E. Rogaeva, J. Keith, L. Zinman, M. C. Tartaglia, N. J. Cairns, C. Cruchaga, B. Ghetti, J. Kofler, O. L. Lopez, T. G. Beach, T. Arzberger, J. Herms, L. S. Honig, J. P. Vonsattel, G. M. Halliday, J. B. Kwok, C. L. White, M. Gearing, J. Glass, S. Rollinson, S. Pickering-Brown, J. D. Rohrer, J. Q. Trojanowski, V. Van Deerlin, E. H. Bigio, C. Troakes, S. Al-Sarraj, Y. Asmann, B. L. Miller, N. R. Graff-Radford, B. F. Boeve, W. W. Seeley, I. R. A. Mackenzie, J. C. van Swieten, D. W. Dickson, J. M. Biernacka, R. Rademakers, Genome-wide analyses as part of the international FTLD-TDP whole-genome sequencing consortium reveals novel disease risk factors and increases support for immune dysfunction in FTLD. Acta Neuropathol. (2019), doi:10.1007/s00401-019-01962-9.
28. M. van Blitterswijk, B. Mullen, A. Wojtas, M. G. Heckman, N. N. Diehl, M. C. Baker, M. DeJesus-Hernandez, P. H. Brown, M. E. Murray, G. Y. R. Hsiung, H. Stewart, A. M. Karydas, E. Finger, A. Kertesz, E. H. Bigio, S. Weintraub, M. Mesulam, K. J. Hatanpaa, C. L. White, M. Neumann, M. J. Strong, T. G. Beach, Z. K. Wszolek, C. Lippa, R. Caselli, L. Petrucelli, K. A. Josephs, J. E. Parisi, D. S. Knopman, R. C. Petersen, I. R. Mackenzie, W. W. Seeley, L. T. Grinberg, B. L. Miller, K. B. Boylan, N. R. Graff-Radford, B. F. Boeve, D. W. Dickson, R. Rademakers, Genetic modifiers in carriers of repeat expansions in the C90RF72 gene. Mol. Neurodegener. (2014), doi:10.1186/1750-1326-9-38.
29. J. M. Vidal-Taboada, A. Lopez-Lopez, M. Salvado, L. Lorenzo, C. Garcia, N. Mahy, M. J. Rodriguez, J. Gamez, UNC13A confers risk for sporadic ALS and influences survival in a Spanish cohort. J. Neurol. (2015), doi:10.1007/s00415-015-7843-z.
30. B. Yang, H. Jiang, F. Wang, S. Li, C. Wu, J. Bao, Y. Zhu, Z. Xu, B. Liu, H. Ren, X. Yang, UNC13A variant rs12608932 is associated with increased risk of amyotrophic lateral sclerosis and reduced patient survival: a meta-analysis. Neurol. Sci. (2019), doi:10.1007/s10072-019-03951-y.
31. H. H. G. Tan, H. J. Westeneng, H. K. van der Burgh, M. A. van Es, L. A. Bakker, K. van Veenhuijzen, K. R. van Eijk, R. P. A. van Eijk, J. H. Veldink, L. H. van den Berg, The Distinct Traits of the UNC13A Polymorphism in Amyotrophic Lateral Sclerosis. Ann. Neurol. (2020), doi:10.1002/ana.25841.
32. F. P. Diekstra, P. W. J. van Vught, W. van Rheenen, M. Koppers, R. J. Pasterkamp, M. A. van Es, H. J. Schelhaas, M. de Visser, W. Robberecht, P. Van Damme, P. M. Andersen, L. H. van den Berg, J. H. Veldink, UNC13A is a modifier of survival in amyotrophic lateral sclerosis. Neurobiol. Aging (2012), doi:10.1016/j.neurobiolaging.2011.10.029.
33. D. J. Schaid, W. Chen, N. B. Larson, From genome-wide associations to candidate causal variants by statistical fine-mapping. Nat. Rev. Genet. (2018), , doi:10.1038/s41576-018-0016-z.
34. M. D. Gallagher, A. S. Chen-Plotkin, The Post-GWAS Era: From Association to Function. Am. J. Hum. Genet. (2018), , doi:10.1016/j.ajhg.2018.04.002.
35. R. J. Pruim, R. P. Welch, S. Sanna, T. M. Teslovich, P. S. Chines, T. P. Gliedt, M. Boehnke, G. R. Abecasis, C. J. Willer, D. Frishman, in Bioinformatics (2011).
36. W. Van Rheenen, A. Shatunov, A. M. Dekker, R. L. McLaughlin, F. P. Diekstra, S. L. Pulit, R. A. A. Van Der Spek, U. Vδsa, S. De Jong, M. R. Robinson, J. Yang, I Fogh, P. T. C. Van Doormaal, G. H. P. Tazelaar, M. Koppers, A. M. Blokhuis, W. Sproviero, A. R. Jones, K. P. Kenna, K. R. Van Eijk, O. Harschnitz, R. D. Schellevis, W. J. Brands, J. Medic, A. Menelaou, A. Vajda, N. Ticozzi, K. Lin, B. Rogelj, K. Vrabec, M. Ravnik-Glava, B. Koritnik, J. Zidar, L. Leonardis, L. D. Groselj, S. Millecamps, F. Salachas, V. Meininger, M. De Carvalho, S. Pinto, J. S. Mora, R. Rojas-Garcia, M. Polak, S. Chandran, S. Colville, R. Swingler, K. E. Morrison, P. J. Shaw, J. Hardy, R. W. Orrell, A. Pittman, K. Sidle, P. Fratta, A. Malaspina, S. Topp, S. Petri, S. Abdulla, C. Drepper, M. Sendtner, T. Meyer, R. A. Ophoff, K. A. Staats, M. Wiedau-Pazos, C. Lomen-Hoerth, V. M. Van Deerlin, J. Q. Trojanowski, L. Elman, L. McCluskey, A. N. Basak, C. Tunca, H. Hamzeiy, Y. Parman, T. Meitinger, P. Lichtner, M. Radivojkov-Blagojevic, C. R. Andres, C. Maurel, G. Bensimon, B. Landwehrmeyer, A. Brice, C. A. M. Payan, S. Saker-Delye, A. Dürr, N. W. Wood, L. Tittmann, W. Lieb, A. Franke, M. Rietschel, S. Cichon, M. M. Nöthen, P. Amouyel, C. Tzourio, J. F. Dartigues, A. G. Uitterlinden, F. Rivadeneira, K. Estrada, A. Hofman, C. Curtis, H. M. Blauw, A. J. Van Der Kooi, M. De Visser, A. Goris, M. Weber, C. E. Shaw, B. N. Smith, O. Pansarasa, C. Cereda, R. Del Bo, G. P. Comi, S. D′Alfonso, C. Bertolin, G. Soraru, L. Mazzini, V. Pensato, C. Gellera, C. Tiloca, A. Ratti, A. Calvo, C. Moglia, M. Brunetti, S. Arcuti, R. Capozzo, C. Zecca, C. Lunetta, S. Penco, N. Riva, A. Padovani, M. Filosto, B. Muller, R. J. Stuit, I. Blair, K. Zhang, E. P. McCann, J. A. Fifita, G. A. Nicholson, D. B. Rowe, R. Pamphlett, M. C. Kiernan, J. Grosskreutz, O. W. Witte, T. Ringer, T. Prell, B. Stubendorff, I. Kurth, C. A. Hubner, P. Nigel Leigh, F. Casale, A. Chio, E. Beghi, E. Pupillo, R. Tortelli, G. Logroscino, J. Powell, A. C. Ludolph, J. H. Weishaupt, W. Robberecht, P. Van Damme, L. Franke, T. H. Pers, R. H. Brown, J. D. Glass, J. E. Landers, O. Hardiman, P. M. Andersen, P. Corcia, P. Vourc'H. V. Silani, N. R. Wray, P. M. Visscher, P. I. W. De Bakker, M. A. Van Es, R. Jeroen Pasterkamp, C. M. Lewis, G. Breen, A. Al-Chalabi, L. H. Van Den Berg, J. H. Veldink, Genome-wide association analyses identify new risk variants and the genetic architecture of amyotrophic lateral sclerosis. Nat. Genet. (2016), doi:10.1038/ng.3622.
37. H. B. Schmidt, A. Barreau, R. Rohatgi, Phase separation-deficient TDP43 remains functional in splicing. Nat. Commun. (2019), doi:10.1038/s41467-019-12740-2.
38. P. T. Nelson, D. W. Dickson, J. Q. Trojanowski, C. R. Jack, P. A. Boyle, K. Arfanakis, R. Rademakers, I. Alafuzoff, J. Attems, C. Brayne, I. T. S. Coyle-Gilchrist, H. C. Chui, D. W. Fardo, M. E. Flanagan, G. Halliday, S. R. K. Hokkanen, S. Hunter, G. A. Jicha, Y. Katsumata, C. H. Kawas, C. D. Keene, G. G. Kovacs, W. A. Kukull, A. I. Levey, N. Makkinejad, T. J. Montine, S. Murayama, M. E. Murray, S. Nag, R. A. Rissman, W. W. Seeley, R. A. Sperling, C. L. White, L. Yu, J. A. Schneider, Limbic-predominant age-related TDP-43 encephalopathy (LATE): Consensus working group report. Brain. 142 (2019), pp. 1503-1527.
39. W. J. Kent, C. W. Sugnet, T. S. Furey, K. M. Roskin, T. H. Pringle, A. M. Zahler, a. D. Haussler, The Human Genome Browser at UCSC. Genome Res. (2002), doi:10.1101/gr.229102.
40. Zhang, Y. J. et al. Aberrant cleavage of TDP-43 enhances aggregation and cellular toxicity. Proc. Natl. Acad. Sci. U.S.A (2009) doi:10.1073/pnas.0900688106.
41. Maury, Y. et al. Combinatorial analysis of developmental cues efficiently converts human pluripotent stem cells into multiple neuronal subtypes. Nat. Biotechnol. (2015) doi:10.1038/nbt.3049.
42. Prudencio, M. et al. Repetitive element transcripts are elevated in the brain of C9orf72 ALS/FTLD patients. Hum. Mol. Genet. 26, 3421-3431 (2017).
43. Hansen, K. D., Brenner, S. E. & Dudoit, S. Biases in Illumina transcriptome sequencing caused by random hexamer priming. Nucleic Acids Res. (2010) doi:10.1093/nar/gkq224.
44. Prudencio, M. et al. Misregulation of human sortilin splicing leads to the generation of a nonfunctional progranulin receptor. Proc. Natl. Acad. Sci. U.S.A (2012) doi:10.1073/pnas. 1211577110.
45. Nana, A. L. et al. Neurons selectively targeted in frontotemporal dementia reveal early stage TDP-43 pathobiology. Acta Neuropathol. (2019) doi:10.1007/s00401-018-1942-8.

The various embodiments described above and in Appendix A can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 63/171,522, filed on Apr. 6, 2021, and U.S. Provisional Patent Application No. 63/312,808, filed on Feb. 22, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of reducing expression of a UNC13A cryptic exon splice variant in a cell comprising administering a UNC13A cryptic exon splice variant specific inhibitor, wherein:
(a) the UNC13A cryptic exon splice variant comprises a cryptic exon between exon 20 and exon 21 of the UNC13A cryptic exon splice variant mature mRNA transcript; and
(b) the UNC13A cryptic exon splice variant specific inhibitor comprises an antisense oligonucleotide.

2. The method of claim 1 wherein the cryptic exon comprises the base sequence of SEQ ID NO:5 or SEQ ID NO:6.

3. The method of claim 1 or 2, wherein the UNC13A cryptic exon splice variant comprises SEQ ID NO:7 or SEQ ID NO:8.

4. The method of any one of claims 1-3, wherein the UNC13A cryptic exon splice variant specific inhibitor comprises an antisense oligonucleotide that is complementary to:
(a) the 5′ end of the cryptic exon having a sequence set forth in SEQ ID NO:641; or (b) the 3′ end of the cryptic exon having a sequence set forth in SEQ ID NO:642.

5. The method of any one of claims 1-4, wherein the UNC13A cryptic exon splice variant specific inhibitor comprises an antisense oligonucleotide that is complementary to:
(a) the 5′ end of the cryptic exon having a sequence set forth in SEQ ID NO:643; or (b) the 3′ end of the cryptic exon having a sequence set forth in SEQ ID NO:644.

6. The method of any one of claims 1-3, wherein the UNC13A cryptic exon splice variant specific inhibitor comprises an antisense oligonucleotide that is complementary to:
(a) the exon 20 splice donor site region in a preprocessed mRNA encoding UNC13A;
(b) the cryptic exon splice acceptor site region in a preprocessed mRNA encoding UNC13A;
(c) the cryptic exon splice donor site region in a preprocessed mRNA encoding UNC13A; or
(d) the exon 21 splice acceptor site region in a preprocessed mRNA encoding UNC13A.

7. The method of claim 6, wherein:
(a) the exon 20 splice donor site region in the preprocessed mRNA encoding UNC13A comprises or consists of SEQ ID NO:12;
(b) the cryptic exon splice acceptor site region in the preprocessed mRNA encoding UNC13A comprises or consists of SEQ ID NO:91;
(c) the cryptic exon splice donor site region in the preprocessed mRNA encoding UNC13A comprises or consists of SEQ ID NO:220; or
(d) the exon 21 splice acceptor site region in the preprocessed mRNA encoding UNC13A comprises or consists of SEQ ID NO:299.

8. The method of any one of claims 1-7, wherein the antisense oligonucleotide has 15-40 bases.

9. The method of claim 8, wherein the antisense oligonucleotide has 20-30 bases.

10. The method of claim 8, wherein the antisense oligonucleotide has 18-25 bases.

11. The method of claim 8, wherein the antisense oligonucleotide has 18-22 bases.

12. The method of any one of claims 1-11, wherein the antisense oligonucleotide has a base sequence that has at least 80%, 85%, 90%, or 95% identity to any one of SEQ ID NOS:13-90, 92-219, 221-298, 300-377, and 423-640.

13. The method of claim 12, wherein the antisense oligonucleotide has a base sequence comprising or consisting of any one of SEQ ID NOS: 13-90, 92-219, 221-298, 300-377, and 423-640.

14. The method of claim 13, wherein the antisense oligonucleotide has a base sequence comprising or consisting of any one of SEQ ID NOS:423-432, 439-443, 491-498, 502-507, and 513-514.

15. The method of any one of claims 1-14, wherein the antisense oligonucleotide:
(a) has 18-30 bases, 18-25 bases, or 18-22 bases that are complementary to SEQ ID NO:650;
(b) has 18-30 bases, 18-25 bases, or 18-22 bases that are complementary to SEQ ID NO: 651;
(c) has 18-30 bases, 18-25 bases, or 18-22 bases that are complementary to SEQ ID NO:652;
(d) has 18-30 bases, 18-25 bases, or 18-22 bases that are complementary to SEQ ID NO:653; or
(e) has 18-21 bases that are complementary to SEQ ID NO:654.

16. The method of any one of claims 1-15, wherein the antisense oligonucleotide is a modified antisense oligonucleotide.

17. The method of claim 16, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

18. The method of any one of claims 1-17, wherein the cell is within a subject.

19. The method of any one of claims 1-18, wherein the subject is identified is having an UNC13A gene mutation in intron 20-21, optionally wherein the UNC13A gene mutation comprises rs12608932 (hg38 chrl9:17.641,880 A→C), rs12973192 (hg38 chrl9: 17,642,430 C→G), rs56041637 (hg38 chrl9:17,642,033-17,642,056 0-2 CATC repeats→3-5 CATC repeats), and rs62121687 (hg38 chrl9:17,642,351 C→A), or any combination thereof.

20. A method of reducing phosphorylated TAR-DNA binding protein-43 (TDP-43) in a cell comprising administering a UNC13A cryptic exon splice variant specific inhibitor, wherein:
(a) the UNC13A cryptic exon splice variant comprises a cryptic exon between exon 20 and exon 21 of the UNC13A cryptic exon splice variant mature mRNA transcript; and
(b) the UNC13A cryptic exon splice variant specific inhibitor comprises an antisense oligonucleotide.

21. The method of claim 20 wherein the cryptic exon comprises the base sequence of SEQ ID NO:5 or SEQ ID NO:6.

22. The method of claim 20 or 21, wherein the UNC13A cryptic exon splice variant comprises SEQ ID NO:7 or SEQ ID NO:8.

23. The method of any one of claims 20-22, wherein the UNC13A cryptic exon splice variant specific inhibitor comprises an antisense oligonucleotide that is complementary to:
(a) the 5′ end of the cryptic exon having a sequence set forth in SEQ ID NO:641; or (b) the 3′ end of the cryptic exon having a sequence set forth in SEQ ID NO:642.

24. The method of any one of claims 20-23, wherein the UNC13A cryptic exon splice variant specific inhibitor comprises an antisense oligonucleotide that is complementary to:
(a) the 5′ end of the cryptic exon having a sequence set forth in SEQ ID NO:643; or (b) the 3′ end of the cryptic exon having a sequence set forth in SEQ ID NO:644.

25. The method of any one of claims 20-22, wherein the UNC13A cryptic exon splice variant specific inhibitor comprises an antisense oligonucleotide that is complementary to:
(a) the exon 20 splice donor site region in a preprocessed mRNA encoding UNC13A;
(b) the cryptic exon splice acceptor site region in a preprocessed mRNA encoding UNC13A;
(c) the cryptic exon splice donor site region in a preprocessed mRNA encoding UNC13A; or
(d) the exon 21 splice acceptor site region in a preprocessed mRNA encoding UNC13A.

26. The method of claim 25, wherein:
(a) the exon 20 splice donor site region in the preprocessed mRNA encoding UNC13A comprises or consists of SEQ ID NO:12;
(b) the cryptic exon splice acceptor site region in the preprocessed mRNA encoding UNC13A comprises or consists of SEQ ID NO:91;
(c) the cryptic exon splice donor site region in the preprocessed mRNA encoding UNC13A comprises or consists of SEQ ID NO:220; or
(d) the exon 21 splice acceptor site region in the preprocessed mRNA encoding UNC13A comprises or consists of SEQ ID NO:299.

27. The method of any one of claims 16-26, wherein the antisense oligonucleotide has 15-40 bases.

28. The method of claim 27, wherein the antisense oligonucleotide has 20-30 bases.

29. The method of claim 27, wherein the antisense oligonucleotide has 18-25 bases.

30. The method of claim 27, wherein the antisense oligonucleotide has 18-22 bases.

31. The method of any one of claims 16-30, wherein the antisense oligonucleotide has a base sequence that has at least 80% identity to any one of SEQ ID NOS: 13-90, 92-219, 221-298, 300-377, and 423-640.

32. The method of claim 31, wherein the antisense oligonucleotide has a base sequence comprising or consisting of any one of SEQ ID NOS: 13-90, 92-219, 221-298, 300-377, and 423-640.

33. The method of claim 32, wherein the antisense oligonucleotide has a base sequence comprising or consisting of any one of SEQ ID NOS:423-432, 439-443, 491-498, 502-507, and 513-514.

34. The method of any one of claims 16-33, wherein the antisense oligonucleotide:
(a) has 18-30 bases, 18-25 bases, or 18-22 bases that are complementary to SEQ ID NO:650;
(b) has 18-30 bases, 18-25 bases, or 18-22 bases that are complementary to SEQ ID NO: 651;
(c) has 18-30 bases, 18-25 bases, or 18-22 bases that are complementary to SEQ ID NO:652;
(d) has 18-30 bases, 18-25 bases, or 18-22 bases that are complementary to SEQ ID NO:653; or
(e) has 18-21 bases that are complementary to SEQ ID NO:654.

35. The method of any one of claims 16-34, wherein the antisense oligonucleotide is a modified antisense oligonucleotide.

36. The method of claim 35, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

37. The method of any one of claims 16-36, wherein the cell is within a subject.

38. A method of treating TAR-DNA binding protein-43 (TDP-43) proteinopathy in a subject comprising administering a UNC13A cryptic exon splice variant specific inhibitor to the subject, wherein:
(a) the UNC13A cryptic exon splice variant comprises a cryptic exon between exon 20 and exon 21 of the UNC13A cryptic exon splice variant mature mRNA transcript; and
(b) the UNC13A cryptic exon splice variant specific inhibitor comprises an antisense oligonucleotide.

39. The method of claim 38 wherein the cryptic exon comprises SEQ ID NO:5 or SEQ ID NO:6.

40. The method of claim 38 or 39, wherein the UNC13A cryptic exon splice variant comprises SEQ ID NO:7 or SEQ ID NO:8.

41. The method of any one of claims 38-40, wherein the UNC13A cryptic exon splice variant specific inhibitor comprises an antisense oligonucleotide that is complementary to:
(a) the 5′ end of the cryptic exon having a sequence set forth in SEQ ID NO:641; or (b) the 3′ end of the cryptic exon having a sequence set forth in SEQ ID NO:642.

42. The method of any one of claims 38-41, wherein the UNC13A cryptic exon splice variant specific inhibitor comprises an antisense oligonucleotide that is complementary to:
(a) the 5′ end of the cryptic exon having a sequence set forth in SEQ ID NO:643; or (b) the 3′ end of the cryptic exon having a sequence set forth in SEQ ID NO:644.

43. The method of any one of claims 38-42, wherein the UNC13A cryptic exon splice variant specific inhibitor comprises an antisense oligonucleotide that is complementary to:
(a) the exon 20 splice donor site region in a preprocessed mRNA encoding UNC13A;
(b) the cryptic exon splice acceptor site region in a preprocessed mRNA encoding UNC13A;
(c) the cryptic exon splice donor site region in a preprocessed mRNA encoding UNC13A; or
(d) the exon 21 splice acceptor site region in a preprocessed mRNA encoding UNC13A.

44. The method of claim 43, wherein:
(a) the exon 20 splice donor site region in the preprocessed mRNA encoding UNC13A comprises or consists of SEQ ID NO:12;
(b) the cryptic exon splice acceptor site region in the preprocessed mRNA encoding UNC13A comprises or consists of SEQ ID NO:91;
(c) the cryptic exon splice donor site region in the preprocessed mRNA encoding UNC13A comprises or consists of SEQ ID NO:220; or
(d) the exon 21 splice acceptor site region in the preprocessed mRNA encoding UNC13A comprises or consists of SEQ ID NO:299.

45. The method of any one of claims 38-44, wherein the antisense oligonucleotide has 15-40 bases.

46. The method of claim 45, wherein the antisense oligonucleotide has 20-30 bases.

47. The method of claim 45, wherein the antisense oligonucleotide has 18-25 bases.

48. The method of claim 45, wherein the antisense oligonucleotide has 18-22 bases.

49. The method of any one of claims 38-48, wherein the antisense oligonucleotide has a base sequence that has at least 80% identity to any one of SEQ ID NOS:13-90, 92-219, 221-298, 300-377, and 423-640.

50. The method of claim 49, wherein the antisense oligonucleotide has a base sequence comprising or consisting of any one of SEQ ID NOS:13-90, 92-219, 221-298, 300-377, and 423-640.

51. The method of claim 50, wherein the antisense oligonucleotide has a base sequence comprising or consisting of any one of SEQ ID NOS:423-432, 439-443, 491-498, 502-507, and 513-514.

52. The method of any one of claims 38-51, wherein the antisense oligonucleotide:
(a) has 18-30 bases, 18-25 bases, or 18-22 bases that are complementary to SEQ ID NO:650;
(b) has 18-30 bases, 18-25 bases, or 18-22 bases that are complementary to SEQ ID NO: 651;
(c) has 18-30 bases, 18-25 bases, or 18-22 bases that are complementary to SEQ ID NO:652;
(d) has 18-30 bases, 18-25 bases, or 18-22 bases that are complementary to SEQ ID NO:653; or
(e) has 18-21 bases that are complementary to SEQ ID NO:654.

53. The method of any one of claims 38-52, wherein the antisense oligonucleotide is a modified antisense oligonucleotide.

54. The method of claim 53, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

55. The method of any one of claims 38-54, wherein the TDP-43 proteinopathy comprises amyotrophic lateral sclerosis (ALS), frontotemporal dementia (FTD), Alzheimer's Disease, hippocampal sclerosis, Parkinson's disease, Perry Syndrome, Huntington disease, chronic traumatic encephalopathy, or a combination thereof.

56. A method of treating a subject that has been identified as having a UNC13A gene mutation in intron 20-21 comprising administering an UNC13A cryptic exon splice variant specific inhibitor to the subject, wherein:
(a) the UNC13A cryptic exon splice variant comprises a cryptic exon between exon 20 and exon 21 of the UNC13A cryptic exon splice variant mature mRNA transcript; and
(b) the UNC13A cryptic exon splice variant specific inhibitor comprises an antisense oligonucleotide.

57. The method of claim 55, wherein the UNC13A gene mutation comprises rs12608932 (hg38 chrl9:17.641,880 A→C), rs12973192 (hg38 chrl9: 17,642,430 C→G), rs56041637 (hg38 chrl9:17,642,033-17,642,056 0-2 CATC repeats→3-5 CATC repeats), and rs62121687 (hg38 chrl9:17,642,351 C→A), or any combination thereof

58. The method of claim 56 or 57, wherein the subject has decreased expression of TDP-43.

59. The method of any one of claims 56-58 wherein the cryptic exon comprises the base sequence of SEQ ID NO:5 or SEQ ID NO:6.

60. The method of any one of claims 56-59, wherein the UNC13A cryptic exon splice variant comprises SEQ ID NO:7 or SEQ ID NO:8.

61. The method of any one of claims 56-60, wherein the UNC13A cryptic exon splice variant specific inhibitor comprises an antisense oligonucleotide that is complementary to:
(a) the 5′ end of the cryptic exon having a sequence set forth in SEQ ID NO:641; or (b) the 3′ end of the cryptic exon having a sequence set forth in SEQ ID NO:642.

62. The method of any one of claims 56-61, wherein the UNC13A cryptic exon splice variant specific inhibitor comprises an antisense oligonucleotide that is complementary to:
(a) the 5′ end of the cryptic exon having a sequence set forth in SEQ ID NO:643; or (b) the 3′ end of the cryptic exon having a sequence set forth in SEQ ID NO:644.

63. The method of any one of claims 56-62, wherein the UNC13A cryptic exon splice variant specific inhibitor comprises an antisense oligonucleotide that is complementary to:
(a) the exon 20 splice donor site region in a preprocessed mRNA encoding UNC13A;
(b) the cryptic exon splice acceptor site region in a preprocessed mRNA encoding UNC13A;
(c) the cryptic exon splice donor site region in a preprocessed mRNA encoding UNC13A; or
(d) the exon 21 splice acceptor site region in a preprocessed mRNA encoding UNC13A.

64. The method of claim 63, wherein:
(a) the exon 20 splice donor site region in the preprocessed mRNA encoding UNC13A comprises or consists of SEQ ID NO:12;
(b) the cryptic exon splice acceptor site region in the preprocessed mRNA encoding UNC13A comprises or consists of SEQ ID NO:91;
(c) the cryptic exon splice donor site region in the preprocessed mRNA encoding UNC13A comprises or consists of SEQ ID NO:220; or
(d) the exon 21 splice acceptor site region in the preprocessed mRNA encoding UNC13A comprises or consists of SEQ ID NO:299.

65. The method of any one of claims 56-64, wherein the antisense oligonucleotide has 15-40 bases.

66. The method of claim 65, wherein the antisense oligonucleotide has 20-30 bases.

67. The method of claim 65, wherein the antisense oligonucleotide has 18-25 bases.

68. The method of claim 65, wherein the antisense oligonucleotide has 18-22 bases.

69. The method of any one of claims 56-68, wherein the antisense oligonucleotide has a base sequence that has at least 80% identity to any one of SEQ ID NOS:13-90, 92-219, 221-298, 300-377, and 423-640.

70. The method of claim 69, wherein the antisense oligonucleotide has a base sequence comprising or consisting of any one of SEQ ID NOS: 13-90, 92-219, 221-298, 300-377, and 423-640.

71. The method of claim 70, wherein the antisense oligonucleotide has a base sequence comprising or consisting of any one of SEQ ID NOS:423-432, 439-443, 491-498, 502-507, and 513-514.

72. The method of any one of claims 56-71, wherein the antisense oligonucleotide:
(a) has 18-30 bases, 18-25 bases, or 18-22 bases that are complementary to SEQ ID NO:650;
(b) has 18-30 bases, 18-25 bases, or 18-22 bases that are complementary to SEQ ID NO: 651;
(c) has 18-30 bases, 18-25 bases, or 18-22 bases that are complementary to SEQ ID NO:652;
(d) has 18-30 bases, 18-25 bases, or 18-22 bases that are complementary to SEQ ID NO:653; or
(e) has 18-21 bases that are complementary to SEQ ID NO:654.

73. The method of any one of claims 56-72, wherein the antisense oligonucleotide is a modified antisense oligonucleotide.

74. The method of claim 73, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

75. The method of any one of claims 56-74, wherein the subject has a TDP-43 proteinopathy, optionally wherein the TDP-43 proteinopathy comprises amyotrophic lateral sclerosis (ALS), frontotemporal lobar degeneration (FTLD), primary lateral sclerosis (PLS), progressive muscular atrophy (PMA), facial onset sensory and motor neuronopathy (FOSMN), hippocampal sclerosis (HS), limbic-predominant age-related TDP-43 encephalopathy (LATE), cerebral age-related TDP-43 with sclerosis (CARTS), Guam Parkinson-dementia complex (G-PDC), Guan ALS (G-ALS), Multisystem proteinopathy (MSP), Perry disease, Alzheimer's disease (AD), and chronic traumatic encephalopathy (CTE), or a combination thereof.

76. The method of any one of claims 38-75, further comprising administering to the subject a STMN2 cryptic splice variant specific inhibitor.

77. The method of claim 76, wherein the STMN2 cryptic splice variant comprises cryptic exon 2a.

78. The method of claim 76 or 77, wherein the STMN2 cryptic splice variant specific inhibitor comprises an inhibitory nucleic acid, peptide, antibody, binding protein, small molecule, ribozyme, or aptamer.

79. The method of any one of claims 76-78, wherein the STMN2 cryptic splice variant specific inhibitor targets cryptic exon 2a.

80. The method of any one of claims 76-79, wherein the STMN2 cryptic splice variant specific inhibitor is an antisense oligonucleotide, optionally wherein the antisense oligonucleotide is a modified antisense oligonucleotide.

81. The method of claim 80, wherein the antisense oligonucleotide is complementary to: the exon 1 splice donor site region in a preprocessed mRNA encoding STMN2 or the cryptic exon 2a splice acceptor site region in a preprocessed mRNA encoding STMN2.

82. A pharmaceutical composition comprising an antisense oligonucleotide having 15-40 bases and comprising a base sequence that has at least 80% identity to any one of SEQ ID NOS: 13-90, 92-219, 221-298, 300-377, and 423-640, and a pharmaceutically acceptable excipient.

83. The pharmaceutical composition of claim 82, wherein the antisense oligonucleotide has a base sequence comprising or consisting of any one of SEQ ID NOS: 13-90, 92-219, 221-298, 300-377, and 423-640.

84. The pharmaceutical composition of claim 83, wherein the antisense oligonucleotide has a base sequence comprising or consisting of any one of SEQ ID NOS:423-432, 439-443, 491-498, 502-507, and 513-514.

85. A pharmaceutical composition comprising an antisense oligonucleotide having:
(a) 18-30 bases, 18-25 bases, or 18-22 bases that are complementary to SEQ ID NO:650;
(b) 18-30 bases, 18-25 bases, or 18-22 bases that are complementary to SEQ ID NO: 651;
(c) 18-30 bases, 18-25 bases, or 18-22 bases that are complementary to SEQ ID NO:652;
(d) 18-30 bases, 18-25 bases, or 18-22 bases that are complementary to SEQ ID NO:653; or
(e) 18-21 bases that are complementary to SEQ ID NO:654;
and a pharmaceutically acceptable excipient.

86. The pharmaceutical composition of any one of claims 82-85, wherein the antisense oligonucleotide has 18-25 bases.

87. The pharmaceutical composition of claim 86, wherein the antisense oligonucleotide has 18-22 bases.

88. The pharmaceutical composition of claim 82-85, wherein the antisense oligonucleotide has 20-30 bases.

89. The pharmaceutical composition of any one of claims 82-88, wherein the antisense oligonucleotide is a modified antisense oligonucleotide.

90. The pharmaceutical composition of claim 89, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

91. The pharmaceutical composition of any one of claims 82-90, wherein the antisense oligonucleotide is complementary to:
(a) the 5′ end of the cryptic exon having a sequence set forth in SEQ ID NO:641; or (b) the 3′ end of the cryptic exon having a sequence set forth in SEQ ID NO:642.

92. The pharmaceutical composition of any one of claims 82-91, wherein the UNC13A cryptic exon splice variant specific inhibitor comprises an antisense oligonucleotide that is complementary to:
(a) the 5′ end of the cryptic exon having a sequence set forth in SEQ ID NO:643; or (b) the 3′ end of the cryptic exon having a sequence set forth in SEQ ID NO:644.

93. A modified antisense oligonucleotide having 15-40 bases and comprising a base sequence that has at least 80% identity to any one of SEQ ID NOS: 13-90, 92-219, 221-298, 300-377, and 423-640.

94. The modified antisense oligonucleotide of claim 93, wherein the antisense oligonucleotide has a base sequence comprising or consisting of any one of SEQ ID NOS: 13-90, 92-219, 221-298, 300-377, and 423-640.

95. The modified antisense oligonucleotide of claim 94, wherein the antisense oligonucleotide has a base sequence comprising or consisting of any one of SEQ ID NOS:423-432, 439-443, 491-498, 502-507, and 513-514.

96. The modified antisense oligonucleotide of any one of claims 93-95, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.

97. A modified antisense oligonucleotide having 15-40 bases, wherein wherein the base sequence is complementary to:
(a) the 5′ end of the cryptic exon having a sequence set forth in SEQ ID NO:641; or (b) the 3′ end of the cryptic exon having a sequence set forth in SEQ ID NO:642.

98. The modified antisense oligonucleotide of claim 97, wherein the antisense oligonucleotide that is complementary to:
(a) the 5′ end of the UNC13A cryptic exon having a sequence set forth in SEQ ID NO:643; or
(b) the 3′ end of the UNC13A cryptic exon having a sequence set forth in SEQ ID NO:644.

99. The modified antisense oligonucleotide of claim 97 or 98, wherein the antisense oligonucleotide:
(a) has 18-30 bases, 18-25 bases, or 18-22 bases that are complementary to SEQ ID NO:650;
(b) has 18-30 bases, 18-25 bases, or 18-22 bases that are complementary to SEQ ID NO: 651;
(c) has 18-30 bases, 18-25 bases, or 18-22 bases that are complementary to SEQ ID NO:652;
(d) has 18-30 bases, 18-25 bases, or 18-22 bases that are complementary to SEQ ID NO:653; or
(e) has 18-21 bases that are complementary to SEQ ID NO:654.

100. The modified antisense oligonucleotide of any one of claims 97-99, wherein the antisense oligonucleotide has a base sequence comprising or consisting of any one of SEQ ID NOS:423-432, 439-443, 491-498, 502-507, and 513-514.

101. The modified antisense oligonucleotide of any one of claims 93-100, wherein the antisense oligonucleotide has 18-25 bases.

102. The modified antisense oligonucleotide of claim 101, wherein the antisense oligonucleotide has 18-22 bases.

103. The modified antisense oligonucleotide of any one of claims 93-100, wherein the antisense oligonucleotide has 20-30 bases.

104. A kit comprising an UNC13A cryptic exon splice variant specific antisense oligonucleotide having 15-40 bases and comprising a base sequence that has at least 80% identity to any one of SEQ ID NOS: 13-90, 92-219, 221-298, 300-377, and 423-640.

105. The kit of claim 104, wherein the antisense oligonucleotide has a base sequence comprising or consisting of any one of SEQ ID NOS: 13-90, 92-219, 221-298, 300-377, and 423-640.

106. The kit of claim 105, wherein the antisense oligonucleotide has a base sequence comprising or consisting of any one of SEQ ID NOS:423-432, 439-443, 491-498, 502-507, and 513-514.

107. The kit of any one of claims 104-106, wherein the antisense oligonucleotide has 18-25 bases.

108. The kit of claim 107, wherein the antisense oligonucleotide has 18-22 bases.

109. The kit of any one of claims 104-108, wherein the antisense oligonucleotide has 20-30 bases.

110. The kit of any one of claims 104-109, wherein the antisense oligonucleotide is a modified antisense oligonucleotide.

111. The kit of any one of claims 104-110, wherein the modified antisense oligonucleotide comprises a 2′OMe antisense oligonucleotide, 2′ O-Methoxyethyl antisense oligonucleotide, phosphorothioate antisense oligonucleotide, or LNA antisense oligonucleotide.
